SEQUENCE LISTING (91 sequences)

SEQ ID NO: 1 - DNA, 537 bp, Homo sapiens
atggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtatgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaa

SEQ ID NO: 2 - DNA, 537 bp, Homo sapiens
atggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtttgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaa

SEQ ID NO: 3 - DNA, 153 bp, Homo sapiens
cggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttaccta

SEQ ID NO: 4 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICMIAVMVVFIILVIGVCTCCHPLRKRRKRKKKEEEMETLGKDITPINEDIEETNIA

SEQ ID NO: 5 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 6 - protein, 50 aa, Homo sapiens
RKGHFSRCPKQYKHYCIKGRCRFVVAEQTPSCVCDEGYIGARCERVDLFY

SEQ ID NO: 7 - DNA, 1196 bp, Homo sapiens
ggcacgagggtagccagggtgccagcctgggaagtagtttcgtttccttctgcctccgggattagtttccaggcaccctctcaggcgcccgaggcccgggaagggggcgaagaaggagggagacttgtctaggggctgcccggcccggcagagcggggttgatggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtatgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaaaaggctatgaagttacctccaggttggtggcaagctgcaaagtgccttgctcatttgaaaatggacagaatgtgtctcaggaaaacagctagtagacatgaattttaaataatgtatttactttttatttgcaactttagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaagaaaagtagaaaggaggttaggtcttaggaattgaattaataataaagctaccatttatcaagcatttaccatgtgctaataagtttgaaatatattatttcctttattcctttcagcaatccatgagatagctattataatcctcatttcctacatatggaaacagggccaaagaagtcaagtcaaataatctaatccagattt

SEQ ID NO: 8 - DNA, 1271 bp, Homo sapiens
cagcgtggaggctccaaggaccaagtcctgcgcctctttggcggggtgtgtgcaggaggaggggggataaataggaggctccctcctcccggcgacattcacggagccggccggcctcccgccctgggtgtttccctgccttgtagccagggtgccagcctgggaagtagtttcgtttccttctgcctccgggattagtttccaggcaccctctcaggcgcccgaggcccgggaagggggcgaagaaggagggagacttgtctaggggctgcccggcccggcagagcggggttgatggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtttgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaaaaggctatgaagttacctccaggttggtggcaagctgcaaagtgccttgctcatttgaaaatggacagaatgtgtctcaggaaaaacagctagtagacatgaattttaaataatgtatttactttttatttgcaactttagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaagaaaagtagaaaggaggttaggtcttaggaattgaattaataataaagctaccatttatcaagcatttaccatgtgctaataagtttgaaatatattatttcctttattcctttcagcaatccatgagatagctattataatcctc

SEQ ID NO: 9 - DNA, 153 bp, Homo sapiens; identical to SEQ ID NO: 3

SEQ ID NO: 10 - DNA, 390 bp, Homo sapiens
atggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtccctcttcggaaacgtcgtaaaagaaagaagaaagaagaagaaatggaaactctgggtaaagatataactcctatcaatgaagatattgaagagacaaatattgcttaa

SEQ ID NO: 11 - protein, 129 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVPLRKRRKRKKKEEEMETLGKDITPINEDIEETNIA

SEQ ID NO: 12 - DNA, 243 bp, Homo sapiens
gatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttactag

SEQ ID NO: 13 - protein, 80 aa, Homo sapiens
DGNSTRSPETNGLLCGDPEENCAATTTQSKRKGHFSRCPKQYKHYCIKGRCRFVVAEQTPSCVCDEGYIGARCERVDLFY

SEQ ID NO: 14 - DNA, 246 bp, Homo sapiens
atggatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttactag

SEQ ID NO: 15 - DNA, 1038 bp, Artificial Sequence
  Description of Artificial Sequence Synthetic construct
atggacccaacagccccgggtagcagtgtcagctccctgccgctgctcctggtccttgccctgggtcttgcaattctccactgtgtggtagcagatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatctgtgagaaaggctactttggggctcggtgtgagcgagtggacctgttttacggatccgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - DNA, 1041 bp, Artificial Sequence
  Description of Artificial Sequence Synthetic construct
atggacccaacagccccgggtagcagtgtcagctccctgccgctgctcctggtccttgccctgggtcttgcaattctccactgtgtggtagcagatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatctgtgagaaaggctactttggggctcggtgtgagcgagtggacctgttttacggatccgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatga

SEQ ID NO: 17 - DNA, 1038 bp, Artificial Sequence
  Description of Artificial Sequence Synthetic construct
atggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacggatccgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 81 aa, Homo sapiens
MDGNSTRSPETNGLLCGDPEENCAATTTQSKRKGHFSRCPKQYKHYCIKGRCRFVVAEQTPSCVCDEGYIGARCERVDLFY

SEQ ID NO: 19 - protein, 80 aa, Homo sapiens
DGNSTRSPETNGLLCGDPEENCAATTTQSKRKGHFSRCPKQYKHYCIKGRCRFVVAEETPSCVCDEGYIGARCERVDLFY

SEQ ID NO: 20 - protein, 80 aa, Mus musculus
DGNTTRTPETNGSLCGAPGENCTGTTPRQKVKTHFSRCPKQYKHYCIHGRCRFVVDEQTPSCICEKGYFGARCERVDLFY

SEQ ID NO: 21 - protein, 48 aa, Mus musculus
THFSRCPKQYKHYCIHGRCRFVVDEQTPSCICEKGYFGARCERVDLFY

SEQ ID NO: 22 - protein, 60 aa, Homo sapiens
MDGNSTRSPETNGLLCGDPEENCAATTTQSKRKGHFSRCPKQYKHYCIKGRCRFVVAEQT

SEQ ID NO: 23 - protein, 177 aa, Mus musculus
MDPTAPGSSVSSLPLLLVLALGLAILHCVVADGNTTRTPETNGSLCGAPGENCTGTTPRQKVKTHFSRCPKQYKHYCIHGRCRFVVDEQTPSCICEKGYFGARCERVDLFYLQQDRGQILVVCLIVVMVVFIILVIGVCTCCHPLRKHRKKKKEEKMETLDKDKTPISEDIQETNIA

SEQ ID NO: 24 - protein, 178 aa, Homo sapiens; identical to SEQ ID NO: 5

SEQ ID NO: 25 - protein, 345 aa, Artificial Sequence
  Description of Artificial Sequence Synthetic construct
MDPTAPGSSVSSLPLLLVLALGLAILHCVVADGNTTRTPETNGSLCGAPGENCTGTTPRQKVKTHFSRCPKQYKHYCIHGRCRFVVDEQTPSCICEKGYFGARCERVDLFYGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 346 aa, Artificial Sequence
  Description of Artificial Sequence Synthetic construct
MDPTAPGSSVSSLPLLLVLALGLAILHCVVADGNTTRTPETNGSLCGAPGENCTGTTPRQKVKTHFSRCPKQYKHYCIHGRCRFVVDEQTPSCICEKGYFGARCERVDLFYGSEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 27 - protein, 345 aa, Artificial Sequence
  Description of Artificial Sequence Synthetic construct
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 108 bp, Homo sapiens
tgccccaagcaatacaagcattactgcatcaaagggagatgccgcttcgtggtggccgagcagacgccctcctgtgtctgtgatgaaggctacattggagcaaggtgt

SEQ ID NO: 29 - protein, 36 aa, Homo sapiens
CPKQYKHYCIKGRCRFVVAEQTPSCVCDEGYIGARC

SEQ ID NO: 30 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 31 - protein, 645 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 32 - protein, 640 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 33 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 34 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 35 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 36 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 37 - protein, 152 aa, Mus musculus
MALGVLIAVCLLFKAMKAALSEEAEVIPPSTAQQSNWTFNNTEADYIEEPVALKFSHPCLEDHNSYCINGACAFHHELKQAICRCFTGYTGQRCEHLTLTSYAVDSYEKYIAIGIGVGLLISAFLAVFYCYIRKRCINLKSPYIICSGGSPL

SEQ ID NO: 38 - protein, 133 aa, Homo sapiens
MALGVPISVYLLFNAMTALTEEAAVTVTPPITAQQADNIEGPIALKFSHLCLEDHNSYCINGACAFHHELEKAICRCFTGYTGERCEHLTLTSYAVDSYEKYIAIGIGVGLLLSGFLVIFYCYIRKRYEKDKI

SEQ ID NO: 39 - protein, 36 aa, Homo sapiens
CPDSHTQFCFHGTCRFLVQEDKPACVCHSGYVGARC

SEQ ID NO: 40 - protein, 40 aa, Homo sapiens
CAEKEKTFCVNGGECFMVKDLSNPSRYLCKCPNEFTGDRC

SEQ ID NO: 41 - protein, 40 aa, Homo sapiens
CAEKEKTFCVNGGECFMVKDLSNPSRYLCKCQPGFTGARC

SEQ ID NO: 42 - protein, 36 aa, Homo sapiens
CLRKYKDFCIHGECKYVKELRAPSCICHPGYHGERC

SEQ ID NO: 43 - protein, 36 aa, Homo sapiens
CSSDMNGYCLHGQCIYLVDMSQNYCRCEVGYTGVRC

SEQ ID NO: 44 - protein, 37 aa, Homo sapiens
CKLRKGNCSSTVCGQDLQSHLCMCAEGYALSRDRKYC

SEQ ID NO: 45 - protein, 36 aa, Homo sapiens
CAFWNHGCTLGCKNTPGSYYCTCPVGFVLLPDGKRC

SEQ ID NO: 46 - protein, 36 aa, Homo sapiens
CPRNVSECSHDCVLTSEGPLCFCPEGSVLERDGKTC

SEQ ID NO: 47 - protein, 36 aa, Homo sapiens
CLYQNGGCEHICKKRLGTAWCSCREGFMKASDGKTC

SEQ ID NO: 48 - protein, 34 aa, Homo sapiens
CAPVGCSMYARCISEGEDATCQCLKGFAGDGKLC

SEQ ID NO: 49 - protein, 24 aa, Homo sapiens
KCINTEGGYVCRCSEGYQGDGIHC

SEQ ID NO: 50 - protein, 36 aa, Homo sapiens
CQLGVHSCGENASCTNTEGGYTCMCAGRLSEPGLIC

SEQ ID NO: 51 - protein, 37 aa, Homo sapiens
CPLSHDGYCLHDGVCMYIEALDKYACNCVVGYIGERC

SEQ ID NO: 52 - protein, 36 aa, Homo sapiens
CNAEFQNFCIHGECKYIEHLEAVTCKCQQEYFGERC

SEQ ID NO: 53 - protein, 41 aa, Mus musculus
FSHPCLEDHNSYCINGACAFHHELKQAICRCFTGYTGQRCE

SEQ ID NO: 54 - protein, 41 aa, Homo sapiens
FSHLCLEDHNSYCINGACAFHHELEKAICRCFTGYTGERCE

SEQ ID NO: 55 - protein, 50 aa, Homo sapiens
VVSHFNDCPDSHTQFCFHGTCRFLVQEDKPACVCHSGYVGARCEHADLLA

SEQ ID NO: 56 - protein, 71 aa, Homo sapiens
TSHLVKCAEKEKTFCVNGGECFMVKDLSNPSRYLCKCPNEFTGDRCQNYVMASFYKHLGIEFMEAEELYQK

SEQ ID NO: 57 - protein, 65 aa, Homo sapiens
SHLVKCAEKEKTFCVNGGECFMVKDLSNPSRYLCKCQPGFTGARCTENVPMKVQNQEKAEELYQK

SEQ ID NO: 58 - protein, 86 aa, Homo sapiens
DLQEADLDLLRVTLSSKPQALATPNKEEHGKRKKKGKGLGKKRDPCLRKYKDFCIHGECKYVKELRAPSCICHPGYHGERCHGLSL

SEQ ID NO: 59 - protein, 46 aa, Homo sapiens
VSITKCSSDMNGYCLHGQCIYLVDMSQNYCRCEVGYTGVRCEHFFL

SEQ ID NO: 60 - protein, 53 aa, Homo sapiens
NSDSECPLSHDGYCLHDGVCMYIEALDKYACNCVVGYIGERCQYRDLKWWELR

SEQ ID NO: 61 - protein, 98 aa, Homo sapiens
SVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSK

SEQ ID NO: 62 - protein, 51 aa, Mus musculus
LKFSHPCLEDHNSYCINGACAFHHELKQAICRCFTGYTGQRCEHLTLTSYA

SEQ ID NO: 63 - protein, 51 aa, Homo sapiens
LKFSHLCLEDHNSYCINGACAFHHELEKAICRCFTGYTGERCEHLTLTSYA

SEQ ID NO: 64 - protein, 76 aa, Homo sapiens
LENSTSPLSADPPVAAAVVSHFNDCPDSHTQFCFHGTCRFLVQEDKPACVCHSGYVGARCEHADLLAVVAASQKKQ

SEQ ID NO: 65 - protein, 80 aa, Homo sapiens
ACQALENSTSPLSADPPVAAAVVSHFNDCPDSHTQFCFHGTCRFLVQEDKPACVCHSGYVGARCEHADLLAVVAASQKKQ

SEQ ID NO: 66 - protein, 247 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKR

SEQ ID NO: 67 - protein, 242 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKR

SEQ ID NO: 68 - protein, 136 aa, Homo sapiens
LERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTT

SEQ ID NO: 69 - protein, 143 aa, Homo sapiens
ALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTT

SEQ ID NO: 70 - protein, 142 aa, Homo sapiens
LVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTT

SEQ ID NO: 71 - protein, 138 aa, Homo sapiens
ESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTT

SEQ ID NO: 72 - protein, 88 aa, Homo sapiens
VLSTTVIPSCIPGESSDNCTALVQTEDNPRVAQVSITKCSSDMNGYCLHGQCIYLVDMSQNYCRCEVGYTGVRCEHFFLTVHQPLSKE

SEQ ID NO: 73 - protein, 85 aa, Homo sapiens
TTVIPSCIPGESSDNCTALVQTEDNPRVAQVSITKCSSDMNGYCLHGQCIYLVDMSQNYCRCEVGYTGVRCEHFFLTVHQPLSKE

SEQ ID NO: 74 - protein, 82 aa, Homo sapiens
IPSCIPGESSDNCTALVQTEDNPRVAQVSITKCSSDMNGYCLHGQCIYLVDMSQNYCRCEVGYTGVRCEHFFLTVHQPLSKE

SEQ ID NO: 75 - protein, 1032 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQK

SEQ ID NO: 76 - protein, 1019 aa, Homo sapiens
KFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQK

SEQ ID NO: 77 - protein, 174 aa, Homo sapiens
AGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSK

SEQ ID NO: 78 - protein, 172 aa, Homo sapiens
LDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSK

SEQ ID NO: 79 - protein, 177 aa, Homo sapiens
HYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSK

SEQ ID NO: 80 - protein, 91 aa, Mus musculus
ALSEEAEVIPPSTAQQSNWTFNNTEADYIEEPVALKFSHPCLEDHNSYCINGACAFHHELKQAICRCFTGYTGQRCEHLTLTSYAVDSYEK

SEQ ID NO: 81 - protein, 79 aa, Homo sapiens
AAVTVTPPITAQQADNIEGPIALKFSHLCLEDHNSYCINGACAFHHELEKAICRCFTGYTGERCEHLTLTSYAVDSYEK

SEQ ID NO: 82 - protein, 111 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFY

SEQ ID NO: 83 - DNA, 336 bp, Homo sapiens
atggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttactag

SEQ ID NO: 84 - protein, 111 aa, Mus musculus
MDPTAPGSSVSSLPLLLVLALGLAILHCVVADGNTTRTPETNGSLCGAPGENCTGTTPRQKVKTHFSRCPKQYKHYCIHGRCRFVVDEQTPSCICEKGYFGARCERVDLFY

SEQ ID NO: 85 - DNA, 336 bp, Mus musculus
atggacccaacagccccgggtagcagtgtcagctccctgccgctgctcctggtccttgccctgggtcttgcaattctccactgtgtggtagcagatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatctgtgagaaaggctactttggggctcggtgtgagcgagtggacctgttttactag

SEQ ID NO: 86 - protein, 80 aa, Mus musculus; identical to SEQ ID NO: 20

SEQ ID NO: 87 - DNA, 243 bp, Mus musculus
gatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatctgtgagaaaggctactttggggctcggtgtgagcgagtggacctgttttactag

SEQ ID NO: 88 - protein, 81 aa, Mus musculus
MDGNTTRTPETNGSLCGAPGENCTGTTPRQKVKTHFSRCPKQYKHYCIHGRCRFVVDEQTPSCICEKGYFGARCERVDLFY

SEQ ID NO: 89 - DNA, 246 bp, Mus musculus
atggatgggaacacaaccagaacaccagaaaccaatggctctctttgtggagctcctggggaaaactgcacaggtaccacccctagacagaaagtgaaaacccacttctctcggtgccccaagcagtacaagcattactgcatccatgggagatgccgcttcgtggtggacgagcaaactccctcctgcatctgtgagaaaggctactttggggctcggtgtgagcgagtggacctgttttactag

SEQ ID NO: 90 - protein, 10 aa, Artificial Sequence
  Description of Artificial Sequence Synthetic His tag
  This sequence may encompass 3 to 10 His residues
HHHHHHHHHH

SEQ ID NO: 91 - protein, 6 aa, Artificial Sequence
  Description of Artificial Sequence Synthetic 6xHis tag
HHHHHH

(19) United States
(12) Patent Application Publication
(10) Pub. No.: US 2007/0054851 A1
(43) Pub. Date: Mar. 8, 2007

(54) Methods of and compositions for stimulation of glucose uptake into muscle cells and treatment of diseases

(76) Inventors: Junyu Lin, Palo Alto, CA (US); Srinivas Kothakota, Pacfica, CA (US); Ge Wu, La Canada, CA (US); Stephen Doberstein, San Francisco, CA (US); Thomas Brennan, Saratoga, CA (US); Lorianne Masuoka, Oakland, CA (US); Minmin Qin, Pleasanton, CA (US); Shannon Marshall, San Francisco, CA (US); Yan Wang, Redwood City, CA (US); Diane Hollenbaugh, Mountain View, CA (US); Lewis Thomas Williams, Mill Valley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/442,244

(22) Filed: May 30, 2006

Related U.S. Application Data
(60) Provisional application No. 60/685,702, filed on May 27, 2005.
(60) Provisional application No. 60/701,490, filed on Jul. 22, 2005.
(60) Provisional application No. 60/701,964, filed on Jul. 22, 2005.
(60) Provisional application No. 60/702,065, filed on Jul. 22, 2005.
(60) Provisional application No. 60/733,791, filed on Nov. 7, 2005.
(60) Provisional application No. 60/736,866, filed on Nov. 16, 2005.
(60) Provisional application No. 60/778,169, filed on Feb. 27, 2006.
(60) Provisional application No. 60/800,443, filed on May 16, 2006.

(51) Int. Cl. A61K 38/18

(52) U.S. Cl. 514/12

(57) ABSTRACT

The present invention relates to therapeutic uses of ErbB ligands, including betacellulin. The therapeutic uses include methods of using ErbB ligand family compounds alone, or in conjunction with other agents, for reducing blood glucose levels, treating Type I and Type II diabetes, obesity, muscle wasting diseases, and cardiotoxicity.

### CROSS REFERENCE TO RELATED APPLICATIONS

This application claims the benefit of the following applications filed in the United States Patent and Trademark Office: U.S. Provisional Application No. 60/685,702, filed May 27, 2005; U.S. Provisional Application No. 60/701,490, filed Jul. 22, 2005; U.S. Provisional Application No. 60/701,964, filed Jul. 22, 2005; U.S. Provisional Application No. 60/702,065, filed Jul. 22, 2005; U.S. Provisional Application No. 60/733,791, filed Nov. 7, 2005; U.S. Provisional Application No. 60/736,866, filed Nov. 16, 2005; U.S. Provisional Application No. 60/778,169, filed Feb. 27, 2006; U.S. Provisional Application 60/800,443 filed May 16, 2006; and the PCT Application entitled “Methods of and Compositions for Stimulating Glucose Uptake Into Muscle Cells and Treatment of Diseases,” filed May 30, 2006, the disclosures of all of which are herein incorporated by reference in their entireties.

### FIELD OF THE INVENTION

The present invention relates to therapeutic uses of the ErbB ligand family proteins, also known as epidermal growth factors (EGFs). The therapeutic uses include methods of using ErbB ligand compounds singly, in combination, and/or in conjunction with other agents, for glycemic control, stimulation of glucose uptake into muscle cells, and treatment of diseases.

### BACKGROUND OF THE INVENTION

Glucose is the major form in which diet-derived carbohydrates absorbed from the intestinal tract are presented to the cells of the human body. Glucose is the only fuel used to any significant extent by several specialized cells in mammals (e.g., white muscle cells), and it is the major fuel used by the brain. Indeed, the capacity to store and/or synthesize glucose, for example through the processes of glycogenolysis (i.e., breakdown of glycogen in the liver and skeletal muscle) and gluconeogenesis (e.g., synthesis from amino acids), is crucial for human survival. Moreover, glucose is so important to these specialized cells and the brain that several of the major tissues of the body (i.e., muscle, liver, fat and kidney) work together to ensure a continuous supply of this essential cellular substrate.

Two of the most prevalent metabolic diseases, obesity and diabetes, are linked to acute or sustained breakdowns in the glucose supply chain. Often, these diseases arise because of an impaired cellular capacity to sense and/or uptake glucose, a process which is largely regulated by insulin and glucagon. But both obesity and diabetes can also be the result of dysregulated glucose metabolism. In turn, obesity and diabetes are contributing factors in the development of major medical problems, including atherosclerosis, heart failure, hypertension, small vessel disease, kidney failure, limb amputation, and blindness. Various clinical trials indicate that the long-term risk of these complications can be reduced through optimal glycemic control, together with rigorous control of blood pressure, diet and physical activity.

Hyperglycemia, or elevation of blood glucose levels beyond about 130 md/dL in humans, is a common and severe illness associated with adverse outcomes; it is a risk factor for complications from stroke, myocardial infarction, vascular and cardiac surgery, and is associated with increased mortality, both in the critically ill and the trauma patient. On the other hand, strict glucose control improves the outcomes of, for example, cardiac surgery, myocardial infarction and intensive care unit treatment (Van der Berghe et al., NEJM, 354:449-461, (2006)).

Most often, hyperglycemia is present in the context of diabetes. However, hyperglycemia in the absence of diabetes (e.g., stress hyperglycemia) has also been described, and typically refers to plasma glucose levels above about 200 md/dL in humans (about 11.1 mmol/liter). Some of the mechanisms for stress hyperglycemia are well known. For example, excess of counterregulatory hormones (e.g., epinephrine, glucagon, cortisol, growth hormone) and cytokines (TNFα, migration-inhibitory factor/MIF) during acute illness, frequently result in insulin resistance. Furthermore, many hospitalized patients are insulin deficient for a variety of other reasons such as, for example, chronic kidney disease, acute physiologic stress, pancreatitis, hypothermia, and hypoxemia. Excess dextrose infusion is also an often-overlooked contributor to hyperglycemia, particularly in patients undergoing total parenteral nutrition or enteral nutritional support. Stress hyperglycemia increases the risk of death, congestive heart failure, and cardiogenic shock after myocardial infection, and increases in-hospital mortality after ischemic shock (as described in Hirsch, I. B., J Clin Endocrinol Metab. 87:975-977 (2002)).

In recent clinical trials, strict control of glucose levels in patients admitted to the surgical intensive care unit (ICU), significantly reduced morbidity and mortality. Medical complications, such as severe infections and organ failure, were also reduced. Several potential mechanisms were proposed to explain the benefits of strict glucose control, including prevention of immune dysfunction, reduction of system inflammation, and protection of both the endothelium, and of mitochondrial structure and function (discussed in Van den Berghe et al., NEJM, 345: 1359-1367 (2001); Van der Berghe et al., NEJM, 354: 449-461, (2006)). Since the original 2001 trial in the surgical ICU (Van den Berghe et al., NEJM, 345: 1359-1367 (2001), elevations in blood glucose among the critically ill, a marker previously ignored or described as adaptive, have become a major therapeutic target.

Improved glycemic control reportedly also reduces the risks of early microvascular complications, such as retinopathy, nephropathy, and neuropathy, in patients with diabetes, 18.2 million of whom reside in the U.S. alone. Nevertheless, around 3.2 million deaths a year (six deaths every minute) are still attributable to complications of hyperglycemia and/or diabetes, which includes both Type I and II diabetes, and metabolic syndrome. Recently, the World Health Organization (WHO) declared that a diabetes epidemic is underway (Smyth and Heron, Nature Medicine 12: 75-80 (2006); the WHO Report “Preventing Chronic Diseases: a Vital Investment” (2005)). In 1985, an estimated 30 million people worldwide had diabetes. However, by 1995, this number had risen to 135 million. In 2005, an estimated 217 million people worldwide suffered from diabetes, and the WHO predicts that by 2030 this number will grow beyond 366 million.

Two major concerns of this global diabetes crisis are (i) that much of the increase in diabetes-associated morbidity, mortality, and economic burden (Yach, D. et al., Nature Medicine 12: 62-66 (2006)) will occur in developing countries such as India and China, due to population growth, ageing, unhealthy diets, obesity and sedentary lifestyles, and (ii) that there is a growing incidence of Type II diabetes—which accounts for about 90% of all cases—at a younger age. In the US, Japan, and other developed countries, most people with diabetes are above the age of retirement. On the other hand, in developing countries those most frequently affected are in the middle, productive years of their lives, aged between 35 and 64. Overall, direct health care costs of diabetes range from 2.5% to 15% of annual health care budgets, depending on local diabetes prevalence and the sophistication of the treatment available. The costs of lost-production may be as much as five times the direct health care cost, according to WHO estimates derived from 25 Latin American countries.

Accordingly, diabetes is a urgent and multifactorial disease that represents a major public health threat. Type II diabetes is generally caused by a combination of insulin deficiency and insulin resistance. Indeed, those with the disease share a group of clinical symptoms, including chronic hyperglycemia and increased insulin resistance in tissues with insulin-stimulated glucose transport (insulin-target tissues): muscle, liver, and adipose tissue. Insulin resistance is a major contributor to the progression of the disease and to complications of diabetes, such as diabetic neuropathy, diabetic retinopathy, metabolic syndrome and muscle wasting.

Insulin resistance reportedly is defined as an impaired effect of a certain amount of insulin in target tissues (e.g., muscle, fat and liver). A major consequence of insulin resistance is altered carbohydrate metabolism. In muscle, insulin-stimulated glucose transport and the first step in glucose metabolism (phosphorylation of glucose at carbon 6) both become impaired. The rate of glycogen synthesis can also be reduced. In fat, insulin resistance appears as impaired glucose uptake but also as an impaired suppression of lipolysis. In the liver, higher insulin concentrations than normal become needed to suppress glucose production. Environmental factors like physical inactivity, a high-energy and high-fat diet, smoking and stress, strongly interact with a genetic predisposition to promote the development of diabetes. However, the primary factors responsible for the development of insulin resistance remain unknown.

Until recently, the prevailing view was that insulin resistance was mainly caused by primary defects in insulin target cells. However, it now seems more likely that systemic neuroendocrine dysregulation also plays a major role in the development of insulin resistance (Buren and Eriksson, Diabetes Metab Res Rev 21:487-494 (2005); Pocai, A., et al., Nature 434: 1026-1031 (2005); Seeley and Tschöp, Nature Medicine 12:47-49 (2006)). Given the global obesity and diabetes epidemics, and the inability of the available drugs to address these diseases adequately, there in an unmet need to identify other agents that can influence glucose uptake and metabolism for the treatment of both diseases.

### SUMMARY OF THE INVENTION

The present invention provides compositions, kits and methods that can be used to treat subjects that would benefit from stimulating glucose or amino acid uptake into muscle cells, promoting cell survival or inhibiting apoptosis of muscle cells, inducing utrophin expression, inhibiting muscle wasting or increasing muscle mass, reducing HbA_{1c}, reducing hypoglycemia associated with insulin administration, reducing the basal blood glucose level, and/or acutely reducing the elevated blood glucose level in the subject.

The present invention is directed to pharmaceutical compositions comprising a concentration of betacellulin or an active variant or fragment thereof sufficient to acutely reduce the blood glucose level in a subject without inducing hypoglycemia and a pharmaceutically acceptable carrier.

In some embodiments of the invention, the composition comprises along-acting betacellulin fusion protein comprising a betacellulin polypeptide and a fusion partner or an active variant or fragment thereof, wherein the betacellulin fusion protein has an extended half-life in a subject when compared to the betacellulin polypeptide alone. For example, the long-acting betacellulin fusion protein can have an extended half-life that comprises at least 0.5 hr, at least 1 hr, at least 2 hr, at least 3 hr, at least 4 hr, or at least 5 hr longer than the half-life of the betacellulin polypeptide alone.

Non-limiting examples of the fusion partner in a long-acting betacellulin fusion protein can be a polymer, a polypeptide, a succinyl group, or an active variant or fragment of any of these. For example, the polymer comprises a polyethylene glycol moiety either permanently or reversibly covalently attached to the betacellulin polypeptide. The fusion partner polypeptide, for example, can comprise an immunoglobulin fragment, albumin, or an oligomerization domain. In one embodiment, the immunoglobulin fragment comprises an Fc fragment.

The pharmaceutical composition can be provided in a kit. Non-limiting examples of the kits provided in the invention are those comprising: (a) a pharmaceutical composition comprising a polypeptide of the ErbB ligand family or an active variant or fragment thereof, or a long-acting fusion protein comprising a polypeptide of the ErbB ligand family or an active variant or fragment thereof and a fusion partner, wherein the fusion protein has an extended half-life in a subject when compared to the ErbB ligand polypeptide alone; and a pharmaceutically acceptable carrier; and (b) instructions for administration into a subject in need of such a composition.

The kit can contain instructions that describe one or more several uses for the composition(s) contained therein. For example, there can be instructions for use of the composition for acutely reducing elevated blood glucose levels, for inhibiting muscle wasting or increasing muscle mass in the subject, for increasing glucose or amino acid uptake into the cardiac muscle of the subject, for treating obesity, and/or for the use of the composition for treating the subject in an emergency setting.

The kit can further comprise a vial or cartridge. The vial or cartridge can comprise from about 50 micrograms/milliliter to about 100 micrograms/milliliter of ErbB ligand polypeptide. Optionally, the vial or cartridge comprises from about 100 micrograms/milliliter to about 1 milligram/milliliter of ErbB ligand polypeptide. In other embodiments, the vial or cartridge comprises from about 1 milligram/milliliter to about 5 milligrams/milliliter of ErbB ligand polypeptide; or from about 5 milligrams/milliliter to about 500 milligrams/milliliter of ErbB ligand polypeptide; or from about 100 milligrams/milliliter to about 400 milligrams/milliliter of ErbB ligand polypeptide; or even from about 200 milligrams/milliliter to about 300 milligrams/milliliter ErbB of ligand polypeptide.

In one embodiment, the vial or cartridge comprises a single dose of ErbB ligand polypeptide with a volume of about 0.5 milliliters, about 1.0 milliliter, or about 1.5 milliliters. In one embodiment, the vial or cartridge comprises a single dose, a double dose, or a triple dose of the ErbB ligand polypeptide, wherein each dose has a volume of about 0.5 milliliters, about 1.0 milliliter, or about 1.5 milliliters. The vial or cartridge can also comprise ErbB ligand in solid form, including, but not limited to freeze-dried polypeptide.

The invention also provides kits further comprising at least one second agent, wherein the second agent is an anti-diabetic agent.

The invention provides several methods for treating a disease. In one embodiment, the invention provides a method of treating a disease in a subject comprising: (a) providing a polypeptide of the ErbB ligand family; and (b) administering the polypeptide to the subject, wherein the subject has normal pancreatic function and/or a normal insulin level and would benefit from stimulating glucose or amino acid uptake into muscle cells, promoting cell survival or inhibiting apoptosis of muscle cells, inducing utrophin expression, inhibiting muscle wasting or increasing muscle mass, reducing HbA_{1c}, reducing hypoglycemia associated with insulin administration, reducing the basal blood glucose level, and/or acutely reducing the elevated blood glucose level in the subject. Optionally, the invention also provides a method of treatment further comprising: (c) administering at least one second agent, wherein the second agent is another therapeutic agent.

In one embodiment, the polypeptide of the ErbB ligand family comprises betacellulin or an active variant or fragment thereof. Alternatively, the polypeptide of the ErbB ligand family comprises a long-acting ErbB ligand fusion protein comprising a polypeptide of the ErbB ligand family or an active variant or fragment thereof and a fusion partner, wherein the ErbB ligand fusion protein has an extended half-life in a subject when compared to the ErbB ligand polypeptide alone.

The disease can comprise an elevated blood glucose level, obesity, Type I or Type II diabetes, a condition selected from acute hyperglycemia, incipient diabetic ketoacidosis, diabetic ketoacidosis, and diabetic coma. The disease can also be selected from muscle wasting associated with diabetic amyotrophy or other metabolic myopathy, cachexia, AIDS wasting, disuse atrophy, sarcopenia, rhabdomyolysis, myositis, diaphragmatic weakness due to muscular disorder, and muscular dystrophy. The muscle cells affected by the polypeptide can be skeletal, cardiac, and smooth muscle cells.

Administration of the polypeptide can be at least once a day, at least two times a day, or at least three times a day. In one embodiment, the polypeptide is administered at a dose sufficient to produce a euglycemic level of blood glucose. In one embodiment, the polypeptide is administered in an amount sufficient to lower fasting blood glucose and/or lower the HbA_{1c} level in the subject.

In one embodiment, the amount is sufficient for increasing glucose or amino acid uptake by the cardiac muscle of the subject for treatment of cardiac disease, and the cardiac disease is selected from ischemia, congestive heart failure, myocardial infarction, and induced cardiotoxicity. Induced cardiotoxicity includes that which is induced by chemotherapy and that which is virally induced.

The subject can be treated in an emergency setting. Emergency settings include an emergency room, an intensive care setting, a setting wherein the subject is acutely ill, and a setting wherein the subject is suffering from a condition selected from respiratory failure, cardiac failure, kidney failure, diabetic ketoacidosis, and another life-threatening condition.

The method of treatment can comprise administering the polypeptide orally, subcutaneously, intravenously, transdermally, intraperitoneally, by inhalation, by implantation, intradermally, intramuscularly, intracardially, nasally, and/or by rectal suppository. The polypeptide can be administered as a composition comprising a collagen or a gel.

The polypeptide is administered at a dose sufficient to produce a blood concentration of the polypeptide in a range from about 1 nanomolar to about 10 nanomolar or from about 10 nanograms/milliliter to about 100 nanograms/milliliter in the subject.

One or more doses of the polypeptide can be administered at or about meal time. For example, the polypeptide can be administered within about 120 minutes, about 90 minutes, about 60 minutes, about 30 minutes, about 15 minutes, or about 5 minutes before or after a meal; or during a meal.

The benefit which the subject derives from the methods of treatment of the invention can comprise acute reduction of elevated blood glucose level. The acute reduction can occur within about 1 minute to about 120 minutes; within about 2 minutes to about 90 minutes; within about 3 minutes to about 60 minutes; within about 4 minutes to about 30 minutes; or within about 5 minutes to about 15 minutes.

The polypeptide is administered in one or more doses, selected from a dose comprising from more than about 50 micrograms to less than about 2 milligrams, greater than about 2 milligrams to less than about 10 milligrams, and greater than about 10 milligrams to about 500 milligrams.

In one embodiment, the dose comprises from about 100 milligrams to about 400 milligrams. In another embodiment, the dose comprises from about 200 milligrams to about 300 milligrams.

In one embodiment, the polypeptide is administered in one or more doses. The weight of the subject is measured in kilograms, and each dose comprises from about 0.01 milligrams/kilogram to about 5 milligrams/kilogram. In one embodiment, the dose comprises from about 0.1 milligrams/kilogram to about 2 milligrams/kilogram. In another embodiment, the dose is from about 0.2 milligrams/kilogram to about 1 milligram/kilogram. In another embodiment, the dose is from about 0.3 milligrams/kilogram to about 0.9 milligrams/kilogram. The dose can also be from about 0.4 milligrams/kilogram to about 0.8 milligrams/kilogram, or from about 0.5 milligrams/kilogram to about 0.7 milligrams/kilogram. In one embodiment, the dose comprises no more than 1 milligram/kilogram.

The polypeptide can also be administered in one or more doses, each comprising from about 1 microgram/kilogram to about 10 milligrams/kilogram. In one embodiment, the polypeptide is administered in one or more doses, each comprising from about 10 micrograms/kilogram to about 1 milligram/kilogram.

The second agent can comprise an anti-diabetic agent. The second agent can be administered orally, subcutaneously, intravenously, transdermally, intraperitoneally, by inhalation, by implantation, intradermally, intramuscularly, intracardially, nasally, and/or by rectal suppository.

Furthermore, the second agent can be administered before, after, or at the same time as the polypeptide. The second agent can be selected from metformin, an insulin secretagogue, a glucosidase inhibitor, a PPAR gamma agonist, and a dual gamma/alpha-PPAR agonist.

In one embodiment, the insulin secretagogue is selected from a sulfonylurea and a meglitinide. In one embodiment, the second agent is selected from insulin, an insulin analogue, a co-secreted agent, pramlinitide, and a DPP4 antagonist. In another embodiment, the second agent comprises a glucagon-like peptide. The glucagon-like peptide can comprise, for example, exenatide.

### BRIEF DESCRIPTION OF THE FIGURES AND THE APPENDIX

Brief Description of the Figures

FIG. 1 shows a flow chart of a high-throughput method used to screen known and unknown substances for significant effects on cell impedance, which is a measure of the cellular response to those substances.

FIG. 2 shows a flow chart of a high-throughput method used to screen test substances (such as, for example, secreted proteins present in conditioned media of cells transfected with a cDNA from a cDNA library of secreted proteins, and recombinant proteins) for an effect on a characterized hormone response.

FIG. 3 shows that agents that affect the insulin-signaling pathway decreased the cell index in L6 cells. Insulin, insulin-like growth factor I (IGF-I), insulin-like growth factor II (IGF-II), and platelet-derived growth factor BB (PDGF-BB) each decreased the cell index at a concentration of 100 nM over 120 min. Growth differentiation factor-8 (GDF-8), (growth hormone (GH), and basic fibroblast growth factor (bFGF or FGF-2), on the other hand, had no effect on the cell index in L6 cells.

FIG. 4 shows that the EC₅₀ of insulin (FIG. 4A), IGF-I (FIG. 4B), and IGF-II (FIG. 4C) in L6 cells, when measured by the RT-CES™ system, are similar to published EC₅₀ values obtained using uptake of ³H-deoxyglucose as a measurement. The EC₅₀ of insulin was about 41 nM, IGF-I was about 102 pM, and IGF-II was about 2.9 nM, as quantitated by cell index/impedance assay described in Example 4.

FIG. 5 shows the EC₅₀ of insulin (FIG. 5A), IGF-I (FIG. 5B), and IGF-II (FIG. 5C) in primary human skeletal muscle cells using the RT-CES™ system. The EC₅₀ of insulin was approximately 8.3 nM, which indicates that the primary skeletal muscle cells were approximately five-fold more sensitive to insulin than the L6 cell line. The EC₅₀ of IGF-I was approximately 270 pM; the EC₅₀ of IGF-II was approximately 2.7 nM, as further described in Example 6.

FIG. 6 (panels A and B) shows the results of an high-throughput screening of human skeletal muscle cells with secreted factors for agents that increase impedance, as further described in Example 8. FIG. 6A shows the results of an impedance assay for testing agents that have an effect on impedance of human primary skeletal muscle cells. The results are plotted as the normalized cell index at a single time point (30 minutes) measured at 30 min after treatment with the agents. Columns 1-12 and rows A-H refer to the grid of wells in the 96 well plate. Betacellulin (arrow) is contained in well G3, and causes an increase in cell index. Well H4 contains the internal positive control insulin growth factor-I (IGF-I). Well D6 contains interleukin 4 (IL-4). Well H3 contains fibroblast growth factor-1 (FGF-1). Well D10 contains Semaphorin 3F. Well H10 contains PDGF-C. Well D8 contains endothelin 3. Wells 12A-D contain the external positive control 10 nM IGF-I. No data are shown with respect to wells 1E-H and 2A-D. FIG. 6B shows the results of screening human skeletal muscle cells with secreted factors for agents that alter the cell's impedance response to insulin, as further described in Example 8. The data were plotted as a single time point at 30 minutes after insulin addition, in a 96 well plate layout. Betacellulin (well G3), fibroblast growth factor-18 (FGF 18) and FGF 1 were identified as agents that increase the impedance response to insulin. Well H4 contains the internal positive control IGF-I and wells 12A-D are 10 nM IGF-I contain the external positive control.

FIG. 7 shows the time course of the change in cell index in primary human skeletal muscle cells exposed to betacellulin (100 nM) or insulin (1 uM), as further described in Example 9. The effect on cell index was normalized and compared to that of cells incubated for 24 hours in the absence of either insulin or betacellulin (control).

FIG. 8 shows the change in cell index in primary human skeletal muscle cells, pre-incubated with either purified betacellulin (100 nM) or insulin (1 uM), and then treated with insulin, as further described in Example 10. The effect on cell index was normalized and compared to that of cells incubated for 24 hours in the absence of either insulin or betacellulin, and then treated with insulin (control).

FIG. 9 shows the cell impedance change induced by ErbB ligand polypeptides, as further described in Example 11. 1 uM insulin and 100 pM of each of epidermal growth factor (EGF), betacellulin (BTC), Epigen, transforming growth factor-alpha (TGF-alpha), amphiregulin (AR), epiregulin (EPR), heparin-binding EGF (HB-EGF), neuregulin 1-alpha (NRG1-a), and neuregulin 1-beta (NRG1-b) were tested. Among those tested, EGF and betacellulin produced the highest increase in cell index, approximating that caused by insulin, and at doses (100 pM) several orders of magnitude lower than insulin (1 microM).

FIG. 10 shows that betacellulin stimulated glucose uptake in primary human skeletal muscle cells, as further described in Example 12. Both insulin and betacellulin increased glucose uptake in a dose-dependent manner. Betacellulin was more potent, as it increased glucose uptake at lower concentrations than insulin. The EC₅₀ of insulin was measured to be approximately 27 nM, while the EC₅₀ of betacellulin was measured to be approximately 43 pM.

FIG. 11 shows the potentiating effect of betacellulin on insulin action on primary human skeletal muscle cells as reflected by its effect on glucose uptake, as assayed by the ³H-deoxyglucose uptake method, further described in Example 13. Cells were treated with 100 nM betacellulin, 10 pM betacellulin, 100 pM insulin, or a combination of 100 pM insulin and 10 pM betacellulin. The combination induced glucose uptake to a greater degree than either 100 pM insulin or 10 pM betacellulin alone.

FIG. 12 shows that betacellulin increased insulin-stimulated glucose uptake by primary human skeletal muscle cells in a dose-dependent manner, as further described in Example 14. Both 10 pM (top) and 1 pM (bottom) concentrations of betacellulin increased glucose uptake.

FIG. 13A and FIG. 13B show that glucose uptake was stimulated by ErbB ligand polypeptides, as further explained in Example 15. FIG. 13A shows the relative glucose uptake stimulated by BTC, EGF, HB-EGF, and TGF-alpha, while FIG. 13B shows the relative glucose uptake stimulated by AR, EPR, Epigen, NRG1-alpha (NRG1-a), and NRG1-beta (NRG1-b).

FIG. 14 shows the clearance rate of betacellulin from the plasma of wild-type normal C57BL/6J mice after intravenous injection of 0.5 mg of betacellulin per kg body weight of mice into the tail vein of the mice, as further described in Example 17. Under these conditions, betacellulin has an in vivo half-life of about 32 min.

FIG. 15A and FIG. 15B show the plasma clearance rates of betacellulin after subcutaneous injection (FIG. 15A) versus after intravenous injection (FIG. 15B), into wild-type normal C57BL/6J mice, of 0.05 mg/kg of betacellulin, as further described in Example 18. An increase in the duration of betacellulin bioavailability was observed following subcutaneous injection as compared to intravenous administration.

FIG. 16 illustrates the plasma levels and clearance rates of betacellulin after subcutaneous administration of 0.8 mg/kg weight and 0.05 mg/kg weight, respectively, in C57BL/6J mice, as further described in Example 19. Results show that, at the 0.8 mg/kg dose, the plasma level of betacellulin reached a peak of about 120 nM at about 120 min post administration; and at the 0.05 mg/kg dose, betacellulin reached a peak of about 0.6 nM at about 30 min post-administration.

FIG. 17A and FIG. 17B illustrate the effect of subcutaneous administration of betacellulin on both blood glucose levels (FIG. 17A) and plasma betacellulin levels (FIG. 17B) in normal wild-type C57BL/6J mice, under fasting conditions, as further described in Example 20. Betacellulin reduced blood glucose in a dose-dependent manner, with rapid kinetics.

FIG. 18A (wild type normal mice) and FIG. 18B (db mice, animal model of diabetes) illustrate the effect of betacellulin on postprandial plasma glucose levels, as further described in Example 21. The results show that, under these conditions, db (diabetic) mice are more sensitive to betacellulin than normal mice in that only the db mice experienced significant decrease in postprandrial glucose levels upon betacellulin treatment.

FIG. 19 depicts the structure of the vector used for long-term expression of recombinant human betacellulin in mice via hydrodynamic tail-vein transfection of betacellulin cDNA. The vector comprises the following parts: alpha-antitrypsinPro corresponds to an alpha1-antitrypsin promoter with an apoe enhancer; Human FIX corresponds to intron 1 of the human factor IX gene; BT represents the cDNA for human betacellulin; and poly represents a bovine polyA tail.

FIG. 20 (panels A, B, C, and D) illustrates the effects of long-term betacellulin expression (i.e., extended increase in circulating betacellulin plasma levels (FIG. 20A)), in db mice on their fasting glucose (FIG. 20B), HbA_{1c} levels (FIG. 20C), and plasma insulin levels (FIG. 20D), as further explained in Example 22. Circulating betacellulin levels were significantly higher than normal as long as 18 days after cDNA injection, which resulted in preventing a rise in fasting glucose levels over the course of the test. This “chronic” increase in betacellulin was also accompanied by a decrease in HbA_{1c} and insulin levels.

FIG. 21 illustrates the relative effect of subcutaneous administration of ErbB ligands (betacellulin, EGF, HB-EGF, NRG-1) on blood glucose levels in diabetic (db) mice, as further described in Example 23. The two controls were saline and diluted acetic acid (which was used to solubilize the ErbB ligands, with the exception of BTC, which was solubilized in saline). Under these conditions, betacellulin has the most potent effect on reducing blood glucose, and it does so with the most rapid kinetics.

FIG. 22 illustrates the effect of varying the amount and the timing of the dose of betacellulin on its ability to lower postprandial glucose levels, as further described in Example 24. The results show that the effect of betacellulin on postprandial glucose levels is more dependent on the timing of the administration (relatively to the consumption of glucose/sugar) than it is on the overall, cumulative dose of betacellulin.

FIGS. 23A and 23B illustrate the pharmacokinetic profile of betacellulin in rats after intravenous (FIG. 23A) and subcutaneous administration (FIG. 23B), as further described in Example 25. The results show that betacellulin is rapidly cleared from the blood with a half-life of around 60 min, depending on the route of administration.

FIGS. 24A and 24B illustrate the additive effect of combining betacellulin with GLP1 (i.e., mimicking a combination therapy regimen with an insulinotropic drug), as further described in Example 26. The results show that GLP1 and insulin have an additive effect on lowering postprandial glucose levels.

FIG. 25 (panels A, B and C) illustrate the additive effect of combining betacellulin with metformin (mimicking a combination therapy regimen with an hypoglycemic agent that inhibits hepatic gluconeogenesis and enhances peripheral glucose uptake and utilization), as further explained in Example 27. The results show that the combination is more effective at lowering postprandial glucose levels than either metformin or betacellulin alone.

FIG. 26 illustrates the additive effect of combining betacellulin with insulin, mimicking the therapeutic effect of such combination on postprandial blood glucose levels, as further explained in Example 28. The results show that betacellulin enhances the effect of a drug which acts directly on insulin receptors (i.e., insulin) and works additively with it to reduce postprandial blood glucose levels.

FIGS. 27A and 27B illustrate the additive effect of combining betacellulin with a long-acting insulin analog (namely, glargine), as further explained in Example 29. The results show that such combination results in a more effective postprandial control, which works better both acutely and in maintaining a lower basal glucose level than either agent alone.

FIG. 28 illustrates a comparison between glucose uptake by isolated rat plantaris muscle in situ in response to either insulin or betacellulin administration, as further described in Example 30. Results show that 5 nM of betacellulin improves glucose uptake in situ when compared to 12 nM insulin.

FIG. 29 illustrates a comparison between amino acid uptake by primary human skeletal muscle cells treated with insulin and with betacellulin, as further explained in Example 31. Results show that betacellulin improved the uptake of a ¹⁴C-labeled alanine analog, relative to insulin, at doses between 10⁻¹¹ M and 10⁻⁸ M.

FIG. 30 illustrates the effect of several ErbB ligand family members (10 nM) on the ability of primary human skeletal muscle cells to upregulate utrophin expression in vitro. The graph shows that betacellulin (BTC), EGF, and NRG1-alpha (NRG1-a) all upregulated utrophin expression in primary human skeletal muscle cells, relative to control cells maintained in serum-free medium, as further described in Example 32.

FIG. 31 illustrates the effects of different ErbB ligand family members (at 100 pM) on utrophin expression by primary human skeletal muscle cells in vitro, as further described in Example 33. Results show that, at this concentration, BTC and TGF-alpha induced the highest level of utrophin expression. HB-EGF, EGF, and Epiregulin (EPR) also induced a higher level of utrophin expression relative to that measured in the control-treated cells.

FIG. 32 illustrates the effect of betacellulin and insulin in lipogenesis in vitro, by primary rat adipocytes. As further described in Example 34, betacellulin does not stimulate lipogenesis in isolated adipocytes.

FIG. 33 illustrates the effect of betacellulin on ErbB/EGF receptor phosphorylation. As further described in Example 35, betacellulin biological activity (OD₄₅₀) is associated with EGF receptor activation in a dose-dependent manner.

FIG. 34 shows that, similarly to what was observed for human skeletal muscle cells, betacellulin stimulates glucose uptake into cardiomyocytes, as further explained in Example 36.

FIG. 35A illustrates the results of phosphorylated Akt (pAkt) assays (FIG. 35A.1 and FIG. 35A.3, left panel) and of phosphorylated ERK (PERK) assays (FIG. 35A.2 and FIG. 35A.3, right panel) of rat neonatal cardiomyocytes treated with different doses of various recombinant proteins, as further described in Example 37. Rat neonatal cardiomyocytes were treated with different recombinant human proteins for 15 min followed by luminex-based pAkt, pERK and pSTAT3 detection. The doses represented are: 100 ng/ml for the first bar, 33 ng/ml for the second bar, 11 ng/ml for the third bar, and 0 ng/ml (i.e. control treatment without any recombinant protein added) for the fourth bar, starting from left portion of each figure. The height of the bar (y-axis) represents the luminescent signal readout. Both BTC and NRG1-beta1 increased pAkt level dramatically (FIG. 35A.1 and FIG. 35A.3 left panel), whereas both HB-EGF and NRG1-alpha increased pAkt level to a relatively lesser extent. Epiregulin, BTC, and NRG1-beta1 increased pERK level (FIG. 35A.2 and FIG. 35A.3, right panel), and TGF-alpha, HB-EGF, NRG1-alpha, and EGF also enhanced pERK level, but to a lesser extent. None of the tested proteins in this experiment showed effects on pSTAT activation. FIG. 35A.3 showed the dose-dependent effects of BTC and NRG1-beta1 on pAkt (FIG. 35A.3, left panel) and pERK (FIG. 35A.3, right panel) levels (represented as expression) after neonatal cardiomyocytes were treated with increasing doses of these proteins.

FIG. 35B illustrates the effect of various recombinant proteins on the survival of neonatal cardiomyocytes exposed to starvation (FIG. 35B.1), ischemia (FIG. 35B2), or cardiotoxic drugs (FIG. 35B.3), as described in Example 37. Betacellulin increased the survival or viability of cells exposed to either nutrient deprivation (starvation) or oxygen deprivation (ischemia). FIG. 35B.3 illustrates the results of a cell viability assay on cardiomyocytes exposed to the cardiotoxic drug doxorubicin in the presence of betacellulin, as further explained in Example 37. The results show that betacellulin enhanced the survival of cardiomyoctes in the presence of doxorubicin, in a dose-dependent manner.

FIG. 36 illustrates the results of an impedance assay on human primary skeletal muscle cells using a betacellulin splice variant as the stimulating agent (BTC SV), as further explained in Example 38. The results show that, unlike mature betacellulin, a betacellulin splice variant lacking a portion of the C-terminal domain is not able to stimulate an increase or decrease in cell index, as measured by the impedance assay.

FIG. 37 illustrates the effect of the BTC SV on glucose uptake by human primary skeletal muscle cells, as further explained in Example 39. The results show that a betacellulin splice variant lacking the C-terminal domain is not able to stimulate glucose uptake under these conditions.

FIGS. 38A and 38B illustrate results of an interim analysis of the effects of daily injections of betacellulin in db mice, as further explained in Example 22. The results confirm a dose-dependent beneficial effect on long-term glycemic control as measured by HbA_{1c} and fasting blood glucose.

FIG. 39 shows the amino acid alignment of betacellulin CLN00902377_expressed_Met (mature human betacellulin, corresponding to residues 32-111, preceded by a Met residue); betacellulin NP_{—}001720_NM_{—}001729; SEQ. ID NOS. 3, 14, 17, and 18 from U.S. Pat. No. 5,886,141; and SEQ ID NOS. 1 and 2 from U.S. Pat. No. 6,232,288. The alignment was performed by the freeware CLUSTAL FORMAT for T-COFFEE Version_{—}1.37, CPU=0.00 sec, SCORE=75, Nseq=8, Len=178.

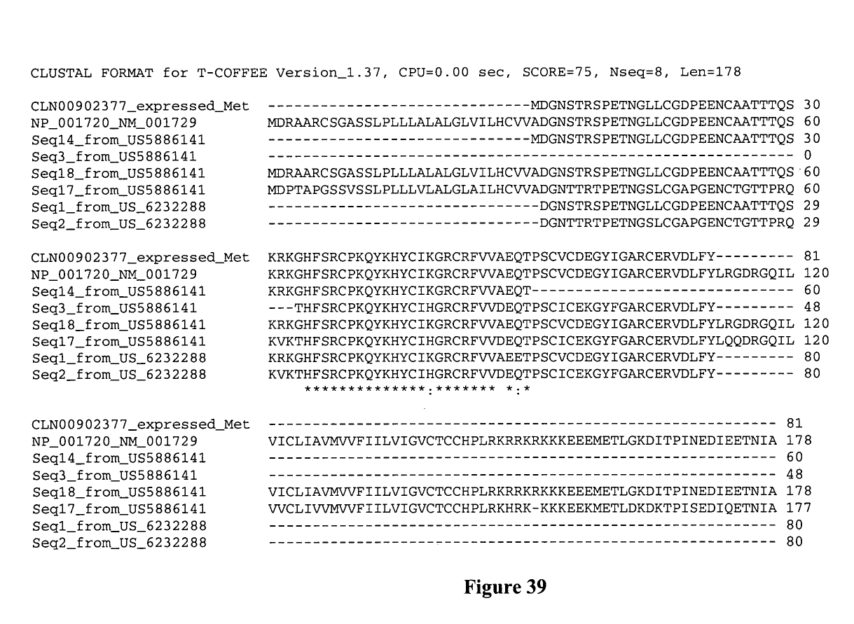

FIG. 40 shows the amino acid alignment of betacellulin 22218788_{—}33871113, betacellulin NP_{—}001720_NM_{—}001729, and betacellulin 15079597_{—}15079596. The alignment was performed by CLUSTAL FORMAT for T-COFFEE Version_{—}1.37, CPU=0.00 sec, SCORE=76, Nseq=3, Len=178.

FIG. 41 shows the results of a Western blot-based analysis of betacellulin in the plasma at 2 min, 30 min, 2 hr, and 18 hr after injection of betacellulin-Fc fusion protein (BTC-Fc), PEGylated betacellulin (PEG-BTC), and unmodified betacellulin (BTC). PEG-BTC and BTC-Fc were cleared from mouse plasma significantly more slowly than unmodified betacellulin.

### DETAILED DESCRIPTION OF THE INVENTION

A. Definitions

Unless defined herein, terms used herein have their ordinary meanings, and can be further understood in the context of the specification.

The terms “polypeptide” and “protein” are used interchangeably to refer to a polymer of amino acid residues, and are not limited to a minimum length. Thus, peptides, oligopeptides, dimers, multimers, and the like, whether produced biologically, recombinantly, or synthetically and whether composed of naturally occurring or non-naturally occurring amino acids, are included within the definition. Both full-length proteins and fragments thereof are encompassed by the definition. The terms also include co-translational (e.g., signal peptide cleavage) and post-translational modifications of the polypeptide, such as, for example, dissulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications may be deliberate, as through site-directed mutagenesis, or may be accidental, such as through mutations of hosts that produce the proteins, or errors due to PCR amplification or other recombinant DNA methods. Recombinant, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide with which it is associated in nature. The term recombinant as used with respect to a protein or polypeptide, means a polypeptide produced by expression of a recombinant polynucleotide. The term recombinant as used with respect to a host cell means a host cell into which a recombinant polynucleotide has been introduced.

As used herein, an “ErbB ligand” refers to a molecule in which at least a portion of the molecule comprises an ErbB ligand (i.e., a member of the EGF-like family of proteins which bind one or more ErbB receptors) or a fragment thereof. Non-limiting examples of ErbB ligands are betacellulin (BTC), epidermal growth factor (EGF), Epigen, amphiregulin (AR), transforming growth factor alpha (TGF-α), heparin-binding EGF (HB-EGF), epiregulin (EPR), and any of the multiple neuregulin isoforms and splice variants (e.g., NRG-1, NRG-2, NRG-3, or NRG-4). A receptor is defined by the International Union of Pharmacology Committee on Receptor Nomenclature and Drug Classification (NC-IUPHAR) as a protein, or a complex of proteins, which recognizes physiologically relevant ligands that can regulate the protein to mediate cellular events.

A “ligand” is any molecule that binds to a specific site on another molecule, including but not limited to receptors. For example, a ligand may be an extracellular molecule that, upon binding to another molecule, usually initiates a cellular response, such as activation of a signal transduction pathway.

A “fragment” is any portion or subset of the corresponding polypeptide or polynucleotide molecule. Thus, for example, a “fragment of albumin” refers to a polypeptide subset of albumin and a “fragment of Fc” refers to a polypeptide subset of an Fc molecule. The term “fragment” is not intended to limit the portion or subset to any minimum or maximum length.

A “variant” of an ErbB ligand is meant to refer to a ligand substantially similar in structure and biological activity to either the native ErbB ligand or to a fragment thereof, but not identical to such molecule or fragment thereof. A variant is not necessarily derived from the native molecule and may be obtained from any of a variety of similar or different cell lines. The term “variant” is also intended to include genetic alleles and glycosylation variants. Thus, provided that two ErbB ligands possess a similar structure and biological activity, they are considered variants as that term is used herein even if the composition or secondary, tertiary, or quaternary structure of one of the ligands is not identical to that found in the other.

“Long-acting” in relation to ErbB ligands refers to an ErbB ligand with a pharmacokinetic half-life that is longer than the half-life of the corresponding ErbB ligand alone. Similarly, the term “extended half-life” as used herein is a relative term that refers to a longer pharmacokinetic half-life in one form of a molecule relative to another form. The term “pharmacokinetic half-life” refers to the extent of time that it takes, after administration of the ErbB ligand of interest, for the concentration of the ErbB ligand to decrease to one half of its initial concentration (i.e., that reached upon administration) in the blood, plasma or other specified tissue.

A “fusion polypeptide” is one comprising amino acid sequences derived from two or more different polypeptides. For example, a “long-acting betacellulin fusion protein” is a fusion polypeptide comprising a betacellulin polypeptide, or an active variant or fragment thereof, and a fusion partner, or an active variant or fragment thereof. The fusion polypeptide hence comprises the protein of interested linked (e.g., recombinantly or by synthetic methods) to a second polypeptide, termed a “fusion partner.” Examples of commonly used fusion partners include, inter alia, albumin, Fc molecules, polypeptides comprising oligomerization domains, and various domains of the constant regions of the heavy or light chains of a mammalian immunoglobulin.

The terms “albumin” and “albumin molecule” refer to any one of a group of proteins that are soluble in water and moderately concentrated salt solution, and that are coagulable on heating. Suitable albumins will be familiar to those skilled in the relevant art. In addition, these proteins may be modified by proteolysis, sequence modification using molecular biological methods, and by binding to lipids or carbohydrates.

The term “Fc molecule” as used herein includes native and mutein forms of polypeptides derived from the Fc region of an antibody comprising any or all of the CH domains of the Fc region. As defined herein, an Fc molecule that is defective in effector function is one that does not induce antibody-dependent cell-mediated cytoxicity (ADCC). An antibody or an immunoglobulin is a protein that is capable of recognizing and binding to a specific antigen. Antibodies can generated by the immune system, synthetically, or recombinantly, and include polyclonal and monoclonal antibody preparations, as well as preparations including hybrid antibodies, altered antibodies, chimeric antibodies, hybrid antibody molecules, F(ab′)₂ and F(ab) fragments; Fv molecules (for example, noncovalent heterodimers), dimeric and trimeric antibody fragment constructs; minibodies, human antibodies, humanized antibody molecules, and any functional fragments obtained from such molecules, wherein such fragments retain specific binding. Antibodies are commonly known in the art. Antibodies may recognize, for example, polypeptide or polynucleotide antigens. The term includes active fragments, including for example, an antigen-binding fragment of an immunoglobulin, a variable and/or constant region of a heavy chain, a variable and/or constant region of a light chain, a complementarity-determining region (cdr), and a framework region. An antibody CH3 domain refers to the CH3 portion of an Fc molecule. Truncated forms of such polypeptides containing the hinge region that promotes dimerization are also included.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids, and the like, where the polymers can be naturally occurring or synthetic.

The term “succinyl group” as used herein refers to the acyl residue derived from succinic acid or (1,4-dioxobutyl)-1-carboxylic acid.

The term “oligomerization domain” refers to a portion of a fusion partner at which the formation of an oligomer may occur; i.e., there is sufficient structure to allow oligomerization. The oligomers can be of any subunit stoichiometry, including, for example dimerization and tetramerization domains. The oligomerization domain may comprise a coiled-coil domain (such as a tetranectin coiled-coil domain, a coiled-coil domain in a cartilage oligomeric matrix protein, an angiopoietin coiled-coil domain, or a leucine zipper domain), a collagen or a collagen-like domain (such as collagen, mannose-binding lectin, lung surfactant protein A, lung surfactant protein D, adiponectin, ficolin, conglutinin, macrophage scavenger receptor, or emilin), or a dimeric immunoglobulin domain (such as an antibody CH3 domain).

A “composition” or “pharmaceutical composition” herein refers to a composition that usually contains an excipient, such as a pharmaceutically acceptable carrier that is conventional in the art and that is suitable for administration into a subject for therapeutic, diagnostic, or prophylactic purposes. It can include a cell culture, in which the polypeptide or polynucleotide is present in the cells and/or in the culture medium. In addition, compositions for topical (e.g., oral mucosa, respiratory mucosa) and/or oral administration can form solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st ed.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents.

As used herein, the term “kit” refers to components packaged or marked for use together. For example, a kit can contain an ErbB ligand (e.g., betacellulin), another antidiabetic agent (e.g., a difference ErbB ligand), and a carrier, and these three components be in three separate containers. In another example, a kit can contain any two components in one container, and a third component and any additional components in one or more separate containers. Optionally, a kit further contains instructions for combining and/or administering the components so as to formulate a composition (e.g., a composition that increases glucose uptake and/or amino acid uptake into muscle cells) suitable for administration to a subject (e.g., an acutelly ill subject, a diabetic subject, a subject suffering from a cardiac disease).

The term “meal” refers to the food served and eaten at one time. The term encompasses both “meals” consumed at any of the occasions for eating food that occur by custom or habit at more or less fixed times (e.g., breakfast, lunch, dinner), as well as “meals” consumed at any other occasion (e.g., snacks).

A “disease” is a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

The terms “muscular disorders” or “muscular diseases” are intended to encompass muscular and neuromuscular disorders, including muscle wasting cachexia, sarcopenia, rhabdomyolysis, diaphragmatic weakness, and the like. Some of the muscular disorders are characterized by a destabilization or improper organization of the plasma membrane of specific cell types and include, but are not limited to, muscular dystrophies (MDs). MDs are a group of genetic degenerative myopathies characterized by weakness and muscle atrophy without nervous system involvement. The three main types of MD are pseudohypertrophic (Duchenne, Becker), limb-girdle (LGMD), and facioscapulohumeral. Several muscular dystrophies and muscular atrophies are characterized by a breakdown of the muscle cell membrane, i. e., they are characterized by leaky membranes resulting from a mutation in dystrophin. some of which can be treated by compensatory overexpression of utrophin. The term “muscular disorder” further encompasses Welander distal myopathy (WDM), Hereditary Distal Myopathy, Benign Congenital Hypotonia, Central Core disease, Nemaline Myopathy, and Myotubular (centronuclear) myopathy, as well as muscle wasting, sarcopenia, and muscular atrophies. Non-limiting examples of muscular atrophies are those resulting from AIDS-related wasting, from denervation (loss of contact by the muscle with its nerve) due to nerve trauma; degenerative, metabolic (e.g., metabolic myopathies, diabetic amyotrophy) or inflammatory neuropathy (e.g., Guillian Barre syndrome), peripheral neuropathy, and damage to nerves caused by environmental toxins or drugs; muscle atrophies that result from denervation due to a motor neuronopathy, including adult motor neuron disease, Amyotrophic Lateral Sclerosis (ALS or Lou Gehrig's disease); infantile and juvenile spinal muscular atrophies, and autoimmune motor neuropathy with multifocal conduction block; muscle atrophies that result from chronic disuse, including disuse atrophy stemming from conditions including, but not limited to: paralysis due to stroke, spinal cord injury; skeletal immobilization due to trauma (such as fracture, sprain or dislocation) or prolonged bed rest; and muscle atrophies resulting from metabolic stress or nutritional insufficiency, including, but not limited to, the cachexia of cancer and other chronic illnesses, rhabdomyolysis, and endocrine disorders such as, but not limited to, disorders of the thyroid gland and diabetes.

As used herein, the term “cardiovascular disorder” includes a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include congenital heart defects (e.g., atrioventricular canal defects), hypertension, atherosclerosis, coronary artery spasm, coronary artery disease, valvular disease, ischemia, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, long-QT syndrome, congestive heart failure, sinus node dysfunction, atrial flutter, myocardial infarction, coronary artery spasm, arrhythmias, and cardiomyopathies.

“Cardiotoxicity” includes clinical (e.g., clinical heart failure) and subclinical (e.g., abnormalities measured by diagnostic techniques) damage to the heart and/or the cardiovascular system (e.g., myocardial damage). “Induced cardiotoxicity” encompasses, inter alia, viral-induced cardiotoxicity, therapeutically-induced cardiotoxycity, heart damage caused by administration of otherwise therapeutic drugs such as, for example, viral-based drugs, anthracyclines/anthracycline analogs (e.g. doxorubicin, adriamycin) used in the treatment of cancer, cyclic antidepressants, calcium channel blockers, beta-blockers, oral contraceptives, anti-arrhythmic drugs, and digoxin.

The terms “subject,” “individual,” “host,” and “patient” are used interchangeably herein to refer to a living animal, including a human and a non-human animal. The subject may, for example, be an organism possessing immune cells capable of responding to antigenic stimulation, or possessing cells responding to stimulatory and inhibitory signal transduction through cell surface receptor binding. The subject can be a mammal, such as a human or a non-human mammal, for example, non-human primates, dogs, cats, pigs, cows, sheep, goats, horses, rats, and mice. The term “subject” does not preclude individuals that are entirely normal with respect to a disease, or normal in all respects, and includes both diabetic and nondiabetic subjects.

“Treatment” or “treating” as used herein, covers any administration or application of remedies for disease in a mammal, including a human, and includes inhibiting the disease. It includes arresting disease development and relieving the disease, such as by causing regression or restoring or repairing a lost, missing, or defective function, or by stimulating an inefficient or absent process. Herein, “treatment” also includes one or more of acute reduction of blood glucose level, regulation of basal level of glucose; or increase in survival, glucose uptake, amino acid uptake, utrophin expression, or glucose level in the muscle cells in a subject, or its muscle mass. A therapeutic agent is any agent used for treatment of a condition.

A “vial,” is used broadly herein, and is synonymous with cartridge, blister, and the like, and refers to any drug-packaging device that is designed and suitable for sealed and sterile storage, shipping, and handling of small (e.g., single-dosage, or multiple-dosage) quantities of pharmaceutical compositions (i.e., drugs).

Definitions for terms particularly relevant to blood glucose are set forth as follows.

The term “chronically effective serum level” as used herein refers to long-term maintenance of the serum level of a substance sufficient to regulate a serum component such as blood glucose, such as at least over a period of a day, or over one, two, or three days, or over a week, or over a month, or over a year.

The term “euglycemic level” is synonymous with normoglycemic level and refers to a normal level of blood glucose level, i.e., a blood glucose level in the range of about 50 to about 110 mg/dL.

The term “hypoglycemia” refers to a clinical conditions in which the adult human subject presents a blood glucose level below about 40-60 mg/dL (less than 2.2 mmol/l). Hypoglycemia in infants has been described by Comblath and Schwartz as whole blood glucose less than 30 mg/dL in term infants and 20 mg/dL in preterm infants (Comblath, M. and Schwartz, R., J. Pediatr. Endocrinol., 6: 113-129 (1993). Glucose concentrations in plasma or serum may be 10-15% higher than whole blood (Schwartz R. P., J. Pediatr.; 131:171-173 (1997)). In mice, the term hypoglycemia refers to blood glucose levels below about 50 mg/dL.

The term “hyperglycemia” refers to a blood glucose level in adult human subjects about or above 120 mg/dL (7 mmol/L). “Acute hyperglycemia” refers to a transient state in which a subject exhibits a blood glucose level of at least about 10 mmol/L. Other animals, such as mice, also exhibit hyperglycemic levels, as would be recognized by those in the art.

The term “diabetes,” as used herein, refers to a disease defined by the presence of chronically elevated blood glucose levels (hyperglycemia); the term includes all known forms of diabetes such as, for example, Type I and Type II diabetes, as well as variety of other types of diabetes (sometimes referred to as secondary diabetes), which are caused by various illnesses or medications. Depending on the primary process involved (e.g., destruction of pancreatic beta cells or development of peripheral insulin resistance), these types of secondary diabetes behave similarly to Type I or Type II diabetes. The most common are diseases of the pancreas that destroy the pancreatic beta cells (e.g., hemochromatosis, pancreatitis, cystic fibrosis, pancreatic cancer), hormonal syndromes that interfere with insulin secretion (e.g., pheochromocytoma) or cause peripheral insulin resistance (e.g., acromegaly, Cushing syndrome, pheochromocytoma), and diabetes induced by drugs (e.g., phenytoin, glucocorticoids, estrogens). The term also includes metabolic syndrome and pre-diabetic conditions.

The term “diabetic ketoacidosis” refers to a state of absolute or relative insulin deficiency in a subject aggravated by ensuing hyperglycemia, dehydration, and acidosis-producing derangements in intermediary metabolism. The most common causes of diabetic ketoacidosis (DKA) are underlying infection, disruption of insulin treatment, and new onset of diabetes. DKA is typically characterized by hyperglycemia over 300 mg/dL, low bicarbonate (<15 mEq/L), and acidosis (pH<7.30) with ketonemia and ketonuria.

The term “Type I diabetes” is synonymous with insulin-dependent diabetes (IDM), insulin-dependent diabetes mellitus (IDDM), growth-onset diabetes, type 1 diabetes, DM, diabetes, Type I DM, childhood diabetes, childhood diabetes mellitus, childhood-onset diabetes, childhood-onset diabetes mellitus, diabetes in childhood, diabetes mellitus in childhood, juvenile-onset diabetes, juvenile-onset diabetes mellitus, ketosis-prone diabetes, autoimmune diabetes mellitus, brittle diabetes mellitus, chamber-pot dropsy, thirst disease, sugar disease, sugar sickness. Type I diabetes mellitus can occur at any age and typically is characterized by the marked inability of the pancreas to secrete insulin because of autoimmune destruction of the beta cells. It commonly occurs in children, with a fairly abrupt onset. However, newer antibody tests have allowed for the identification of more people with the new-onset adult form of Type I diabetes mellitus called latent autoimmune diabetes of the adult (LADA). The distinguishing characteristic of a patient with Type I diabetes is that, if his or her insulin is withdrawn, ketosis and eventually ketoacidosis develop. Therefore, these patients are dependent on exogenous insulin.

The term “Type II diabetes” is synonymous with type 2 diabetes, non-insulin dependent diabetes mellitus (NIDDM), and adult-onset diabetes. Currently, because the epidemic of obesity and inactivity in children, Type II diabetes is occurring at younger ages. Although Type II diabetes typically affects individuals older than 40 years, it has been diagnosed in children as young as 2 years of age who have a family history of diabetes. Type II diabetes is characterized by peripheral insulin resistance with an insulin-secretory defect that varies in severity. For Type II diabetes to develop, both defects must exist: all overweight individuals have insulin resistance, but only those with an inability to increase beta-cell production of insulin develop diabetes. In the progression from normal glucose tolerance to abnormal glucose tolerance, postprandial glucose levels first increase. Eventually, in hepatic gluconeogenesis increases, resulting in fasting hyperglycemia. About 90% of patients who develop Type II diabetes are obese. Maturity-onset diabetes of the young (MODY) is a form of Type II diabetes.

The term “diabetic coma” refers to a medical emergency in which a person is comatose (unconscious) because the blood glucose levels are either too low or too high; the coma is usually the result of one of three acute complications of diabetes, namely (i) severe diabetic hypoglycemia, (ii) advanced diabetic ketoacidosis advanced enough to result in unconsciousness from a combination of severe hyperglycemia, dehydration and shock, and exhaustion, and (iii) hyperosmolar nonketotic coma in which extreme hyperglycemia and dehydration alone are sufficient to cause unconsciousness.

Subjects in “acutely ill settings” encompass, inter alia, medical patients with congestive heart failure, respiratory illness, infectious or inflammatory diseases, as well as postoperative, trauma, head-injury, burn, and medical intensive care unit (ICU)-patients.

An “antidiabetic agent” or an “anti-diabetic agent,” as used herein, is a substance that permits control of the level of glucose (sugar) in the blood (i.e., is useful in glycemic control). The activity of an antidiabetic agent can be assessed in vitro and in vivo by methods standard in the art such as, for example, by measuring its effect on blood glucose levels and/or hemoglobin A1c (HbA_{1c}) levels. Non-limiting examples of antidiabetic agents include insulin, insulin mimetics, insulin analogues, biguanides (e.g. metformin, phenformin), meglitinides (e.g. repaglinide), biguanide/glyburide combinations (e.g., Glucovance®), oral hypoglycemic agents (including inhaled agents that lower glucose levels), insulin secretagogues, incretins, insulin sensitizers (e.g., metformin, glitazones, and thiazolidinediones), alpha-glucosidase inhibitors (e.g., acarbose or miglitol), sulfonylureas (e.g., glimepiride, glyburide, gliclazide, chlorpropamide and glipizide), beta-cell secretagogues, glucagon-like peptide (GLP-1 and GLP-2), GLP-1 analogs (e.g., acylated GLP-1, CJC-1131, LY307 161 SR) administered with or without dipeptidyl peptidase IV (DPP-IV) inhibitors, DPP-IV inhibitors, thiazolidinediones (e.g., troglitazone, rosiglitazone and pioglitazone), PPAR-α agonists, PPAR-γ agonists, PPAR-α/γ dual agonists, glycogen phosphorylase inhibitors, inhibitors of fatty acid binding protein (aP2), sodium glucose co-transporter 2 (SGLT2) inhibitors, and non-steroidal anti-inflammatory agents (e.g., salicylates) that enhance glucose-induced insulin release. Dipeptidyl peptidase IV (DPP-4) is a membrane bound non-classical serine aminodipeptidase which is located in a variety of tissues (intestine, liver, lung, kidney) as well as on circulating T-lymphocytes (where the enzyme is known as CD-26). It is responsible for the metabolic cleavage of certain endogenous peptides (GLP-1(7-36), glucagon) in vivo and has demonstrated proteolytic activity against a variety of other peptides (GHRH, GIP, NPY, GLP-2, VIP) in vitro.

The term “reducing hypoglycemia associated with insulin administration in a subject” as used herein refers to avoiding, minimizing or averting exposing a subject to hypoglycemia resultant from insulin administration; such avoidance, reduction, or minimization can be achieved by, for example, providing to a subject a non-insulin treatment that subsequently reduces or eliminates the subject's additional need/demand for insulin.

“Normal insulin level” includes physiologically normal insulin levels, as well as any normal insulin level that has been achieved by treatment with any agent, including treatment with an antidiabetic agent.

As used herein, the term “insulin” means the insulin of any species, including, but not limited to, the following species: human, cow, pig, sheep, horse, dog, chicken, duck or whale. The insulin can be provided by natural, synthetic, or genetically engineered sources, and it can be monomeric and/or polymeric (e.g, hexameric), a lente insulin and/or a Neutral Protamine Hagedorn (NPH) insulin.

As used herein, the term “insulin analog” means insulin wherein one or more of the amino acids have been replaced while retaining some or all of the activity of the insulin; it also includes fatty acid acylated insulins such as, for example, those described in Guthrie, R. Clinical Diabetes 19:66-70 (2001)). Insulin analogs may be obtained by various means, as will be understood by those skilled in the art. For example, certain amino acids may be substituted for other amino acids in the insulin structure without appreciable loss of interactive binding capacity with structures such as, for example, receptors, antigen-binding regions of antibodies or binding sites on substrate molecules. As the interactive capacity and nature of insulin defines its biological functional activity, certain amino acid sequence substitutions can be made in the amino acid sequence, and the resulting protein remain a polypeptide with like properties. Non-limiting examples of insulin analogs include insulin glargine, insulin Lys-Pro/lispro (e.g., Humalog®; Eli Lilly and Company), insulin detemir, insulin aspart (e.g., NovoLog®; Novo Nordisk, Princeton, N.J.), NN304 (ε-LysB29-myristoyl, des [B30] human insulin), and fatty acid modified [Ne-palmitoyl Lys (B29)]-human insulin.

The terms “insulin mimetic” or “insulino-mimetic,” as used herein, refer to molecules, some of which are synthetic molecules, that react with insulin receptors (and thereby mimic the action of insulin), and lead to a reduction in blood glucose levels and/or increase insulin sensitivity. Non-limiting examples of such compounds can be found at Srivastava A K and Mehdi M Z., Diabet Med. 22(1):2-13 (2005), some of which comprise selenium, sulfonylureas (e.g. Amaryl), or vanadium. Insulin mimetics can have a variety of pharmacokinetic, activity, and bioavailability profiles, and include both short-acting and long-acting compounds.

“Insulin secretagogues” are drugs that increase endogenous insulin secretion. Endogenous insulin secretion can be assessed by, for example, measuring the levels of endogenous circulating insulin C-peptide in the blood, which is a product of proinsulin processing during its cellular expression. Some insulin secretagogues work by acting on K/ATP channels on the surface of the pancreatic beta-cells; they can vary in many aspects, such as their dependency on glucose concentrations, and in that some act rapidly but for a short time, whereas others act more slowly but for prolongued periods. The insulin secretagogues include the sulphonylureas, meglitinides, and D-phenylalanine derivatives, the rapid-acting insulin secretagogues nateglinide and repaglinide, and the like.

A “co-secreted agent” is a molecule that is secreted at the same time or at nearly the same time as another secreted protein or agent. Secreted proteins are generally capable of being directed to the endoplasmic reticulum (ER), secretory vesicles, or the extracellular space as a result of a secretory leader, signal peptide, or leader sequence. They may be released into the extracellular space, for example, by exocytosis or proteolytic cleavage, regardless of whether they comprise a signal sequence. A secreted protein can, in some circumstances, undergo processing to a mature polypeptide. Secreted proteins may comprise leader sequences of amino acid residues, located at the amino-terminus of the polypeptide and extending to a cleavage site, which, upon proteolytic cleavage, result in the formation of a mature protein. The leader sequence can be the sequence endogenous to the protein as it is encoded by its gene, or it can be a leader sequence from another protein (i.e. heterologous signal/leader sequence), which is operably linked to the sequence encoding the mature protein.

The description herein is put forth to provide those of ordinary skill in the art with a detailed description of how to make and how to use the present invention, and is not intended to limit the scope of what the inventors regard as their invention, nor is it intended to represent that the experiments set forth are all or the only experiments performed.

While the present invention is described with reference to the specific embodiments thereof, it should be understood by those skilled in the art that various changes may be made and equivalents may be substituted without departing from the true spirit and scope of the invention. In addition, many modifications can be made to adapt to a particular situation, material, composition of matter, process, process step or steps, to the objective, spirit, and scope of the present invention. All such modifications are intended to be within the scope of the claims appended hereto.

Unless defined otherwise, the meanings of all technical and scientific terms used herein are those commonly understood by one of ordinary skill in the art to which this invention belongs.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges including either or both of the upper and lower limits of the range, unless specifically excluded from the stated range.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The specification is most thoroughly understood in light of the cited references, all of which are hereby incorporated by reference in their entireties.

B. Regulation of Glucose Uptake, Disposal and Metabolism

B.1. Glucose Uptake by Insulin-Responsive Tissues and Cells

Normally, skeletal muscle is the principal site of glucose uptake under insulin-stimulated conditions, accounting for approximately 75% of glucose disposal following glucose infusion. Insulin responses are initiated through the binding to and activation of an insulin receptor at the cell surface. Once activated, the insulin receptor phosphorylates a number of signaling proteins, including insulin receptor substrates (IRSs).

There are many downstream events after insulin receptor activation. Ultimately, glucose uptake in muscles is accomplished by translocation of a glucose transporter (GLUT4) to the cell surface, which involves activation of a phosphoinositide 3-kinase (PI3K) by an IRS. In Type II diabetic patients, the skeletal muscles do not effectively respond to insulin, becoming insulin resistant. Reportedly, this resistance is partly caused by defects in the insulin-signaling pathway; some of these defects appear to be reversible. Thus, in Type II diabetic patients, one of the major defects in glucose regulation is the reduced level of glucose transport in the skeletal muscle after insulin stimulation.

Currently, thiazolidinediones are the only drug class of insulin sensitizers that promote skeletal muscle glucose uptake. However, thiazolidinediones cause hepatotoxicity, fluid retention, and potential exacerbation of heart failure in some patients.

B.2. Non-Insulin Dependent Glucose Uptake by Cells

In addition to insulin-mediated glucose uptake (IMGU), glucose uptake and disposal in humans also occurs as a result of non-insulin-mediated glucose uptake (NIMGU). In normal individuals, approximately 75% of glucose disposal under euglycemic conditions occurs as a result of NIMGU, primarily in the central nervous system and, to a lesser extent, in other tissues such as the splanchnic bed, blood cells, the peripheral nerves, and skeletal muscle (see Meneilly G S et al., Diabetes Care 24:1951-1956 (2001), and references therein). Under hyperglycemic conditions, the proportion of NIMGU occurring in skeletal muscle increases substantially, and the quantitative importance of NIMGU to overall glucose disposal is similar to the quantitative importance of IMGU. In insulin-resistant conditions, such as diabetes, approximately 80% of glucose uptake after a meal occurs as a result of NIMGU. The mechanisms of non-insulin mediated glucose disposal, however, are largely unknown.

In skeletal muscle, it appears that there are at least two alternative pathways of glucose uptake that can compensate for the lack of insulin signaling, including the Igflr-mediated pathway (Shefi-Friedman L et al., Am. J. Physiol. Endocrinol. Metab. 281:E16-E24 (2001)) and the contraction-activated signaling (Wojtaszewski J F et al. J. Clin. Invest. 104:1257-64 (1999)). There are at least two alternative pathways regulating GLUT4 translocation, and leading to glucose uptake and utilization in muscle, independently of insulin. Contraction is a powerful trigger to GLUT4 translocation through activation of the AMP-activated kinase. Muscle Igflr signals through IRSs proteins and PI3-kinase to stimulate GLUT4 translocation via activation of Akt and other inositoltrisphosphate (PIP₃)-dependent kinases, such as PKC isoforms.

C. High-Throughput Screening for Effects on Insulin-Signaling Pathway

Cultured cells are electrically active and their electrical resistance can be measured by growing the cells in assay wells equipped with microelectronic sensors. A commercially available cell-electrode impedance measuring system is the real-time, cell electronic system (RT-CES™ System) from ACEA Bioscience, Inc., (San Diego, Calif.).

The system comprises a multiwell tissue culture plate with integrated microelectronic sensors coupled to an impedance analyzer, which is, in turn, is coupled to a computer. It has been described in U.S. Patent Application Publication US 2004/0152067 A1. When a cell or the fluid in the well connects to electrodes in the sensor, the impedance analyzer measures the impedance resulting from alternating voltage applied across the electrodes. Cells seeded in the wells attach to the electrodes and change the resistance between the electrodes. Changes in the electrical resistance of the cells caused, for instance, by stimulation of a signaling pathway by binding of a ligand to its receptor, are measured as changes in impedance (Abassi et al., J. Immuno. Meth., 292: 195-205 (2004); Giaever et al., Proc. Nat'l. Acad. Sci., 81: 3761-3764 (1984)).

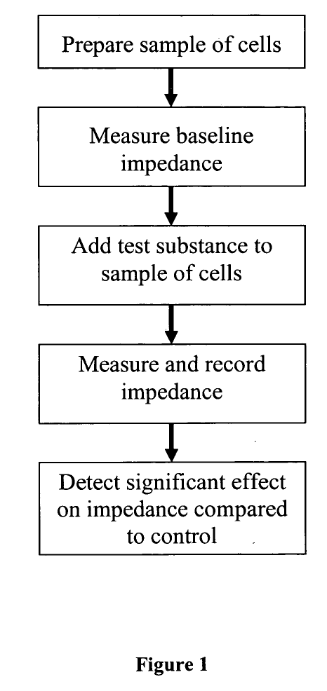

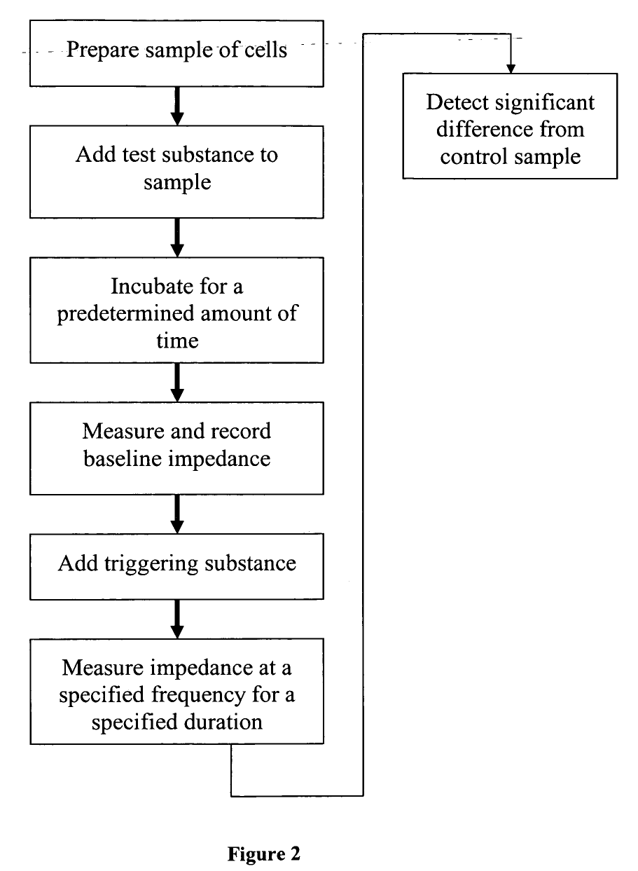

Impedance-measuring systems have been used for monitoring cell proliferation, cell toxicity, and receptor-ligand interaction. The RT-CES System calculates a normalized change in impedance resulting from the cells adhering to the microelectrodes and provides a baseline reading. The electrical response of the cells upon ligand addition can be measured in real time by adding the ligands to be tested to the culture well (Abassi et al., J. Immunol., Meth. 292: 195-205 (2004)). The overall steps of operating the real-time commercially available cell-electrode impedance-measuring system (RT-CES™ System) from ACEA Bioscience, Inc. (San Diego, Calif.) are depicted in FIG. 1 and FIG. 2.

The invention provides results obtained by further modifications of the method generally used by the RT-CES™ System. In general, rather than measuring only the change in the cell index after adding factors to the cell, the cells were instead incubated with test factors for 24 hours, and then insulin was added and the cell index was monitored for a response (FIG. 2).

D. Identification of ErbB Ligands as Insulin Modulators Using a High-Throughput Screening Method to Assess Effects on Insulin-Signaling Pathway

Using the modified impedance assay described above, several compounds were identified as affecting the insulin-signaling pathway and glucose uptake, as further described in the “Examples” section. One of these compounds is betacellulin, a protein in the ErbB ligand family.

As such, the present invention relates to ErbB ligand polypeptides and methods of using ErbB ligand polypeptides to treat hyperglycemia, diabetes and diseases which result (at least in part) from impaired glucose transport and/or metabolism. The invention accordingly provides compositions, and pharmaceutical combinations of compositions, comprising ErbB ligand polypeptides, and methods of using such compositions to stimulate glucose uptake.

D.1. ErbB Receptors and the ErbB Ligand Family of Proteins

The family of ligands for the ErbB receptors (herein referred to as the “ErbB ligand family,” and its members as ErbB ligands) is named after the cellular homologue of the viral erb gene, which in turn is one of three first RNAs of seven replication-defective leukaemia virus (DLV) strains originally identified as having the capacity to transform erythroblasts (hence the name erb) (Roussel, M. et al., Nature, 281: 452-5 (1979)).

Epidermal growth factor (EGF) is the prototype member of the ErbB ligand family. EGF binds the human EGF-Receptor 1 (HER1/ErbB1/EGFR) tyrosine kinase. Three other mammalian genes encoding receptors structurally similar to HER1 (ErbB1) have been identified and named HER2 (or ErbB2), HER3 (or ErbB3), and HER4 (or ErbB4). What the ErbB ligands have in common is the EGF domain, a consensus sequence of six spatially conserved cysteine (C) residues (CX7 CX4-5 CX10-13 CXCX8 C) that form three intramolecular dissulfide bonds (C1 to C3, C2 to C4, and C5 to C6). EGF contains six copies of the EGF domain. The other ErbB ligand family members contain only one, and one EGF domain is both necessary and sufficient for binding to and activation of a HER/ErbB. In addition to their ability to promote wound-healing, human genetic studies and targeted mutations in animal models indicate that EGF/HER complex family contains key players in multiple other biological processes. For example, EGFs dictate both neuronal and epithelial lineage differentiation during embryogenesis and some variants reportedly associate with schizophrenia, whereas sustained and inappropriate self-activation of HERs reportedly mediates signaling pathways that promote both epithelial cell survival and growth as well as angiogenesis in a significant proportion of lung and breast tumors.

Currently, the mammalian EGF family of ligands includes three groups of proteins. Group 1 members are capable of activating cells singly expressing HER1/ErbB1. These are: EGF (Savage et al., J. Biol. Chem., 247: 7612-7621 (1972)), transforming growth factor-a (TGF-α) (Marquardt et al., Science, 223: 1079-1082 (1984)), Epigen (Strachan L. et al. J Biol Chem. 276:18265-18271 (2001)), and amphiregulin (Shoyab et al., Science, 243: 1074-1076 (1989)). Group 2 members can activate cells singly expressing either HER1/ErbB1 or HER4/ErbB4, and includes heparin-binding EGF-like growth factor (HB-EGF) (Higashiyama et al., Science, 251: 936-939 (1991)), epiregulin (Toyoda et al., J. Biol. Chem., 270: 7495-7500 (1995)), and betacellulin (BTC) (Shing et al., Science, 259: 1604-1607 (1993)). Group 3 is the largest, and its members are capable of activating cells singly expressing either the HER3/ErbB3 or the HER4/ErbB4 receptor; this group includes the neuregulin (NRG) subfamily, which in humans is the product of four genes: NRG1 (Marchionni et al., Nature, 362: 312-318 (1993), NRG2 (Higashiyama et al., J. Biochem. 122(3):675-80 (1997); Chang et al., Nature, 387: 509-512 (1997); Carraway et al., Nature, 387: 512-515 (1997)), NRG3 (Zhang et al., Proc. Natl. Acad. Sci. (USA), 94:9562-9567(1997)), and NRG4 (Harari et al., Oncogene, 18: 2681-2689 (1999)). No direct HER2/ErbB2 ligand has been identified to date, although HER2/ErbB2 reportedly is indirectly activated by NRGs and BTC (Harris, C. R. et al., Exp. Cell Res., 284:2-13 (2003)) upon heterodimerization with other HER family members.

D.2. Betacellulin

As noted above, betacellulin is one example of an ErbB ligand protein which the inventors identified as a modulator of cellular insulin response. Betacellulin is a type I membrane protein that is translated as a transmembrane precursor molecule and proteolytically cleaved to a mature extracellular soluble form (for more details, see Example 41). The protease ADAM 10 can effect betacellulin shedding to the soluble form (Sanderson M. P. et al., J. Biol. Chem., 280: 1826-1837 (2005)). Betacellulin exists primarily as a monomer. The molecule folds into a configuration comprising an A loop, a B loop, and a C loop. The C loop is involved in receptor binding. Soluble mature betacellulin comprises 80 amino acids. The human betacellulin gene is located on chromosome 4 at band 4q13-q21.

Betacellulin contains one EGF-like domain, and its carboxyl terminal has approximately 50% homology with transforming growth factor-alpha (TGF-alpha). Betacellulin acts on epidermal growth factor receptors, though the exact receptors it may be working on in intestinal epithelial cells are unclear—perhaps ErbB1 or ErbB4 (Jones, J. T. et al., FEBS Letters, 447: 227-231 (1999)). A similar role has been reported for neuregulin-1 (also called heregulin beta1), which is also an ErbB ligand (Suarez, E. et al., J. Biol. Chem., 18257-18264 (2001)).

The inventors herein have discovered that betacellulin has a direct effect on muscle cells with the ensuing promotion of glucose uptake (e.g., skeletal muscle and cardiac muscle), survival, inhibition of apoptosis, utrophin expression, increase in muscle mass and other anabolic activities; and/or on insulin levels, or a combination of all of these activities, all of which are different from any prior described use of such protein.

E. Molecules, Compositions, their Therapeutic Applications and Methods of Use

E.1. Use of ErbB Ligands for Glycemic Control and to Treat Diseases that are Related to Glucose Transport and/or Glucose Metabolism

In a healthy individual, the beta-cells of the pancreatic islets of Langerhans produce insulin, which is required by the body for glucose metabolism, and is secreted in response to an increase in blood glucose concentration (e.g., after a meal, also referred to as the postprandial period). The insulin promotes both cellular uptake of glucose as well as metabolism of the incoming glucose, and temporarily halts the liver's conversion of glycogen and lipids to glucose, thereby allowing the body to support metabolic activity between meals. The Type I diabetic, however, has a reduced ability or absolute inability to produce insulin due to beta-cell destruction (e.g. autoimmune disease), and therefore needs to replace the insulin via multiple daily administrations (e.g. injections or insulin pumps). More common than Type I diabetes is Type II diabetes, which is characterized by insulin resistance and increasingly impaired pancreatic beta-cell function. Type II diabetics may still produce insulin, but they may also require insulin replacement therapy. Insulin resistance is a major contributor to progression of the disease and to many complications of diabetes, such as heart disease, muscle wasting and neuronal disease. Insulin resistance occurs, at least in part, because of a malfunction of the insulin-signaling pathway.

Type II diabetics typically exhibit a delayed response to increases in blood glucose levels. While normal persons usually release insulin within 2-3 min following the consumption of food, Type II diabetics may not secrete endogenous insulin for several hours after consumption. As a result, endogenous glucose production continues after consumption (Pfeiffer, Am. J. Med., 70: 579-88 (1981)), and the patient experiences hyperglycemia due to elevated blood glucose levels.

Most early stage Type II diabetics currently are treated with oral agents, but with little success. Subcutaneous injections of insulin are also rarely effective in providing insulin to Type II diabetics and may actually worsen insulin action because of delayed, variable, and shallow onset of action. It has been shown, however, that if insulin is administered intravenously with a meal, early stage Type II diabetics experience the desired shutdown of hepatic glucogenesis and exhibit increased physiological glucose control. In addition, their free fatty acids levels fall at a faster rate than without insulin therapy. While possibly helpful in treating Type II diabetes, intravenous administration of insulin is arguably an ineffective solution, as it is not safe or feasible for patients to intravenously administer insulin at every meal.

Insulin has pluripotent effects and may induce deleterious consequences, not just from causing hypoglycemia but also through other biologic actions. However, few other therapeutic proteins that can increase skeletal muscle glucose uptake have been identified to date. Thus, the current invention provides that a molecule (e.g., ErbB ligand) that can increase glucose uptake, dependently and/or independently of insulin receptors, can be beneficial to patients with Type II diabetes, who are either resistant to insulin or have impaired insulin sensitivity. Type I diabetic patients would also benefit from such a molecule because, even though their muscle cells can be responsive to insulin, the side effects of insulin or other diabetic agents are undesirable and, at times, even dangerous. By increasing the uptake of glucose independently of insulin, Type I diabetic patients would decrease their need for antidiabetic agents (e.g., agents for glycemic control), and therefore decrease the morbidity associated with those agents.

Diabetes, along obesity, is a metabolic disorder and as such can be accompanied by muscle wasting. Further, it has been reported that end stage renal disease patients with diabetes mellitus are more prone to muscle wasting and are at a high risk of hospitalization. The presence of diabetes mellitus is the most significant independent predictor of lean body mass loss in renal replacement therapy (Pupim, L. B. et al., Kidney Int., 68: 2368-2374 (2005)). Thus, it would be advantageous to ameliorate muscle wasting in this population of patients by improving their glycemic control and/or treating their diabetes.

Additionally, muscle wasting occurs in other subjects, such as in cancer patients, patients suffering from muscular dystrophy or sarcopenia in the aged population. It has been reported that cachexia affects nearly half of cancer patients, causing the clinical manifestations of anorexia, muscle wasting, weight loss, early satiety, fatigue, and impaired immune response. Cachexia is reportedly not reversed by increased caloric intake, signifying more complex mechanisms than simply caloric deficiency. It would be advantageous if muscle wasting could be prevented or ameliorated in this patient population (Esper, D. H. and Harb, W. A., Nutr. Clin. Pract., 20: 369-376 (2005)).

As such, the invention provides an ErbB ligand comprising a polypeptide sequence, wherein the polypeptide is betacellulin (BTC), epidermal growth factor (EGF), Epigen, amphiregulin (AR), transforming growth factor alpha (TGF-α), heparin-binding EGF (HB-EGF), epiregulin (EPR), or a neuregulin (NRG-1, NRG-2, NRG-3, or NRG-4); or an active variant or fragment of any of these. Some of these polypeptides are those comprising the sequences listed in SEQ. ID. NO. 4-6, 11, 13, 18-24, 27-89.

In one embodiment, the ErbB ligand enhances glucose uptake by muscle cells (e.g. skeletal muscle, heart muscle, smooth muscle cells); i.e., the ErbB ligand causes an increase in glucose uptake into muscle cells (e.g. skeletal, heart muscle, smooth muscle cells).

According to one embodiment, the activity of the ErbB ligand may also comprise sensitizing a cell to insulin, in other words, increasing a cell's sensitivity to insulin. Thus, for example, a cell's sensitivity to insulin may increase upon/after exposure to the ErbB ligand where a cell's response to a given amount of insulin increases relative to a prior measurement of the cell's response to the same amount of insulin.

In one embodiment, the ErbB ligand decreases insulin levels in a treated subject and may reduce the subject's need for insulin.

In another embodiment, the ErbB ligand improves amino acid uptake by muscle cells (e.g. skeletal, heart muscle, smooth muscle cells).

In an embodiment, the ErbB ligand upregulates utrophin expression in muscle cells (e.g. skeletal, heart muscle, smooth muscle cells).

According to one embodiment, the ErbB ligand is a long-acting ErbB ligand comprising (i) a first molecule that comprises an activity of the ErbB ligand and a (ii) second molecule that confers an extended half-life to the first molecule in a subject.

According to one embodiment, the first molecule of this long-acting ErbB ligand interacts with an ErbB receptor, such as ErbB1 or ErbB4 receptor. The interaction means that the two molecules form a complex that is relatively stable under physiologic conditions. Moreover, an ErbB receptor, such as an ErbB1 receptor and an ErbB4 receptor, is a receptor that specifically interacts with one or more ErbB ligands and/or fragments thereof.

In one embodiment, the long-acting ErbB ligand has an extended half-life in the subject that is at least 0.5 hours, or 1 hour, or 2 hours, or 3 hours, or 4 hours, or 5 hours longer than the half-life of the first molecule.

In one embodiment, the second molecule of the long-acting ErbB ligand comprises a polymer, a polypeptide, a succinyl group, or an albumin molecule.

In one embodiment, the polypeptide comprises a portion of an Fc molecule.

In one embodiment, the albumin molecule comprises an albumin, one or more fragments of albumin, a peptide that binds albumin, a molecule that conjugates with a lipid, or another molecule that binds albumin. In one embodiment, to bind means that two or more molecules form a complex that is relatively stable under physiologic conditions. In other words, a molecule forms a complex with albumin that is relatively stable under physiologic conditions. Conjugate is defined to encompass a molecule that is bound, either covalently or noncovalently, to another molecule. In one embodiment, for example, the albumin molecule is bound to a lipid molecule. The expression “another molecule that binds albumin” as used in this context refers to any molecule other than a peptide that binds albumin.

In one embodiment, the polymer comprises a polyethylene glycol moiety (PEG). Optionally, the polyethylene glycol moiety is either a branched or linear chain polymer. Furthermore, even if the polymer (e.g., PEG) is, directly or indirectly, covalently bound to the polypeptide, such covalent bond may be either permanent or transient/reversible.

In one embodiment, upon administration of the long-acting ErbB ligand to a subject, the polymer is released from the polypeptide (i.e., the drug); the kinetics and the conditions of such release may vary with physiological and pathological paramenters such as plasma, cellular and tissue pH, redox potential, and the like. Non-limiting examples of methods for transiently, or reversibly, pegylating drugs, including polypeptide-based drugs, are provided in U.S. Pat. No. 4,935,465 (issued in Jun. 19, 1990) and U.S. Pat. No. 6,342,244 (issued Jan. 29, 2002); and in U.S. published applications number US2006/0074024. One skilled in the art would typically find more details about PEG-based reagents in, for example, published applications WO2005047366, US2005171328, and those listed on the NEKTAR PEG Reagent Catalog® 2005-2006 (Nektar Therapeutics, San Carlos, Calif.).

In one embodiment, the second molecule of the long-acting ErbB ligand comprises an oligomerization domain. In one embodiment, the second molecule of the long-acting ErbB ligand comprises a molecule with improved receptor binding in a lysosome. Improved receptor binding refers to increased binding (i.e., increased affinity or avidity) to the receptor relative to the ErbB ligand alone.

Betacellulin: Expression and Purification

In one embodiment, the ErbB ligand is betacellulin. In one embodiment, the betacellulin is isolated human betacellulin, optionally an active fragment of human betacellulin, either modified or unmodified. The modification can include addition of an N-terminal Methionine residue for facilitation of expression in a prokaryotic expression system such as in E. coli. One skilled in the art would be familiar with several methods for producing betacellulin. In one embodiment, recombinant rat betacellulin can be purified as described by Dunbar et al. at the Cooperative Research Centre for Tissue Growth and Repair, CSIRO Health Sciences and Nutrition, Adelaide, Australia (Dunbar, A. J. et al., J. Mol. Endo. 27:239-247 (2001)); and by Folkman and Shing in U.S. Pat. No. 5,328,986. For example, rat betacellulin can be expressed in, and purified from, E. coli using a cleavable fusion protein strategy. Insoluble fusion protein can be collected as inclusion bodies and dissolved in urea under reducing conditions, re-folded, and purified by gel filtration chromatography and C₄ RP-HPLC. Both full-length and a truncated fragment of betacellulin can be obtained by proteolytically cleaving the fusion protein with Factor Xa; the biologically active fragment can be separated from full-length betacellulin by heparin-affinity chromatography.

In one embodiment, betacellulin can also be expressed in mammalian cells (e.g. CHO cells, 293 cells, PerC6® cells (Crucell, Netherlands)). In another embodiment, betacellulin can be isolated from mammalian tissues. It has been reported that betacellulin is synthesized by several tissue types, including pancreas, small intestine, kidney, and liver tissue, and tumor cell types, including a mouse beta tumor and the MCF-7 cell line (Sasada, R. et al., Biochem. Biophys. Res. Comm. 190:1173-1179 (1993)). High levels of expression have been observed in the pancreas and small intestine.

DNA Mutations and Amino Acid Sequence Variants

The present invention further relates to variants of the nucleic acid molecules of the present invention, which encode portions, analogs, or derivatives of the ErbB ligands of the invention.

Thus, non-limiting examples of a fragment, derivative, or analog of the ErbB ligands of the invention can be (i) one in which one or more of the amino acid residues are substituted with one or more conserved or non-conserved amino acid residue(s); such a substituted amino acid residue may or may not be one encoded by the genetic code; (ii) one in which one or more of the amino acid residues includes a substituent group; (iii) one in which the mature polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide, a leader or secretory sequence, a sequence employed to express or purify the above form of the polypeptide, or a proprotein sequence. Such fragments, derivatives, and analogs are deemed to be within the scope of those skilled in the art from the teachings herein.

In one embodiment, ErbB ligand variants can occur naturally, which encompasses splice variants (see, for example, Ogata, T. et al. Endocrinology 146: 4673-81. (2005); Dunbar A J and Goddard C., Growth Factors 18:169-75 (2000)); as well as natural allelic variants. Allelic variants include one of several alternate forms of a gene occupying a given locus on a chromosome of an organism, as described in, for example, Genes II, Lewin, B., ed., John Wiley & Sons, New York (1985), and the products of recombination. In one embodiment, non-naturally occurring variants can also be produced using mutagenesis techniques known in the art.

Accordingly, in one embodiment, allelic variants include those produced by nucleotide substitutions, deletions, or additions. The substitutions, deletions, or additions can involve one or more nucleotides. The variants can be altered in coding regions, non-coding regions, or both. Alterations in the coding regions can produce conservative or non-conservative amino acid substitutions (discussed in more detailed below), deletions or additions. These can take the form of silent substitutions, additions, or deletions which do not alter the properties or activities of the described ErbB ligand, or portions thereof.

In an embodiment, the invention provides nucleic acid molecules encoding mature ErbB ligands, including those with cleaved signal peptide or leader sequences. One embodiment includes an isolated nucleic acid molecule comprising a polynucleotide having a nucleotide sequence at least 70% identical, at least 80% identical, at least 90% identical, or at least 95% identical to one or more of the ErbB ligands of the invention (e.g., betacellulin), or a biologically active fragment of one or more of such ligands.

In one embodiment, a biologically active fragment of an ErbB ligand is one having structural, regulatory, or biochemical functions of a naturally occurring molecule or any function related to or associated with a cellular, metabolic or physiological process. Biologically active polynucleotide fragments are those exhibiting activity similar, but not necessarily identical to, an activity of a polynucleotide of the present invention.

In one embodiment, a biologically active polypeptide or fragment thereof includes one that can participate in a biological reaction, including, but not limited to, activation of one or more ErbB receptors, increase impedance in human skeletal muscle cells, modulation of a cellular response to insulin, stimulation of glucose uptake and/or amino acid uptake by muscle cells, upregulation of utrophin expression in muscle cells, promoting muscle cell survival, inhibiting muscle cell apoptosis, increasing muscle mass, in vivo glycemic control, regulation of HemoglobinA1c plasma levels, or a combination of any of the above. In another embodiment, a biologically active polypeptide is one that can serve as an epitope or immunogen to stimulate an immune response, such as production of antibodies; or that can participate in modulating the immune response. In one embodiment, the biological activity can include an improved desired activity, or a decreased undesirable activity.

In addition, in another embodiment, an entity demonstrates biological activity when it participates in a molecular interaction with another molecule, such as hybridization, when it has therapeutic value in alleviating a disease condition, when it has prophylactic value in inducing an immune response, when it has diagnostic and/or prognostic value in determining the presence of a molecule, such as a biologically active fragment of a polynucleotide that can, for example, be detected as unique for the polynucleotide molecule, or that can be used as a primer in a polymerase chain reaction.

A polynucleotide having a nucleotide sequence at least, for example, 95% identical to a reference nucleotide sequence encoding a ErbB ligand is one in which the nucleotide sequence is identical to the reference sequence except that it may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

In one embodiment, whether any particular nucleic acid molecule is at least 70%, 80%, 90%, or 95% identical to the ErbB ligands of the invention including betacellulin can be determined conventionally using known computer programs such as the Bestfit program (Wisconsin Sequence Analysis Package, Version 8 for Unix, Genetics Computer Group, Madison, Wis.). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In one embodiment, one or more of the nucleic acid molecules are at least 70%, 80%, 90%, or 95% identical to the ErbB ligands of the invention, including betacellulin, irrespective of whether they encode a polypeptide having an ErbB ligand activity as described herein. Even where a particular nucleic acid molecule does not encode a polypeptide having activity, one of skill in the art would know how to use the nucleic acid molecule, for instance, as a hybridization probe or a polymerase chain reaction (PCR) primer. Uses of the nucleic acid molecules of the present invention that do not encode a polypeptide having activity include, inter alia, isolating the gene or allelic variants thereof in a cDNA library; and in situ hybridization (for example, fluorescent in situ hybridization (FISH)) to metaphase chromosomal spreads to provide the precise chromosomal location of the ErbB ligand genes, as described in Verna et al., Human Chromosomes: A Manual of Basic Techniques, Pergamon Press, New York (1988); and Northern blot analysis for detecting their betacellulin mRNA expression in specific tissues.

In another embodiment, one or more nucleic acid molecules have sequences at least 70%, 80%, 90%, or 95% identical to a nucleic acid sequence of an ErbB ligand (such as betacellulin) and encode a polypeptide having polypeptide activity, that is, a polypeptide exhibiting activity similar but not necessarily identical, to an activity of the ErbB ligands of the invention, as defined above. In one embodiment, for example, the ErbB ligands of the present invention can stimulate glucose and/or amino acid uptake by muscle cells (e.g. skeletal, heart muscle, smooth muscle cells), utrophin expression, or both.

In another embodiment, and due to the degeneracy of the genetic code, one of ordinary skill in the art will immediately recognize that a large number of the nucleic acid molecules having a sequence at least 70%, 80%, 90%, or 95% identical to the nucleic acid sequence of one or more of the ErbB ligands of the invention will encode a polypeptide having activity. In fact, since multiple degenerate variants of these nucleotide sequences encode the same polypeptide, this will be clear to the skilled artisan even without performing the above described comparison assay. It will be further recognized in the art that a reasonable number of nucleic acid molecules that are not degenerate variants will also encode a polypeptide having activity. Thus, the skilled artisan is fully aware of amino acid substitutions that are either less likely or not likely to significantly affect protein function (for example, replacing one aliphatic amino acid with a second aliphatic amino acid), as further described below.

In one embodiment, protein engineering can be employed to improve or alter the characteristics of the ErbB ligands of the invention. Recombinant DNA technology known to those skilled in the art can be used to create novel mutant proteins or “muteins” including single or multiple amino acid substitutions, deletions, additions, or fusion proteins. In one embodiment, such modified polypeptides can show desirable properties, such as enhanced activity or increased stability. In one embodiment, such modified polypeptides can be purified in higher yields and show better solubility than the corresponding natural polypeptide, at least under certain purification and storage conditions. In one embodiment, non-limiting examples of betacellulin muteins are given in U.S. Pat. No. 6,825,165 (for example, SEQ ID NO. 1, 2, and 38 referred to therein).

In one embodiment the invention provides that, for many proteins, including the extracellular domain of a membrane associated protein or the mature form(s) of a secreted protein such as an ErbB ligand, one or more amino acids can be deleted from the N-terminus or C-terminus without substantial loss of biological function. One skilled in the art knows that, for instance, Ron et al., J. Biol. Chem., 268:2984-2988 (1993), reported modified KGF proteins that had heparin binding activity even if 3, 8, or 27 amino-terminal amino acid residues were missing. Similarly, many examples of biologically functional C-terminal deletion muteins are known. For instance, interferon gamma increases in activity as much as ten fold when 8-10 amino acid residues are deleted from the carboxy terminus of the protein, see, for example, Dobeli et al., J. Biotechnology, 7:199-216 (1988).

In one embodiment, even if deletion of one or more amino acids from the N-terminus or C-terminus of a protein results in modification or loss of one or more biological functions of the protein, other biological activities may still be retained. Thus, the ability of the shortened protein to induce and/or bind to antibodies which recognize the complete or mature from of the protein generally will be retained when less than the majority of the residues of the complete or mature protein are removed from the N- or C-terminus. Whether a particular polypeptide lacking N- or C-terminal residues of a complete protein retains such immunologic activities can be determined by routine methods described herein and otherwise known in the art. Accordingly, in one embodiment, the present invention further provides polypeptides having one or more residues deleted from the amino terminus of the amino acid sequences of the ErbB ligands of the invention.

In one embodiment, it also will be recognized by one of ordinary skill in the art that some amino acid sequences of the ErbB ligand polypeptides of the invention can be varied without significant effect on the structure or function of the protein. If such differences in sequence are contemplated, it should be remembered that there will be critical areas on the protein which determine activity.

In one embodiment, the invention includes variations of the ErbB ligands which show substantial ErbB ligand activity as described herein or which include regions of the ErbB ligands such as the protein portions discussed below. Such mutants include deletions, insertions, inversions, repeats, and type substitutions, selected according to general rules known in the art, so as have little effect on activity. For example, guidance concerning how to make phenotypically silent amino acid substitutions is provided in Bowie, J. U. et al., Science, 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections, or screens, to identify sequences that maintain functionality.

These studies report that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, et al., supra, and the references cited therein. Typically seen as conservative substitutions are the replacements, one for another, among the aliphatic amino acids Ala, Val, Leu, and Ile; hydrophobic substitutions Leu, Iso, and Val, interchange of the hydroxyl residues Ser and Thr, exchange of the acidic residues Asp and Glu, substitution between the amide residues Asn and Gln, exchange of the basic residues Lys, His, and Arg, replacements between the aromatic residues Phe, Trp, and Tyr, and between small amino acid substitutions Ala, Ser, Thr, Met, and Gly.

In one embodiment, amino acids involved in ErbB ligand functions can be identified by methods known in the art, such as site-directed mutagenesis or alanine-scanning mutagenesis, see, for example, Cunningham, B. C. and Wells, J. A., Science, 244:1081-1085 (1989). The latter procedure introduces single alanine mutations. In one embodiment, the resulting mutant molecules are then tested for biological activity including, but not limited to, receptor binding, or in vitro or in vivo promotion of glucose uptake by muscle cells (e.g. skeletal, heart muscle, smooth muscle cells), and up-regulation of utrophin expression in muscle cells.

In one embodiment, substitutions of charged amino acids with other charged or neutral amino acids can produce proteins with highly desirable improved characteristics, such as less aggregation. Aggregation may not only reduce activity but also be problematic when preparing pharmaceutical formulations, because, for example, aggregates can be immunogenic, Pinckard, R. N. et al., Clin. Exp. Immunol., 2:331-340 (1967); Robbins, D. C. et al., Diabetes, 36:838-845 (1987); Cleland, J. L. et al., Crit. Rev. Therapeutic Drug Carrier Systems, 10:307-377 (1993).

In one embodiment, replacing amino acids can also change the selectivity of the binding of a ligand to cell surface receptors. For example, Van Ostade, X. et al., Nature, 361:266-268 (1993) describes mutations resulting in selective binding of TNF-α to only one of the two known types of TNF receptors. In one embodiment, sites that are important for ligand-receptor binding can also be determined by structural analysis such as crystallization, nuclear magnetic resonance, or photoaffinity labeling, for example, Smith, L. J. et al., J. Mol. Biol., 224:899-904 (1992) and de Vos, A. M. et al., Science, 255:306-312 (1992).

In one embodiment, applying some of these common principles to the ErbB ligand betacellulin, we note that the sequence includes eight cysteine residues, located at amino acid positions number 7, number 28, number 69, number 77, number 82, number 93, number 95, and number 104. In one embodiment, the invention provides mutant betacellulin molecules with one or more cysteine residues mutated to, for example, serine residues. In one embodiment, these constructs can be cloned into any expression suitable vector, as known in the art, for example, the pTT5-G vector.

In another embodiment, analyzing these muteins provides an understanding of the disulfide bond pattern of betacellulin and may identify a protein with improved properties, for example, improved expression and secretion from mammalian cells, decreased aggregation of the purified protein, and the potential to produce active recombinant betacellulin, when expressed in E. coli.

Fusion Polypeptides

As discussed above, the inventors have found that betacellulin increases glucose and amino acid uptake into muscle cells, and has applications in treatment of different diseases, such as Type I and Type II diabetes. It can therefore be desirable to increase the half-life of betacellulin in vivo to produce a more sustained in vivo activity. Gene manipulation techniques have enabled the development and use of recombinant therapeutic proteins with fusion partners that impart desirable pharmacokinetic properties. Several different fusion partners have been used to produce fusion molecules. For example, recombinant human serum albumin fused with synthetic heme protein has been reported to reversibly carry oxygen (Chuang, V. T. et al., Pharm Res., 19:569-577 (2002)). The long half-life and stability of human serum albumin (HSA) makes it an attractive candidate for fusion to short-lived therapeutic proteins (U.S. Pat. No. 6,686,179). Thus, in one embodiment, the fusion partner comprises albumin. The albumin can include human serum albumin or a peptide that binds to or conjugates with a lipid or other molecule that binds albumin. These fusion partners can include any variant of or any fragment of such.

The Fc receptor of human immunoglobulin G subclass 1 (IgG1) has also been used as a fusion partner for a therapeutic molecule. It has been recombinantly linked to two soluble p75 tumor necrosis factor (TNF) receptor molecules. This fusion protein has been reported to have a longer circulating half-life than monomeric soluble receptors, and to inhibit TNF-alpha-induced proinflammatory activity in the joints of patients with rheumatoid arthritis (Goldenberg, M. M. Clin Ther., 21:75-87 (1999)). This fusion protein has been used clinically to treat rheumatoid arthritis, juvenile rheumatoid arthritis, psoriatic arthritis, and ankylosing spondylitis (Nanda, S. and Bathon, J. M., Expert Opin. Pharmacother., 5:1175-1186 (2004)). Thus, in one embodiment, the fusion partner can comprise an Fc fragment.

Fusion partners have also been produced comprising the first two domains of the human CD4-polypeptide and various domains of the constant regions of the heavy or light chains of mammalian immunoglobulins. See, for example, EP A 394,827; Traunecker, A. et al., Nature, 331:84-86 (1988). Fusion molecules that have a disulfide-linked dimeric structure due to the IgG part can also be more efficient in binding and neutralizing other molecules than, for example, a monomeric ErbB ligand polypeptide or polypeptide fragment alone. See, for example, Fountoulakis, M. et al., J. Biochem., 270:3958-3964 (1995).

Thus, the invention provides polypeptide fusion partners for ErbB ligands. In one embodiment, the fusion partners may be part of a fusion molecule, for example, a polynucleotide or polypeptide, which represents the joining of all or portions of more than one gene. As such, the invention can provide a nucleic acid molecule with a second nucleotide sequence that encodes a fusion partner. This second nucleotide sequence can be operably linked to the first nucleotide sequence. For example, a fusion protein can be the product obtained by splicing strands of recombinant DNA and expressing the hybrid gene.

In one embodiment, a fusion molecule can be made by genetic engineering, for example, by removing the stop codon from the DNA sequence of a first protein, then appending the DNA sequence of a second protein in frame. The DNA sequence will then be expressed by a cell as a single protein. In one embodiment, this is accomplished by cloning a cDNA into an expression vector in frame with an existing gene. The invention also provides fusion proteins with heterologous and homologous leader sequences, fusion proteins with a heterologous amino acid sequence, and fusion proteins with or without N-terminal methionine residues. The fusion partners of the invention can be either N-terminal fusion partners or C-terminal fusion partners.

In one embodiment, fusion polypeptides can be secreted from the cell by the incorporation of leader sequences that direct the protein to the membrane for secretion. These leader sequences can be specific to the host cell, and are known to skilled artisans; they are also cited in the references. Thus, the invention includes appropriate restriction enzyme sites for cloning the various fusion polypeptides into the appropriate vectors. In addition to facilitating the secretion of these fusion proteins, the invention provides for facilitating their production. This can be accomplished in a number of ways, including producing multiple copies, employing strong promoters, and increasing their intracellular stability, for example, by fusion with beta-galactosidase.

In one embodiment, the fusion partners can include linkers, i.e., fragments of synthetic DNA containing a restriction endonuclease recognition site that can be used for splicing genes. These can include polylinkers, which contain several restriction enzyme recognition sites. A linker may be part of a cloning vector. It can be located either upstream or downstream of the therapeutic protein, and it can be located either upstream or downstream of the fusion partner.

In one embodiment, protein expression systems known in the art can produce fusion proteins that incorporate ErbB ligand polypeptides. In one embodiment, the native form of the ErbB ligand have a shorter half-life than it is desirable for a given therapeutic use. In another embodiment, the invention provides for a long-acting ErbB ligand comprising a first molecule with ErbB ligand activity and a second molecule that confers an extended half-life to the first molecule.

In one embodiment, the first molecule can comprise any ErbB ligand family protein, or one or more of its fragments, which can be purchased from suppliers such as R&D System (Minneapolis, Minn.). In one embodiment, the first molecule can, for example, be an ErbB ligand, or a fragment thereof, for example one chosen from the molecules listed in Tables 1 through 4 of Example 41, or in the Appendix.

In one embodiment, the second molecule can facilitate production, secretion, and/or purification of the fusion molecule. In one embodiment, second molecules suitable for use in the invention include, for example, a polymer, a polypeptide, a succinyl group, or an albumin molecule. In one embodiment, the second molecule can comprise an oligomerization domain or a molecule with improved receptor binding in a lysosome.

In one embodiment, a long-acting ErbB ligand polypeptide of the invention can be prepared by attaching polypeptides or branch point amino acids to the ErbB ligand polypeptide. For example, the polypeptide may be a carrier protein that serves to increase the circulation half-life of the ErbB ligand polypeptide (i.e., in addition to the advantages achieved via an ErbB ligand fusion molecule). In one embodiment, such polypeptides do not create neutralizing antigenic response, or other adverse responses. Such polypeptides can be selected from serum album (such as human serum albumin), an additional antibody or portion thereof, for example the Fc region, or other polypeptides, for example poly-lysine residues. As described herein, the location of attachment of the polypeptide may be at the N-terminus, or C-terminus, or other places in between, and also may be connected by a chemical linker moiety to the selected ErbB ligand.

Such modified polypeptides can show, for example, enhanced activity or increased stability. In addition, they may be purified in higher yields and show better solubility than the corresponding natural polypeptide, at least under certain purification and storage conditions. In one embodiment, a human serum albumin-ErbB ligand fusion molecule may be prepared as described herein and as further described in U.S. Pat. No. 6,686,179.

In one embodiment, the invention also provides for facilitating the purification of these fusion proteins. Fusion with a selectable marker can, for example, facilitate purification by affinity chromatography. For example, fusion with the selectable marker glutathione S-transferase (GST) produces polypeptides that can be detected with antibodies directed against GST, and isolated by affinity chromatography on glutathione-sepharose; the GST marker can then be removed by thrombin cleavage. Polypeptides that provide for binding to metal ions are also suitable for affinity purification. For example, a fusion protein that incorporates Hisₙ, where n is between three and ten, inclusive, for example, a 6xHis-tag can be used to isolate a protein by affinity chromatography using a nickel ligand.

Other Polymer-Based Modifications, Derivatizations, Pegylations

According to one embodiment, conjugates of the ErbB ligands can be prepared using glycosylated, non-glycosylated or de-glycosylated ErbB ligand and fragments or variants thereof. Suitable chemical moieties for derivatization of ErbB ligand and variants of ErbB ligand include, for example, polymers, such as water soluble polymers described herein.

In one embodiment, polymers, including water soluble polymers, are useful in the present invention as the polypeptide to which each polymer is attached will not precipitate in an aqueous environment, such as a physiological environment. In one embodiment, polymers employed in the invention will be pharmaceutically acceptable for the preparation of a therapeutic product or composition. One skilled in the art will be able to select the desired polymer based on such considerations as whether the polymer/protein conjugate will be used therapeutically and, if so, the desired dosage, circulation time and resistance to proteolysis.

In one embodiment, polymers (e.g., water soluble polymers) can be of any molecular weight. In one embodiment, polymers can be branched or unbranched. In one embodiment, the polymers each can have an average molecular weight of between about 2 kDa to about 100 kDa. In another embodiment, the average molecular weight of each polymer is between about 5 kDa and about 50 kDa. In another embodiment, the average molecular weight of each polymer is between about 12 kDa and about 25 kDa. Generally, the higher the molecular weight or the more branches, the higher the polymer:protein ratio. In an embodiment, other sizes may be used, depending on the desired therapeutic profile, for example the duration of sustained release; the effects, if any, on biological activity; the ease in handling; the degree or lack of antigenicity and other known effects of a polymer on a modified ErbB ligand of the invention.

In one embodiment, suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly(β-amino acids) (either homopolymers or random copolymers), poly(n-vinyl pyrrolidone) polyethylene glycol, polypropylene glycol homopolymers (PPG) and other polyakylene oxides, polypropylene oxide/ethylene oxide copolymers, polyoxyethylated polyols (POG) (for example, glycerol) and other polyoxyethylated polyols, polyoxyethylated sorbitol, or polyoxyethylated glucose, colonic acids or other carbohydrate polymers, Ficoll or dextran and mixtures thereof.

In one embodiment, polyethylene glycol encompasses any of the forms that have been used to derivatize other proteins, such as mono-(C1-C10) alkoxy- or aryloxy-polyethylene glycol. In one embodiment, polyethylene glycol propionaldehyde may have advantages in manufacturing due to its stability in water.

In one embodiment, polymers employed in the present invention are attached to an ErbB ligand of the invention with consideration of effects on functional or antigenic domains of the polypeptide. In one embodiment, chemical derivatization can be performed under any suitable condition used to react a protein with an activated polymer molecule. In one embodiment, activating groups that can be used to link the polymer to the active moieties include the following: sulfone, maleimide, sulfhydryl, thiol, triflate, tresylate, azidirine, oxirane and 5-pyridyl.

In one embodiment, one (or more) polymers is attached to an ErbB ligand polypeptide of the invention at the alpha (α) or epsilon (ε) amino groups of amino acids. In one embodiment, the polymer(s) is(are) attached to a reactive thiol group. In one embodiment, the polymer(s) is(are) attached to any reactive group of the protein that is sufficiently reactive to become attached to a polymer group under suitable reaction conditions. Thus, in one embodiment, a polymer can be covalently bound to an ErbB ligand polypeptide of the invention via a reactive group, such as a free amino or carboxyl group. In one embodiment, the amino acid residues having a free amino group may include lysine residues and the N-terminal amino acid residue. In one embodiment, amino acids having a free carboxyl group may include aspartic acid residues, glutamic acid residues and the C-terminal amino acid residue. In one embodiment, amino acids having a reactive thiol group include cysteine residues.

In one embodiment, the invention provides methods of preparing ErbB ligands conjugated with polymers, including ErbB ligand fusion molecules conjugated with polymers, such as water soluble polymers, including: (a) reacting a protein with a polymer under conditions whereby the protein becomes attached to one or more polymers and (b) obtaining the reaction product.

Reaction conditions for each conjugation are well known by those skilled in the art, and may be selected from any of those known in the art or those subsequently developed, but should be selected to avoid or limit exposure to reaction conditions such as temperatures, solvents, and pH levels that would inactivate the protein to be modified. In general, the optimal reaction conditions for the reactions will be determined case-by-case based on known parameters and the desired result. For example, the larger the ratio of polymer:protein conjugate, the greater the percentage of conjugated product. The optimum ratio (in terms of efficiency of reaction in that there is no excess unreacted protein or polymer) can be determined by factors such as the desired degree of derivatization (for example, mono-, di-, tri- etc.), the molecular weight of the polymer selected, whether the polymer is branched or unbranched and the reaction conditions used. In one embodiment, the ratio of polymer (for example, PEG) to ErbB ligand polypeptide will range from 1:1 to 100:1. Molar ratios of activated polymer to protein of 2000:1 can also be used, depending on the concentration of the protein.

In one embodiment, one or more purified polymer conjugates can be prepared from each mixture by standard purification techniques, including among others, dialysis, salting-out, ultrafiltration, ion-exchange chromatography, gel filtration chromatography and electrophoresis.

In one embodiment, one may specifically prepare an N-terminal chemically modified protein. One may select a polymer by, for example, its molecular weight and/or its branching, the proportion of polymers to protein (or peptide) molecules in the reaction mix, the type of reaction to be performed, and the method of obtaining the selected N-terminal chemically modified protein. The method of obtaining the N-terminal chemically modified protein preparation (i.e., separating this moiety from other monoderivatized moieties if necessary) may be by purification of the N-terminal chemically modified protein material from a population of chemically modified protein molecules.

In one embodiment, selective N-terminal chemical modification can be accomplished by reductive alkylation that exploits differential reactivity of different types of primary amino groups (lysine versus the N-terminal) available for derivatization in a particular protein. In one embodiment, the present invention contemplates the chemically derivatized ErbB ligand polypeptide to include mono- or poly- (for example, 2-4) PEG moieties. “Pegylation” may be carried out by any of the pegylation reactions known in the art. There are a number of PEG attachment methods available to those skilled in the art. See, for example, U.S. Pat. No. 4,935,465 (issued in Jun. 19, 1990) and U.S. Pat. No. 6,342,244 (issued Jan. 29, 2002); U.S. published applications number US2006/0074024 EP 0 401 384; Malik, F. et al., Exp. Hematol., 20:1028-1035 (1992); Francis, Focus on Growth Factors, 3(2):4-10 (1992); EP 0 154 316; EP 0 401 384; WO 92/16221; WO 95/34326; and the other publications cited herein that relate to pegylation.

Pegylation by acylation generally involves reacting an active ester derivative of polyethylene glycol with an ErbB ligand polypeptide of the invention. In one embodiment, the activated PEG ester is PEG esterified to N-hydroxysuccinimide (NHS). In one embodiment, the linkage between the therapeutic protein and a polymer such as PEG is an amide, carbamate, urethane, and the like. See, for example, Chamow, S. M. Bioconjugate Chem., 5 (2):133-140 (1994). Pegylation by acylation will generally result in a poly-pegylated protein. In one embodiment, the resulting product is substantially only (for example, >95%) mono, di- or tri-pegylated. In another embodiment, some species with higher degrees of pegylation can be formed in amounts depending on the specific reaction conditions used.

Pegylation by alkylation generally involves reacting a terminal aldehyde derivative of PEG with the protein in the presence of a reducing agent. For the reductive alkylation reaction, the polymer(s) selected should have a single reactive aldehyde group. An exemplary reactive PEG aldehyde is polyethylene glycol propionaldehyde, which is water stable, or mono C1-C10 alkoxy or aryloxy derivatives thereof, see for example, U.S. Pat. No. 5,252,714.

Compositions

In one embodiment, the invention provides for a pharmaceutical composition comprising one or more polypeptides that stimulate glucose uptake into muscle cells (e.g. skeletal, heart muscle, smooth muscle cells) for treatment of a disease, and a pharmaceutically acceptable carrier, wherein one of the polypeptides is betacellulin.

In one embodiment, the invention provides for a pharmaceutical composition comprising one or more polypeptides that stimulate glucose uptake into muscle cells (e.g. skeletal, heart muscle, smooth muscle cells) for treatment of a disease, and a pharmaceutically acceptable carrier, wherein one of the polypeptides is an ErbB ligand.

In one embodiment, the invention provides for a pharmaceutical composition comprising a polypeptide that stimulates amino acid uptake into muscle cells (e.g. skeletal, heart muscle, smooth muscle cells) for treatment of a disease, and a pharmaceutically acceptable carrier or vehicle, wherein the polypeptide is an ErbB ligand.

In one embodiment, the invention provides for a pharmaceutical composition comprising a polypeptide that stimulates utrophin expression in muscle cells (e.g. skeletal, heart muscle, smooth muscle cells) for treatment of a disease, and a pharmaceutically acceptable carrier or vehicle, wherein the polypeptide an ErbB ligand.

In one embodiment, the invention provides for a pharmaceutical composition comprising a polypeptide that exhibits a significant anabolic effect in the muscle cells and/or muscle tissue of a subject, thereby changing the subject's body composition. In one embodiment, the subject's body composition changes by increasing skeletal muscle mass and reducing visceral fat. In one embodiment, such pharmaceutical composition can therefore prove useful as a human performance optimization agent. In one embodiment, such pharmaceutical composition can be used as a treatment for obesity, a condition frequently associated with diabetes.

In one embodiment, an ErbB ligand is a polypeptide that exhibits anabolic effect in the muscle.

Excipients and Formulations

In some embodiments, the compositions are provided in formulation with pharmaceutically acceptable carriers, a wide variety of which are known in the art. Gennaro, A. R. (2003) Remington: The Science and Practice of Pharmacy with Facts and Comparisons: DrugfactsPlus. 20th ed. Lippincott Williams & Williams; Ansel, H. C., et al., eds. (2004) Pharmaceutical Dosage Forms and Drug Delivery Systems 8th ed. Lippincott Williams & Wilkins; Kibbe, A. H., ed. (2000) Handbook of pharmaceutical Excipients, 3^{rd} ed. Pharmaceutical Press. “Pharmaceutically acceptable carriers,” such as vehicles, adjuvants, excipients, encapsulating material, auxiliary substances, or diluents, are readily available to the public. Moreover, pharmaceutically acceptable auxiliary substances, such as pH adjusting and buffering agents, tonicity adjusting agents, stabilizers, wetting agents and the like, are readily available to the public. Suitable vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents.

The U.S. Department of Health and Human Services of the Food and Drug Administration provides guidelines for estimating starting doses that are applicable for initial clinical trials on the basis of results obtained with animal tests. Thus, the publication “Guidance for Industry and Reviewers: Estimating the Safe Starting Does in Clinical Trials for Therapeutics in Adult Healthy Volunteers” (published in December 2002) can be used, along with other guidelines available to those of skill in the art, in order to properly design the concentration and dosages of the compositions provided in the invention.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, intranasal, rectal, enteral, parenteral, topical (e.g. gastrointestinal mucosa, oral mucosa, eye mucosa, respiratory mucosa), intraperitoneal, intradermal, transdermal, intramuscular, subcutaneous, intravenous, intra-arterial, intracardiac, intraventricular, intracranial, intratracheal, intrathecal administration, and the like; or otherwise by implanted catheter or pump, or provided via inhalation.

Agents that can be administered by injection refer to a formulation of the agent that will render it appropriate for parenteral administration, for example, intravenous, intraperitoneal, subcutaneous, intramuscular, intrathecal, intraorbital, intracapsular, intraspinal, intrasternal injection, or for local injection to a site of injury, damage or disorder. The injectable agent may comprise additionally to an effective amount of agent any pharmaceutically and/or physiologically acceptable solution, such as phosphate buffered saline that may be chosen by the physician handling the case according to standards known in the art. Thus, the subject compositions can be formulated into preparations in solid, semi-solid, liquid or gaseous forms, such as tablets, capsules, powders, granules, ointments, solutions, suppositories, injections, inhalants, and aerosols.

Agents for oral administration (i.e., an “oral agent”) can form solutions, suspensions, tablets, pills, granules, capsules, sustained release formulations, oral rinses, or powders. For oral preparations, the agents, polynucleotides, and polypeptides can be used alone or in combination with appropriate additives, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents. In addition, in an embodiment the composition may be administered intranasally using an inhalant. This composition will be formulated to allow for administration of pharmaceutically effective amounts to the lungs while minimizing damage to pulmonary tissue.

In one embodiment, the ErbB ligand family proteins (including all their variants and modifications described above), including betacellulin and the neuregulins, can also be delivered in time-release formulations (e.g. lipid and amino acid-based microspheres and microparticles) or delivery devices.

In one embodiment, the delivery device allows for local delivery to muscle cells, such as, local delivery to the cardiac muscle. In one embodiment, the local delivery to muscle cells is achieved using a catheter-based delivery system. In one embodiment, the delivery device involves remove magnetic steering. A non-limiting example of deliver device assisted by magnetic steering is a system comprising a Stereotaxis Niobe® Magnetic navigation system (Sterotaxis Inc., Maple Grove, Minn.), a Noga XP™ Cardiac Navigation system, and a magnetically enabled injection catheter. In one embodiment, the delivery system delivers the composition (e.g., a composition comprising one or more ErbB ligands) directly to one of the ventricles of the subject.

In one embodiment, the compositions include compositions which comprise a gel matrix, such as, for example, one of the hydrogel matrices known to those of skill in the art. Non-limiting examples of gel matrices include a collagen matrix which can comprise a poloxamer or an alginate.

In one embodiment, the ErbB ligand (e.g., betacellulin, long-acting betacellulin fusion protein) is formulated for oral delivery. Non-limiting examples of formulations that can be used for delivery of betacellulin and/or other ErbB ligands, include those formulations prepared for delivery of drugs via inhaler pumps, or via any other device for delivery of powders or aerosols which are known to those skilled in the art, such as those prepared by methods similar to those described in U.S. Pat. Nos. 5,740,794, 5,997,848, 6,051,256, 6,737,045, RE37872, and RE38385; or those described in U.S. Pat. Nos. 5,352,461, 5,503,852, 6,071,497, and 6,331,318; and in U.S. Published Applications 20040096403, 20060040953, each of which is incorporated herein by reference in its entirety for all that it teaches regarding diketopiperazines and diketopiperazine-mediated drug delivery. In one embodiment, the ErbB ligand (e.g. betacellulin) is delivered to the lung via an inhaler. In one embodiment, the ErbB ligand (e.g., betacellulin) is delivered to the lung via an inhaler in a powder formulation.

In one embodiment, the ErB ligand is formulated for oral delivery as a pill, capsule, or an equivalent thereof, which is absorbed through a gastrointestinal membrane. For example, the ErbB ligand (e.g., betacellulin) is formulated for oral delivery using one of the methods described in U.S. Pat. No. 7,005,141, 6,906,030, 6,663,898.

In one embodiment, the invention provides ErbB ligands that are formulated for the purposes of being provided (e.g., sold, stored, manufactured, prescribed, and the like) as parts of a kit. A kit refers to components packaged or marked for use together. In one embodiment, the invention provides a kit containing an ErbB ligand (e.g., betacellulin), optionally another antidiabetic agent (e.g., a difference ErbB ligand), and a carrier, and these two or three components be in two or three separate containers. In another example, a kit can contain any two components in one container, and a third component and any additional components in one or more separate containers. Optionally, a kit further contains instructions for combining and/or administering the components so as to formulate a composition (e.g., a composition that increases glucose uptake and/or amino acid uptake into muscle cells) suitable for administration to a subject (e.g., an acutelly ill subject, a diabetic subject, a subject suffering from a cardiac disease).

The following methods and excipients are merely exemplary and are in no way limiting.

Actual methods of preparing dosage forms are known, or will be apparent, to those skilled in the art (see Gennaro, A. R. (2003) Remington: The Science and Practice of Pharmacy with Facts and Comparisons: DrugfactsPlus. 20th ed.; and University of the Sciences in Philadelphia (2005) Remington: The Science and Practice of Pharmacy with Facts and Comparisons, 21st ed.). The composition or formulation to be administered will, in any event, contain a quantity of the agent adequate to achieve the desired state in the subject being treated.

In one embodiment, therapeutic formulations that comprise betacellulin and/or another of the ErbB ligands of the invention can be prepared for storage by mixing these proteins, having the desired degree of purity, with optional physiologically acceptable carriers, excipients, or stabilizers (Remington's Pharmaceutical Sciences, supra), in the form of lyophilized cake, dry powder, suspensions, aqueous solutions, and the like. In one embodiment, acceptable carriers, excipients or stabilizers are nontoxic to recipient subjects at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, lactose, mannose, or dextrins; chelating agents such as EDTA; sugar alcohols such as mannitol or sorbitol; salt-forming counterions such as sodium; and/or nonionic surfactants such as Tween, Pluronics or polyethylene glycol.

In one embodiment, one or more of the protein(s) described herein (e.g., betacellulin) can be complexed or bound to a polymer to increase its/their circulatory half-life for therapeutic administration. Non-limiting examples of polyethylene polyols and polyoxyethylene polyols useful for this purpose include polyoxyethylene glycerol, polyethylene glycol, polyoxyethylene sorbitol, polyoxyethylene glucose, or the like. In one embodiment, the glycerol backbone of polyoxyethylene glycerol is the same backbone occurring in, for example, animals and humans in mono-, di-, and triglycerides.

Methods of Use in Treatment of Diabetes, Hyperglycemia or Other Diseases

In one embodiment, the invention provides a method of treating diabetes by use of ErbB ligand polypeptides. The ErbB ligand family transmits signals through the ErbB receptors (for example, ErbB1, ErbB2, ErbB3, and ErbB4). In one embodiment, because the ErbB ligand family can stimulate glucose uptake in human muscle cells using this different signaling pathway, ErbB ligand polypeptides can be used for glycemic control. In particular, ErbB ligand polypeptides can be used to treat disorders in which insulin sensitivity is diminished or absent, such as Type II diabetes. Moreover, administration of ErbB ligand polypeptides such as betacellulin to patients with either Type I or Type II diabetes should improve glucose tolerance, irrespective of whether they are hyperinsulinemic (i.e., typical fasting insulin levels found in hyperinsulinism are above 20 μU/ml; when resistance is severe, levels can exceed 100 μU/ml), hypoinsulinemic (i.e., lower than normal insulin levels), or euinsulinemic (i.e., normal insulin levels). For example, ErbB ligand polypeptides like betacellulin will improve glucose tolerance, thereby reducing hyperglycemia, in diabetic patients with elevated levels of circulating insulin, but who fail to respond adequately to increasing levels of either endogenous or exogenously administered insulin due to insulin resistance. This patient population is separate and distinct from those patients who are insulin dependent and under adequate glycemic control or who can be brought into adequate glycemic control through increasing levels of endogenous or exogenous insulin.

Different ErbB ligand family members have different properties in stimulating glucose uptake. Some have very high receptor affinities, whereas others have low affinities but high maximum stimulated glucose uptake. Their receptor selectivity/specificity, bioavailability, kinetics, clearance rates, among other factors, can also vary. As such, different properties of this family members will increase the options for both short-term and long-term glycemic control (e.g. treatment of Type I and Type II diabetes).

In an embodiment, the invention provides compositions comprising betacellulin, which stimulate the uptake of glucose and amino acids into muscle cells without an increase of the uptake of one or both of these into fat cells. Thus, in one embodiment, betacellulin treatment does not lead to an increase in body fat as insulin or steroid treatment sometimes do.

Patients with all forms of diabetes mellitus have impaired glucose tolerance that in many instances is only partially treated by oral hypoglycemic agents (for example, sulfonylureas or PPAR gamma agonists) or proteins (for example, insulin, pramlintide acetate, or exenatide). In one embodiment, the invention provides a treatment for Type I or Type II diabetes, by further improving glucose tolerance.

Thus, the invention also provides for a method of glycemic control and/or treating diabetes (either Type I or Type II) in a subject by providing a composition comprising one or more of betacellulin (BTC), epidermal growth factor (EGF), Epigen, amphiregulin (AR), transforming growth factor alpha (TGF-α), heparin-binding EGF (HB-EGF), epiregulin (EPR), or a neuregulin (NRG-1, NRG-2, NRG-3, or NRG-4), or a biologically active fragment thereof; and administering a therapeutically effective amount of the composition to the subject more than once to “attain” (i.e., reach or achieve) or “maintain” (i.e., keep or continue at an existing level) a chronically effective serum level.

In one embodiment, only ErbB ligand polypeptides are administered, constituting monotherapy. Non-limiting examples of such composition are those that comprise one or more of betacellulin (BTC), epidermal growth factor (EGF), Epigen, amphiregulin (AR), transforming growth factor alpha (TGF-α), heparin-binding EGF (HB-EGF), epiregulin (EPR), or a neuregulin (for example, NRG-1, NRG-2, NRG-3, or NRG-4); all of these proteins can be present with or without a fusion partner.

The invention further provides for administration of pharmaceutical combinations of one or more compositions comprising an ErbB ligand and a pharmaceutically acceptable excipient. In one embodiment, the ErbB ligand of the pharmaceutical combination is betacellulin (BTC), epidermal growth factor (EGF), Epigen, amphiregulin (AR), transforming growth factor alpha (TGF-α), heparin-binding EGF (HB-EGF), epiregulin (EPR), or a neuregulin (for example, NRG-1, NRG-2, NRG-3, or NRG-4); or any fragment of variant thereof. In one embodiment, the ErbB ligand is a long-acting ErbB ligand. In one embodiment, one of the compositions further comprises another glucose-uptake stimulating molecule (different from the first molecule), such as insulin or any other molecule that stimulates glucose uptake, and which composition is combined (i.e. administered in conjunction, or before, after or concurrently with the first composition) with the composition comprising a first molecule that stimulates glucose uptake.

In one embodiment, the method of treating diabetes can treat a subject who is resistant to insulin. In one embodiment, the treatment can also result in reducing or delaying the need for insulin, reducing the need for an antidiabetic agent, and/or improving glucose homoeostasis. Reducing the need for an agent refers to decreasing the dosage of the agent necessary to achieve adequate glucose homeostasis. In one embodiment, the dosage may be decreased through, for example, decreasing the amount of agent administered at one time, by decreasing the frequency of administration, or both. Delaying the need for insulin refers to decreasing the frequency of insulin required to achieve adequate glucose homeostasis. In one embodiment, the need for insulin may be delayed because, for example, the subject maintains adequate glucose homeostasis for longer periods of time. Improving glucose homeostasis refers to improving the ability of the subject to maintain physiologically normal or near normal glucose levels, minimizing abnormal variations of glucose levels (for example, hypoglycemia and hyperglycemia).

In one embodiment, the invention sets forth a method of maintaining glucose homeostasis through small frequent dosages to achieve chronically effective serum levels and/or to acutely reduce serum glucose levels. In one embodiment, small frequent dosages are desirable because diabetics (both Type I and Type II) are unable to maintain normal glucose levels throughout the day. Glucose levels vary depending on factors such as food intake, daily activity, and exercise. Thus, where, for example, the patient expects to increase caloric intake or increase exercise activity, the treatment may be adjusted accordingly. As such, diabetic patients often test blood glucose three to four times per day, for example, upon waking up in the morning, before breakfast, before lunch, and before dinner. In one embodiment, before a meal, patients may determine how much glucose they expect to consume, and then vary the treatment accordingly. Thus, one embodiment provides a method for rapid reduction in glucose levels, within about 15 to about 90 min to thereby control post-prandial glucose. These methods contrast with other methods that disclose administering betacellulin to induce the regeneration of pancreatic insulin-producing beta-cells; furthermore, such methods would typically not require small frequent dosages of varying amounts.

In an embodiment, the dose of the glucose-lowering composition comprising one or more ErbB ligands such as, for example, betacellulin, can be adjusted based on a first glucose measurement, and then subsequently confirmed and/or potentially readjusted within 1 week (more preferably, 1 day) based on a re-measurement of, for example, both glucose levels and/or insulin levels. In one embodiment, the dosing can also be multiple times during the day, at least two or three times, for example, and the dose could be different at different times based on fluctuations in glucose levels measured at various times during the day. Thus, the dose could be administered within 2 hours of a meal or less, for example, within about 90, 60, 30, or 15 min of a meal, or during a meal.

In one embodiment, ErbB ligands (alone or in combination with other glucose-lowering and/or antidiabetic agents) can be used in the treatment of patients in the emergency or intensive care setting. In one embodiment, patients who are gravely ill from conditions including myocardial infarction, respiratory failure, congestive heart failure or other life-threatening conditions frequently experience acute severe hyperglycemia (Van den Berghe et al., 2001; Van den Berghe et al., 2006; supra). These patients have better outcomes when their hyperglycemia is treated aggressively, but are more vulnerable to the negative consequences of hypoglycemia as is associated with aggressive insulin treatment regimens. In one embodiment, patients can be treated with betacellulin (or other ErbB ligands alone or in combination therapy) in these (or other) acutely ill settings to prevent improve clinical outcome while reducing or eliminating insulin use thereby reducing the incidence of insulin induced hypglycemia. For this reason, in one embodiment, betacellulin can be administered in the ambulance or other non-hospital setting, where intravenous insulin would be too dangerous to be administered by a paramedic, and regular insulin would be too slow if given subcutaneously.

In an embodiment, the compositions are used in treatment and/or glycemic control in a setting of acute glucose decompensation. For example, patients who become severely hyperglycemic and who are at risk for diabetic ketoacidosis (DKA), or are in DKA, could use ErbB ligands (e.g. betacellulin with a short onset of action, for example, about 15-90 min) with very quick onset of action to return to a safer glucose range without the risk of hypoglycemia.

In addition to monotherapy, the invention further provides for combination therapy particularly with betacellulin administered in a short-acting form (onset of action within 15-30 min, duration of action 30-120 min). Such an acute combination may include agents such as, for example, insulin, insulin muteins such as lispro or glargine, or GLP-1 analogs such as exenatide or DPP IV inhibitors to acutely control blood glucose. Such acute control can prevent serious complications of severe, acute hyperglycemia such as diabetic ketoacidosis, diabetic coma, or incipient diabetic ketoacidosis.

In one embodiment, the dose of betacellulin can be adjusted on the basis of the severity of acute hyperglycemia obtained with each dose of betacellulin, or on the basis of longer term glucose levels. The later can include, for example, weekly measurements of blood glucose and/or measurements of hemoglobin A1c. The hemoglobin A1c test (also called H-b-A-one-c) is a simple lab test whose results are a measure of the average blood glucose over the previous three months. The hemoglobin Alc test shows if a person's blood sugar is close to normal or too high. It is an accepted test for monitoring long-term control of basal glucose level.

In one embodiment, ErbB ligands (e.g., betacellulin alone or in combination with other glucose-lowering agents), can be used to alleviate and/or reduce complications resulting from the use of insulin. For example, betacellulin can be co-administered with either long or short acting insulin to reduce the fluctuations in daily blood sugar, particularly in the post-prandial setting. The limited duration of action of betacellulin would allow the patient to reduce his or her short acting insulin dose at the time of a meal, thereby reducing the incidence of insulin-related hypoglycemic events. Patients taking mealtime insulin are at risk for hypoglycemia should a meal be missed following the dose of mealtime insulin. In one embodiment, betacellulin (alone or with other agents described herein) can also be used in lieu of mealtime insulin. In one embodiment, and due to betacellulin's lack of association with hypoglycemia (blood sugar <70 mg/dL) in euglycemic subjects (subjects in which the euglycemic level of blood glucose is about 50-110, it is predicted that the patient would not experience hypoglycemia with betacellulin monotherapy even if the meal is missed following the dose of betacellulin.

In one embodiment, the method of glycemic control (e.g. in treating diabetes) comprises administering a therapeutically effective amount of a composition comprising an ErbB ligand family member, such as betacellulin (BTC), with a second agent. These second agents, which may be termed “antidiabetic agents,” refer to a substance administered in addition to a first agent to treat diabetes, wherein the antidiabetic agent is a different molecule from the first agent. The different antidiabetic agents may comprise a hormone, a growth factor, a cytokine, or a chemokine. In one embodiment, the different antidiabetic agent comprises insulin, or betacellulin (BTC), or epidermal growth factor (EGF), or Epigen, or amphiregulin (AR), or transforming growth factor alpha (TGF-α), or heparin-binding EGF (HB-EGF), or epiregulin (EPR), or a neuregulin (NRG-1, NRG-2, NRG-3, or NRG-4), or a biologically active fragment thereof, with or without a fusion partner. In an embodiment, when the composition comprising betacellulin is administered in combination with neuregulin 1, at least one of betacellulin or neuregulin 1 comprises a fusion partner.

In one embodiment, the invention provides for a method of glycemic control (e.g., in treating diabetes) further comprising the oral administration of one or more antidiabetic agents before, after, or at the same time as the administration of the ErbB ligand. In one embodiment, these agents can comprise, for example, “metformin” (i.e., Glucophage®, a biguanide class antidiabetic agent), an insulin secretagogue, a glucosidase inhibitor, or a PPAR alpha-agonist. An insulin secretagogue is any drug composition that stimulates, participates in the stimulation of, or potentiates, the secretion of insulin by the pancreatic beta-cells. Insulin secretagogues include insulinotropic agents and insulin secretion or release potentiators, such as “sulfonylurea,” “meglitinide,” and “glucagon-like peptide.”

In one embodiment, the invention comprises the administration by injection of one or more second agents before, after, or at the same time as the ErbB ligand. These injectable agents include insulin, an insulin analogue, a cosecreted agent, “pramlintide” (i.e., Symlin®), synthetic human amylin), or a “DPP4 antagonist” (i.e., an inhibitor of dipeptidyl peptidase-IV protease). Moreover, the injectable agent may be administered in combination with a glucagon-like peptide, such as “exenitide.”

In an embodiment, the invention comprises the administration of an immunomodulatory agent as a second agent before, after, or at the same time as the ErbB ligand. An immunomodulatory agent is any of one or more substances that act to modulate the immune system of the subject being treated herein. The immunomodulatory agent may comprise an antibody such as an anti-CD3 antibody or an active variant thereof. An anti-CD3 antibody is any antibody that binds CD3 on T-lymphocytes. The antibody may also comprise a humanized monoclonal interleukin (IL)-2-R-alpha antibody such as daclizumab. To modulate refers to the production, either directly or indirectly, of an increase or a decrease, a stimulation, inhibition, interference, or blockage in a measured activity when compared to a suitable control. A modulator of a polypeptide or polynucleotide refers to a substance that affects (for example, increases, decreases, stimulates, inhibits, interferes with, or blocks) a measured activity of the polypeptide or polynucleotide, when compared to a suitable control.

In one embodiment, the immunomodulatory agent comprises a small molecule. Small molecules can be, inter alia, any chemical or other moiety, other than polypeptides and nucleic acids, that can act to affect biological processes. Small molecules can include any number of therapeutic agents presently known and used, or can be small molecules synthesized in a library of such molecules for the purpose of screening for biological function(s).

In one embodiment, the small molecule is “FK506” (i.e., Tacromilus, Fujimycin), which blocks T cell proliferation in vitro by inhibiting the generation of several lymphokines, especially IL-2, or “rapamycin” (i.e., Sirolimus, Rapamune), which blocks the ability of T-cells to proliferate in response to IL-2 stimulus. The immunomodulatory agent may also comprise sirolimus or a “suppressor of T- or B-cell activity or activation” (i.e., an agent that decreases the activity of T- or B-cell activity or activation, such as, for example, cyclosporine).

In the next series of embodiments, ErbB Ligands are useful for treating other disorders related to glucose metabolism, such as metabolic syndrome, obesity, muscle wasting and neural cell damage.

In one embodiment, the invention provides for a method of increasing muscle mass in a subject, comprising obtaining a composition containing an unmodified or a long-acting ErbB ligand and administering a therapeutically effective amount of the composition to the subject to increase muscle mass and thereby treat muscle wasting. As with the treatment methods described above, these methods may include both monotherapy and therapy accompanied by one or more agents (i.e. combination therapy). An increase in muscle mass refers to the increase in skeletal muscle cells and tissue through, for example, myocyte proliferation. The muscle wasting may be due to diabetic amyotrophy, or other metabolic myopathies, cachexia, AIDS-related wasting, disuse atrophy such as sarcopenia, or muscular dystrophy, such as Duchenne muscular dystrophy.

In one embodiment, the invention provides a method of ameliorating dystrophies caused by impaired function or reduced expression of the protein dystrophin, comprising obtaining and administering a therapeutically effective amount of certain ErbB ligands, for example betacellulin, to up-regulate the expression of utrophin in skeletal muscle cells, for example in human myoblasts. In one embodiment, administration of betacellulin increases muscle mass in subjects in need of such treatment by providing an anabolic function. In one embodiment, administration of betacellulin reduces muscle damage in subjects in need of such treatment by compensating for a loss of dystrophin with an induction of utrophin. In one embodiment, administration of betacellulin improves muscle function by increasing glucose and/or amino acid uptake into muscle cells.

In one embodiment, betacellulin's anabolic effect on cardiomyocytes can reduce cardiomyopathy associated with muscular diseases.

In another embodiment, the invention provides ErbB ligands for promoting the survival of cardiac muscle, and/or inhibiting the apoptosis of cardiac muscle, exposed to stress or damaging conditions. Non-limiting examples of stress/damaging conditions which could result in cardiac muscle cell death are nutrient and oxygen deprivation, and exposure to cardiotoxic drugs. Cardiotoxic drugs are well known to those of skill in the art of heart disease, and include several chemotherapeutic agents such as anthracyclins.

Obesity is another example of a metabolic disorder which, according to the invention, can be treated by ErbB ligands. In one embodiments, ErbB ligands promote glucose uptake and amino acid uptake into muscle cells without increase production of fat (i.e., without lipogenesis). In one embodiment, promotion of amino acid and/or glucose uptake by muscle cells can stimulate metabolic rate of a subject, thereby promoting catabolism and/or breakdown of adipose tissue.

Chronic hyperglycemia also leads to non-enzymatic glycation of matrix proteins, for example, in the vascular wall and the myocardium, producing advanced glycation end products (AGEs), and reactive oxygen species. AGEs promote cross-linkage of adjacent collagen polymers, leading to a loss of collagen elasticity and subsequently, diminished compliance of the blood vessels, as well as the heart muscle (myocardium), leading to heart failure. In one embodiment, by reducing glucose levels, the invention also provides methods of ameliorating heart failure, by alleviating damage to muscle and vessels caused by, for example, glucose-induced deposition of collagen in the tissue matrix, interstitial and perivascular fibrosis, increased left ventricular (LV) wall thickness and increased LV mass.

In one embodiment, the invention provides a method of regenerating or maintaining the integrity of neural cells in a subject, comprising obtaining a composition containing a long-acting ErbB ligand and administering a therapeutically effective amount of the ligand, for example, betacellulin to the subject to regenerate or maintain the integrity of neural cells. One considers an amount to be therapeutically effective if that amount will produce a desirable result upon administration; this will vary depending on various factors, such as the dosage to be administered and the route of administration. Furthermore, maintaining the integrity of a cell or population of cells means to maintain the condition of the cells by, for example, preventing cell injury or death. In one embodiment, the method treats a subject suffering from central nervous system disease, such as stroke, Alzheimer's disease, or Parkinson's disease. As with the treatment methods described above, these methods may include both monotherapy and therapy accompanied by one or more other agents.

The examples, which are intended to be purely exemplary of the invention, and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (for example, amounts and temperature), but some experimental errors and deviations should be accounted for. Unless indicated otherwise, parts are parts by weight, molecular weight is average molecular weight, temperature is in degrees Centigrade, and pressure is at or near atmospheric. Examples and references are given below to illustrate the present invention in further detail, but the scope of the present invention is not limited by these examples. Any variations in the exemplified articles which occur to the skilled artisan are intended to fall within the scope of the present invention. Experiments can be done with other ErbB family members, alone or in combination with other molecules (e.g., insulin, insulin mimetics, incretins, among others). Further examples of such combinations can be found throughout the specification, but would also be known to those of ordinary skill in the art in light of the present disclosure.

### EXAMPLES

### Example 1

### Cell Index in Response to Insulin Decreased in a Dose-Dependent Manner in L6 Muscle Cells

Our earlier impedance experiments (schematized in FIG. 1 and FIG. 2) showed that insulin decreased the cell index in a dose-dependent manner in rat L6 muscle cells, as measured in an impedance assay. That is, as the insulin concentration increased, the cell index decreased. The impedance assay was run using an RT-CES™ 16× device (ACEA Bioscience, Inc., San Diego, Calif.) substantially according to manufacturer's instructions, except where otherwise indicated. Briefly, each well of each 96 well plate was coated with 0.1% gelatin, and about 10⁴ rat L6 muscle cells (obtained from American Type Culture Collection “ATCC,” Manassas, Va., USA) were seeded into each well in alpha-minimum Eagle's medium containing 10% (v/v) fetal bovine serum, 100 units/ml penicillin G, 100 μg/ml streptomycin, and 0.25 μg/ml amphotericin B (hereafter, the “growth medium”). The cells were incubated overnight in a cell culture incubator at 37° C. and 5% CO₂. The next day, the growth medium was removed and was replaced with 135 μl of serum-free medium per well. The cells were again incubated for six hr. Insulin (human insulin, 100 units/ml; Eli Lilly and Company, Indianapolis, Ind.) in serum-free medium (15 μl) was then added to each well, and the cell index was measured immediately after addition of insulin. Insulin concentrations ranging from about 0.1 pM to about 1.0 μM, including doses of about 0.1 pM, 1 pM, 10 pM, 100 pM, 1 nM, 10 nM, 100 nM and 1.0 μM, were tested in triplicate. The cell index, as a measure of the changing impedance, was calculated by the RT-CES™ 16× device software. This example showed that the cell index decreased promptly, within a few min, after the addition of insulin to the L6 cells, and that the impedance assay can detect cell responses to changes in insulin concentrations. Basic fibroblast growth factor (“bFGF”) (Cat#234-FSE/CF) R&D Systems, Minneapolis, Minn.) was similarly tested (at concentrations between 0.1 pM and 1 μM) and was found to have no effect.

### Example 2

### Other Factors that Affect Insulin Signaling also Decrease Cell Index in L6 Cells

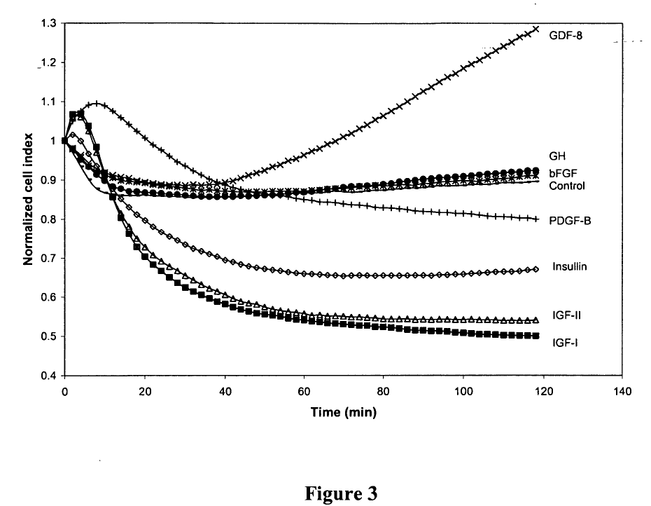

Our experiments further showed that other factors that affect the insulin-signaling pathway also decreased the cell index in L6 cells (measured in an impedance assay), an shown in this Example. L6 cells were plated in an RT-CES™ 16× device as described in Example 1. The tested factors were added separately to cells in the wells in 15 μl serum-free medium in place of insulin, as described in Example 1, at a concentration of about 100 nM each. Serum-free medium was used as a control. Cell index was measured in triplicate immediately after addition of factors. Thereafter, the measurement was continued over 120 min The results of this test, represented in FIG. 3, showed that insulin-like growth factors I (Cat# 291-G1) and II (Cat# 291-G2) (R&D Systems, Minneapolis, Minn.) decreased cell index to a greater extent than insulin 100 nM). Human PDGF-BB (Cat# 220-BB) (R&D Systems, Minneapolis, Minn.) also decreased cell index, but to a lesser extent than insulin (Eli Lilly and Company, Indianapolis, Ind.). Recombinant mouse GDF-8 (Cat# 788-G8; R&D Systems, Minneapolis, Minn.), which does not affect the insulin-signaling pathway, increased the cell index. No significant effect was observed for GH (recombinant human Growth Hormone; Cat# 1067-GH) and bFGF (R&D Systems, Minneapolis, Minn.).

### Example 3

### Pre-Incubation of Cells with Insulin, IGF-I, IGF-II, or PDGF-BB Inhibits a Subsequent Insulin-Induced Cell Index Response in L6 Cells

In Example 2, we showed that insulin and other factors involved in the insulin signaling pathway decreased the cell index in an impedance assay tested on L6 cells. We then tested the effect of pre-incubating the L6 cells with insulin, or with other factors that modulate the insulin-signaling pathway, on a subsequent response to insulin. This test was conducted as described in Example 1, but with either insulin, or with IGF-I, IGF-II, GDF-8, bFGF, PDGF-BB, or GH (R&D Systems, Minneapolis, Minn.), respectively, each at a final concentration of about 100 nM. Serum-free medium was used as a control. The cells were incubated with the factors for about 24 hr. After the 24 hr incubation, a baseline cell index was measured. Then, insulin was added to each well at a final concentration of about 100 nM and the cell index was measured immediately in triplicate. Results showed that the typical decrease in cell index previously exhibited by L6 cells when exposed to insulin was either not observed, or was significantly minimized, when L6 cells were pre-incubated with factors that affect the insulin-signaling pathway. In other words, pretreatment with either insulin, IGF-I, IGF-II, or PDGF-BB followed by insulin, all resulted in a higher cell index than insulin treatment alone, indicating that the insulin response in L6 cells pretreated with such factors was inhibited. On the other hand, no such impairment was observed upon L6 cell pretreatment with serum-free medium, GH, bFGF, or GDF-8. Thus, we found in this test that preincubating the L6 cells in this manner inhibited these cells from responding to a subsequent insulin stimulus, as measured by the impedance assay.

### Example 4

### Measurement of EC₅₀ of Insulin, IGF-I and IGF-II in an Impedance Assay in L6 Cells

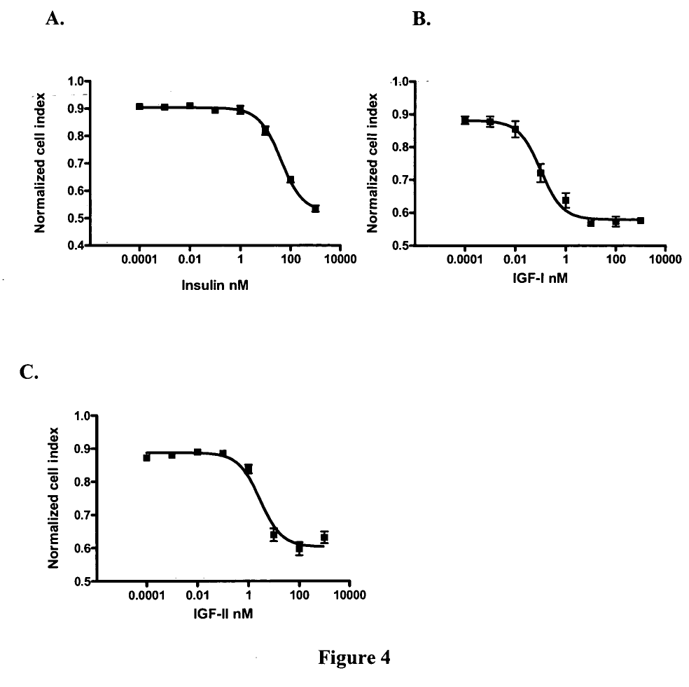

We tested the effective concentrations of insulin, IGF-I, and IGF-II, that results in 50% of the maximal effect (EC₅₀) as measured by the impedance assay and compared it to published EC₅₀ values as obtained by the ³H-deoxyglucose method (Hundal H S et al., Biochem J. 297: 269-295 (1994)) and found them to be about the same. In this test, rat L6 muscle cells (ATCC) were prepared for the impedance assay as described in Example 1. Insulin (FIG. 4A), IGF-I (FIG. 4B), or IGF-II (FIG. 4C), each in concentrations varying from 10⁻¹³ M to 10⁻⁶ M, was added to separate wells as in Examples 2 and 3. The cell index was measured in triplicate after 30 min of incubation with these factors. The impedance measurements showed that the EC₅₀ of insulin was approximately 41 nM; the EC₅₀ of IGF-I was approximately 102 pM; and the EC₅₀ of IGF-II was approximately 2.9 nM, all consistent with published values.

### Example 5

### Insulin Increases Cell Index in Human Primary Muscle Cells

We next tested the insulin response of a different cell type, namely primary human skeletal muscle cells (Cambrex, East Rutherford, N.J.), in the impedance assay. We found that insulin affected the cell index of primary human skeletal muscle cells in a dose-dependent manner, but in an opposite manner different from that in which it affects L6 cells. As the insulin concentration increased, the cell index increased as well. The cells were prepared for the impedance assay substantially as described in Example 1, and impedance was similarly measured using an RT-CES™ 16× device coated with 0.1% gelatin. Thus, about 3×10⁴ primary human skeletal muscle cells were seeded into each well in a growth medium for these cells (DMEM supplemented with 25 mM HEPES, 10% fetal calf serum, 2 mM glutamine, 0.5% chick embryo extract, 100 U/ml penicillin, 100 μg/ml streptomycin, and 0.25 μg/ml amphotericin B; the medium and supplements were also obtained from Cambrex). The cells were incubated overnight at 37° C. in 5% CO₂. The next day, the growth medium was replaced with 135 microliter of serum-free medium per well, and the cells were incubated for another six hr. Insulin (Eli Lilly and Company, Indianapolis, Ind.) in serum-free medium (15 microliter) was then added to each well and the cell index was measured immediately after addition of insulin. Insulin concentrations in increasing 10-fold increments, from 10⁻¹³ M to 10⁻⁶ M were tested in triplicate.

This test showed that, in primary human skeletal muscle cells, insulin increased the cell index in a dose-dependent manner. The highest cell index observed was at the highest insulin concentration tested, namely 10⁻⁶ M. At the lowest three concentrations, the insulin effect, as determined by the cell index, appeared to be about the same.

### Example 6

### EC₅₀ of Insulin, IGF-I, and IGF-II in Primary Skeletal Muscle Cells as Measured by the Impedance Assay is Consistent with Published Values

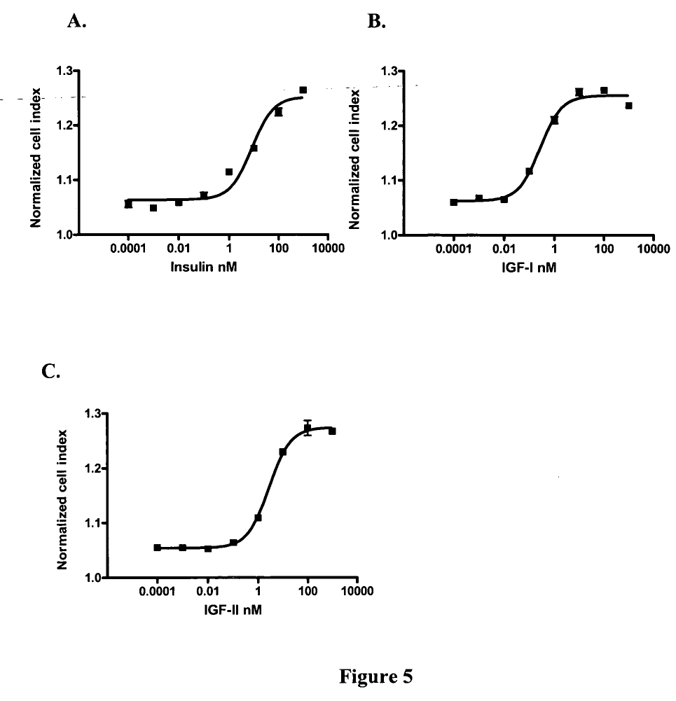

We measured the EC₅₀ of insulin, IGF-I, and IGF-II in primary human skeletal muscle cells (Cambrex, East Rutherford, N.J.) in an impedance assay. Cells were prepared as described in Example 5. Dose-response curves for insulin (FIG. SA), IGF-I (FIG. 5B), and IGF-II (FIG. 5C) were generated using concentrations ranging from about 0.1 pM to about 1.0 microM; each was tested in triplicate at 30 min. The EC₅₀ of insulin was found to be approximately 8.3 nM (see FIG. 5), showing that human primary skeletal muscle cells exhibited approximately five-fold greater sensitivity to insulin than did cultured L6 muscle cells (EC₅₀=41 nM, shown in Example 4 and FIG. 4). In human primary skeletal muscle cells, the EC₅₀ of IGF-I was approximately 270 pM (lower sensitivity to IGF-1 than L6 cells) and the EC₅₀ of IGF-II was approximately 2.7 nM (similar sensitivity to IGF-II in L6 cells).

### Example 7

### IGF-II Inhibits Insulin Response in Primary Muscle Cells

As discussed in Example 3, preincubation of rat L6 cells with IGF-II inhibited a subsequent response to insulin, and we demonstrate herein that IGF-II has a similar effect on primary human skeletal muscle cells.

Primary human skeletal muscle cells (Cambrex, East Rutherford, N.J.) were prepared for an impedance assay as described in Example 5. IGF-II (R&D Systems, Minneapolis, Minn.) was added in concentrations ranging from about 10⁻¹³ M to about 10⁻⁶ M. Cells were incubated for 24 hr at 37° C. and in 5% CO₂, as described before. After 24 hr, a baseline cell index was recorded. Then, insulin was added to a final concentration of about 100 nM and the cell index was measured at 30 min in triplicate. Results of this test showed that, as the pre-incubation concentration of IGF-II increased, the effect of insulin on the cell index decreased. In other words, IGF-II pretreatment impaired, i.e. lowered the magnitude of the increase in cell index which was previously observed when human primary skeletal muscle cells were exposed to insulin alone. These results indicate that the pre-incubation of primary skeletal muscle cells with IGF-II, a factor that plays a role in the insulin signaling pathway, inhibits the insulin response in primary human skeletal muscle cells as was the case in L6 cells.

### Example 8

### High-Throughput Screening of Modulators of Insulin Responses

Since the earlier experiments showed that the impedance assay was capable of identifying factors that affected a cell's response to insulin, we used the impedance assay to identify other factors, in a high throughput manner, that would influence the insulin signaling pathway. First, however, we screened for factors that affect cell impedance in human primary skeletal muscle cells. Then, we screened for factors that not only had an effect on cell impedance when used alone, but also were able to affect the cell impedance changes imposed by insulin treatment (i.e., modulators of insulin responses).

Primary human skeletal muscle cells (Cambrex, East Rutherford, N.J.) were prepared for the impedance assay as described in the previous examples. The cells were permitted to attach to the plate, and the plate was incubated overnight as previously described. The steps that followed varied according to the purpose of the test.

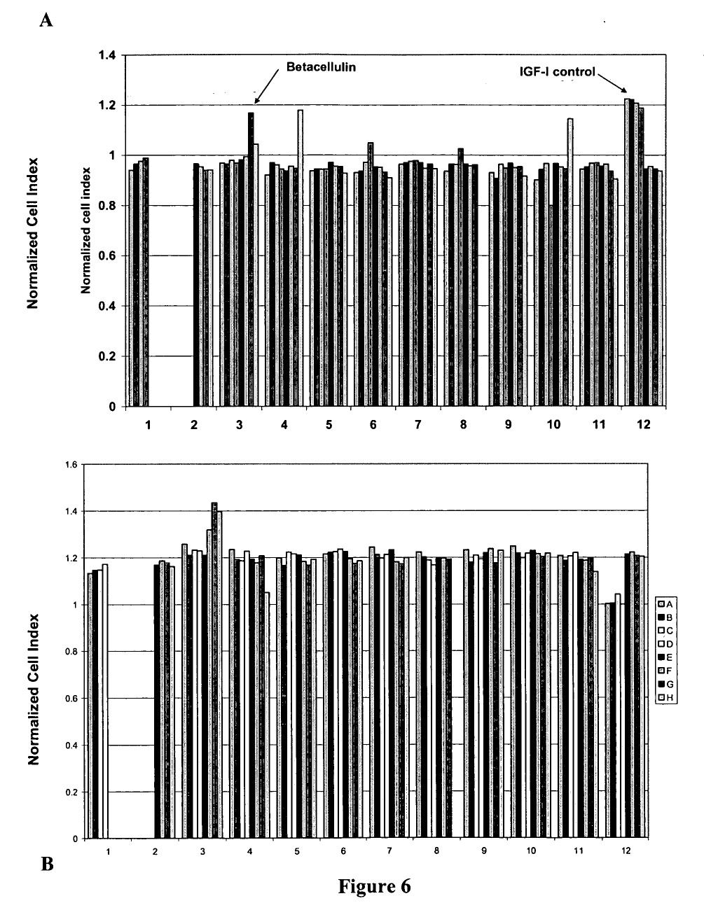

To test a panel of agents for their effects on cell impedance/cell index, the baseline impedance was established after an overnight incubation. The growth medium was removed and serum-free medium was added. The cells were then incubated for another six hours in serum-free medium. The baseline was established by measuring impedance at two-minute intervals over a four-minute period. After establishing a baseline, the serum-free medium was replaced with 40 microliter of medium comprising the test agents to be screened, one protein to each well. The impedance of each well was measured every 2 min for a total of 30 min. The results of the 30 min measurements are show in FIG. 6A. Insulin-like growth factor I (IGF-I), at a concentration of 10 nM, was used as a positive control. Columns 1-12 and rows A-H refer to the grid of wells in the 96 well plate. Betacellulin (arrow) is contained in well G3. Well H4 contains the internal positive control insulin growth factor-1 (IGF-1). Well D6 contains interleukin 4 (IL-4). Well H3 contains fibroblast growth factor-I (FGF-1). Well D10 contains Semaphorin 3F. Well H10 contains PDGF-C. Well D8 contains endothelin 3. Wells 12A-D contain the external positive control 10 nM IGF-I. No data are shown with respect to wells 1E-H and 2A-D. These results show that betacellulin induced a significant change in cell index in human primary skeletal muscle cells, as measured by this impedance assay.

To test a panel of agents for their effect on insulin-mediated changes in cell impedance, the cells were treated as follows. After the overnight incubation, the growth medium was removed and serum-free medium was added. The cells were then incubated for another six hours in serum-free medium, and a baseline impedance measurement was obtained. After the baseline measurements, 40 microliter of each test agent was added to each respective well and the cells were incubated overnight. The next day, a new baseline measurement was taken to establish a pre-insulin baseline impedance value. Insulin was then added to each well to a concentration of 200 nM and impedance was measured every 2 min for total of 30 min. Screening was performed in a cell culture incubator at 37° C. and 5% CO₂. The measurement of each well was normalized to the pre-insulin baseline. Insulin-like growth factor I (IGF-I), which we had earlier found to decrease the magnitude of the insulin-mediated increase in cell index when used to pretreat these cells at a concentration of 10 nM, was used as a control. Two series of screening experiments were done for this test: one with media conditioned by cells expressing cDNAs for different secreted proteins (from an internal cDNA library), and one with different purified recombinant proteins. The results for the experiment done with conditioned media are shown in FIG. 6B. The measurement of each well were normalized to its last measurement of the new baseline. The data were plotted at the single time point at 30 minutes in a 96 well plate layout. Betacellulin is located in the well G3. Well F3 is FGF18. Well H4 is internal positive control IGF-I. Well H3 is FGF1. Wells 12A-D are 10 nM IGF-I, used as an external positive control. There are no data on well 1E-H and 2A-D.

The results from the two series of experiments, done with either purified recombinant proteins (purified human recombinant betacellulin at 100 nM, purchased from R&D Systems, Inc.; Minneapolis, Minn., Cat# 261-CE) or with conditioned media containing a variety of secreted proteins (including betacellulin), both showed that betacellulin increased the cell index/impedence response of human primary skeletal muscle cells to insulin. In other words, in human primary skeletal muscle cells, the magnitude of the increase in cell index caused by insulin was higher if the cells had been pretreated with betacellulin. The recombinant betacellulin was characterized as a soluble mature human betacellulin DNA sequence that was expressed in E. coli, referencing Sasada, R. et al., BBRC, 190: 1173 (1993), and having 80 amino acid residues and a molecular mass of 9.5 kDa.

### Example 9

### Time Course of the Impedance Changes Induced by Betacellulin

We compared the impedance changes over time caused by treatment of primary human skeletal muscle cells with either betacellulin or insulin. Primary human skeletal muscle cells were prepared for the impedance assay as previously described. Baseline impedance was established at 0, 2, and 4 min. About 1 microM insulin (Eli Lilly and Company, Indianapolis, Ind.), about 100 nM betacellulin, and a mock control, respectively, were separately added to each respective wells and impedance measurements were continued for about 30 min at two-minute intervals. As previously done, results are expressed as normalized cell index, normalized to the baseline value prior to the addition of insuln or betacellulin.

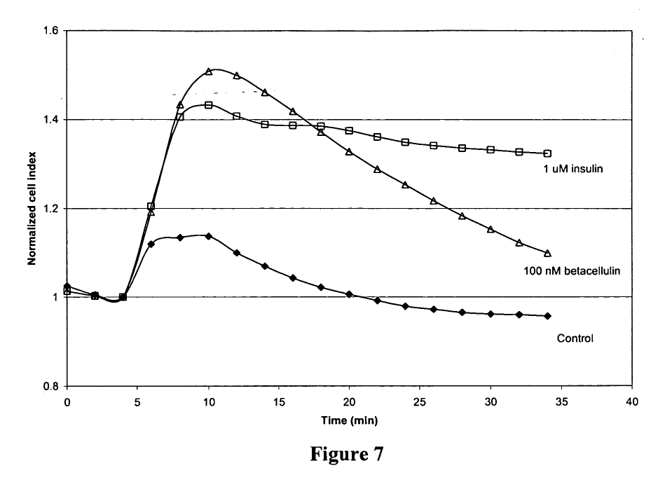

The results, depicted in FIG. 7, showed that insulin (1 microM) treatment induced an increase in the cell index in the initial 6-10 min. Thereafter, the cell index remained elevated for approximately 30 min, decreasing only slightly over that time period. Betacellulin treatment, on the other hand, induced a higher increase in cell index than did insulin in the initial 10-20 min, peaking at about 10 min. Thereafter, the cell index of the betacellulin-treated cells decreased and fell below that of the insulin-treated cells between about 17-35 min, although it remained still higher than that of the control cells, which exhibited lower cell indices than betacellulin- and insulin-treated cells at all time points. These results show that betacellulin affects human muscle cells in a way that differs from insulin over time, and that betacellulin, unlike insulin, has a rapid onset (within about 5-10 min) of action and a short duration of activity.

### Example 10

### Pre-Incubation of Skeletal Muscle Cells with Purified Betacellulin Increases the Muscle Cell Response to Insulin

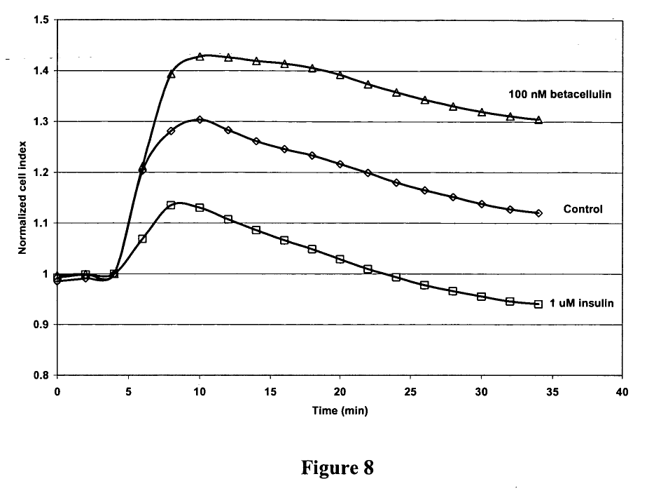

We tested the kinetics of the effect of pre-incubation of primary human muscle cells with 100 nM of purified betacellulin in their ability to respond to subsequent exposure to insulin. To this end, we compared the betacellulin pretreatment with pretreatment with 1 microM of insulin over the period of 35 min. We found that pre-incubation of the cells with betacellulin (for 24 hr) increased their subsequent response to insulin as early as 10 min after stimulation with insulin; moreover, this effect lasted for the entire 30 min of the experiment, as shown in FIG. 8. The opposite effect was observed with insulin pretreatment.

In this test, primary human skeletal muscle cells were prepared and the impedance assay was conducted as previously described. A baseline impedance measurement was taken before pre-treatment with betacellulin or insulin. Insulin (Eli Lilly and Company, Indianapolis, Ind.), betacellulin (R&D Systems, Minneapolis, Minn.), or a mock control (serum-free medium), was added to the respective wells and impedance measurements were made at a frequency of 1 measurement every 2 min over a period of 30 min. Cells were incubated for 24 hr with the test substance. A pre-insulin baseline impedance measurement was then taken. Next, insulin was added to each well to a final concentration of about 200 nM, and impedance measurements were made at two-minute intervals for 30 min. Results from this test showed that pre-incubation with 1 microM insulin inhibited the subsequent response of the cells to insulin, whereas pre-incubation with 100 nM betacellulin increased the response of the cells to insulin. This experiment indicates that betacellulin, unlike IGF-2, which we showed in an earlier experiment (see Example 8) to inhibit a subsequent insulin response, is likely to be complementary to insulin in its activity.

### Example 11

### ErbB Ligand Family Members Induce Variable Impedance Changes

With our finding that betacellulin, like insulin and other factors that are involved in the insulin signaling pathway, induces impedance changes in human muscle cells, we set out to test whether other members of the ErbB ligand family have similar effects. In this test, we found that some members of the ErbB ligand family induced impedance changes in primary human skeletal muscle cells (Cambrex) that differed among the family members over the period tested of about 35 min. The ErbB ligand polypeptides we tested were purchased from R&D Systems (Minneapolis, Minn.) and included TGF-α(Cat. #239-A), NRG1-alpha (NRG1-α) EGF domain (Cat. # 296-HR), NRG1-beta (NRG1-β) EGF domain (Cat. #396-HB), HB-EGF (Cat. # 259-HE), Epiregulin (Cat. #1195-EP), EGF (Cat. # 236-EG), Amphiregulin (Cat. # 262-AR), Betacellulin (Cat. #261-CE), and Epigen (Cat. #1127-EP).

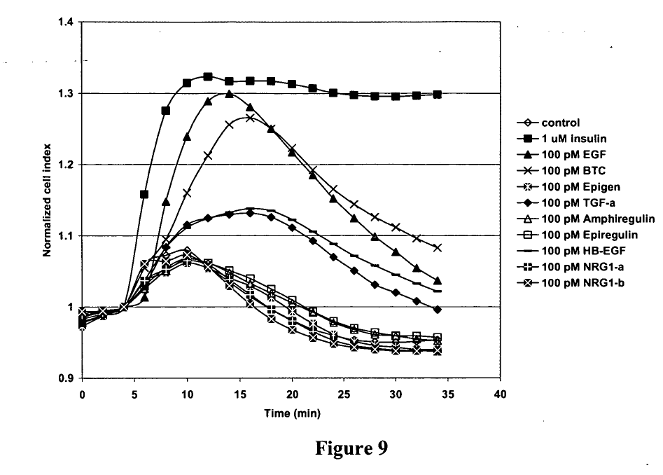

Primary human skeletal muscle cells were prepared for the impedance assay as before, except that about 90 microliter instead of about 135 microliter of serum-free medium was used in the six hr incubation step. After incubation, the baseline was measured 3 times every 2 min (0, 2 and 4 min). After the baseline measurement, about 10 microliter of each substance to be tested were added into triplicate wells, each substance to be tested being at a concentration of about 100 pM, and the impedance of each well was measured every 2 min for a total of 30 min. The measurement of each well was normalized to its last baseline measurement. About 1 microM of insulin was used as a positive control and serum-free medium was used as the negative control. Results are shown in FIG. 9.

The results of this test show that several members of the ErbB ligand family could induce increases in cell impedance in primary human skeletal muscle cells in a manner similar to that of betacellulin (BTC). Of the ErbB ligand polypeptides tested, EGF (black triangles), BTC (X), HB-EGF (−), and TGF-α (black diamonds) all displayed greater impedance changes over time than that induced by the control. While the response to insulin reached a peak at about 10 min after the start of the impedance measurement, and was sustained over the entire test period, the response to EGF, BTC, HB-EGF, and TGF-α showed a rapid rise, and peaked at about 14 min, 16 min, 16 min, and 16 min, respectively, and decreased just as rapidly thereafter. The remaining ErbB ligand polypeptides, epiregulin, amphiregulin, and Epigen behaved about the same as the negative control. Impedance changes induced by NRG1-α and NRG1-β were slightly below the control. This experiment showed that, at the concentration of the ErbB ligands tested (100 pM), EGF and betacellulin produced the highest effect of increase in cell index, followed by HB-EGF and TGF-alpha. Other ErbB ligands may also have activity.

### Example 12

### Stimulation of Glucose Uptake into Skeletal Muscle Cells by Insulin and Betacellulin

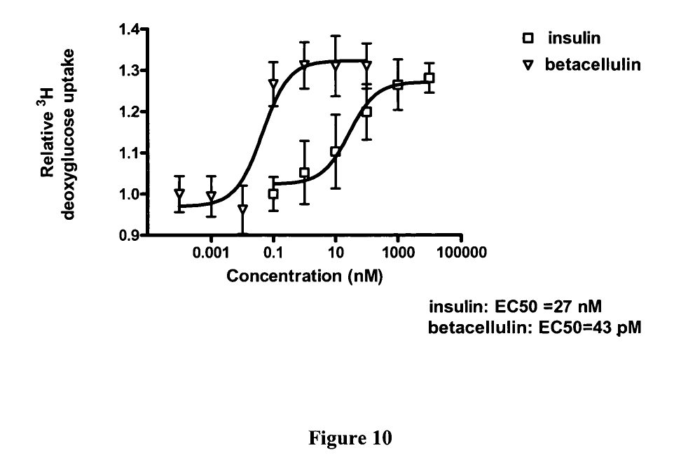

After discovering that betacellulin induced changes in impedance in primary human skeletal muscle cells, as did insulin, we tested whether betacellulin, like insulin, would stimulate glucose uptake. Our results, as shown in FIG. 10, demonstrate that betacellulin stimulated glucose uptake into these cells with greater potency than insulin.

The method of directly measuring glucose uptake most often used and, accordingly, referred to as the “gold standard,” uses radioactive non-metabolic ³H deoxyglucose, for example, as measured by Suarez E. et al., J. Biol. Chem., 275:18257-18264 (2001). The rate of glucose uptake is measured as a rate of incorporation of radioactive ³H deoxyglucose, for example, into muscle cells (Sweeney, G. et al., J. Biol. Chem., 274:10071-10078 (1999)).

In this test, primary human skeletal muscle cells (Cambrex) were prepared as described above and serum-starved for 5 hr. They were then incubated with either insulin, at concentrations ranging from about 10⁻¹¹ to about 10⁻⁴ M (Eli Lilly and Company, Indianapolis, Ind.) or betacellulin, at concentrations ranging from about 10⁻¹³ to 10⁻⁶ M (R&D Systems, Minneapolis, Minn.) for 20 min. Control cells had no such growth factor additions (i.e., no insulin, no betacellulin). The medium was then replaced with 50 microliter glucose-free medium containing 1 microCi ³H-deoxyglucose in a 10 microM solution of unlabeled deoxyglucose. The cells were incubated with the radiolabeled glucose for 15 min, and then washed three times with ice-cold phosphate buffered saline (“PBS”). The cells were then lysed by constant shaking for 10 min with 1 ml of 0.05 N sodium hydroxide (NaOH) and the radioactivity was determined by the PerkinElmer TopCount microplate scintillation counter (PerkinElmer Life And Analytical Sciences Inc., Wellesley, Mass.). The results were plotted relatively to the glucose uptake measured in non-treated control cells. The EC₅₀ of insulin was determined to be approximately 27 nM while the EC₅₀ of betacellulin was determined to be approximately 43 pM, showing that betacellulin was more potent than insulin in stimulating glucose uptake into these muscle cells.

### Example 13

### Combined Effect of Insulin and Betacellulin on Glucose Uptake by Human Skeletal Muscle Cells

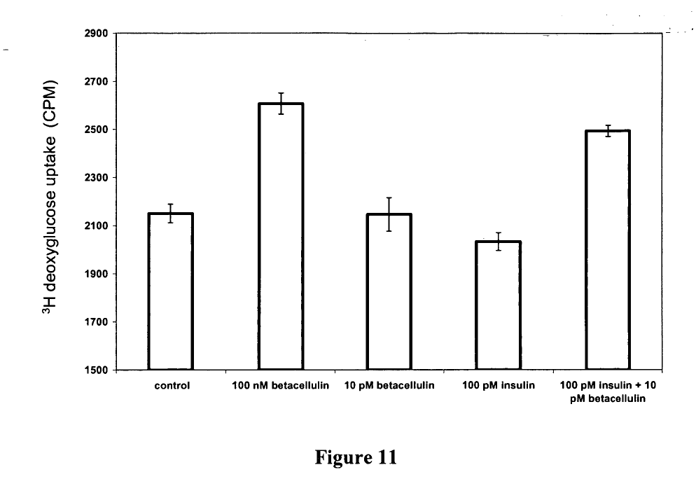

We tested the effect of combining betacellulin and insulin on glucose uptake in primary human skeletal muscle cells, to determine whether there would be any additive effect. We found in this test that a combination of a low concentration of betacellulin, at 10 pM, and a low concentration of insulin, at 100 pM, increases glucose uptake synergistically in primary human skeletal muscle cells, when compared to betacellulin alone or insulin alone, as shown in FIG. 11.

In this test, radioactive glucose uptake was measured as described in Example 12. Betacellulin at 100 nM increased ³H-deoxyglucose uptake to about 2600 cpm from about 2150 cpm for that of control. Betacellulin at 10 pM and insulin at 100 pM insulin behaved substantially as the control. In contrast, the combination of 10 pM betacellulin and 100 pM insulin significantly increased glucose uptake to about 2500 cpm in primary human skeletal muscle cells.

### Example 14

### Betacellulin Enhances Insulin-Stimulated Glucose Uptake in Skeletal Muscle Cells in a Dose-Dependent Manner

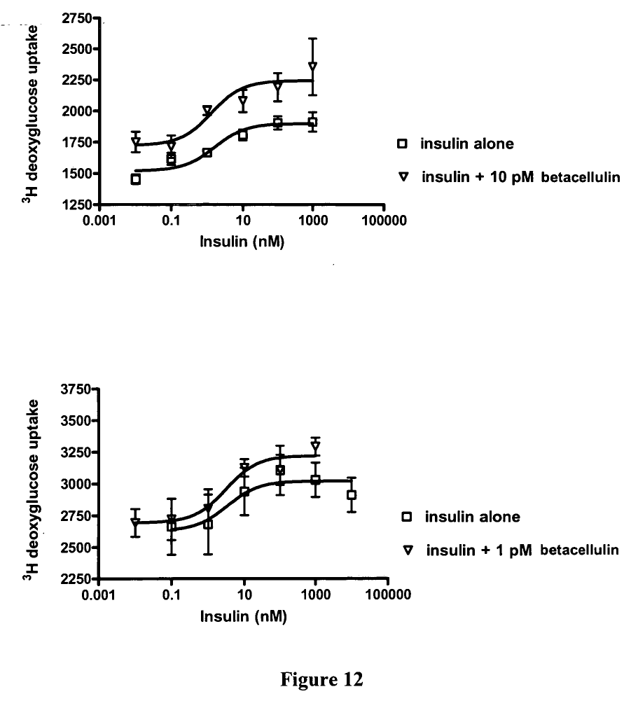

We tested betacellulin at 10 pM (FIG. 12, top) as well as betacellulin at 1 pM (FIG. 12, bottom) in combination with varying concentrations of insulin. Our results confirmed that betacellulin had an additive effect to that of insulin, increasing insulin-stimulated glucose uptake in a dose-dependent manner, as shown in FIG. 12. In this experiment, glucose uptake measurements were performed as described in Example 12. We found that betacellulin did not change the EC₅₀ of insulin. However, it increased the magnitude of the glucose uptake by primary human skeletal muscle cells stimulated by insulin, even at a concentration of 1.0 pM.

### Example 15

### Stimulation of Glucose Uptake into Skeletal Muscle Cells by ErbB Ligand Family Proteins

We further tested other ErbB ligand family members for their ability to stimulate glucose uptake into muscle cells, as measured by the radioactive glucose uptake assay described in Example 12. We found that all the ErbB ligand polypeptides tested stimulated increase in glucose uptake in the muscle cells to varying degrees.

The ErbB ligands were all purchased from R&D Systems, Inc. (Minneapolis, Minn.) and include: (1) Betacellulin (“BTC) (Cat# 261-CE), an 80 amino acid residue protein expressed in E. coli from a DNA encoding the soluble mature human betacellulin protein sequence, as described in Sasada, R. et al. BBRC 190: 1173 (1993) and having a predicted molecular mass of about 9.5 kDa; (2) Epidermal Growth Factor (“EGF”) (Cat# 236-EG), a 54 amino acid residue protein that is the N-terminal methionyl form of the mature human EGF protein expressed in E. coli from a DNA sequence that encoded the mature human EGF protein (Asn 971-Arg 1023), as described in Accession # P01133 and Bell, G. I. et al., Nucleic Acids Res. 14(21): 8427-8446 (1986) and having a predicted molecular mass of about 6 kDa; (3) Heparin-binding EGF (“HB-EGF”) (Cat#259-HE), an 86 amino acid mature recombinant protein generated by removal of the 62 amino acid residue signal and propeptide sequence produced by expressing a DNA sequence encoding the N-terminal 148 amino acid residues of human HB-EGF precursor in Sf21 insects cells using a baculovirus expression system, as described in Higashiyama, S. et al., Science 251: 936 (1991) and having a predicted molecular mass of about 9.5 kDa. However, this recombinant protein was noted to be heterogeneously O-glycosylated and migrated as an approximately 12 kDa protein in SDS-PAGE; (4) TGF-alpha (“TGF-α”) (Cat# 239-A), a 50 amino acid residue recombinant protein expressed in E. coli from a DNA sequence encoding the mature human TGF-α protein sequence, as described in Derynck, R. et al., Cell 38: 287-297 (1984) and having a predicted molecular mass of about 6 kDa; (5) NRG1-alpha (“NRG1-α”) (Cat# 296-HR), a 65 amino acid residue recombinant protein expressed in E. coli from a DNA sequence encoding the EGF domain of Heregulin α, amino acid residues 177-241, as described in Holmes, W. E. et al. Science 256: 1205 (1992) and having a predicted molecular mass of about 7 kDa; (6) amphiregulin (“AR”) (Cat# 262-AR), a 98 amino acid residue recombinant human protein expressed in E. coli from a DNA sequence encoding the 98 amino acid residue form of mature human amphiregulin corresponding to amino acid residues Ser 101 to Lys 198, as described in Plowman, G. D. et al. Mol. Cell. Biol. 10:1969 (1990), having a predicted molecular mass of about 11 kDa; (7) epiregulin (“EPR”) (Cat# 1195-EP), a 47 amino acid residue methionyl form of recombinant human epiregulin expressed in E. coli from a DNA sequence encoding the mature chain of human epiregulin Val 63-Leu 108 (Accession number XP_{—}003511) and having a predicted molecular mass of about 5.4 kDa; (8) Epigen (Cat# 1127-EP), a 51 amino acid residue form of recombinant mouse Epigen expressed in E. coli from a DNA sequence encoding the functional internal peptide of mouse Epigen amino acid residues 53-103 and having a molecular mass of about 5.9 kDa; and (9) NRG1-beta (“NRG1-β”) (Cat# 396-HB), a 71 amino acid residue recombinant protein expressed in E. coli from a DNA sequence encoding the EGF domain of Heregulin beta, amino acid residues 176-246, as described in Holmes, W. E. et al., Science 256: 1205-1210 (1992) and having a molecular mass of about 8 kDa.

In this experiment, primary human skeletal muscle cells were treated as described in Example 12. Cells were serum-starved for 5 hr. Then, different concentrations of the ErbB ligand polypeptides, varying from about 10⁻¹³ M to about 10⁻⁷ M, were each added to separate wells of cells in serum-free medium, except that only medium was added to the control cells. The cells were then incubated at 37° C. for 20 min, after which the medium was completely removed and 50 microliter of glucose-free medium with 1 μCi of ³H-deoxyglucose in 10 microM deoxyglucose was added to each well. Cells were labeled for 15 min after which the labeling medium was removed and the cells washed with ice-cold PBS three times. Cells were then lysed by constant shaking for 10 min with 1 ml of 0.05 N sodium hydroxide and radioactivities were counted by a PerkinElmer TopCount microplate scintillation counter. Results were plotted as relative ³H-deoxyglucose uptake, as compared to the control, and as a function of the concentration of the ErbB ligand protein being tested.

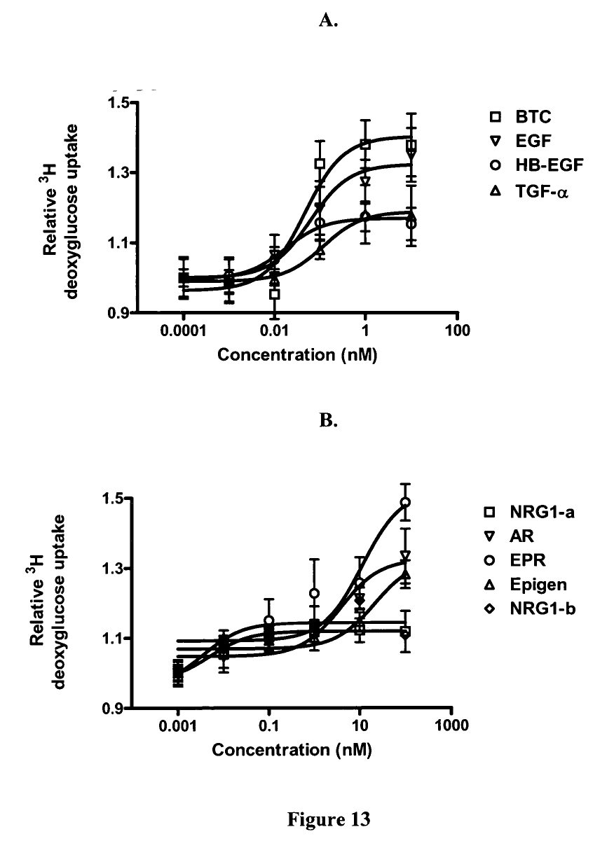

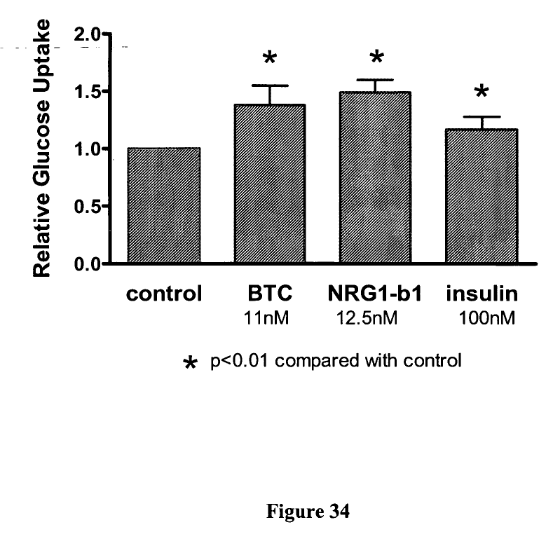

As shown in FIG. 13A, betacellulin, EGF, HB-EGF, and TGF-α stimulated glucose uptake with EC₅₀s from about 10 pM to about 100 pM. FIG. 13B shows that AR, EPR, and Epigen each stimulated glucose uptake with EC₅₀s in the nanomolar range. The EC₅₀ of betacellulin and EGF were about 46 pM and about 60 pM, respectively, much lower than that of insulin which, as seen in FIG. 10, was about 27 nM. In contrast, the EC₅₀ of epiregulin (EPR), amphiregulin (AR), and Epigen, respectively, were about 4 to 20 nM, which fall in about the same log range as the EC₅₀ of insulin. Hence, among the ErbB ligand family, betacellulin, EGF, HB-EGF, and TGF-α, epiregulin, amphiregulin, and Epigen all showed significant induction of glucose uptaken primary human skeletal muscle cells, and did so to a similar or better extent than insulin. Although the NRG1-α (alpha) and NRG1-β (beta) did not show significant stimulation of glucose uptake in this experiment, it is possible that this cellular system is less sensitive to these molecules. As shown in a later experiment (Example 36, FIG. 34), NRG1-β1 did induce glucose uptake by rat neonatal cardiomyocytes.

### Example 16

### Production of Recombinant Human Betacellulin

Recombinant human betacellulin cDNA may be expressed in a number of different conventional expression systems, whether in eukaryotic cells or prokaryotic, to produce the recombinant protein, using methods such as those described in U.S. Pat. No. 5,886,141.

In order to obtain larger amounts of betacellulin for in vivo testing, we produced recombinant human betacellulin by conventional techniques by expression of a pET24/BTC expression vector in E. coli (hereafter referred to as “BTC made internally from E. coli expression”). First, we created a BTC construct in the vector pET24(+) (Novagen, EMD Biosciences Inc, San Diego, Calif.) without the His-Tag (which was removed during subcloning), which encoded an active recombinant human betacellulin fragment corresponding to amino acid residues Asp³²-Tyr¹¹¹ preceded by an initial methionine (Met) residue. The vector was transformed into E. coli Rosetta™(DE3) cells (Novagen) according to conventional methods. Individual transformants were isolated and grown according to the pET24 vector manufacturer's instructions (see pET System Manual, 10^{th} and 11^{th} Editions, Novagen). The BTC was then purified from inclusion bodies in bacterial lysates by affinity chromatography on ToyoPearl AF-Blue resin, followed by hydrophobic interaction chromatography on Phenyl-Sepharose 6 Fast Flow (high sub). Details of the process are provided below. All standard chemicals were obtained from Sigma-Aldrich Chemical Co. (St. Louis, Mo.).

In the initial fermentation step, Rosetta™ (DE3) cells were grown in Luria Bertani (LB) broth (supplemented with 50 μg/ml of kanamycin and 34 μg/ml of chloramphenicol) at 37° C. in standard bacterial fermentation vessels, with agitation, to an optical density of about 5 at the wavelength of about 600 nm. This was followed by 4 hr of induction of expression of rhBTC protein in the presence of 1 mM isopropyl β-D-thiogalactopyranoside (Sigma Chemical Co., St. Louis, Mo.).

The process of harvesting and solubilization of inclusion bodies to obtain the BTC protein was done as follows. BTC, produced as insoluble inclusion bodies in the bacteria, was purified as follows. Cells were harvested by centrifugation and the cell pellets resuspended in 20 mM Tris-HCl at pH 8.0 containing 10 mM EDTA and 1% Triton X-100 in a volume of that was equal to 0.1 volume of the initial culture medium. Thereafter, cells were lysed by pressure homogenization (with a Microfluidizer), and the inclusion bodies (IB) recovered by centrifugation at 20,000×g for 15 min at 4° C. The IB pellets were washed twice with the same volume of 20 mM Tris-HCl at pH 8.0 containing 10 mM EDTA and 1% Triton X-100 and resuspended to 3 mg of pellet per ml of solubilization buffer (100 mM Tris-HCl at pH8.0 containing 7 M guanidine hydrochloride and 5 mM dithiothreitol). The BTC protein was extracted from the IB by incubation at 4° C. for an average of one hour without agitation.

The next step entailed re-folding of the recombinant BTC, which proceeded as follows. After extraction, the solubilized protein concentration was adjusted to 2.5 mg/ml and diluted 25-fold further with refolding buffer (50 mM Tris-HCl at pH 8.0 containing 2 M urea, 0.5 mM oxidized glutathione, 1 mM reduced glutathione, and 0.1 M arginine) and incubated for approximately 20 hr at 4° C., during which period the BTC was renatured or refolded. Refolding was terminated by adjusting the pH to 5.0 with concentrated 3 M sodium acetate (pH 4.75). The refolded BTC protein was dialyzed against phosphate buffered saline (PBS) (without calcium and magnesium) diluted 1:3 in purified water. The dialyzate containing the refolded BTC was clarified by centrifugation at 5,000×g.

Next, BTC was purified by chromatography. Refolded BTC was applied to a Toyopearl AF-Blue HC-650 column (1.6 cm×20 cm) (Tosoh Bioscience LLC, Montgomeryville, Pa.) equilibrated with 10 mM potassium phosphate buffer pH 7.0 buffer containing 50 mM NaCl (Buffer A). Proteins were eluted at 3 ml/min with a continuous gradient of Buffer A to Buffer B (10 mM potassium phosphate buffer at pH 7.0 containing 1.5 M NaCl) established over 20 column volumes (i.e., a linear gradient of 0 to 1.5 M NaCl). The desired BTC-containing fractions were collected and pooled. Ammonium Sulfate was added to a final concentration of 1.3M for further purification by hydrophobic interaction chromatography over a Phenyl Sepharose™ 6 FF/high sub (1.6 cm×20 cm) (GE Healthcare, Piscataway, N.J.) equilibrated with 10 mM potassium phosphate buffer at pH 7.0 containing 1.5 M NH₄SO₄ (Buffer C). The BTC protein was eluted with a continuous gradient of Buffer C to Buffer D (10 mM potassium phosphate buffer pH 7.0 containing 50 mM NaCl) established over 25 column volumes at the flow rate of 3 ml/min. The fraction containing the purified BTC protein (as determined by conventional SDS-PAGE and Coomassie blue/Silver Stain protein visualization techniques) was concentrated by tangential flow filtration and the concentrate was dialyzed against PBS (without Ca²⁺/Mg²⁺).

Removal of endotoxin was accomplished by further purification by Cellufine™ ET clean (Chisso Corporation, Tokyo, Japan) chromatography (Sakata, M. et al. American Biotechnol. Lab. 20:36 (2002)) following the manufacturer's instructions. Briefly, the dialyzed BTC was applied to a Cellufine™ ET clean column (10×0.9 cm (I.D.); 9.6 ml) equilibrated with PBS, and collected in the flow through at the flow rate of 0.5 ml/min. The final BTC solution (in PBS without Ca²⁺/Mg²⁺) typically contained less than 2 E.U./mg of protein, as assessed by the Limulus amoebocyte lysate (LAL) assay (Cambrex, Walkersville, Md.).

### Example 17

### Clearance of Betacellulin by Normal Mice

We injected betacellulin intravenously into normal mice and observed the plasma level of betacellulin over a period of about 60 min. Betacellulin (R&D Systems, Minneapolis, Minn.) was administered as a single intravenous dose of 0.5 mg per kg of body weight of mice (i.e., 0.5 mg/kg) into wild-type normal C57BL/6J mice (9 weeks old, male, from Charles River Laboratories, MA). Serum concentrations of betacellulin were monitored by an enzyme-linked immunosorbant assay (ELISA) (from R&D Systems, Minneapolis, Minn.) from blood collected from the tail vein at various time points (5 min through 60 min post betacellulin administration). The recombinant betacellulin we injected was of recombinant human origin, and the ELISA assay we used does not detect mouse betacellulin (less than 0.125% cross-reactivity as per manufacturer). Hence, we were able to specifically measure the clearance rate of the injected human betacellulin.

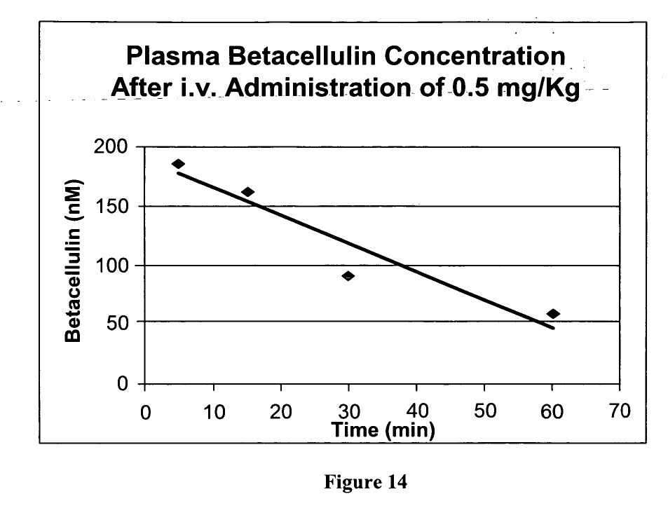

Results (see FIG. 14), plotted as nM of betacellulin in the plasma of the mice as a function of time (in min), show that betacellulin was detectable at about 5 min after administration at a level of about 180 nM, and decreased to just over 150 nM at about 15 min, then to about 100-120 nM at about 30 min, and to about 50 nM at about 60 min, with a half-life of about 32 min in these animals. This experiment showed that the circulating half-life of human recombinant betacellulin was approximately 32 min in normal C57BL/6J mice. Each data point represents an average of measurements in three mice.

### Example 18

### Subcutaneous Administration of Betacellulin Extends its Bioavailability Relative to Intravenous Administration

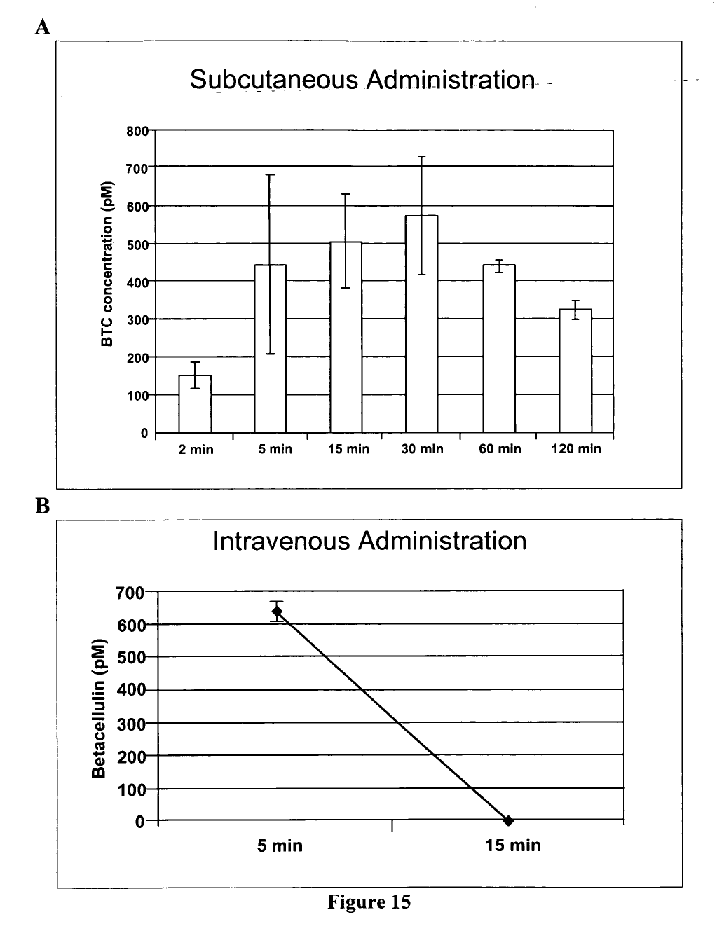

We compared the residence time of betacellulin when injected intravenously to that injected subcutaneously in normal C57BL/6J mice. As shown in FIGS. 15A and 15B, we found that subcutaneous administration of betacellulin in vivo resulted in a dramatic increase in the duration of bioavailability, compared to intravenous administration.

In this test, wild-type normal C57BL/6J mice (9 weeks old, male, from Charles River Laboratories, MA) were injected either subcutaneously (s.c.) or intravenously (i.v.) through the tail vein with a single dose of betacellulin at 0.05 mg/kg (from R&D Systems, Minneapolis, Minn.). Blood samples were collected from the tail vein of each mouse at time points of approximately 2-, 5-, 15-, 30-, 60- and 120 min. Results (FIG. 15A) show that the subcutaneous administration of betacellulin produced detectable plasma levels of betacellulin at about 2 min after administration at a level of about 150 pM, and increased to about 440 pM at about 5 min, to just over 500 pM at about 15 min, and peaked at about 575 pM at about 30 min. Plasma betacellulin then decreased to about 440 pM at about 60 min, and to about 320 pM at about 120 min.

In contrast, mice injected with betacellulin at 0.05 mg/kg dose intravenously (FIG. 15B) showed a plasma level of about 620 pM in about 5 min after administration. Betacellulin was cleared from the plasma of these animals in about 15 min at which time, no betacellulin was detectable. Hence, a dramatic increase in the duration of betacellulin bioavailability was obtained from subcutaneous injection as compared to intravenous administration. However, betacellulin was present in the blood of the i.v. injected mice at a higher level much earlier than that measured in mice injected with betacellulin subcutaneously (s.c.). Each data point represents an average measurement in three mice.

### Example 19

### Peak Plasma Concentrations and Clearance Rates of Betacellulin were Dose-Dependent after Subcutaneous Administration

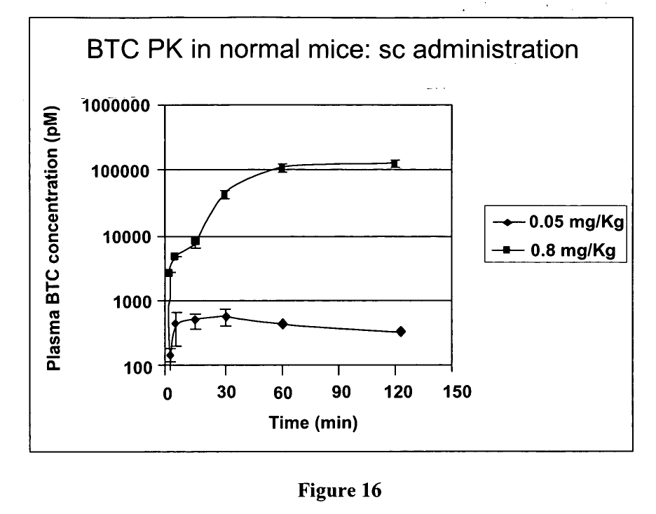

We examined the plasma levels of betacellulin when injected subcutaneously at two different doses into normal C57BL/6J mice. The results are shown in FIG. 16. We found that in vivo circulating human recombinant betacellulin concentrations as high as 120 nM could be reached in mice and maintained for as long as 120 min or more after subcutaneous (s.c.) administration of a 0.8 mg/kg dose. In this test, wild-type normal C57BL/6J mice were injected subcutaneously (s.c.) with the a single dose of betacellulin at either 0.05 mg/kg or 0.8 mg/kg. Blood samples were collected from the tail vein at about 2-, 5-, 15-, 30-, 60- and 120 min post injection and analyzed for betacellulin levels by ELISA as before. After subcutaneous administration of betacellulin, plasma levels of betacellulin reached a peak of about 120 nM at between about 60 to about 120 min post-administration for mice injected with 0.8 mg/kg weight. At the 0.05 mg/kg s.c. dose, betacellulin reached a peak of about 0.6 nM at about 30 min post administration. For reference, the circulating level of betacellulin in normal human plasma is about 3 pM. Each data point represents an average of measurements in three mice.

### Example 20

### Betacellulin Lowers Blood Glucose in Normal Mice in a Dose-Dependent Manner

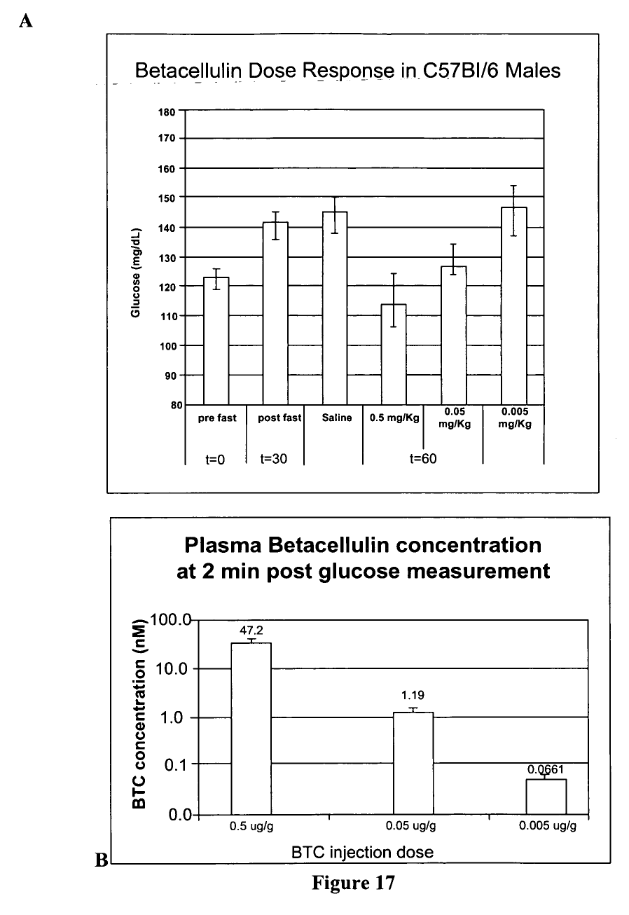

We examined the blood glucose level of normal C57BL/6J mice treated with different doses of betacellulin after fasting. Results are shown in FIG. 17. We found that betacellulin reduced blood glucose levels in fasting animals in a dose-responsive manner with rapid kinetics. In this test, wild-type normal C57BL/6J mice were fasted by taking away the food at time 0, and 30 min later injecting the animals subcutaneously with a single dose of saline, or betacellulin at either 0.005 mg/kg, or 0.05 mg/kg, or 0.5 mg/kg. Blood samples were collected from the tail vein of these mice at time points 0 min (pre-fast), 30 min later (post-fast) before injection of betacellulin or saline, and 30 min after such injection (t=60 min). The blood samples were analyzed both for betacellulin levels (by ELISA) and for whole blood glucose using an automatic glucose monitor (One Touch II; Lifescan Inc., Milipitas, Calif., USA). FIG. 17A shows a pre-fast glucose level of about 123 mg/dL and a post-fast glucose level of about 142 mg/dL. At time t=60 min, blood glucose level of the saline-treated mice averaged about 145 mg/dL; the blood glucose level of the mice treated with 0.5 mg/kg of betacellulin averaged about 115 mg/dL; the blood glucose level of the mice treated with 0.05 mg/kg of betacellulin averaged about 127 mg/dL; and the blood glucose level of the mice treated with 0.005 mg/kg of betacellulin averaged about 146 mg/dL.

Plasma betacellulin levels were measured 2 min post glucose measurements. The results are shown in FIG. 17B. At the 0.5 μg/g (i.e., 0.5 mg/kg) dose of betacellulin, plasma betacellulin level was about 47.2 nM; at the 0.05 μg/g (i.e., 0.05 mg/kg) dose of betacellulin, plasma level of betacellulin was about 1.19 nM; and at the 0.005 μg/g (i.e., 0.005 mg/kg) dose of betacellulin, the plasma level of betacellulin was about 0.0661 nM. Hence, betacellulin reduced blood glucose in a fasted normal animal in dose-dependent manner, and with rapid kinetics. Each data point represents an average of measurements in six mice.

### Example 21

### Postprandial Glucose Lowering Effects of Betacellulin

In an earlier set of experiments (Examples 12-14, FIGS. 10 through 12), we showed that betacellulin and various other members of the ErbB ligand family stimulated glucose uptake into skeletal muscle in vitro. Those studies indicated that the betacellulin effect was dose-dependent and that betacellulin was more potent than insulin at promoting glucose uptake by cultured primary human skeletal muscle cells. Glucose tolerance tests (GTT tests), conducted in diabetic (“db”) and normal mice, were used to understand the effect of betacellulin on blood glucose levels in vivo.

We used db mice (Mouse Genome Informatics (MGI) accession number 1856009) as a model of diabetes (as described in Hummel K P et al., Science 153(740):1127 (1966) and normal C57BL/6J mice as a normal control non-diabetic strain. The db mice have long been tested as amodel of human diabetes (Hunt CE et al., Fed Proc. 35(5):1206-17 (1976)). We obtained the male db mice from the Harlan Laboratories at 7-8 weeks of age (C57BL/Ks, DIABETIC Type II, C57BL/KsOlaHsd-Lepr^{db} mice; Harlan Laboratories, IN) and the C57BL/6J mice were obtained from the Jackson Laboratories at 7-8 weeks of age (C57BL/6J, strain number 000664; The Jackson Laboratories, Bar Harbor, Me.). All mice were allowed to acclimate for 1 week prior to the initiation of testing. Betacellulin was prepared internally from expression in E. coli; betacellulin activity in each lot was confirmed either by impedance assays or by the ErbB receptor phosphorylation assay, as described in Example 35).

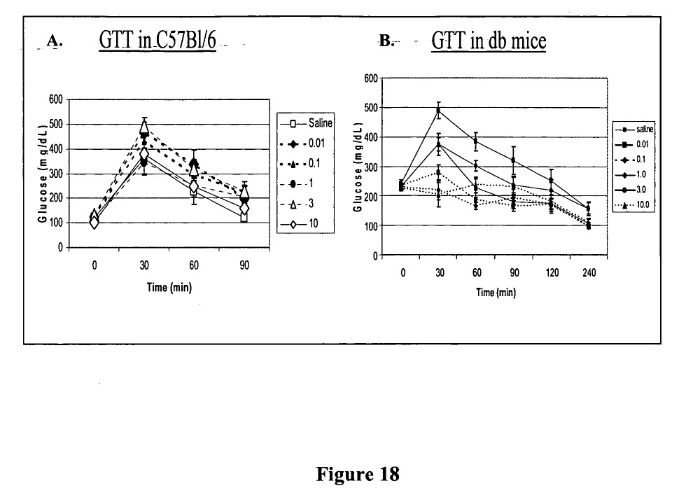

On the day of testing, the mice were fasted for five hours starting at 7 AM. Baseline (fasting) blood glucose measurements were taken at the five-hour fasting time point (that is, time 0 min). For each strain, the mice were distributed into six treatment groups based on their fasting glucose measurements. There were eight mice per treatment group for each strain. Immediately after sorting the mice into groups, 0.25 ml of betacellulin (BTC) or saline was administered by a subcutaneous injection followed immediately by an intraperitoneal injection of 0.25 ml of glucose. The C57BL/6J mice and db mice were administered 4 g/kg and 0.75 g/kg of glucose, respectively. The six equivalent treatment groups for both the db mice and the C57BL/6J mice were: saline, 0.01 mg/kg BTC, 0.1 mg/kg BTC, 1.0 mg/kg BTC, 3.0 mg/kg BTC, and 10.0 mg/kg BTC. Following administration of glucose, blood glucose measurements from tail veins were performed at multiple time points for up to four hours. Blood glucose measurements were performed with a Bayer Ascensia glucometer. The results of the test are shown in FIG. 18. Each data point represents an average of eight mice.

For the C57BL/6J mice (FIG. 18A), the results show that for saline, 0.01 mg/kg BTC, 0.1 mg/kg BTC, 1.0 mg/kg BTC, 3.0 mg/kg BTC, and 10 mg/kg BTC groups, respectively, the blood glucose was approximately 115 mg/dL at baseline (time 0), 410 mg/dL at 30 min, 280 mg/dL at 60 min, and 190 mg/dL at 90 min. There was no significant difference (as determined by the t-test) between any of the saline and the BTC treated groups for C57BL/6J mice.

For the db mice (FIG. 18B), the results show that all the BTC-treated groups had a blood glucose level of approximately 220 mg/dL at baseline. The blood glucose of the saline treated group increased to approximately 500 mg/dL at 30 min, and then decreased to about 390 mg/dL at 60 min, then to about 310 mg/dL at 90 min, then to about 250 mg/dL at 120 min, then to about 170 mg/dL at 240 min The blood glucose of the 0.01 mg/kg BTC treated group increased to approximately 380 mg/dL at 30 min, then decreased to about 300 mg/dL at 60 min, then to about 230 mg/dL at 90 min, then to about 210 mg/dL at 120 min, then to about 170 mg/dL at 240 min. The blood glucose of the 0.1 mg/kg BTC treated group increased to approximately 380 mg/dL at 30 min, then decreased to about 220 mg/dL at 60 min, then to about 200 mg/dL at 90 min, then to about 190 mg/dL at 120 min, then to about 100 mg/dL at 240 min. The blood glucose of the 1.0 mg/kg BTC treated group was approximately 280 mg/dL at 30 min, then decreased to about 200 mg/dL at 60 min, then to about 190 mg/dL at 90 min and 120 min, then to about 100 mg/dL at 240 min. The blood glucose of the 3.0 mg/kg BTC treated group was approximately 205 mg/dL at 30 min, then decreased to about 170 mg/dL at 60 min, then about 190 mg/dL at 90 min, then 170 mg/dL at 120 min, then about 100 mg/dL at 240 min. The blood glucose of the 10.0 mg/kg treated group was approximately 205 mg/dL at 30 min, then about 220 mg/dL at 60 min and 90 min, then about 170 mg/dL at 120 min, then about 100 mg/dL at 240 min. The glucose level of the mice in the BTC treatment groups was significantly different (as determined by a t-test) from that of the mice in the saline treated group.

This test showed that there was significant glucose lowering effect by betacellulin in diabetic db mice after a glucose burst as shown in a GTT, but there was no significant glucose lowering effect by the use of betacellulin in normal C57BL/6J mice. The glucose lowering effect was dose-dependent between 0.01 mg/kg and 10 mg/kg range. The results of this experiment indicate that the dose of betacellulin is a factor to consider in achieving rapid and significant glycemic control after a glucose excursion, such as after meals. Since the db mouse reportedly is a useful model of human diabetes, this experiment also indicates that betacellulin will be effective in treating patients who are insulin-resistant.

### Example 22

### Chronic Treatment with Betacellulin Resulted in Reduced Hemoglobin A1_{c} and Insulin

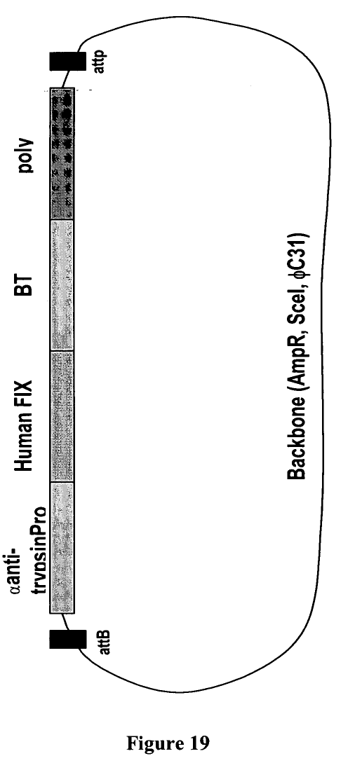

In this experiment, we tested the effect of chronic exposure of animals to betacellulin in vivo. We used a vector obtained from the laboratory of Dr. Mark Kay at Stanford University (Stanford, Calif. 94305), as described by the Kay laboratory in Hum. Gene Ther. 16(1): 126-31 (2005); Hum. Gene Ther. 16(5): 558-70 (2005); and WO 04/020605 to deliver the betacellulin gene. We modified this vector by insertion of cDNA encoding betacellulin as the gene of interest, placing it after the human Factor IX intron. This vector has the structure depicted in FIG. 19. The modified vector was injected into the animals via their tail veins (as described in more detail below), using the hydrodynamic tail vein injection method, as reported in Liu, F. et al., Gene Therapy 6: 1258-1266 (1999) and U.S. Pat. No. 6,627,616.

In an earlier experiment (Example 21, FIG. 18), we showed that acute administration of betacellulin to diabetic (“db”) mice resulted in an acute improvement in postprandial glycemic control, as demonstrated by a GTT test after administration of a bolus injection of betacellulin. The American Diabetes Association recommends measurement of hemoglobin A_{1c} (HbA_{1c}) several times a year as a way to monitor long-term care of persons with diabetes (see Goldstein, D. E. et al., Diabetes Care 27: 1761-1773 (2004). HbA_{1c} is formed by the glycation of hemoglobin Ao and is proportional to the level of glucose in the blood over a period of several weeks. Therefore, HbA_{1c} measurements are useful for understanding the long term therapeutic value of diabetic treatment modalities.

We delivered the human betacellulin cDNA expression vector (“DNA construct”), made as described above, by tail vein injection to db mice (10 control mice and 18 betacellulin-treated mice) and monitored several glycemic parameters for three weeks. The db mice were obtained from Harlan Laboratories at approximately 7-8 weeks of age and subsequently tested after about three weeks of acclimation in our facility. The betacellulin cDNA expression vector was designated construct # CLN00908052. All blood glucose measurements were performed with a Bayer Ascensia glucometer. HbA_{1c} was assayed from whole blood using blood from the tail veins of the db mice, with a Bayer DCA 2000 reagent kit and reader. Insulin was assayed from plasma using an ELISA kit from Crystal Chem Inc. (Cat# 90060; Downers Grove, Ill.). Betacellulin was assayed from plasma using an ELISA kit from R&D Systems (Cat# DY261).

The Betacellulin group was treated with betacellulin by injection with 4.2 ml of Ringer's saline containing 100 μg of the DNA construct on day 0. The Control or Saline group was injected with Ringer's saline on day 0. Expression of betacellulin was measured on days 5 and 18. Fasting blood glucose levels (after four hours of fasting) were determined on days 0, 7, 14 and 21. HbA_{1c} level was measured on days 0, 7, 14, and 21. Insulin level was measured on day 11.

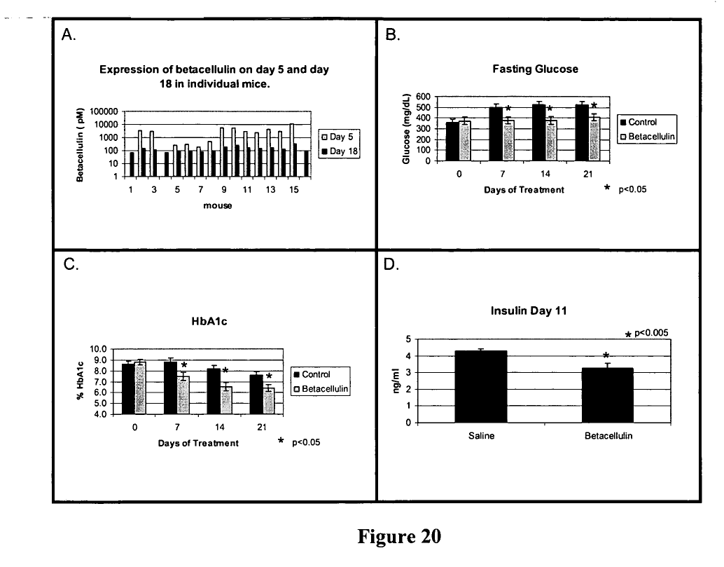

The results of the test are shown in FIG. 20. FIG. 20A shows that a significant amount of betacellulin, ranging from over 100 pM to about 10,000 pM, was observed in 13 out of 16 db mice by day 5, with 3 of the 16 db mice not showing any detectable expression. However, by day 18, 16 out of 16 animals exhibited betacellulin expression at about 100 pM. The results showed that db mice could effectively express human betacellulin at high levels that persist for at least 18 days.

FIG. 20B shows fasting glucose levels (4 hours) of about 350 mg/dL for both the Betacellulin group and the Control group at the start of the test (day 0). The mice in the Control group exhibited a high level of fasting blood glucose, reaching about 500 mg/dL by day 7, and maintaining this level through days 14 and 21, when the test was discontinued. In contrast, the mice in the Betacellulin group substantially maintained their fasting blood glucose level at about 350 mg/dL to 400 mg/dL level through days 7, 14 and 21. The difference in blood glucose levels between the Betacellulin group and the Control group was statistically significant (p<0.05). Thus, betacellulin treatment resulted in preventing a rise in fasting glucose over the course of the test period, compared to saline controls.

FIG. 20C shows relatively high HbA_{1c} levels in both groups of mice at the onset of the test (day 0), that is, about 9%. By day 7, HbA_{1c} level in mice in the Betacellulin group was significantly lower (about 7.5% as compared to 9%). This effect persisted throughout the duration of the test. By day 14, HbA_{1c} level for the Betacellulin group was about 6.5%, while that for the Control group was about 8%. By day 21, HbA_{1c} level for the Betacellulin group remained about 6.5%, while that for the Control group was about 7.5%. The difference in HbA_{1c} level between the two groups was statistically significant during the course of the test. This test demonstrated that chronic betacellulin treatment was effective at controlling fasting blood glucose in db mice during long-term treatment regimens. Since Hb A_{1c} represents an integrated glucose measurement over time, and fasting blood glucose levels are reflective of basal glucose control, these results demonstrate that betacellulin can control basal glucose levels in diabetic animals. These results also indicate that having a sustained elevated level of blood betacellulin in vivo, such as that achived by subcutaneous injection, for example, may achieve sustained reduction in blood glucose in diabetic subjects over time.

FIG. 20D shows the insulin levels of the db mice in the Control group as compared to those in Betacellulin group, as measured on day 11. The former had a level of about 4 ng/ml, while the latter has a level of about 3 ng/ml, showing that betacellulin treatment resulted in a reduction in plasma insulin levels, a difference that is statistically significant (p<0.005; t-test}). The lower insulin level in the db mice in the Betacellulin group indicates possible increased insulin sensitivity or an “insulin sparing effect” (as discussed in Slama G, Diabete Metab. 17(1 Pt 2): 241-3 (1991)). Insulin sparing could occur due to compensation from betacellulin. Altogether, the data showed that the long term continuous exposure to betacellulin decreased HbA_{1c} levels, indicating improvement in long term glycemic control.

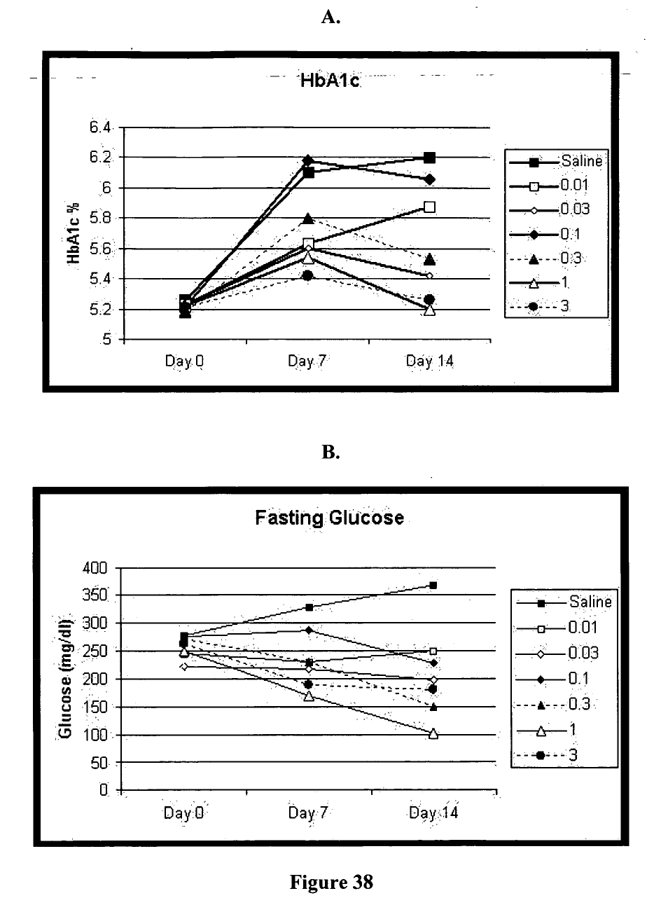

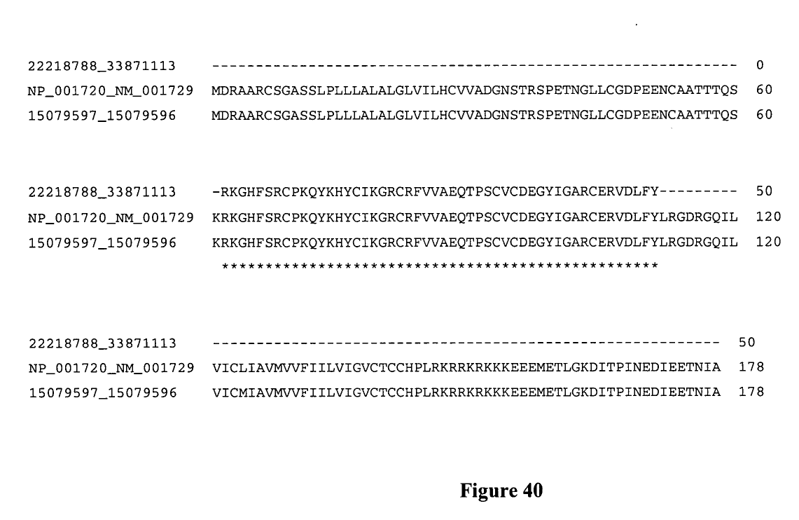

The results of this test were further confirmed in another test, the results of which are shown in FIG. 38. We previously showed (Example 21, FIG. 40) that hydrodynamic transfection of db mice with betacellulin (BTC) cDNA resulted in improved fasting glucose and HbA_{1c} levels, compared to controls. To test if a multiple dosing regimen of BTC protein for several days would result in improved fasting glucose and HbA_{1c} levels, we treated db mice for 14 days with several dosing concentrations. The timing of dosing was at night, as described below, and designed to coincide with the normal feeding time of mice. Male db mice were obtained from Harlan labs at approximately 7-8 weeks of age and subsequently tested after three weeks of acclimation in our facility. Betacellulin was prepared internally from expression in E. coli. The start day of the study was designated as day zero. On day zero, the mice were ten weeks of age, and were sorted into 7 equivalent groups of ten mice, based on their HbA_{1c} levels. The dose groups are shown below in the following chart.

| Group # | Betacellulin Dose | # mice |
|---|---|---|
| 1 | Saline | 10 |
| 2 | 3 mg/Kg | 10 |
| 3 | 1 mg/Kg | 10 |
| 4 | 0.3 mg/Kg | 10 |
| 5 | 0.1 mg/Kg | 10 |
| 6 | 0.03 mg/Kg | 10 |
| 7 | 0.01 mg/Kg | 10 |

Each mouse was dosed three times per day at 7 PM, midnight, and 7 AM, commencing at 7 PM on day 0 and continuing every day with the same dosing schedule through 7 AM on day 14. Fasting glucose and HbA_{1c} levels were measured from all mice on day 0, 7, and 14, after a five hour fast which commenced at 7 AM. All blood glucose measurements were performed with a Bayer Ascensia glucometer. HbA_{1c} was assayed from whole blood with a Bayer DCA 2000 reagent kit and reader.

The HbA_{1c} chart (FIG. 38A) shows that the percent HbA_{1c} of the saline group was approximately 5.2 on day 0, 6.0 on day 7, and 6.2 on day 14. The percent HbA_{1c} of the 0.01 mg/kg dose group was approximately 5.2 on day 0, 5.6 on day 7, and 5.9 on day 14. The percent HbA_{1c} of the 0.03 mg/kg dose group was approximately 5.2 on day 0, 5.6 on day 7, and 5.4 on day 14. The percent HbA_{1c} of the 0.1 mg/kg dose group was approximately 5.2 on day 0, 6.2 on day 7, and 6.1 on day 14. The percent HbA_{1c} of the 0.3 mg/kg dose group was approximately 5.2 on day 0, 5.8 on day 7, and 5.5 on day 14. The percent HbA_{1c} of the 1.0 mg/kg dose group was approximately 5.2 on day 0, 5.5 on day 7, and 5.2 on day 14. The percent HbA_{1c} of the 3.0 mg/kg dose group was approximately 5.2 on day 0, 5.4 on day 7, and 5.2 on day 14.

The fasting glucose chart (FIG. 38B) shows that the fasting glucose levels of the saline group was approximately 260 mg/dL on day 0, 355 mg/dL on day 7, and 375 mg/dL on day 14. The 0.01 mg/kg dose group had a fasting glucose level of approximately 250 mg/dL on day 0, 230 mg/dL on day 7, and 250 mg/dL on day 14. The 0.03 mg/kg dose group had a fasting glucose level of approximately 225 mg/dL on day 0, 220 mg/dL on day 7, and 200 mg/dL on day 14. The 0.1 mg/kg dose group had a fasting glucose level of approximately 275 mg/dL on day 0, 285 mg/dL on day 7, and 230 mg/dL on day 14. The 0.3 mg/kg dose group had a fasting glucose level of approximately 275 mg/dL on day 0, 230 mg/dL on day 7, and 150 mg/dL on day 14. The 1.0 mg/kg dose group had a fasting glucose level of approximately 250 mg/dL on day 0, 170 mg/dL on day 7, and 100 mg/dL on day 14. The 3.0 mg/kg dose group had a fasting glucose level of approximately 265 mg/dL on day 0, 190 mg/dL on day 7, and 180 mg/dL on day 14. Thus, the results of the interim analysis (through day 14) confirmed the existence of a dose-dependent beneficial effect of betacellulin on long-term glycemic control as measured by HbA_{1c} and fasting blood glucose.

### Example 23

### Other EGF Family Members Besides Betacellulin Also Reduced Blood Glucose Levels

Since betacellulin is a member of the EGF family of proteins, we compared several members of the EGF/ErbB family that have different EGF receptor binding profiles, to assess if they too have glucose lowering effects. To test this possibility we measured blood glucose, in fasted db mice, at several time points after administration of the test proteins. Male db mice were obtained from Harlan Laboratories at approximately 7-8 weeks of age and allowed to acclimate for 1 week before initiation of the test. All blood glucose measurements were performed with a Bayer Ascensia Glucometer from a drop of blood obtained by a tail nick. Betacellulin was prepared internally and came from lot #RF17-20. The other EGF family members were obtained from R&D Systems, Inc. (Minneapolis, Minn.): (i) NRG1-α/HRG1-α EGF domain (Cat#296-HR/CF), Lot Number: KC045051. This was reconstituted in 10 mM acetic acid with 0.1% BSA; (ii) HB-EGF (Cat#259-HE/CF), Lot Number: J1165091. This was reconstituted in PBS with 0.1% BSA; and (iii) EGF (Cat#236-EG), Lot Number: HLM135031. This was reconstituted in PBS with 0.1% BSA.

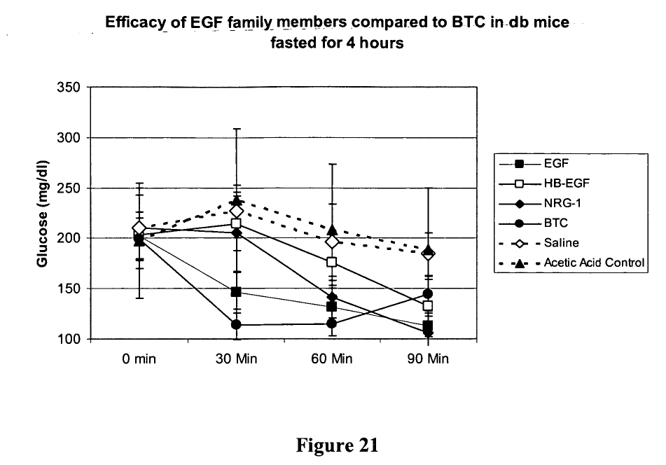

The animals were fasted for four hours followed by a blood glucose measurement at time 0 min, to determine their fasting baseline blood glucose. The mice were then distributed equally into six groups, based on their baseline measurement. The six groups were: EGF, HB-EGF, NRG-1, BTC, Saline, and acetic acid control. Each group consisted of eight mice, except for the acetic acid control group which consisted of five mice. All doses were administered subcutaneously at 1 mg/kg in a volume of 0.25 ml. After administration of the test compound, blood glucose measurements were taken at 30 min, 60 min, and 90 min. No glucose was administered in this test. The results of the test are shown in FIG. 21. Each data point represents an average of all mice in that treatment group.

FIG. 21 shows that at baseline time 0 min, mice in all the groups had a blood glucose value of approximately 204 mg/dL. For the saline treated mice (open diamonds), the blood glucose value averaged approximately 225 mg/dL at 30 min, 195 mg/dL at 60 min, and 185 mg/dL at 90 min. For the acetic acid treated control mice (black triangles), the blood glucose value averaged approximately 235 mg/dL at 30 min, 210 mg/dL at 60 min, and 190 mg/dL at 90 min. For the EGF treated mice (black squares), the blood glucose value averaged approximately 145 mg/dL at 30 min, 130 mg/dL at 60 min, and 115 mg/dL at 90 min. For the HB-EGF treated mice (open squares), the blood glucose value averaged approximately 215 mg/dL at 30 min, 175 mg/dL at 60 min, and 135 mg/dL at 90 min. For the NRG-1 treated mice (black diamonds), the blood glucose value averaged approximately 205 mg/dL at 30 min, 140 mg/dL at 60 min, and 105 mg/dL at 90 min. For the BTC treated mice (black circles), the blood glucose value averaged approximately 115 mg/dL at 30 min, 115 mg/dL at 60 min, and 145 mg/dL at 90 min. In summary, this test showed that BTC, EGF, HB-EGF, NRG-1, were all able to significantly reduce fasting blood glucose levels in db mice, compared to saline and vehicle (acetic acid) controls.

### Example 24

### Glucose Lowering Effect of Betacellulin is Dependent on the Timing of Administration

In an earlier experiment, we showed that treatment of db mice with betacellulin, after fasting and administration of glucose, caused a significant reduction in blood glucose compared to controls (Example 21, FIG. 18). We wanted to determine if this effect (that is, glucose lowering effect) was due to an acute response leading to an immediate uptake of glucose, or whether the response was dependent on long term treatment. To this end, we conducted a test in male db mice, comparing the effect of 0.3 mg/kg betacellulin administered before or concurrent with the administration of glucose.

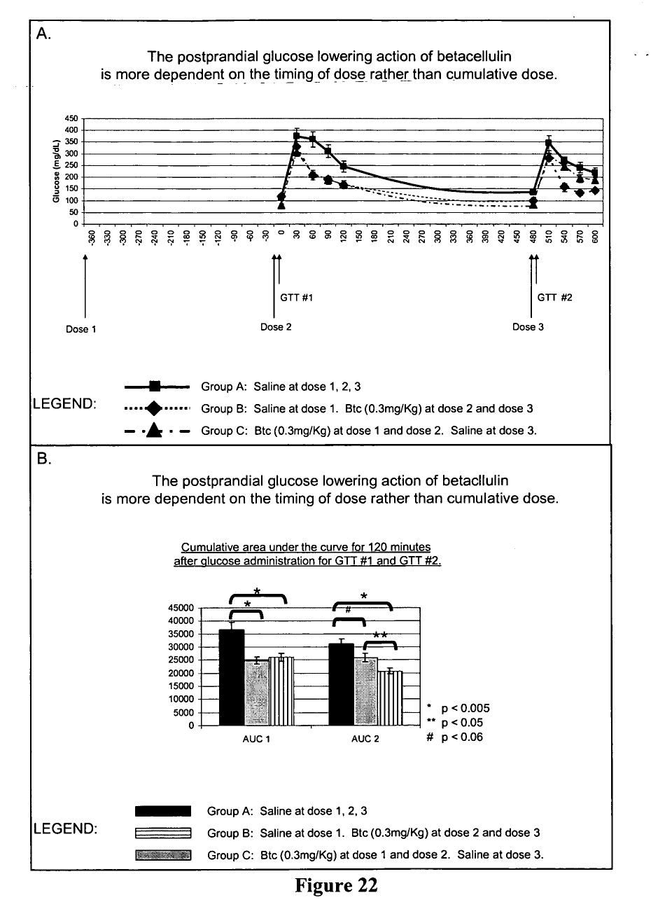

As in the previous examples, BTC was produced at our facility. The db mice were obtained from Harlan Laboratories at approximately 7-8 weeks of age and subsequently tested after 1 week of acclimation in our facility. The mice were distributed into three treatment groups. Each group received three doses of either betacellulin or saline in 0.25 ml per dose, subcutaneously every six hours starting at 4 AM. Also, starting at 4 AM, access to food was restricted for the rest of the testing period. After six hours, at 10 AM, the mice were treated with their second dose of betacellulin or saline and then a glucose tolerance test (“GTT#1”) was administered by injecting 0.75 g/kg of glucose intraperitoneally. Blood glucose was measured at several time points for two more hours. After six more hours, at 4 PM, the mice were treated with their third dose of betacellulin or saline and another glucose tolerance test (“GTT #2) was performed. All blood was obtained from tail nicks, and glucose measurements were performed with a Bayer Ascensia glucometer. Results are shown in FIG. 22. Each data point represents an average of ten mice.

The three groups of mice were: Group A mice were treated with saline at all three doses; Group B mice were treated with saline at Dose 1 and betacellulin at 0.3 mg/kg per dose at Doses 2 and 3; and Group C mice were treated with betacellulin at 0.3 mg/kg per dose at Doses 1 and 2, and saline at Dose 3.

We knew from our PK study (described later) that a dose of 0.3 mg/kg of betacellulin was cleared from the circulation within a six hour time window. Thus, the mice in group C were not expected to have any substantial level of betacellulin remaining in circulation at the time of the second GTT test. FIG. 22A shows that blood glucose level of the Group A mice (black squares) averaged about 110 mg/dL at baseline time 0, just prior to the initiation of GTT#1, peaked at about 375 mg/dL approximately 30 min after, and gradually decreased to about 350 mg/dL at 60 min, to about 300 at 90 min, and to about 250 mg/dL at 120 min, after initiation of GTT#1. Mice in Groups B and C behaved similarly initially, with blood glucose level averaging about 120 mg/dL and 75 mg/dL, respectively, at time 0, and peaking at about 325 mg/dL and 300 mg/dL, respectively, at 30 min, and both decreasing to about 200 mg/dL at 60 min, to about 185 mg/dL at 90 min., and to about 165 mg/dL at 120 min post initiation of GTT#l. Thus, the Group B mice, treated with either a single dose of betacellulin at Dose 2 (black diamonds) or the Group C mice, treated with two doses of betacellulin at Doses 1 and 2 (black triangles), appeared to be similarly effective in reducing blood glucose upon administration of glucose at time 0 in GTT#1.

At time 480 min, 8 hr after initiation of GTT#1 and just prior to the initiation of the second glucose tolerance test (GTT#2), the blood glucose level of the Group A mice (saline control) averaged about 140 mg/dL, the blood glucose level of the Group B mice averaged about 100 mg/dL, and the blood glucose level of the Group C mice averaged about 75 mg/dL. Within about 30 min after initiation of GTT#2 (at time 510 min), blood glucose level of the Group A mice peaked at about 350 mg/dL, that of the Group B and Group C mice both peaked at about 280 mg/dL. Thereafter, beginning at 60 min after initiation of GTT#2 (at time 540 min), a difference can be seen between the Group B and Group C mice. Blood glucose level at 540 min averaged about 275 mg/dL for the Group A mice, about 150 mg/dL for the Group B mice and about 250 mg/dL for the Group C mice. At 570 min post GTT#1, which was 90 min post initiation of GTT#2, the blood glucose level of the Group A mice averaged about 250 mg/dL, that of the Group B mice averaged about 140 mg/dL, and that of the Group C mice averaged about 200 mg/dL. At 600 min, or 120 min after initiation of GTT#2, blood glucose level of the Group A mice remained at an average of about 225 mg/dL, that of the Group B mice averaged about 145 mg/dL and that of the Group C mice averaged about 190 mg/dL. Thus, results show that Group C mice were less effective at clearing glucose during the second GTT test as compared to the Group B mice, but were still slightly more effective at clearing glucose when compared to the saline-treated Group A mice.

FIG. 22B shows the total area under the curve (“AUC1”) in GTT#1 was not significantly different between the Group B and Group C, but each of Group B and Group C was significantly different from the Group A in the AUC1 in GTT#1. Further, the Group B had a significantly lower area under the curve (“AUC2”) for the second GTT (GTT#2) as compared to the Group A or the Group C. Also, we found that although the Group B and the Group C mice received an equivalent total dose of betacellulin during the course of the 14-hour test, the Group C mice did not achieve an equivalent glucose lowering effect during the second GTT.

This experiment indicates that administration of betacellulin concurrent with glucose excursions derived the highest benefit in acute reduction of blood glucose and that an equivalent cumulative dose of betacellulin administered ahead of a glucose excursion the same day in this test was not sufficient to achieve maximal glucose lowering effects. The results of this experiment predicted that, for postprandial applications, the timing of administration of betacellulin and the increase in carbohydrate load should be in close proximity such that betacellulin would be present at therapeutic concentrations in the blood at the time of the anticipated postprandial glucose excursion. Therefore, for postprandial applications, betacellulin should optimally be administered at or around the time of a meal. This rapid-onset, relatively short-lived hypoglycemic effect of betacellulin indicates a distinct pharmacologic effect that cannot be explained by pancreatic islet cell neogenesis or other increase in beta islet cell mass.

### Example 25

### Pharmacokinetic Parameters of Betacellutin in Rats

In an earlier experiment (Example 24, FIG. 22), we showed that treatment of db mice with betacellulin, after fasting and administration of glucose, caused a significant reduction in blood glucose compared to controls. The test indicated that the glucose lowering effects were associated with concurrent administration of glucose and BTC, and that the effects were not associated with random administration of betacellulin. To better understand the relationship of dose timing, a pharmacokinetic (“PK”) test in Sprague Dawley rats was perfomed with betacellulin. The in vivo aspect of the PK test was subcontracted to Northview Pacific Laboratories Inc. (Hercules, Calif.). The rats were males and weighed approximately 250-300 grams. Betacellulin was prepared internally from E. coli.

Betacellulin (BTC), or vehicle, were administered according to a schedule, which was tabulated as follows:

| | | | Dose | Dose | Dose | Blood Collection Time Points | | | | | | | | |
|---|---|---|---|---|---|---|---|---|---|---|---|---|---|---|
| Group # | n | Treatment | Route | (mg/kg) | Volume | 0 | 2 | 15 | 30 | 60 | 120 | 240 | 480 | 1440 |
| 1 | 4 | Vehicle | SC | 0 | 0.5 | X | | X | X | X | X | X | X | X |
| 2 | 4 | BTC | SC | 0.01 | 0.5 | X | | X | X | X | X | X | X | X |
| 3 | 4 | BTC | SC | 0.1 | 0.5 | X | | X | X | X | X | X | X | X |
| 4 | 4 | BTC | SC | 1 | 0.5 | X | | X | X | X | X | X | X | X |
| 5 | 4 | BTC | SC | 10 | 0.5 | X | | X | X | X | X | X | X | X |
| 6 | 4 | Vehicle | iv | 0 | 0.5 | X | X | X | X | X | X | X | X | |
| 7 | 4 | BTC | iv | 0.01 | 0.5 | X | X | X | X | X | X | X | X | |
| 8 | 4 | BTC | iv | 0.1 | 0.5 | X | X | X | X | X | X | X | X | |
| 9 | 4 | BTC | iv | 1 | 0.5 | X | X | X | X | X | X | X | X | |
| 10 | 4 | BTC | iv | 10 | 0.5 | X | X | X | X | X | X | X | X | |

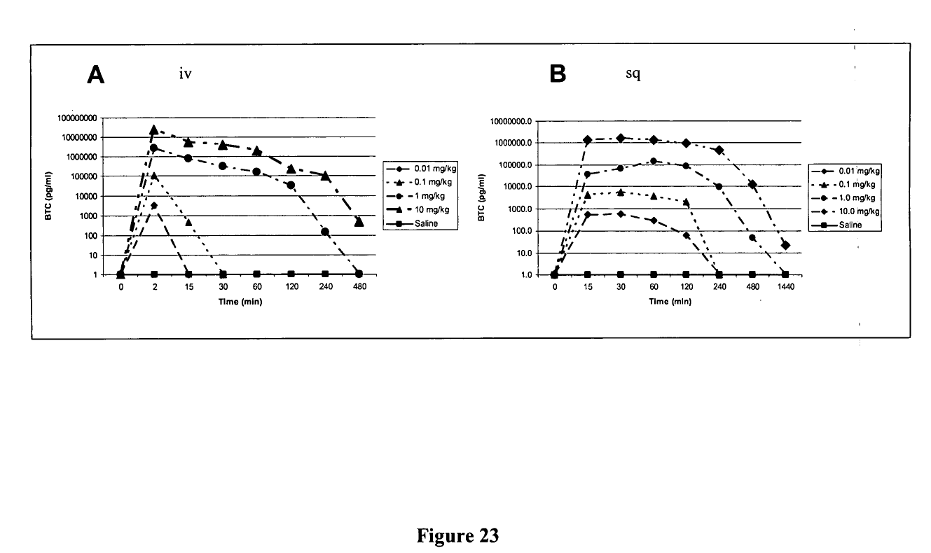

Blood (˜0.5 mL) was collected into vacutainer tubes containing EDTA at the time points outlined. After collection the specimens were centrifuged at approximately 2800 rpm (1000×g) at 2-8° C. for approximately 15 min. Plasma was collected and frozen at −20° C. The samples were shipped to us to determine the amount of betacellulin in the plasma. Betacellulin (BTC) was assayed from plasma using an ELISA kit from R&D Systems, Inc., Cat# DY261 (Minneapolis, Minn.). Results are shown in FIG. 23A (intravenous administration) and FIG. 23B (subcutaneous administration, “sq”) and in the tables below. Each data point represents an average of four rats.

For subcutaneous dosing, the following results were obtained. For doses at 0.01 mg/kg, 0.1 mg/kg. 1.0 mg/kg and 10 mg/kg of betacellulin, respectively, the Tₘₐₓ was reached at approximately 18 min, 32 min, 39 min, and 42 min, respectively; the Cₘₐₓ was reached at approximately 657 pg/ml, 3.7 ng/ml, 166 ng/ml, and 2.1 μg /ml, respectively; the half-life of betacellulin was approximately 26 min, 75 min, 33 min, and 61 min, respectively; and the plasma concentration of betacellulin fell below 100 pg/ml by 120 min, 240 min, 480 min, and 1440 min, respectively.

For intravenous dosing, the following results were obtained. For doses at 0.01 mg/kg, 0.1 mg/kg, 1.0 mg/kg and 10 mg/kg, respectively, the plasma concentration of betacellulin at 2 min after injection was approximately 3.3 ng/ml, 109 ng/ml, 2.7 μg/ml, and 25 μg/ml, respectively; the half-life of betacellulin was approximately 1 min, 2 min, 15 min, and 31 min, respectively; and the plasma concentration of betacellulin was less than 10 pg/ml at 15 min, 30 min, 480 min and 960 min, respectively.

The first series of tables presented below shows the PK results of subcutaneous administration of betacellulin to the rats. The detection limit for betacellulin was >/=10 pM. UD means under detection limit. K means value in thousands.

| | Assay after Saline Administration | | | | | | | | | | | |
|---|---|---|---|---|---|---|---|---|---|---|---|---|
| | BTC (pM) | | | | Insulin (ng/ml) | | | | Glucose (mg/dL) | | | |
| Time | Animal Number | | | | Animal Number | | | | Animal Number | | | |
| (min) | 1 | 2 | 3 | 4 | 1 | 2 | 3 | 4 | 1 | 2 | 3 | 4 |
| 0 | UD | UD | UD | UD | 4.7 | 6.5 | 4.1 | 1.7 | 154 | 182 | 171 | 105 |
| 15 | UD | UD | UD | UD | 7.7 | 3.2 | 2.6 | 2.5 | 264 | 155 | 141 | 105 |
| 30 | UD | UD | UD | UD | 5.3 | 1.3 | 2.1 | 2.0 | 230 | 138 | 148 | 97 |
| 60 | UD | UD | UD | UD | 3.5 | 1.2 | 1.2 | 1.6 | 132 | 120 | 159 | 105 |
| 120 | UD | UD | UD | UD | 4.4 | 1.2 | 2.9 | 2.9 | 146 | 118 | 109 | 143 |
| 240 | UD | UD | UD | UD | 5.4 | 4.5 | 1.3 | 2.0 | 184 | 179 | 123 | 166 |
| 480 | UD | UD | UD | UD | 5.6 | 2.3 | 5.9 | 1.9 | 354 | 193 | 146 | 131 |
| 1440 | UD | UD | UD | UD | 4.9 | 8.5 | 0.9 | 2.0 | 188 | 161 | 126 | 200 |
| | Assay after Administration of 0.01 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 5 | 6 | 7 | 8 | 5 | 6 | 7 | 8 | 5 | 6 | 7 | 8 |
| 0 | UD | UD | UD | UD | 4.0 | 2.5 | 1.7 | 1.5 | 166 | 120 | 228 | 202 |
| 15 | 52 | 95 | 47 | 47 | 4.2 | 1.7 | 1.4 | 1.1 | 134 | 136 | 157 | 195 |
| 30 | 56 | 84 | 45 | 71 | 2.5 | 2.0 | 1.0 | 0.8 | 145 | 93 | 141 | 145 |
| 60 | 36 | 44 | 32 | 20 | 2.2 | 1.7 | 1.2 | 1.0 | 91 | 110 | 132 | 156 |
| 120 | 2 | 15 | 4 | 7 | 3.3 | 2.8 | 0.7 | 0.9 | 149 | 131 | 177 | 183 |
| 240 | UD | UD | UD | UD | 2.1 | 0.7 | 0.3 | 0.1 | 154 | 86 | 115 | 211 |
| 480 | UD | UD | UD | UD | 2.3 | 0.7 | 0.8 | 0.3 | 150 | 112 | 176 | 173 |
| 1440 | UD | UD | UD | UD | 1.7 | 1.7 | 0.8 | 0.8 | 154 | 161 | 171 | 178 |
| | Assay after Administration of 0.1 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 9 | 10 | 11 | 12 | 9 | 10 | 11 | 12 | 9 | 10 | 11 | 12 |
| 0 | UD | UD | UD | UD | 3.6 | 1.3 | 0.2 | 0.7 | 161 | 125 | 137 | 174 |
| 15 | 582 | 445 | 313 | 495 | 1.8 | 1.2 | 0.0 | 1.0 | 140 | 125 | 137 | 152 |
| 30 | 725 | 547 | 494 | 635 | 1.4 | 0.4 | 0.0 | 0.7 | 167 | 131 | 112 | 151 |
| 60 | 410 | 372 | 380 | 430 | 0.7 | 0.3 | 0.4 | 0.6 | 151 | 148 | 114 | 142 |
| 120 | 195 | 204 | 226 | 287 | 1.4 | 0.7 | 0.7 | 0.4 | 131 | 126 | 147 | 160 |
| 240 | UD | UD | UD | UD | 2.7 | 0.5 | 0.4 | 0.3 | 168 | 144 | 173 | 113 |
| 480 | UD | UD | UD | UD | 1.3 | 0.7 | 0.7 | 1.2 | 121 | 162 | 160 | 167 |
| 1440 | UD | UD | UD | UD | 0.9 | 0.6 | 0.9 | 0.8 | 178 | 178 | 172 | 171 |
| | Assay after Administration of 1 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 13 | 14 | 15 | 16 | 13 | 14 | 15 | 16 | 13 | 14 | 15 | 16 |
| 0 | UD | UD | UD | UD | 4.0 | 2.4 | 3.3 | 0.9 | 137 | 149 | 151 | 161 |
| 15 | 5.5K | 3.9K | 3.7K | 2.4K | 0.3 | 0.4 | 0.6 | 0.0 | 143 | 123 | 138 | 123 |
| 30 | 8.3K | 10.9K | 6.1K | 3.3K | 0.0 | 0.0 | 0.1 | 0.0 | 183 | 135 | 138 | 133 |
| 60 | 22.3K | 14K | 17.3K | 11.1K | 0.8 | 0.3 | 0.8 | 0.4 | 237 | 198 | 146 | 184 |
| 120 | 10.9K | 5.6K | 13.9K | 7.6K | 0.1 | 0.4 | 0.4 | 0.5 | 197 | 161 | 109 | 174 |
| 240 | 634 | 973 | 2.2K | 486 | 0.1 | 0.4 | 1.0 | 0.9 | 240 | 127 | 147 | 182 |
| 480 | 6 | 9 | 2 | 6 | 1.1 | 0.7 | 1.2 | 1.3 | 217 | 134 | 168 | 127 |
| 1440 | UD | UD | UD | UD | 2.4 | 1.1 | 1.2 | 1.1 | 272 | 132 | 173 | 209 |
| | Assay after Administration of 10 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 17 | 18 | 19 | 20 | 17 | 18 | 19 | 20 | 17 | 18 | 19 | 20 |
| 0 | UD | UD | UD | UD | 2.3 | 1.5 | 4.6 | 3.4 | 166 | 193 | 220 | 89 |
| 15 | 120K | 205K | 92K | 172K | 0.0 | 0.0 | 0.0 | 0.1 | 90 | 108 | 131 | 90 |
| 30 | 153K | 191K | 189K | 199K | 0.0 | 0.0 | 0.0 | 1.7 | 139 | 139 | 157 | 102 |
| 60 | 119K | 189K | 132K | 169K | 0.0 | 0.2 | 0.2 | 0.7 | 165 | 183 | 164 | 120 |
| 120 | 114K | 106K | 86K | 124K | 0.0 | 0.0 | 1.9 | 1.4 | 136 | 147 | 169 | 147 |
| 240 | 63.8K | 52K | 36K | 50K | 0.2 | 1.0 | 1.3 | 0.7 | 74 | 131 | 150 | 130 |
| 480 | 1.4K | 891 | 1.5K | 1.6K | 0.0 | 1.2 | 1.3 | 0.1 | 111 | 170 | 179 | 118 |
| 1440 | 3 | 3 | 2 | 1 | 1.0 | 1.0 | 0.9 | 1.7 | 133 | 203 | 157 | 91 |

The series of tables presented next show the PK results of intravenous administration of betacellulin to the rats. The detection limit for betacellulin was >/=10 pM. UD means under the detection limit of 0.1 pM. K means value in thousands. NA means not available.

| | Assay after Saline Administration | | | | | | | | | | | |
|---|---|---|---|---|---|---|---|---|---|---|---|---|
| | BTC (pM) | | | | Insulin (ng/ml) | | | | Glucose (mg/dL) | | | |
| Time | Animal Number | | | | Animal Number | | | | Animal Number | | | |
| (min) | 1 | 2 | 3 | 4 | 1 | 2 | 3 | 4 | 1 | 2 | 3 | 4 |
| 0 | 0.1 | 0.1 | 0.1 | 0.1 | 1.1 | 0.7 | 1.5 | 1.7 | 153 | 152 | 142 | 177 |
| 2 | 0.1 | 0.1 | 0.1 | 0.1 | 0.9 | 1.5 | 0.4 | 1.5 | 142 | 139 | 126 | 115 |
| 15 | 0.1 | 0.1 | 0.1 | 0.1 | 0.8 | 0.3 | 0.4 | 1.0 | 138 | 129 | 137 | 134 |
| 30 | 0.1 | 0.1 | 0.1 | 0.1 | 0.4 | 0.3 | 0.2 | 0.8 | 152 | 151 | 127 | 145 |
| 60 | 0.1 | 0.1 | 0.1 | 0.1 | 0.4 | 0.7 | 0.1 | 0.8 | 146 | 177 | 155 | 159 |
| 120 | 0.1 | 0.1 | 0.1 | 0.1 | 0.3 | 0.5 | 1.7 | 0.8 | 137 | 149 | 173 | 127 |
| 240 | 0.1 | 0.1 | 0.1 | 0.1 | 0.3 | 0.2 | 0.6 | 0.9 | 135 | 145 | 187 | 150 |
| 480 | 0.1 | 0.1 | 0.1 | 0.1 | 0.4 | 1.1 | 0.5 | 0.8 | 150 | 169 | 179 | 153 |
| | Assay after Administration of 0.01 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 5 | 6 | 7 | 8 | 5 | 6 | 7 | 8 | 5 | 6 | 7 | 8 |
| 0 | 0.1 | 0.1 | 0.1 | NA | 1.3 | 0.8 | 1.4 | NA | 175 | 146 | 157 | NA |
| 2 | NA | 720.0 | 211.1 | 548.4 | NA | 0.2 | 0.3 | 0.4 | NA | 156 | 155 | 135 |
| 15 | 0.1 | 0.1 | 0.1 | 0.1 | 0.1 | 0.9 | 0.2 | 0.4 | 154 | 147 | 149 | 154 |
| 30 | 0.1 | 0.1 | 0.1 | 0.1 | 0.3 | 0.4 | 0.3 | 0.7 | 145 | 124 | 140 | 145 |
| 60 | 0.1 | 0.1 | 0.1 | 0.1 | 0.2 | 0.3 | 0.3 | 0.4 | 163 | 158 | 178 | 159 |
| 120 | 0.1 | 0.1 | 0.1 | 0.1 | 0.2 | 0.4 | 0.5 | 0.5 | 155 | 155 | 158 | 140 |
| 240 | 0.1 | 0.1 | 0.1 | 0.1 | 0.4 | 0.6 | 0.4 | 0.5 | 158 | 159 | 154 | 148 |
| 480 | 0.1 | 0.1 | 0.1 | 0.1 | 1.0 | 0.3 | 0.4 | 0.4 | 174 | 117 | 157 | 141 |
| | Assay after Administration of 0.1 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 9 | 10 | 11 | 12 | 9 | 10 | 11 | 12 | 9 | 10 | 11 | 12 |
| 0 | 0.1 | 0.1 | 0.1 | 0.1 | 1.8 | 4.0 | 5.6 | 5.3 | 123 | 171 | 172 | 181 |
| 2 | 12K | 11K | 15K | 9K | 0.2 | 0.7 | 1.3 | 0.7 | 144 | 147 | 147 | 161 |
| 15 | 43 | 78 | 67 | 16 | 0.0 | 1.1 | 0.0 | 0.6 | 136 | 152 | 116 | 146 |
| 30 | 0.1 | 0.1 | 0.1 | 0.1 | 0.1 | 0.8 | 0.6 | 0.6 | 140 | 133 | 144 | 167 |
| 60 | 0.1 | 0.1 | 0.1 | 0.1 | 0.4 | 0.7 | 0.8 | 0.6 | 143 | 145 | 175 | 171 |
| 120 | 0.1 | 0.1 | 0.1 | 0.1 | 0.0 | 2.1 | 2.2 | 2.3 | 157 | 140 | 143 | 159 |
| 240 | 0.1 | 0.1 | 0.1 | 0.1 | 1.1 | 0.4 | 1.7 | 1.4 | 207 | 130 | 159 | 168 |
| 480 | 0.1 | 0.1 | 0.1 | 0.1 | 0.4 | 1.5 | 0.9 | 0.7 | 118 | 161 | 150 | 172 |
| | Assay after Administration of 1 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 13 | 14 | 15 | 16 | 13 | 14 | 15 | 16 | 13 | 14 | 15 | 16 |
| 0 | 0.1 | 0.1 | 0.1 | 0.1 | 1.9 | 2.6 | 1.9 | 2.5 | 197 | 151 | 157 | 175 |
| 2 | 304K | 264K | 335K | 303K | 0.5 | 0.2 | 0.5 | 0.3 | 169 | 159 | 148 | 125 |
| 15 | 88K | 81K | 98K | 86K | 0.1 | 0.0 | 0.0 | 0.1 | 120 | 138 | 112 | 161 |
| 30 | 41K | 35K | 40K | 30K | 0.2 | 0.0 | 0.3 | 0.8 | 156 | 116 | 133 | 199 |
| 60 | 21K | 17K | 17K | 13K | 0.5 | 0.8 | 0.4 | 0.2 | 175 | 167 | 138 | 229 |
| 120 | 3.6K | 3.4K | 4.1K | 3.2K | 0.7 | 0.2 | 0.3 | 0.7 | 140 | 137 | 125 | 181 |
| 240 | 12 | 14 | 24 | 15 | 0.7 | 0.8 | 0.6 | 1.1 | 154 | 192 | 166 | 188 |
| 480 | 0.1 | 0.1 | 0.1 | 0.1 | 0.7 | 0.7 | 0.6 | 0.5 | 302 | 151 | 190 | 207 |
| | Assay after Administration of 10 mg/kg of BTC | | | | | | | | | | | |
| | BTC (pM) | | | | Insulin (ng/ml) | | | | Glucose (mg/dL) | | | |
| Time | Animal Number | | | | Animal Number | | | | Animal Number | | | |
| (min) | 17 | 18 | 19 | 20 | 17 | 18 | 19 | 20 | 17 | 18 | 19 | 20 |
| 0 | 0.1 | 0.1 | 0.1 | 0.1 | 0.9 | 1.0 | 0.8 | 0.5 | 153 | 153 | 172 | 137 |
| 2 | 2807K | 2689K | 2849K | 2652K | 0.2 | 0.0 | 0.1 | 0.0 | 161 | 161 | 148 | 166 |
| 15 | 648K | 498K | 660K | 587K | 0.0 | 0.0 | 0.8 | 0.0 | 99 | 99 | 130 | 111 |
| 30 | 457K | 383K | 457K | 461K | 0.3 | 0.0 | 0.4 | 0.0 | 171 | 171 | 164 | 151 |
| 60 | 183K | 209K | 260K | 248K | 0.0 | 0.0 | 0.2 | 0.0 | 161 | 161 | 194 | 177 |
| 120 | 24K | 29K | 24K | 31K | 0.1 | 0.4 | 0.3 | 0.0 | 154 | 154 | 197 | 170 |
| 240 | 11K | 11K | 9.6K | 13K | 0.0 | 0.0 | 0.3 | 0.0 | 146 | 146 | 192 | 121 |
| 480 | 60 | 49 | 23 | 83 | 0.1 | 0.0 | 0.5 | 0.5 | 172 | 172 | 199 | 187 |

In summary, the PK studies showed that, in normal rats, betacellulin was rapidly cleared from the blood and had a circulating half-life of approximately one hour or less depending on the route of administration. The rapid clearance of betacellulin may be one explanation for why we did not see a glucose lowering effect when betacellulin was administered to mice in an asynchronous manner with respect to blood glucose excursions (see previous examples). These results indicate that betacellulin should optimally be present at a pharmacological level in the blood when glucose levels go up, to obtain a significant acute glucose-lowering effect such as in post-prandial applications.

### Example 26

### The Glucose-Lowering Actions of Betacellulin and GLP1 were at Least Additive

Glucagon-like peptide-1 (GLP1) (as reviewed in Holst J J. Diabetologia, 49(2): 253-60 (2006)) and exendin-4 (as reviewed in Triplitt C and Chiquette E., J, Am Pharm Assoc (Wash D.C.), 46(1): 44-52 (2006)) are potent stimulators of insulin secretion, and consequently have significant effects on the regulation of glucose metabolism. Exendin-4 is a peptide isolated from the Gila monster and is a potent agonist of GLP1 receptors. In vitro and in vivo tests by others suggested that both molecules exhibited glucose lowering effects that were dependent on GLP1 receptor-mediated pathways. Both molecules reportedly were effective at lowering blood glucose in rodent models. We showed in Example 22 (FIG. 20D) that betacellulin treatment resulted in a reduction of plasma insulin levels. Hence, we had evidence that betacellulin would enhance the effect of GLP1 receptor agonists in lowering blood glucose through a mechanism that is different from GLP1 receptor-mediate pathways.

To demonstrate that betacellulin would enhance the effect of a drug that acts on GLP1 receptors, we conducted a glucose tolerance test (“GTT”) in male db mice that were treated with either 0.2 mg/kg of GLP1 alone, or betacellulin alone or a combination of both. We used 0.3 mg/kg of betacellulin for administration in this test. The db mice were obtained from Harlan Laboratories at approximately 7-8 weeks of age and subsequently tested after about 1 week of acclimation in our facility. Betacellulin (BTC) was prepared at our facility from expression in an E. coli host. GLP1 was purchased from Sigma-Aldrich Inc. (Cat# G9416). All blood glucose measurements were performed from tail vein nicks with a Bayer Ascensia glucometer.

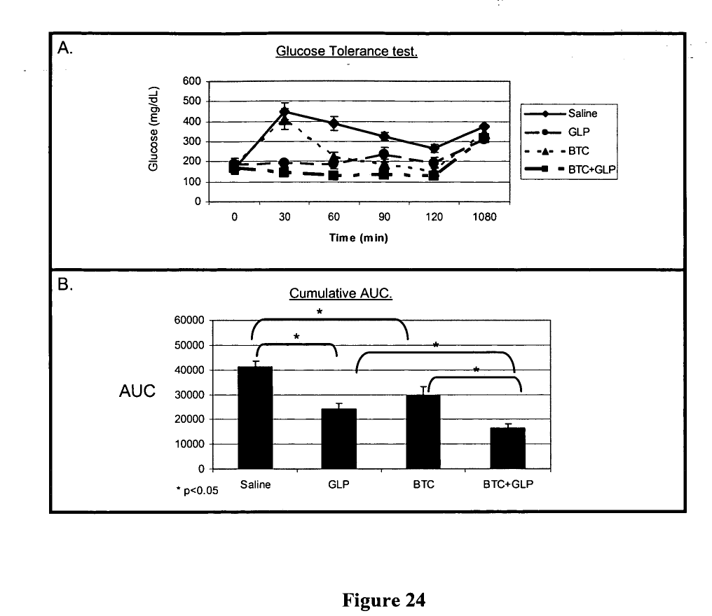

Initially, baseline glucose values at time 0 were obtained following a five hour fast. The mice were then distributed into four groups based on their fasting glucose values. The group makeup was as follows: Seven Group 1 mice were injected with saline (♦/diamonds). Eight Group 2 mice were injected with GLP1 alone (●/circles). Eight Group 3 mice were injected with betacellulin alone (▴/triangles), and seven Group 4 mice were injected with a combination of GLP1 plus BTC (▪/squares). At the onset of the GTT, the mice were injected subcutaneously with the designated drug, just prior to administration of 0.75 mg/kg of glucose intraperitoneally. Glucose measurements were obtained for the following two hours. Each data point represents an average of all the mice in the group. The results of this test are shown in FIG. 24.

FIG. 24A shows the blood glucose level of the saline treated Group 1 started at a baseline of about 175 mg/dL at time 0 and peaked at about 450 mg/dL at 30 min, then dropped to about 400 mg/dL at 60 min, about 325 mg/dL at 90 min, increased again to about 390 mg/dL at 1080 min (i.e., 18 hr.) For the GLP1 treated Group 2 mice, their blood glucose level remained about the same at the 200-230 mg/dL level at times 30 min, 60 min, 90 min and 120 min, and increased to about 325 mg/dL at 1080 min. For the betacellulin treated Group 3 mice, the blood glucose level increased from about 175 mg/dL at time 0 to a peak of about 400 mg/dL at 30 min, and quickly decreased to about 210 mg/dL at 60 min, about 190 mg/dL at 90 min, and about 150 mg/dL at 120 min, but went up to about 325 mg/dL at 1080 min. For the Group 4 mice treated with both GLP1 and betacellulin, the blood glucose level at about 160 mg/dL at time 0, remained low at between about 150 mg/dL to about 125 mg/dL at times 30 min, 60 min, 90 min and 120 min, and then went up to about 310 mg/dL at 1080 min.

FIG. 24B shows the cumulative area under the curve (“AUC”) for 120 min following glucose administration. The differences between the GLP1 treated Group 2 and the saline control Group 1, between GLP 1 treated Group 2 the combination GLP1 and betacellulin treated Group 4, as well as the differences between the betacellulin treated Group 3 and the control Group 1, and between the betacellulin treated Group 3 and the combination GLP1 and betacellulin treated Group 4, are all statistically significant (as determined by a t-test).

These results showed that the db mice, as animal models of diabetes, were responsive to combination treatment with GLP1 and betacellulin. The combination of betacellulin and GLP1 resulted in a greater reduction in blood glucose than either of these drugs alone, showing an additive glucose lowering effect, especially when these drugs were administered concurrently with postprandial glucose excursions. This indicates the glucose lowering effect of betacellulin was at least additive to that mediated by GLP1 receptor-mediated pathways, and that betacellulin treatment added to, but did not interfere with, insulinotropic drugs.

### Example 27

### The Glucose Lowering Effects of Betacellulin and Metformin were at Least Additive

Metformin is a hypoglycemic agent that is used in the treatment of Type II diabetes, as described in Bailey C J Diabetes Care 15(6): 755-772 (1992). According to the package insert, “Metformin decreases hepatic glucose production, decreases intestinal absorption of glucose, and improves insulin sensitivity by increasing peripheral glucose uptake and utilization.” The glucose-lowering effect of Metformin occurs without stimulation of insulin secretion and the presence of insulin is required. Enhancement of insulin action at the post-receptor level occurs in peripheral tissues, such as muscle, where Metformin increases insulin-mediated glucose uptake and oxidative metabolism.

We believed that betacellulin would enhance the effect of Metformin in lowering blood glucose in diabetics and set out to demonstrate this effect. We used male db mice in this test and compared the effect of betacellulin administered at a dose of 1.0 mg/kg alone or in combination with 250 mg/kg of metformin. The db mice were obtained from Harlan Laboratories at approximately 7-8 weeks of age and subsequently were used after 1 week of acclimation in our facility. Betacellulin was prepared at our facility. Metformin was purchased from Sigma-Aldrich Inc. (Cat#D5035). All blood glucose measurements were taken from tail vein nicks and performed with a Bayer Ascensia glucometer. All injections were made in 0.25 ml volume.

The mice were first distributed into two groups based on fasting glucose values. Mice were fasted for 5 hours once for purposes of grouping, before the second fasting 3 days later on, which was done for purposes of the GTT test. One group, the “Metformin Group” with 20 mice, was treated intraperitoneally with 250 mg/kg metformin once a day at 8 AM for three days; the other group, the “Saline Group” with 10 mice, was treated with saline for the same period. Immediately after dosing on the third day, the mice were subjected to a 5 hour fast at the end of which (i.e., at time 0 min) GTT was administered. At the onset of the GTT, the metformin- and saline-treated mouse groups were each split into two subgroups that received subcutaneous injections of either betacellulin (“BTC” at 1 mg/kg), or saline just prior to administration of 0.75 mg/kg of glucose intraperitoneally. The resulting groups were: (i) Metformin-BTC (♦), (ii) Saline-BTC (▴), (iii) Metformin-Saline (●), and (iv) Saline-Saline (▪). The onset of the GTT occurred at approximately 1:00 PM, five hours after the last metformin dose.

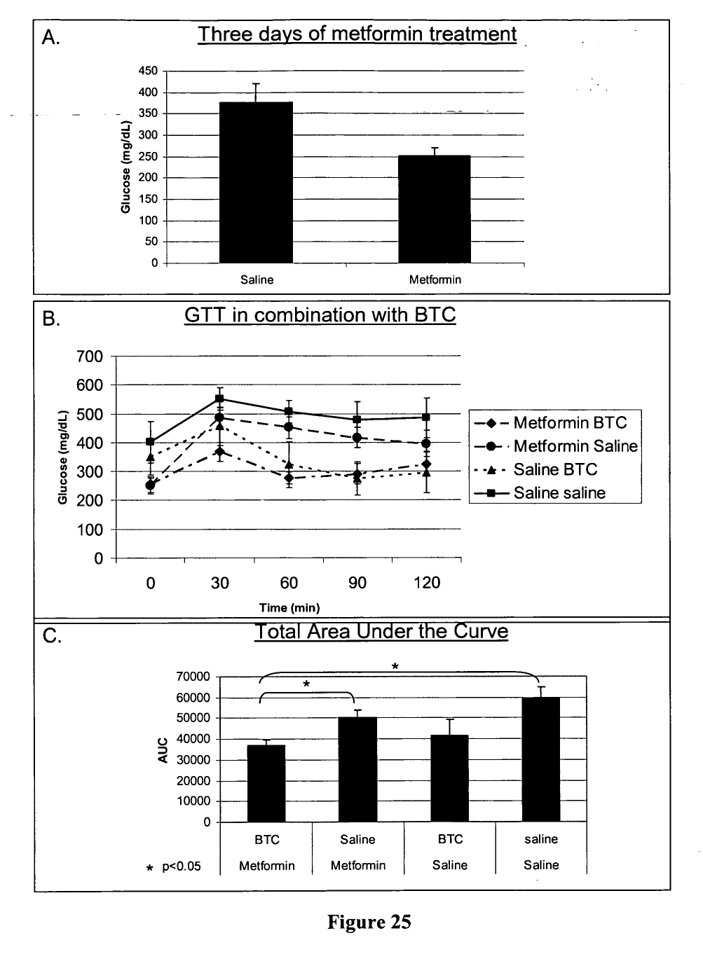

The results of the test are shown in FIG. 25. FIG. 25A shows that after three days of treatment, the fasting blood glucose level of the 20 db mice in the Metformin Group averaged about 250 mg/dL, which was significantly lower than that of the twenty db mice in the Saline Group, which averaged about 375 mg/dL.

FIG. 25B shows that 5 db mice in the Saline Saline Group had the highest average blood glucose level in the GTT, starting at about 400 mg/dL at time 0 min, rising to about 550 mg/dL at 30 min, then decreasing to about 500 mg/dL at 60 min, then to about 475 mg/dL at 90 min, and to about 500 mg/dL at 120 min. Blood glucose level of the 5 db mice in the Saline BTC Group averaged about 350 mg/dL at time 0, and increased to about 450 mg/dL at 30 min, then decreased to about 325 mg/dL at 60 min, and to about 275 mg/dL at 90 min and about 290 mg/dL at 120 min. The ten db mice in the Metformin Saline Group started out with a lower average blood glucose level, at about 250 mg/dL at time 0, then increased to about 500 mg/dL at 30 min, and decreased to about 450 mg/dL at 60 min, and to about 410 mg/dL at 90 min, and to about 400 mg/dL at 120 min. The eight db mice in the Metformin BTC Group performed the best, starting with an average blood glucose level of about 250 mg/dL at time 0, increasing to a high of about 370 mg/dL at time 30 min, then decreased to about 280 mg/dL at 60 min, and to about 290 mg/dL at 90 min, and to about 315 mg/dL at 120 min.

FIG. 25C shows the total AUC for the four different treatment groups. The difference between the Metformin Saline Group and the Metformin BTC Group was statistically significant (p<0.05). The difference between the Metformin Saline Group and the Saline Saline Group was also statistically significant (p<0.05, t-test).

This experiment demonstrated that treatment of diabetic animals with Metformin alone resulted in reduction of fasting blood sugar, but Metformin was not very effective in mediating acute reduction in blood glucose after a glucose excursion (i.e., GTT), as the blood glucose level of the treated db mice remained high (in the 400 mg/dL-500 mg/dL range) over the 120 min of observation. Treatment with betacellulin alone was effective in mediating acute reduction of blood glucose after glucose excursion in a rapid time course (within about 60 min after administration of the bolus of glucose). Treatment of betacellulin in combination with metformin resulted in an acute glucose lowering effect that is at least additive when compared to that of each agent alone, especially when betacellulin was administered concurrently with postprandial glucose excursions, achieving rapid decrease in blood glucose level within about 60 min after administration of a bolus of glucose. These data indicated that combination of betacellulin with an agent that inhibited hepatic gluconeogenesis and enhanced peripheral glucose uptake and utilization resulted in better postprandial glucose control than that achieved with either agent alone.

### Example 28

### The Glucose Lowering Effects of Betacellulin and Insulin are Additive

We found in an earlier test (Example 22, FIG. 20) that betacellulin treatment of db mice resulted in a reduction of plasma insulin levels of the treated mice. We suspected that betacellulin might work through a mechanism that was complementary to the insulin receptor-mediated pathway. To show that this was indeed the case, we conducted a glucose tolerance test (“GTT”) in male db mice that were treated with either 2 U/kg of insulin alone, or betacellulin alone, or a combination of both insulin and betacellulin. We used 0.3 mg/kg of betacellulin for injection in this test.

The db mice were obtained from Harlan Laboratories at approximately 7-8 weeks of age and subsequently tested after about 1 week of acclimation in our facility. Betacellulin was prepared at our facility from expression in an E. coli host. Insulin (Humilin®, Eli Lilly, Indianapolis, Ind.) was purchased from a local pharmacy. All blood glucose measurements were performed with blood from tail vein nicks (about 2 microliter) using a Bayer Ascensia glucometer.

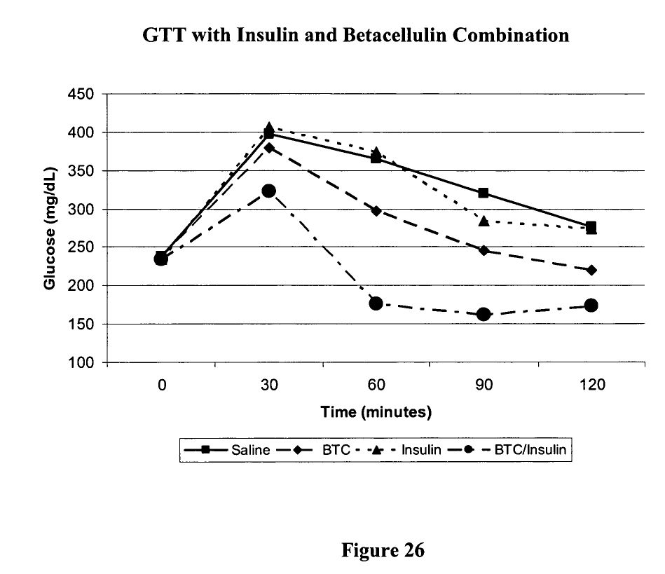

The results of this test are shown in FIG. 26. Baseline glucose values at time 0 were obtained following a five hour fast. The mice were then equally distributed into 4 groups of ten mice based on their fasting glucose values. The group makeup was as follows: Group 1 mice were injected with saline (▪/squares). The Group 2 mice were injected with insulin alone (▴/triangles). The Group 3 mice were injected with betacellulin (“BTC”) alone (♦/diamonds), and the Group 4 mice were injected with a combination of insulin plus BTC (●/circles). At the onset of the GTT, the mice were injected subcutaneously with the designated drug in a volume of 250 microliter, immediately followed by administration of 0.75 mg/kg of glucose intraperitoneally. Glucose measurements were obtained for the following two hours. Each data point represents an average of ten db mice.

FIG. 26 shows the blood glucose level of the saline treated Group 1 started at a baseline of about 230 mg/dL at time 0 and peaked at about 400 mg/dL at 30 min, then dropped to about 360 mg/dL at 60 min, about 320 mg/dL at 90 min, and 275 mg/dL at 120 min. For the insulin treated Group 2 mice, their blood glucose level started at a baseline of about 230 mg/dL at time 0 and peaked at about 400 mg/dL at 30 min, then dropped to about 360 mg/dL at 60 min, about 280 mg/dL at 90 min, and 275 mg/dL at 120 min. For the betacellulin treated Group 3 mice, the blood glucose level started at a baseline of about 230 mg/dL at time 0 and peaked at about 375 mg/dL at 30 min, then dropped to about 300 mg/dL at 60 min, about 250 mg/dL at 90 min, and 220 mg/dL at 120 min. For the Group 4 mice, treated with both insulin and betacellulin, the blood glucose level started at a baseline of about 230 mg/dL at time 0 and peaked at about 320 mg/dL at 30 min, then dropped to about 175 mg/dL at 60 min, about 160 mg/dL at 90 min, and 175 mg/dL at 120 min. The differences between the combination treated group (group 4) and the insulin treated group (group 2), and the combination treated group (group 4) and the betacellulin treated group (group 1), are both statistically significant.

These results show that the db mice, which are animal models of diabetes, behaved as insulin-resistant animals in exhibiting no significant difference in response to insulin treatment alone as compared to the saline-treated controls, with their blood glucose level remaining relatively high (between about 275 mg/dL and about 400 mg/dL) over 120 min after administration of a bolus of glucose. The animals in the betacellulin treated group responded more rapidly to treatment, achieving a significant reduction of blood glucose level by about 60 min, and returning to the pre-GTT level within about 90 min of the glucose administration. The animals treated with a combination of insulin and betacellulin showed the most significant response, achieving a lower than basal level of blood glucose within about 60 min of the glucose administration, which level was maintained over the next 60 min of observation.

Thus, the combination of betacellulin and insulin resulted in a greater reduction in blood glucose than either of these drugs alone, showing at least an additive or a synergistic glucose lowering effect, especially when these drugs were administered concurrently with postprandial glucose excursions. These results indicated that the glucose lowering effect of betacellulin enhanced but did not interfere with that mediated by insulin and/or insulin-receptor mediated pathways.

### Example 29

### The Glucose Lowering Effects of Betacellulin and Glargine were at Least Additive

The normal physiologic pattern of insulin secretion by pancreatic beta cells consists of a sustained basal insulin level throughout the day, superimposed after meals by relatively large bursts of blood insulin that decay over 2 to 3 hours (that is, bolus insulin). Basal glucose control with long-acting insulin drugs is a key component of glucose management for patients with diabetes. Long-acting agents such as insulin glargine provide a steady and reliable level of basal insulin coverage and are beneficial as part of a basal-bolus treatment strategy, as described in Bethel, M. A. and Feinglos, M. N. J. Am. Board Fam. Pract. 18(3): 199-204 (2005). Insulin glargine is an extended-action insulin analog that was created by the recombinant DNA modification of human insulin, as described in Campbell, R. K. et al., Clin. Ther. 23(12): 1938-57 (2001). Alterations in the insulin molecule raise the isoelectric point and cause insulin glargine to precipitate at the injection site, thus slowing absorption. The pharmacodynamic profile of insulin glargine is characterized by the lack of a pronounced peak and a duration of action of approximately 24 hours.

We believed that an additive glucose lowering activity could be obtained when glargine and betacellulin are used in combination. To demonstrate the glucose lowering activity of glargine when used in combination with betacellulin, we conducted a test in male db mice. We compared the effect of 1.0 mg/kg betacellulin administered alone or in combination with glargine. The db mice were obtained from Harlan Laboratories at approximately 7-8 weeks of age and subsequently tested after about 1 week of acclimation in our facility. Betacellulin was prepared at our facility from expression in an E. coli host. Glargine was made by Aventis Pharmaceuticals, Inc. and was obtained from a local pharmacy. All blood glucose measurements were taken using blood from tail vein nicks and performed with a Bayer Ascensia glucometer.

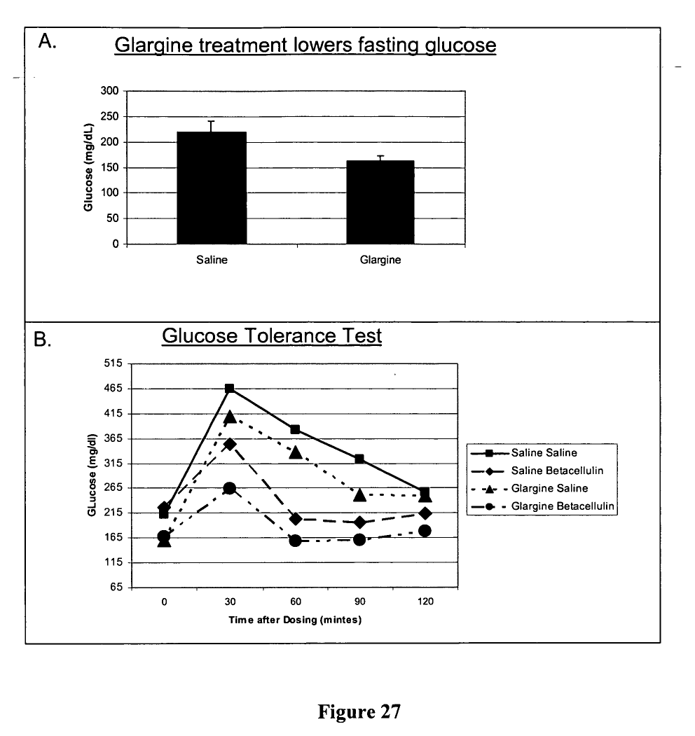

The mice were first distributed into two groups based on fasting glucose values. One group, the Glargine Group was injected intraperitoneally with 250 microliter of glargine at 1 unit/kg once a day for the first three days and then at 3 units/kg once a day for the next 3 days. The other group, the Saline Group, was injected with 250 microliter of every day for six days. Immediately after dosing on the sixth day, the mice were subjected to a five hour fast, at the end of which (i.e., at time 0), they were administered a bolus GTT in combination with either betacellulin or saline. Immediately prior to administration of 0.75 mg/kg of glucose intraperitoneally, at the onset of the GTT, the glargine treated group of mice and the saline treated group of mice were each split into subgroups of 10 mice each that received either 250 microliter of betacellulin at 1 mg/kg or 250 microliter of saline subcutaneously, forming four groups: the Glargine Betacellulin Group (●), Saline Betacellulin Group (▪), the Glargine Saline Group (▴) and the Saline Saline Group (▪). A GTT was conducted by injecting each mouse with 0.75 mg/kg of glucose intraperitoneally. The onset of the GTT occurred at approximately 1:00 PM, five hours after the last glargine dose (and five hours after fasting). The results of the test are shown in FIG. 27.

FIG. 27A shows that after six days of glargine treatment, the db mice in the Glargine Group exhibited a significantly lower level of fasting blood glucose, with about 165 mg/dL, as compared to that in the Saline Group, with about 215 mg/dL of fasting blood glucose level. FIG. 27B shows blood glucose level of the four groups of mice monitored over a period of two hr in a GTT. The mice in the Saline Saline Group had an average blood glucose level of about 215 mg/dL at time 0, which increased to about 465 mg/dL at 30 min, and decreased to about 390 mg/dL at 60 min, and about 325 mg/dL at 90 min and about 255 mg/dL at 120 min. The mice in the Glargine Saline Group started at a lower blood glucose level of about 165 mg/dL at time 0, which increased to about 400 mg/dL at 30 min, then decreased to about 340 mg/dL at 60 min, and about 250 mg/dL at 90 min, and about 250 mg/dL at 120 min. The mice in the Saline Betacellulin Group started at a higher blood glucose level of about 230 mg/dL and increased to about 350 mg/dL, then decreased to about 200 mg/dL at 60 min, and about 195 mg/dL at 90 min, and about 215 mg/dL at 120 min. The mice in the Glargine Betacellulin Group had an average blood glucose level of about 165 mg/dL at time 0. The level increased to about 265 mg/dL at 30 min, and decreased to 165 mg/dL at 60 min, remained at 165 mg/dL at 90 min, and was slightly higher at about 180 mg/dL at 120 min.

Basal release of insulin from the pancreas controls blood glucose levels during the fasting state. Long-acting insulins or other medications that stimulate endogenous basal glucose control are expected to primarily reduce fasting blood sugar and exert relatively minimal effect during acute carbohydrate loads as occurs shortly following a meal. This effect was demonstrated in this experiment, which showed that treatment of diabetic animals with glargine, a long-acting “basal-acting” insulin, resulted in a reduction in fasting blood sugar. In terms of acute reduction of blood glucose level after administration of a bolus of glucose, the Glargine treated mice showed only a modest reduction in blood glucose level in a GTT. Consistent with earlier findings, betacellulin alone was effective in acute reduction of blood glucose after a glucose bolus, rapidly within 60 min of glucose administration, to a pre-glucose dosing level. The combination of glargine and betacellulin combined the benefit effects of both drugs alone, achieving both an acute reduction in blood glucose after a glucose bolus and maintenance of a lower basal blood glucose level. These data indicated that combination of betacellulin with an agent that, in whole or in part, reduced fasting blood sugar resulted in better postprandial glucose control than that achieved with either agent alone.

### Example 30

### Betacellulin Promoted Glucose Uptake into Isolated Rat Plantaris Muscle

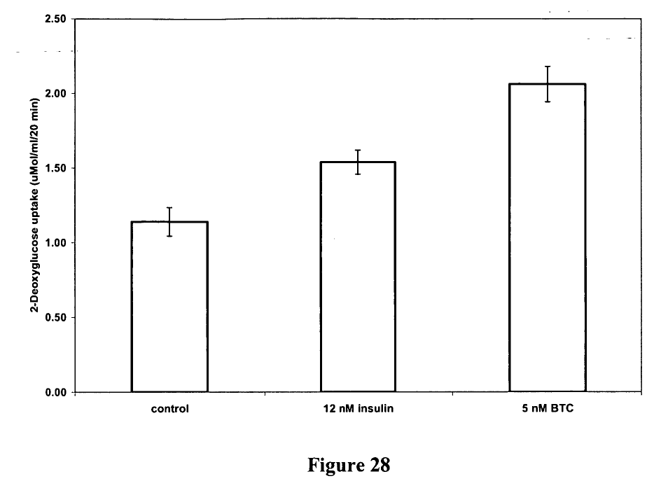

With our finding that betacellulin and other members of the EGF family stimulated glucose uptake into primary human skeletal muscle cells, we tested the effect of betacellulin on other muscle cells. As shown in FIG. 28, we found that betacellulin augmented muscle glucose uptake in situ more effectively in rat plantaris muscle than that induced by insulin in the absence of betacellulin. In this experiment, we used male Sprague-Dawley rats (9 weeks of age), obtained from the Charles River Laboratories (Wilmington, Mass.). Rat plantaris muscles with tendons still attached were isolated from the animal's hindquarter according to published methods, such as described in Wilkes, J. J. et al., Diabetes 52:1904-1909 (2003). Isolated muscles were split in half. The split muscles were placed in a Krebs-Henseleit buffer (KHB) solution containing 32 mmol/l mannitol, 8 mmol/l D-glucose, and 0.1% BSA. Strips were incubated without addition (control) or with either 12 nM insulin or 5 nM betacellulin at 37° C. for 50 min. Before glucose transport measurements, D-glucose was removed by washing the muscles once in glucose-free KHB with 38 mmol/l mannitol and 2 mmol/l pyruvate. For determining 2-deoxyglucose (2-DOG) uptake, muscles were incubated with (4.5 microCi/ml) 2-deoxy-D-[³H]glucose (1 mmol/l) and (1 microCi) ¹⁴C-mannitol (37 mmol/l) for 20 min. Muscles were removed rapidly, blotted, and snap-frozen in dry ice. Muscles were analyzed for ¹⁴C and ³H by boiling for 10 min in 1 ml of water. The rate of glucose uptake was calculated as described by Hansen P. A., J. Appl. Physiol. 76(2):979-985 (1994). Our results showed that betacellulin, at a concentration of about 5 nM, stimulated radioactive glucose uptake at about 2 micromol/ml/20 min. These results indicated that betacellulin was effective in stimulating glucose uptake into plantaris muscle cells and was able to do so at a lower concentration than insulin, suggesting a higher potency than insulin.

### Example 31

### Betacellulin Promoted Amino Acid Uptake by Skeletal Muscle Cells

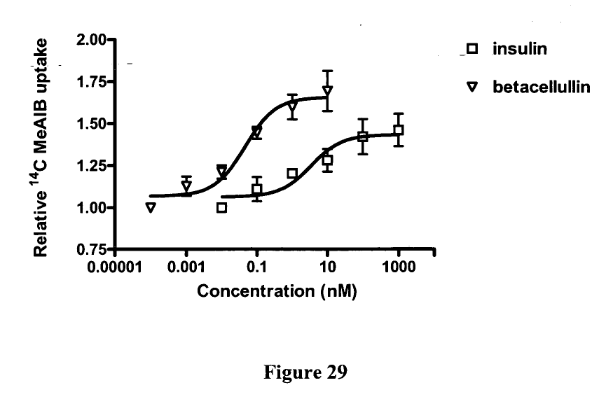

With our finding that betacellulin stimulates glucose uptake into different muscle cells, we decided to determine whether betacellulin possesses other anabolic activities. We tested the ability of betacellulin to stimulate amino acid uptake into muscle cells, since amino acid uptake by skeletal muscle is reportedly reduced during different catabolic conditions, such as diabetes and muscle wasting disorders. We found in this test that betacellulin robustly promoted amino acid uptake by cultured primary human skeletal muscle cells (Cambrex, East Rutherford, N.J.), as shown in FIG. 29.

In this experiment, primary human skeletal muscle cells were seeded onto 96-well plates at a density of 3×10⁴ cells per well in a growth medium as before. The cells were allowed to attach overnight in a cell culture incubator at 37° C. and 5% CO₂. The next day, the growth medium was removed and serum-free medium was added, and the cells were serum-starved for 5 hours. Thereafter, the medium was replaced with HEPES buffered saline (HBS) for 1 hr to deplete the cells of amino acids. Different concentrations of either insulin, from about 10⁻¹¹ M to about 10⁻⁶ M, or human recombinant betacellulin, from about 10⁻¹³ M to about 10⁻⁸ M, (R&D Systems, Inc., Minneapolis, Minn.) in culture medium were added to different wells and incubated for 20 min in a cell culture incubator at 37° C. and 5% CO₂. Control cells were treated with culture medium alone. After incubation, the medium was replaced with 50 microliter of a 10 microM solution of the ¹⁴C-labeled non-metabolizable alanine homologue 2-(methylamino)isobutyric (MeAIB) acid in HBS at the equivalent of 0.1 μCi per well, and the cells were placed back in the cell culture incubator at 37° C. and 5% CO₂ for 15 min. The medium was then removed, the cells were washed three times with ice-cold PBS and then lysed with 0.05 N NaOH. Uptake of the ¹⁴C-labeled amino acid MeAIB was assessed by radioactivity counts of the lysates using a Perkin Elmer TopCount and normalized values were plotted relatively to those of negative control cells.

Each measurement was done in triplicate wells. Results shown in FIG. 29 demonstrated that at all concentrations of insulin and betacellulin tested, betacellulin consistently exhibited a higher potency than insulin in stimulating amino acid uptake into muscle cells.

### Example 32

### Betacellulin Mediated the Upregulation of Utrophin Expression in Muscle Cells

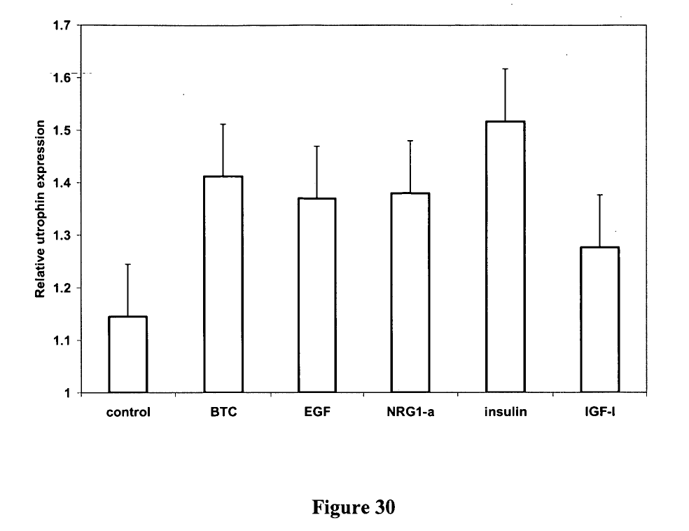

With our finding that betacellulin and other members of the ErbB ligand (EGF) family were able to stimulate glucose and amino acid uptake into muscle cells, we were led to believe that betacellulin and other ErbB family members would likely be useful for treatment of other diseases involving muscles, besides diabetes, such as muscular dystrophies, sarcopenia, muscular atrophies, neuromuscular disorders, at the like. Here, we tested the effect of betacellulin and other ErbB ligand family members for their ability to stimulate the expression of utrophin, a protein that plays an important role in muscular dystrophy. Our results, as shown in FIG. 30, showed that, at the concentration tested, betacellulin and other ErbB ligands/EGF family members, such as EGF and NRG1-alpha (NRG-1α), like insulin, were able to upregulate the expression of utrophin mRNA in primary human skeletal muscle cells (Cambrex, East Rutherford, N.J.).

In this experiment, primary human skeletal muscle cells were seeded onto 96-well plates at a density of 3×10⁴ cells per well in growth medium and allowed to attach overnight in a cell culture incubator at 37° C. and 5% CO₂ as described in earlier examples. The next day, the growth medium was removed and replaced with serum-free medium. Human recombinant betacellulin, EGF, NRG1-α, insulin or IGF-I (all from R&D Systems, MN), each at the same final concentration of 10 nM in serum-free medium, were separately added to cells in different wells, and the cells were incubated for 48 hr. Control cells were treated with serum-free medium alone. After incubation, total cellular RNA was harvested from the cells using RNeasy 96 Kit from Qiagen (Valencia, Calif.). The level of utrophin mRNA in the harvested cells was quantified using the QuantiTect SYBR Green RT-PCR system from Qiagen. The utrophin expression levels were normalized to the expression of house-keeping gene GusB, which was also measured for each treatment condition. NRG1-alpha (NRG1-α) acted as a positive control, as described in Gramolini, A. O. et al., Proc. Natl. Acad. Sci., 96:3223-3227 (1999). At the concentration tested, betacellulin, EGF and NRG1-α, as well as insulin, were more active than control media in stimulating utrophin expression in these cells. Insulin was the most active, about 1.5 fold higher than control. IGF-1 was the least active, at about 1.25 fold more active than control. Each expression level was measured in triplicate wells and the average was plotted as shown in FIG. 30. Thus, the EGFR/ErbB ligand family members were effective in stimulating utrophin expression in primary human skeletal muscle cells.

### Example 33

### Regulation of Utrophin Expression by EGF Family Members is a Dose-Dependent Process

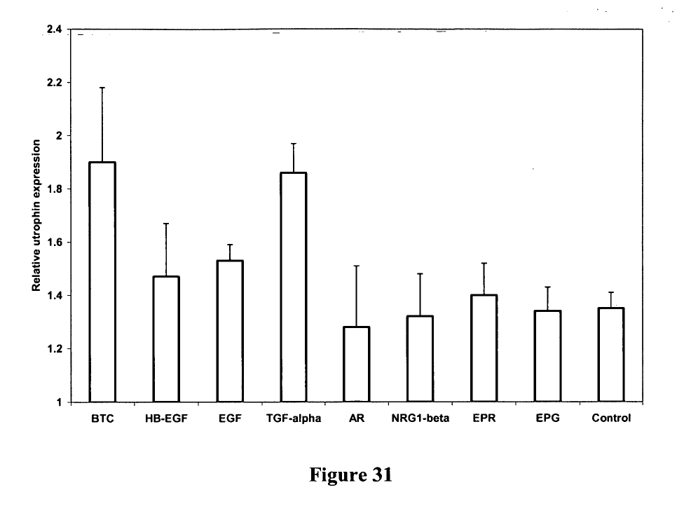

We tested the relative potency of different EGF/ErbB family members in their ability to stimulate utrophin expression in primary human skeletal muscle cells (Cambrex, East Rutherford, N.J.). We found that, at the dose of 100 pM, betacellulin and TGF-alpha (TGF-α) were the most potent in stimulating utrophin expression, as shown in FIG. 31. Next in potency were EGF, HB-EGF and epiregulin.

In this experiment, primary human skeletal muscle cells were seeded on 96-well plates at a density of 3×10⁴ cells per well in growth medium and allowed to attach overnight in a cell culture incubator at 37° C. and 5% CO₂ as before. The next day, the growth medium was removed and was replaced with serum-free medium. Cells in different wells were treated for 48 hr separately with recombinant human betacellulin (“BTC”) or with other ErbB ligand family members at concentrations of 0.1 pM, 1 pM, 10 pM, 100 pM, 1000 pM and 10,000 pM in serum-free medium, with 4 wells per protein per concentration. The ErbB ligand family members tested included: betacellulin, HB-EGF, HB-EGF, TGF-alpha (TGF-α), amphiregulin (“AR”), Neuregulin1-beta (NRG1-β), epiregulin (“EPR”) and Epigen (“EPG”). All the proteins were purchased from R&D Systems, Inc. (Minneapolis, Minn.). Control cells were treated with serum-free medium alone. Cellular RNA was harvested as before (Example 32). The level of utrophin mRNA in cells harvested after the 48 hr treatment was quantified using the QuantiTect SYBR Green RT-PCR system from Qiagen. The utrophin expression levels were normalized to the expression of house-keeping gene GusB, which was also measured for each treatment condition, to generate the relative utrophin expression. Each expression measurement was done in four replicate wells. FIG. 31 shows only the measurements at a 100 pM dose for each protein as averaged.

Results of this test showed that ErbB ligand family members such as betacelluin, HB-EGF, and TGF-α (alpha) stimulated an increase in utrophin expression in primary human skeletal muscle cells at least about 40% above the levels of utrophin in the presence of serum-free medium alone. These three proteins, or polypeptide fragments thereof, produced their maximal effect on primary human skeletal muscle cells at concentrations of approximately 100 pM, 1.0 nM and more than 10 nM, respectively. EGF had a smaller effect on utrophin expression. AR, NRG1-beta, and EPG had little or no effect on utrophin expression in primary human skeletal muscle cells.

### Example 34

### Betacellulin did not Stimulate Lipogenesis in Primary Rat Adipocytes

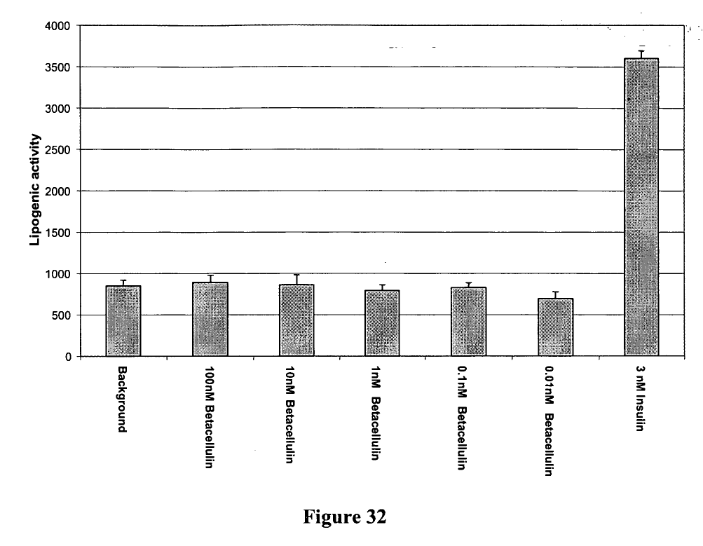

Our findings that betacellulin and other members of the ErbB/EGF family of ligands stimulated glucose and amino acid uptake into muscle cells prompted us to determine whether betacellulin had lipogenic activities. This was measured by determining the incorporation of ³H-glucose into fatty acids. Lipogenic activity was assessed by determining the amount of ³H activity in the organic phase (lipid-containing phase) of the cell extracts. Our results, shown in FIG. 32, demonstrated that betacellulin did not possess any lipogenic activity at the concentrations tested.

In this experiment, we obtained male Sprague-Dawley rats (9 weeks of age) from the Charles River Laboratories (Wilmington, Mass.). Adipocytes were isolated from the animals and were incubated in DMEM with 1% BSA for two hr, using methods standard in the art (see, for example, Moldes, M. et al. Biochem J. 344:873-880 (1999)). Subsequently, the cells were treated with either insulin at 3 nM (positive control), or with betacellulin at various concentrations in the range of about 0.01 nM to 100 nM in DMEM with 1% BSA, or control medium containing DMEM with 1% BSA. If betacellulin stimulated lipogenic activities, ³H-glucose would be converted, at least in part, into fatty acids. The results showed that, unlike insulin which has high lipogenic activities, betacellulin did not stimulate lipogenic activity in isolated adipocytes at any of 0.01 nM, 0.1 nM, 1 nM, 10 nM or 100 nM. Similar experiments can be executed with adipocyte cell lines, such as 3T3 L1 adipocytes from ATCC.

### Example 35

### Betacellulin Activated EGF-Receptor Phosphorylation in HeLa Cells

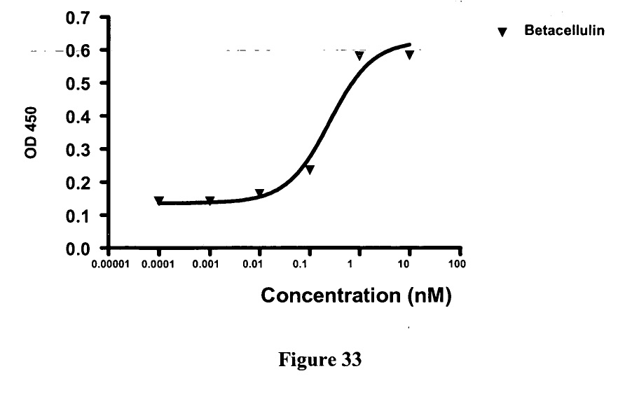

To assay betacellulin activity, we measured the phosphorylation of ErbB receptors by betacellulin. About 3×10⁴ HeLa cells (from ATCC) in 100 microliter of MEM containing 10% fetal bovine serum were plated onto each well of a 96-well plate. The cells were allowed to attach overnight. The next day, culture medium was removed and cells were starved in 90 microliter of serum-free medium for six hr. Cells were then treated with 10 microliter of betacellulin at various concentrations, ranging from 10⁻⁸ M to 10⁻¹³ M, in the starvation medium for 15 min at 37° C. After that, the cells were lysed and phosphorylated receptors (pY1068) were quantified by ELISA (Biosource International Inc., Camarillo). FIG. 33 illustrates the effect of betacellulin on ErbB 1 receptor phosphorylation. We found that betacellulin was able to induce phosphorylation of ErbB1 receptor in a dose-dependent manner. Our results demonstrated that ErbB1 phosphorylation assay in Hela cells provided a convenient way to detect betacellulin activities.

### Example 36

### Betacellulin Stimulated ³H-Deoxyglucose Uptake in Rat Neonatal Cardiomyocytes

We have shown in an earlier experiment that betacellulin, as well as other EGF family members, stimulated glucose uptake into primary human skeletal muscle cells and rat plantaris muscle cells. We further tested whether another type of muscle cells, that is, cardiomyocytes, would respond in the same manner.

Isolation of Rat Neonatal Cardiomyocytes

Rat cardiomyocytes were isolated using a neonatal rat/mouse cardiomyocyte isolation kit purchased from Cellutron Life Technologies (Cat # nc-60631, Highland Park, N.J.), and following the manufacturer's suggested protocol. First, we prepared the working solutions for tissue digestion (D1, D2, and D3 working solutions). Specifically, the D1 working solution was prepared with 5 ml of D1 stock solution and 45 ml of sterile water. Two D2 working solutions were prepared. Each D2 working solution contained 20 ml of D2 stock solution, 28 ml sterile water, and 2 ml of EC (Enzyme Collagenase) buffer; the components were mixed and the D2 solution was filtered with a 0.22 micrometer filter. Two D3 working solutions were prepared. Each D3 working solution contained 25 ml of NS (Neonatal Seeding) medium and one bottle (15 ml) of D3 stock solution, and thus was brought to a final volume of 40 ml. Neonatal rats (Sprague Dawley strain, Charles River Laboratories) were sterilized with 70% ethanol, the chest open, and the hearts removed and placed in cold D1 solution. In a separate culture dish, also containing cold D1 solution, the larger vessels, atria and connective tissue were trimmed away leaving the heart ventricles.

The cut heart ventricles were then transferred to a sterile 30 ml flask containing 12 ml of D2 working solution (approximately 12 ml of solution for about 70-80 neonatal hearts) and the tissues stirred on a stir plate for 12 min at a stir speed between # 2-3 (about 300-600 rpm), (Fisher Scientific, Houston Tex., CAT #: 1150049S) in a 37° C. incubator/oven, during which period the cells were released from the ventricle tissue. The tissue in solution was pipetted up and down, and the supernatant (containing the released cells) was then transferred to a 15 ml sterile round bottom plastic tube and placed in a centrifuge (Kendro, Germany, Cat # 75004377). The supernatant was spun at room temperature at 1200 rpm for 2 min to yield a cell pellet. The cell pellet was resuspended in 5-10 ml of D3 working solution and left at room temperature until the end of isolation procedure. The steps described above with the D2 and D3 working solutions were repeated between 5 to 11 separate times until all of the processed ventricle tissues were digested into cells. The cells recovered from all the ventricles were pooled, filtered with a cell strainer/filter provided in the kit, and the cells were harvested from the top of the filter by moving the pipette around the surface of the filter.

The cells (recovered from about 70 heart ventricles) were subsequently incubated for about 1.5 hr at 37° C. with 5% CO₂ by seeding them onto eight uncoated 100 mm Corning cell culture dishes (Corning Incorporated, Corning N.Y., Cat #: 430167) to remove the fibroblasts (under these conditions, only the fibroblasts attached to the plate whereas the cardiomyocytes remained in suspension). After this period, the media containing the neonatal cardiomyocytes, were collected and the cells were counted. To confirm the cell purity, we performed immunocytochemical staining for sarcomeric alpha actin in an aliquot of the pool of isolated cells following the instructions in the neonatal rat/mouse cardiomyocyte isolation kit. Sarcomeric alpha actin is a marker of cardiomyocytes and does not exist in cardiac fibroblasts.

Next, we seeded rat neonatal cardiomyocytes at 3×10⁴ cells per well in 100 microliter of NS medium (Cellutron Life Technologies, Highland park, N.J., Cat# M-8031) on day 1 in 96-well white/clear bottom tissue culture plate (BD Biosciences, Bedford, Mass., Cat# 353947). The plate was left in the tissue culture hood for 30 min to minimize the edge effect. The plate was then placed in the incubator at 37° C. with 5% CO₂ overnight.

The next day, on day 2, the medium was removed, and 90 microliter of starvation medium, containing 1% BSA in low glucose (5mM) DMEM, was added to each well. The cells were starved for six hr. Then 10 microliter of medium as negative control, or insulin as positive control, or a test protein (BTC, or neuregulin 1-betal (“NRG1-β1”)), was added to each well. After 20 min of incubation, the medium was removed, and 50 microliter of ³H labeling medium was applied to each well. The labeling medium contains ³H-deoxyglucose solution (Cat# NET-331A; PerkinElmer Life And Analytical Sciences Inc., Wellesley, Mass.)) with 1 μCi in 50 microliter labeling medium, 1% BSA, and 10 microM cold deoxyglucose (Sigma, Steinheim, Germany, Cat# D-3179) in glucose-free DMEM. The plate was incubated for 15 min. The labeling medium was then removed, and the cells were washed three times with ice-cold PBS containing calcium and magnesium. After washing, PBS was removed, and 50 microliter of 0.05 N NaOH was applied to each well followed by pipetting up and down to lyse the cells. Then 150 microliter of microscint 40 (Cat# D-6013641; PerkinElmer Life and Analytical Sciences Inc., Wellesley, Mass.) was added to each well very slowly with the tip being stirred when adding the solution. The top of the plate was sealed with sealing tape (Cat# 6005185; PerkinElmer Life and Analytical Sciences Inc., Wellesley, Mass.)), and the bottom of the plate was covered with white Backing tape (PerkinElmer Life and Analytical Sciences Inc., Wellesley, Mass., Cat# 6005199). The signal was counted using TopCount NXT with Windows XP®-based operating software (PerkinElmer Life and Analytical Sciences Inc., Wellesley, Mass.).

Results are shown in FIG. 34. Each bar represents an average of four or more wells per treatment. The height of the bar (y-axis) indicates relative glucose uptake, which is the ratio of glucose uptake of each protein divided by the control, which was set at 1. All three proteins tested (betacellulin, NRG1-β1 (beta1) and insulin) stimulated glucose uptake into the rat neonatal cardiomyocytes at about 1.2 to 1.5 fold as compared to the control. The difference between each of these tested proteins and control was found to be statistically significant (p<0.01).

### Example 37

### Betacellulin Stimulated Phosphorylation of Akt and ERK, and Enhanced the Survival Rat Neonatal Cardiomyocytes

Betacellulin Promoted Phosphorylation of Akt and ERK, but not STAT3, in Rat Neonatal Cardiomyocytes

Neonatal cardiomyocytes, harvested as described in Example 36, were diluted to 6×10⁵ cell/ml in a NS (Neonatal Seeding) medium (Cellutronlife Technologies, Highland Park, N.J., Cat #: M-803 1) and 0.1 millimolar (mM) bromodeoxyuridin (BrdU) solution (Sigma, Steinheim, Germany, Cat# B5002-250 mg). The diluted cells were then plated at a volume of 100 microliters (microliter)/well in 96-well Primaria™ plates (Becton Dickinson, Franklin Lakes, N.J., Cat #: 353872) and incubated at 37° C. with 5% CO₂ overnight on day 1.

The next day (day 2), the media were changed to fresh NS medium containing 0.1 mM BrdU at 150 microliter/well, and the cells were incubated at 37° C. with 5% CO2 overnight. On day 3, the media were changed to starve medium with 150 microliter/well, and the cells were incubated at 37° C. with 5% CO₂. The starve medium contained: DMEM-glc-pry+10 mM HEPES+0.1% BSA+1× Penicillin-Streptomycin. The DMEM-glc-pry contained DMEM without glucose and without pyruvate (Gibco/Invitrogen Corporation, Grand Island, N.Y., Cat # 11966-025). HEPES was purchased from Mediatech Inc., Herndon, Va. (Cat # 25-060-Cl, 1M). Bovine Albumin Fr. V Fatty Acid Free (BSA) was purchased from Serologicals Protein Inc. Kankakee, Ill. (Cat # 82-002-4,), and Penicillin-Streptomycin was purchased from Mediatech Inc., Herndon, Va. (Cat # 30-002-CI, 100×).

On day four after the overnight incubation, the 96 wells of the plates were aspirated and washed with 150 microliter /well of fresh starve media, and an additional 50 microliter of fresh starve media was added to each well. The cells in columns 2-11 of a 96 well plates were subsequently treated by adding 50 microliter of protein conditioned medium. Positive controls of 300 nanogram/mL of rhIGF1, were added to wells A-D of column 1, positive controls of 20 ng/mL of rhLIF were added to wells A-H of column 12, and the negative control (vector only conditioned medium), was added to wells E-H of column 1.

The plates were subsequently incubated at 37° C. with 5% CO₂ for 15 minutes. After the incubation with the different test agents (recombinant proteins), the solutions in the wells were removed by aspiration. The wells were subsequently washed with 150 microliter/well of ice-cold 1×PBS, and 40 microliter of ice-cold Lysis Buffer (Cell Signaling Technology Inc., Beverly, Mass., Cat# 9803) containing 1 mM PMSF (Sigma, Steinheim, Germany, Cat # P7626) was added to each wells. The plates were kept on ice for 10 min. The plates containing the cell lysates were then ready for the Luminex Phosphor-protein Detection Assay.

Luminex Phosphorylated-Protein Detection Assay

The 96-well assay filter plates (Cat# MSBVN1250, Millipore, Molsheim, France) were washed with about 100 microliter of assay buffer, and the buffer subsequently aspirated by vacuum. The assay buffer contained Dulbecco's Phosphate-Buffered Saline (DPBS) without calcium and without magnesium (Mediatech Inc., Herndon, Va., Cat#21-031-CV) and 0.2% BSA (Serologicals Protein Inc. Kankakee, Ill., Cat#82-002-4).

The suspensions of antiphospho-Akt (αpAkt) beads (UpState Inc. Lake Placid, N.Y., Cat # 46-601), antiphospho-ERK (αpERK) beads (UpState Inc. Cat # 46-602), and antiphospho-STAT3 (αpSTAT3) beads (UpState Inc. Cat # 46-623) were diluted in assay buffer with a 1:40 dilution for the αpAkt Beads and a 1:50 dilution for both the αpERK beads and the αpSTAT3 beads. About 25 microliter of a three-bead mixture (containing equal volumes of each αpAkt, αpERK, and αpSTAT3 bead solution) were added to each well of an Assay Filter plate. Additionally, 25 microliter of cell lysates (prepared as described above) were added to each well of the Assay Filter plate. The plates were subsequently incubated on a shaker at 4° overnight in the dark with black lids.

After incubation, the liquid in the wells was aspirated off by vacuum and the wells were each then washed twice with 200 microliter of assay buffer. The biotinylated reporters for αpAkt (UpState Inc. Lake Placid, N.Y., Cat# 46-601), αpERK (UpState Inc. Cat# 46-602), and αpSTAT3 (UpState Inc. Cat# 46-623) were diluted with assay buffer accordingly: a 1:40 dilution for the αpAkt biotinylated reporter and a 1:50 dilution for both the αpERK and αpSTAT3 biotinylated reporters. The prepared biotinylated reporters were mixed and a volume of 25 microliter of the mixed reporters was added to each well after the assay buffer used for the washing step had been aspirated off. The plates were then incubated on a shaker at room temperature for 90 min in the dark. After 90 min, the liquid was aspirated off the wells and the wells washed twice with about 200 microliter of Assay Buffer. Streptavidin-PE (BD PharMingen, San Diego, Calif., Cat # 554061) was subsequently prepared in Assay Buffer at 1:200 dilution, and about 25 microliter of diluted streptavidin-PE was added to each well. The plates were then incubated on a shaker at room temperature for 15 min in the dark. An Enhancer Solution (UpState Inc. Lake Placid, N.Y., Cat # 43-024) was prepared with assay buffer (1:1) and 25 microliter was added to each well. The plates were incubated for 30 min on a shaker at room temperature in the dark. The liquid was aspirated off, and the wells each washed once with 200 microliter of assay buffer. Finally, 100 microliter of assay buffer was added to each well to suspend the beads, and the plates were placed on a shaker at room temperature for 10 min in the dark. The plates were then read on a Luminex Reader using “pAkt, pERK, pSTAT3” Program.

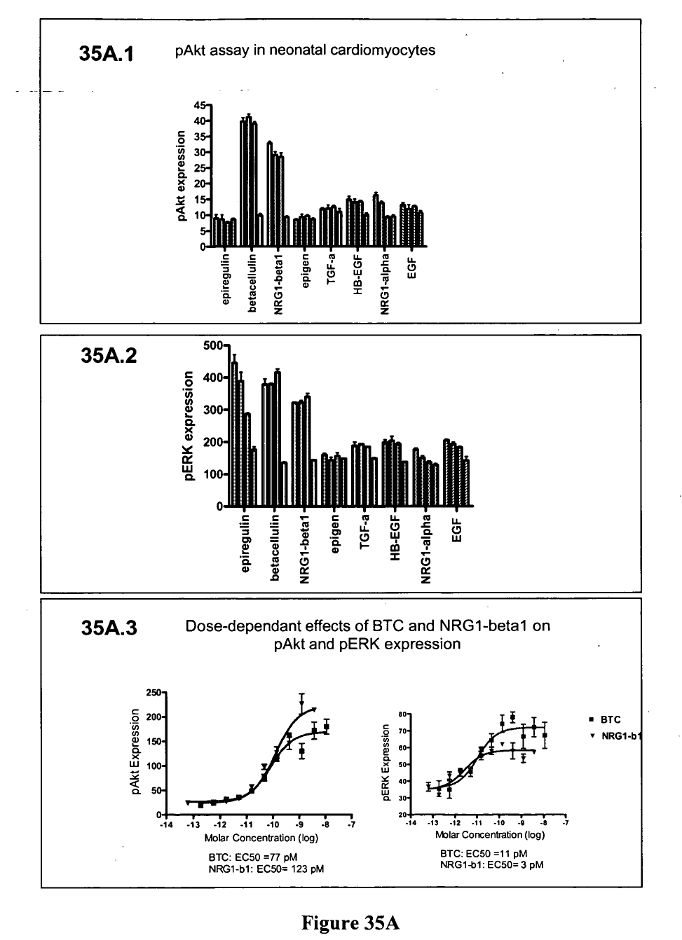

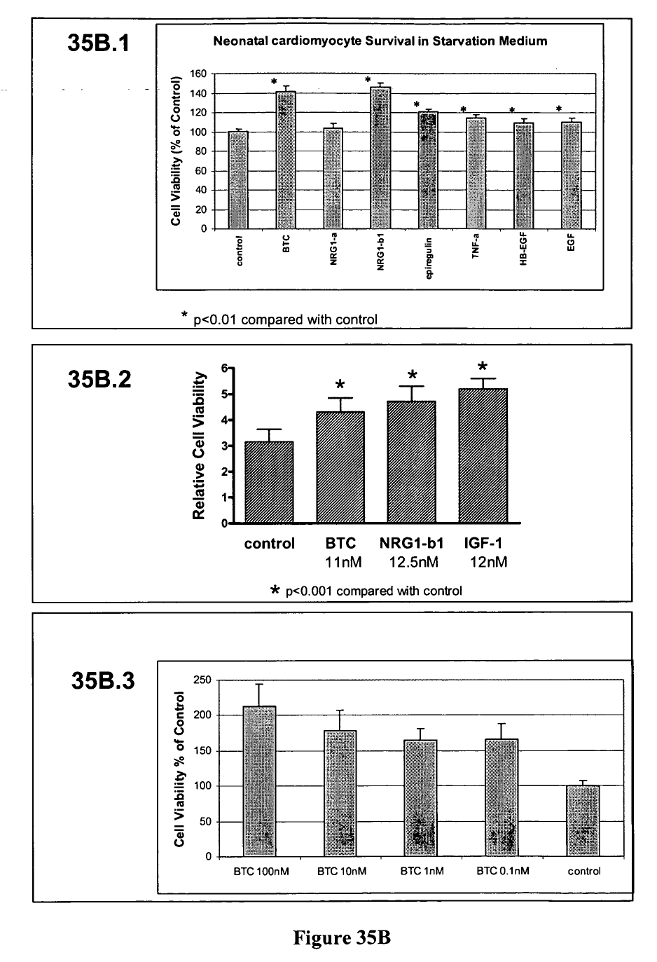

FIG. 35A.1 and FIG. 35A.2 show the results of the pAkt and pERK assay in rat neonatal cardiomyocytes treated with different doses of recombinant proteins, all of which were obtained from R&D Systems, as described in earlier examples. In both FIG. 35A.1 and FIG. 35A.2 each of the four bars for each recombinant protein represent four different doses of each protein, and each bar refers to the average of three replicates. The doses are 100 ng/ml for the first bar, 33 ng/ml for the second bar, 11 ng/ml for the third bar, and 0 ng/ml for the fourth bar, starting from the left. The height of the bar (y-axis) represents the readout of the luminescent signal. The results shown in FIG. 35A.1 indicate that both betacellulin and NRG1-beta1 increased pAkt level (referred to as pAkt expression) to a higher extent than did HB-EGF and NRG1-alpha. The results shown in FIG. 35A.2 indicated that epiregulin, betacellulin, and NRG1-beta1 increase pERK level significantly, and TGF-alpha, HB-EGF, NRG1-alpha, and EGF enhances pERK level only a little bit. None of the tested proteins tested under these conditions showed effects on pSTAT3 activation. The results shown in FIG. 35A.3 indicate that the effects of betacellulin (BTC) and NRG1-beta1 on pAkt and pERK levels (referred to as pAkt and pERK expression) after neonatal cardiomyocytes are dose-dependent. Under these conditions, the EC50 of betacellulin was about 77 pM and about 11 pM for the pAkt and pERK expression, respectively; whereas the EC50 of NRG1-b1 was about 123 pM and about 3 pM for the pAkt and pERK expression, respectively.

Betacellulin Promoted the Survival of Rat Neonatal Cardiomyocytes Exposed to Starvation Conditions

We used the CellTiter-Glo assay (Promega, Madison, Wis., Cat# G7573), according to the manufacturer's instructions, to test the effect of several agents on cardiomyocyte survival under nutrient deprivation (starvation) conditions. On day 1, rat neonatal cardiomyocytes were seeded at 2×10⁴ cells per well in 100 microliter of NS medium (Cellutron Life Technologies, Highland park, N.J., Cat# M-8031) supplemented with 0.1 millimolar (mM) bromodeoxyuridin (BrdU) solution (Sigma, Steinheim, Germany, Cat# B5002) in 96-well Primaria tissue culture plate (Becton Dickinson, Franklin Lakes, N.J., Cat# 353872). The plate was sealed with Breathe Easy Sealing Tape (E&K Scientific, Santa Clara, Calif., Cat # 1796200). The cells were incubated overnight at 37° C. with 5% CO₂.

On the next day (day 2), the medium was changed to 150 microliter of fresh NS medium supplemented with 0.1 mM BrdU. The plate was sealed with sealing tape. The cells were incubated for another 24-48 hr. Subsequently, the cells were treated with different recombinant proteins in 100 microliter of Starve Medium which contained 10 mM HEPES, 0.1% BSA, and 1× Penicillin-Streptomycin in DMEM-glc-pyr. The DMEM-glc-pyr was DMEM without glucose and without pyruvate (Gibco/Invitrogen Corporation, Grand Island, N.Y., Cat# 11966-025). HEPES was purchased from Mediatech Inc., Herndon, Va. (Cat# 25-060-Cl, 1M). Fatty Acid Free Bovine Albumin Fraction V (BSA) was purchased from Serologicals Protein Inc. Kankakee, Ill. (Cat#82-002-4), and Penicillin-Streptomycin was purchased from Mediatech Inc., Herndon, Va. (Cat# 30-002-Cl, 100×). After about 40 hr incubation, about 100 microliter of CellTiter-Glo assay buffer (Promega, Madison, Wis., Cat # G7573) per well was added to the medium, followed by shaking at room temperature in dark for 10 min. A total of 100 microliter of mixture per well was transferred to 96-well ½ area assay plate (Corning Incorporated, Corning, N.Y., Cat#3688), and the luminescent signal was determined by luminescent plate reader Lmax (Molecular Devices Corporation, Sunnyvale, Calif.).

The results of this assay are shown in FIG. 35B.1. Each bar represents a different test agent, and each test agent was measured in six replicates. The cell viability of control is set as 100%. The height of the bar (y-axis) indicates the cell viability percentage of the control; while the viability percentage was calculated with the average ATP luminescent signal of each protein divided by that of control. The proteins labeled with an asterisk (*) namely BTC, NRG1-b1, epiregulin, TNF-alpha, HB-EGF and EGF, all caused a statistically significant increase in cell survival under starvation conditions when compared with control treated cells (p<0.01).

Betacellulin Promoted the Survival of Rat Neonatal Cardiomyocyte Exposed to Ischemic Conditions

To test the effect of several agents on the survival of cardiomyocytes exposed to oxygen deprivation (i.e., ischemic conditions), rat neonatal cardiomyocytes were seeded, on day 1, at 2×10⁴ cells per well in 100 ul of NS medium (Cellutron Life Technologies, Highland park, N.J., Cat# M-8031) supplemented with 0.1 millimolar (mM) bromodeoxyuridin (BrdU) solution (Sigma, Steinheim, Germany, Cat# B5002) in a 96-well Primaria tissue culture plate (Becton Dickinson, Franklin Lakes, N.J., Cat# 353872). The plate was sealed with Breathe Easy Sealing Tape (E&K Scientific, Santa Clara, Calif., Cat# 1796200). The cells were incubated overnight at 37° C. with 5% CO₂. On the next day (day 2), the medium was changed to 150 microliter of fresh NS medium supplemented with 0.1 mM BrdU. The plate was sealed with sealing tape. On day 3, i.e. after an additional overnight incubation at 37° C. with 5% CO₂, the medium was changed to 150 microliter per well of Starve Medium. The plate was sealed with sealing tape. Next, the cells were incubated overnight again at 37° C. with 5% CO₂. On day four, the cells were treated with different recombinant proteins in 100 microliter of Esumi Ischemic Buffer, which contained 137 mM NaCl, 12 mM KCl, 0.9 mM CaCl₂.2H₂O, 4 mM HEPES, 10 mM deoxyglucose, 20 mM sodium lactate, and 0.49 mM MgCl₂, with pH 6.7 in H₂O. The control group of cells did not receive any recombinant protein. After three hours of incubation under ischemic conditions, 100 microliter of CellTiter-Glo assay buffer (Promega, Madison, Wis., Cat# G7573) was added per well to the medium, followed by shaking the plate at room temperature in the dark for 10 minutes. A total of 100 microliter of this mixture per well was transferred to 96-well ½ area assay plate (Corning Incorporated, Corning, N.Y., Cat#3688), and the luminescent signal was determined by luminescent plate reader Lmax.

The results of this test, shown in FIG. 35B.2, showed the effects of recombinant human betacellulin and NRG1-beta1 on the viability, or survival, of rat neonatal cardiomyocytes exposed to ischemic conditions. Recombinant human IGF-1 served as the positive control. Each bar represents treatment with a different test agent, and each treatment included 24 replicates. The height of the bar (y-axis) indicates the relative cell viability (measure of surviving cells) represented by the ATP luminescent signal. All three proteins labeled with an asterisk (*), namely betacellulin, NRG1-b1 and IGF-1, caused a statistically significant increase in cell survival when compared with control-treated cells (p<0.001).

Betacellulin Promoted the Survival of Cardiomyocytes Exposed to Cardiotoxic Agents

Having determined that betacellulin promotes the survival of neonatal cardiomyocytes exposed to either starvation or ischemic conditions, we also decided to test the possibility that betacellulin would protect cardiomyocytes against toxic agents, such as medications that have cardiotoxic side effects (doxorubicin, for example), being used as, for example, chemotherapeutic agents in cancer or other types of treatment.

In this experiment, we seeded rat neonatal cardiomyocytes at 2×10⁴ cells per well in 100 microliter of NS medium (Cellutron Life Technologies, Highland park, N.J., Cat# M-8031) supplemented with 0.1 millimolar (mM) bromodeoxyuridin (BrdU) solution (Sigma, Steinheim, Germany, Cat# B5002) on day 1 in 96-well Primaria tissue culture plate (Becton Dickinson, Franklin Lakes, N.J., Cat# 353872). The plate was sealed with Breathe Easy Sealing Tape (E&K Scientific, Santa Clara, Calif., Cat# 1796200). The cells were incubated overnight at 37° C. with 5% CO2. The next day, day two, the medium was replaced with 150 microliter of fresh NS medium supplemented with 0.1 mM BrdU. The plate was again sealed with sealing tape. After overnight incubation, the medium was replaced with 150 microliter per well of Starve Medium (as in Example 36). The plate was again sealed and the cells were incubated overnight again. The next day, day four, the cells were treated with 50 microliter of 2 microM doxorubicin (Sigma-Aldrich, St. Louis, Mo., Cat# 44583) and 50 microliter of control medium without betacellulin, or with betacellulin (R&D Systems, MN) at varying concentrations of 0.2 nM, 2 nM, 20 nM or 200 nM in Starve Medium to achieve a final concentration of 1 microM doxorubicin and betacellulin concentration of 0 nM, 0.1 nM, 1 nM, 10 nM, or 100 nM, respectively. After about 24 hr of incubation, 100 microliter of CellTiter-Glo assay buffer (Promega, Madison, Wis., Cat# G7573) was added to each well, followed by shaking at room temperature in the dark for about 10 min. A total of 100 microliter of mixture per well was transferred to 96-well ½ area assay plate (Corning Incorporated, Corning, N.Y., Cat#3688), and the luminescent signal was determined by a luminescence plate reader Lmax (Molecular Devices Corporation, Sunnyvale, Calif.).

Results are shown in FIG. 35, which demonstrated the effects of recombinant betacellulin (BTC) on viability of rat neonatal cardiomyocytes in the presence of a cardiotoxic agent. FIG. 35 shows cell viability as a percentage of control as measured by ATP luminescent signal for each concentration of betacellulin tested. Each bar represents an average of three replicates. Betacelulin, at all concentrations, showed a statistically significant protective effect, when compared with control cells (p<0.001). Control was set at 100% viability. At 100 nM, betacellulin showed the highest protective effect, with a cell viability at about 210% of control. At 10 nM, betacellulin produced a cell viability of about 175% of control. At 1 nM and 0.01 nM, respectively, betacellulin produced a cell viability of about 160% of control. This experiment indicates that betacellulin could enhance the survival of cardiomyocytes exposed to cardiotoxic agents.

### Example 38

### A Betacellulin Splice Variant was not Active in the Impedance Assay

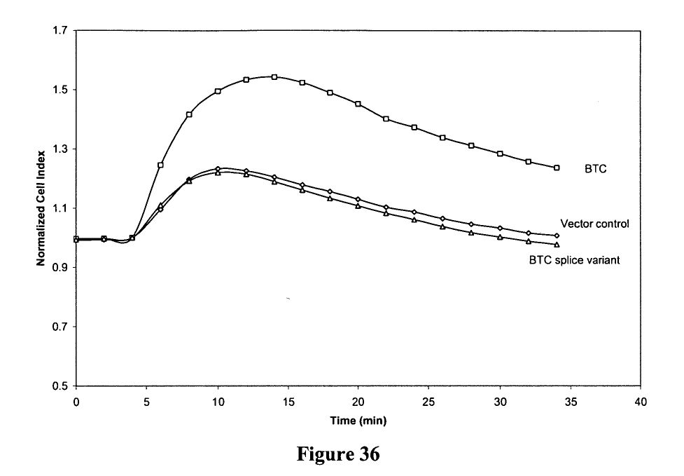

With our finding (in earlier examples) that BTC was active in both in causing an increase in cell index in primary human skeletal muscle cells, and also in augmenting the cell index increase in response to insulin (as measured by the impedance assay), we tested the activity of a betacellulin splice variant (“BTC SV”). This variant differed from the wild-type betacellulin in the C-terminus of the molecule (as described in PCT application WO 06/012707). BTC and BTC SV cDNAs (cloned into the pTT5 vector; Durocher, Y. et al. Nucleic Acids Res 30(2):E9 (2002) were each expressed in 293T cells (ATCC® Number CRL-11268™) and supernatants from these cell cultures after 4 days of culture were used as sources of the proteins in the impedance assay. About 3×10⁴ primary human skeletal muscle cells (Cambrex, East Rutherford, N.J.) were plated onto each well of the impedance plate from ACEA and prepared for the impedance assay as before. Cells were starved in 120 microliter of serum-free medium for 6 hr. Then, 40 microliter of supernatant from 293T cells expressing BTC, or 293T cells expressing the BTC SV, or 293T cells transfected with the vector control were added into each well. Impedance changes were measured using RT-CES from ACEA as previously described in earlier examples. Results are shown in FIG. 36, which shows a plot of Cell Index (normalized to baseline) against time. This experiment showed that BTC conditioned media induced a rapid increase in the normalized cell index. However, the BTC SV conditioned media did not have such effect, showing only the same low level response as the supernatant from the 293T cells tranfected with the vector control. This test indicates that the betacellulin splice variant lacked the stimulatory activity of the wild-type betacellulin.

### Example 39

### A BTC Splice Variant did not Stimulate Glucose Uptake in Primary Human Skeletal Muscle Cells

In view of our finding that the betacellulin splice variant disclosed in WO 06/012707 failed to stimulate an increase in cell index in primary human skeletal muscle cells, we tested this betacellulin splice variant for its ability to stimulate glucose uptake. In this experiment, both wild-type BTC and BTC SV were expressed in 293T cells. About 3×10⁴ primary human skeletal muscle cells from Cambrex were plated onto each well of a 96-well plate and prepared for the impedance assay as before. The primary human skeletal muscle cells were starved in 120 microliter of serum-free medium for six hr. Then 40 microliter of supernatant from either 293T cells expressing the BTC splice variant (BTC conditioned media; collected after four days of expression), or 293T cells transfected with a vector control (control conditioned media, collected after four days of expression of mock/empty vector control) were added into each well for 20 min in 37° C.

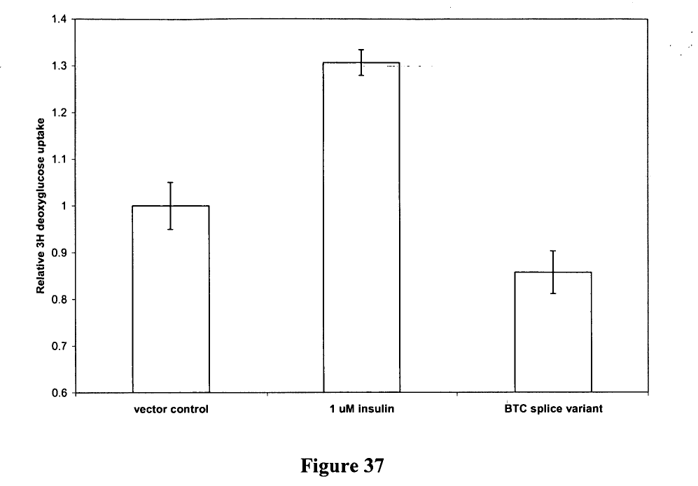

The cells were then labeled with 1 μCi ³H-deoxyglucose for 20 min in 37° C. After labeling, the cells were washed 3 times with ice-cold PBS and lysed with 0.05N NaOH. Radioactivities were counted by Topcount (PerkinElmer, Wellesley, Mass.). The results, depicted in FIG. 37, show that microM insulin, used as a positive control, induced glucose uptake in the human skeletal muscle cells. However, conditioned medium containing the BTC splice variant did not. This experiment demonstrates that the betacellulin splice variant lacked the ability to stimulate glucose uptake into muscle cells, a property that was earlier found in wild-type betacellulin under the same conditions. This experiment also demonstrates the existence of a good correlation between the ability to stimulate an increase in cell index and the ability to stimulate glucose uptake into muscle cells, as betacellulin was able to do both, whereas the betacellulin splice variant was able to do neither.

### Example 40

### Use of Betacellulin to Ameliorate Muscle Function in Subjects with Muscular Diseases, Including Muscular Dystrophy

The Dystrophin-Deficient mdx Mouse Model of Muscular Dystrophy

The dystrophin-deficient mdx mouse carries a mutation in its dystrophin gene and is a widely utilized model of muscular dystrophy (for review, see Chakkalakal, J. V. et al. FASEB J. 19:880-891 (2005)). Dystrophin is normally expressed in skeletal and cardiac muscle. In its absence, the association of the plasma membrane of skeletal and cardiac muscle cells with the surrounding basal lamina is weakened, underlying the pathologies associated with the onset of muscular dystrophies and cardiomyopathies. Consequently, the current invention provides a test that uses the mdx mouse to measure the effect of betacellulin treatment on preventing loss of muscle function, ameliorating muscle function, restoring muscle function or all of the above in subjects with muscular wasting or muscular dystrophies. Similar experiments can be carried out with other ErbB family members, alone or in combination with other molecules. Examples of some of such combinations can be found throughout the specification.

Dystrophin-deficient C57bl/10ScSn-Dmd^{mdx}/J mice, herein referred to as mdx mice, and C57bl/10ScSn control mice can be obtained from The Jackson Laboratory (Bar Harbor, Me., USA). For one study, in order to determine if betacellulin can ameliorate muscular dystrophy, four week-old male mdx mice are treated with various regimens of betacellulin administered subcutaneously in carrier solution, or treated with carrier alone. Alternatively, to determine if betacellulin can prevent muscular dystrophy, betacellulin administration can be initiated at earlier ages, for example, one week after birth, before there is evidence of muscular damage in the mdx mouse model (Tinsley, J. et al. Nat. Med. 4:1441-1444 (1998)). The animals can be injected with betacellulin or other ErbB ligand polypeptides, or with controls, as described in earlier examples. Physiological (mechanical, biochemical and histological) evaluation of the treated muscles can be performed as described in, for example, see Krag, T. O. B. et al., Proc. Natl. Acad. Sci. USA., 101:13856-13860 (2004), or Gillis, J. M. Acta Neurol. Belg., 100:146-150 (2000). Some examples of the invention are provided below, but one skilled in the art would know how to select the appropriate methods and parameters to determine the extent of the effect of betacellulin on the muscle of treated subjects, as well as the appropriate doses and frequency of administration to achieve improvements on their overall lifespan and quality of life (mobility, food consumption).

The effects of betacellulin treatment on glucose uptake, glucose tolerance, amino acid uptake and utrophin expression can also be tested in the mdx model of muscular dystrophy. The experimental details for these analyses are described in Examples 17 through 32. Of note, in the dystrophin-deficient mdx mouse, endogenous utrophin levels in muscle remain elevated soon after birth compared with normal mice. The first signs of muscle fiber necrosis are only detected after the endogenous utrophin levels have decreased to the adult levels (about 1 week after birth).

### Evaluation of Functional Muscle Recovery by Tests of Contractile Properties

### Quantification of Isometric Force Production and Eccentric Contractions

One of the standard evaluation methods for evaluation of functional muscle recovery can be used for determination of the extent of benefic that can be conferred by betacellulin or other members of the ErbB ligand family (hereafter, i.e., hereafter in Example 40, collectively referred to as “betacellulin”) is the mechanical muscle damage susceptibility test. This test is most typically done on the extensor digitorum longus (EDL) muscle, but can also be done on the extensor digitorum longus, plantaris, gastrocnemius, tibialis anterior, diaphragm, and the quadriceps. The mdx mice can be treated with betacellulin for a length of time. At the end of the desired betacellulin-treatment period, mice are anesthetized deeply with sodium pentobarbitone with supplemental doses administered as necessary to prevent any response to tactile stimulation. Freshly dissected muscles, for example the EDL, are weighed and transferred to a force transducer, where they are equilibrated in oxygenated Ringer's solution (pH 7.4) at 25° C. for the duration of the experiment. The EDL muscles are first tied at either end to the force transducer, and then stimulated with platinum field electrodes connected to a stimulator. This submits the muscles to a series of contractions with forced lengthenings called eccentric contractions (ECC). Data are digitized and acquired by a converter and appropriate software, and the eccentric contraction force drop is calculated using the difference of isometric force generation during the first and tenth tetanus of the standard ECC protocol (Krag, T. O. B. et al., Proc. Natl. Acad. Sci., USA. 101: 13856-13860 (2004)).

After completion of the in situ mechanical studies, the EDL muscles can be processed for further analysis. For example, to measure cell membrane damage, the muscles are immersed in 0.5% Procion Orange dye (Sigma-Aldrich, St. Louis, Mo., USA) in oxygenated Ringer's solution (buffered to pH 7.4 with HEPES) for 5 min (the bath is oxygenated continuously with a mixture of 95% O₂ and 5% CO₂ and maintained at 25° C.) and then flash-frozen in isopentane liquid. Frozen sections from each tissue are cut at midlength at −20° C. by using a cryostat, and the percentage of muscle fibers that are stained in the cytoplasm with Procion Orange quantified. Uptake of this low molecular weight dye into muscle fibers will be a direct indicator of damage to the cell membrane.

Another alternative is to process the muscles for histological analysis, for example after being embedded in Tissue-Tek® OCT compound (TissueTek, Sakura Finetek USA, Torrance, Calif.) or other embedding medium and/or flash-frozen, for example, in isopentane pre-cooled in liquid nitrogen. The susceptibility to damage of the mdx EDL upon lengthening contractions has been well characterized, impairing its ability to generate adequate force after a series of ECC (Bogdanovich, S. et al. FASEB J., 19: 543-549 (2005)). This impairment is typically quantified by calculating “force drop,” which is the post-ECC drop in force production. If there is a reduction in the absolute value of the force drop, or if there is an improvement in the post-ECC isometric force generated by the EDL after the treatment with betacellulin, then betacellulin can be said to cause a functional improvement on the treated muscle.

The benefits of betacellulin can also be demonstrated using the diaphragm muscle as described in, for example, Lynch G. S., et al. Am. J. Physiol., 272: C2063-C2068 (1997); and Gregorevic, P. et al. Am. J. Pathol., 161: 2263-2271 (2002). The diaphragm reportedly is the most affected muscle in the mdx mice, and typically shows degeneration and fibrosis earlier than the EDL, usually by 16 weeks (Stedman, H. H. et al., Nature, 352: 536-539 (1991)). At the completion of the betacellulin and control treatments, narrow strips of diaphragm are excised from anesthetized mdx mice, for example, by cutting radially from the central aponeurosis to a short segment of rib and then both ends are attached to the force transducer. The length of each preparation is adjusted to obtain the maximal isometric force. The normalized forces are calculated (force per unit cross-sectional area) and expressed in millinewton/mm² (Tisnley, J. et al., 1998; Stedman, H. H. et al., Nature, 352: 536-539 (1991)).

### Whole Body Tension (WBT)

The overall force of the muscular system of betacellulin-treated mice and control mice can also be monitored by the force developed during a non-invasive “escape test,” which consists of recording the force exerted by the mouse when it escapes the pinching of its tail, the tail having been connected to a force transducer. The highest force peak, or whole body tension (WBT1), and the average of the five highest peaks after repeating the pinching several times over a period of time (in min) are then calculated. The results are normalized to the body weight of the subject and expressed in millinewton/g, and this ratio is the WBT (Tinsley, J. et al. Nat. Med., 4: 1441-1444 (1998)).

### Biochemical and Evaluation of Functional Muscle Recovery by Tests of Creatine Kinase

In muscle diseases, the blood levels of cytoplasmic enzymes released upon damage to the muscle cell membrane (sarcolemmal damage), such as those of creatine kinase (CK), are elevated and in muscular dystrophy their levels can be very high (Bulfield, G. et al., Proc. Natl. Acad. Sci., 81: 1189-1192 (1984); Bogdanovich, S. et al. Nature, 420:418-421 (2002)). In fact, the level of CK in the blood is used as a diagnostic test for muscular dystrophy. Thus, the beneficial effects of betacellulin can also be demonstrated by treating the animals with betacellulin and, at different time points throughout the betacellulin treatment period, serum is collected by centrifugation of blood samples drawn from the mouse tail vein. Serum CK levels are measured using the indirect Sigma Diagnostics Creatine Phosphokinase kit and accompanying standards (Sigma-Aldrich, St. Louis, Mo., USA). A lower serum CK level in betacellulin-treated mice will show protective effect of betacellulin against damage to the muscle.

### Morphological Evaluation of the Muscle after Betacellulin Treatment Percentage of Centrally Nucleated Fibers

The percentage of centrally nucleated fibers is an accepted indicator of the cycles of muscle degeneration-regeneration and is used as an index to monitor the efficiency of gene therapy trials in mdx mice (Gillis, J. M. Acta Neurol. Belg. 100: 146-150 (2000); Bogdanovich, S. et al. FASEB J., 19: 543-549 (2005)). Because mdx muscles constantly regenerate in response to chronic inflammation and muscle damage, they have a much larger percentage of centrally nucleated fibers (CNF) relatively to those of normal mice (Camwath, J. W. and Shotton D. M. J. Neurol. Sci., 80: 39-54 (1987)). Thus, after completion of the desired betacellulin treatment, the animals are anesthetized, and the muscles (for example diaphragm or EDL muscles) excised and flash frozen in liquid nitrogen-cooled isopentane. Frozen sections from each muscle are cut at midlength/midbelly at −20° C. by using a cryostat, subjected to brief fixation (5 min) using ice-cold 100% methanol and either analyzed immediately or stored in an air-tight container at −80° C. until they are processed according to standard protocols for hematoxylin and eosin staining. Sections are imaged by light microscopy and scored for total number of myofibers, as well as for those containing centrally located nuclei. NIH Image processing freeware can be used for morphometric measurements of digitized images. The beneficial effects of betacellulin in ameliorating the pathology of mdx mice, can be demonstrated by a significant reduction in the CNF proportion in betacellulin treated mdx compared to control mdx mice.

### Endurance Time on a Rotarod

The endurance time on a rotating rod (“rotarod”) is a well-described assessment of whole body muscle strength, and mdx mice reportedly have an impaired ability to maintain grip and suspend themselves against gravity in this apparatus (Muntoni, F. et al. J. Neurol. Sci., 120: 71-77 (1993)). The beneficial effects of betacellulin on animals can be demonstrated at different time intervals along the treatment period, and their endurance evaluated at variable speeds (for example, 5 rpm and 10 rpm). For example, a mouse can be placed on a rod of 3.8 cm diameter (Rotarod test, CR-1 Rotamex System, Columbus Instruments). The rod revolves at 5 rpm/minute and can be accelerated to 10 rpm/minute. The time until the mouse falls off the rotating, accelerating rod is determined (mean±SE). Upon the fall, the mouse immediately receives an electrical shock (1 s, 0.2 mA). Each mouse is subjected to five trials per day within a 60-min period. The extent of beneficial effect of betacellulin can be observed by the longer length of time the betacellulin treated mice can stay on the rod before falling.

### Change in Body Weight

The dystrophin-less mdx mice will not usually gain significant weight over weeks and might even lose weight, depending on their age. To determine the effect of betacellulin on body weight, the animals are removed from their cage at different intervals (for example every week from 0 to 14 weeks) before and during the betacellulin treatment and placed on a balance to determine their body weight.

### Histomorphometry Assessment of Muscle Pathology: Muscle Histology, Muscle Length, Muscle Weight and Myofiber Size and Number

To quantify the increase in muscle mass, animals are euthanized and muscles excised and weighed, including the extensor digitorum longus, plantaris, gastrocnemius, tibialis anterior, diaphragm, and the quadriceps. The degree of gain or loss in muscle mass is compared to the degree of gain and loss of body weight observed in control and betacellulin-treated mice. To determine whether the change in muscle mass is due to hypertrophy (increase in cell size), hyperplasia (increase in cell number), or both, further morphometric examination is done on tissue sections. Frozen sections from each muscle are cut at midlength/midbelly at −20° C. by using a cryostat, subject to brief fixation (5 min) using ice-cold 100% methanol and either analyzed immediately or stores in an air-tight container at −80° C. until they are processed according to standard protocols for hematoxylin and eosin staining. Sections are imaged by light microscopy and scored for number and area of myofibers, total number of nuclei, number of nuclei/fiber, infiltration of inflammatory cells and fibrosis, for example. Measurements of whole muscle cross-sectional area (CSA) and single fiber area are also most typically done for the EDL muscle. Frequency histograms can be plotted for betacellulin treated and control animals illustrating the distribution of number of fibers along the single fiber area (um²) (Bogdanovich, S. et al. Nature, 420: 418-421, (2002)).

### Biochemical and Molecular Evaluation of Muscle Pathology after Betacellulin Treatment

To evaluate the beneficial effect of betacellulin on muscle cell pathology at the cellular level, skeletal muscle samples will be tested by immunohistochemistry and immunocytochemical (e.g. immunofluorecence, immunoprecipitation, kinase assays) analysis of several molecules, including ErbB receptors (e.g. identification of the activation and phosphorylation state of each receptor before, during and post-treatment), and some of those molecules that serve as surrogate markers of glucose metabolism (for example, phospho-Gskbeta/alpha, phospho-glycogen synthase, phosphorylated IRSs), cell survival and cell responses to stress (for example, phosphoAkt (Ser473), phospho-p70-S6kinase, phosphoS6-ribosomal protein, phospho FKHR, phosphorylated PI3K (catalytic and regulatory subunits)), cell death (for example, caspase-3 activation, phosphatidyl serine exposure), as well as cell proliferation (for example, phospho-histone-H3(Ser10, mitotic marker), proliferating cell nuclear antigen) and cell cycle markers (for example, p27 and cyclin D1). Similar experiments can be done with any other ErbB family members, variants, and combinations described in more detail throughout the specification.

Evaluation of the effects of betacellulin on muscle utrophin expression can also be done in situ by immunostaining of excised muscles with primary antibodies against utrophin. Visualization of the utrophin signal, including assessment of its expression in muscle fibers versus other cell types, can be done by methods known by those familiar with the art, including either bright-field or fluorescence microscopy through the use of secondary antibodies. The latter can either be complexed to enzymes, such as horseradish peroxidase or alkaline phosphatase, that act on chromogenic substrates visible by bright-field microscopy, or complexed to fluorescent labels such as Cy5 (Jackson Immunoresearch Inc., West Grove, Pa., USA) or Alexa Fluor 488 (Invitrogen, Carlsbad, Calif., USA) visible by fluorescence microscopy.

### Glucose Uptake into Adipose Tissue

Male mice from either control or betacellulin-treated groups (for example, mdx mice or myostatin-treated C57BL/6J mice) are fasted overnight and then injected intravenously through the tail vein with a bolus of 2-deoxy-D-[1, 2-[³H](N)]glucose, herein referred to as 2-[³H]DG, at 250 uCi/kg of mouse weight (Sigma-Aldrich, St. Louis, Mo., USA) in saline, together with insulin when appropriate. Mice are anesthetized and rapidly euthanized 30 min after injection. Epididymal fat pads are then quickly excised from groups of mice at regular intervals, washed, blot dried, weighed, and dissolved in 1 M NaOH at 60° C. Incorporated radioactivity is counted in a scintillation counter (LS3801, Beckman; Fullerton, Calif.). Uptake of 2-[³H]DG will be expressed as counts per minute divided by protein content.

### Effect of Betacellulin on Glucose Uptake by Resting and Contracting Diaphragm Muscle

One skilled in the art would be familiar with the published methods for assessing glucose uptake in diaphragm muscle explants (for example, see Evans, A. A. et al., J. Endocrin., 155: 387-392 (1997)).

Adult male mice (for example, male mdx mice and respective controls, or C57BL/6J mice injected with myostatin plasmids) of between 4 to 12 weeks of age (or at the end of each treatment) are anesthetized and sacrificed by cervical dislocation. The diaphragms are excised together with the phrenic nerves, divided into two hemidiaphragms along the central tendon and pinned down on Sylgard-coated tissue culture plates (Dow-Corning Corporation, Wiesbaden, Germany) containing 5-10 ml of modified Krebs-Henseleit solution (118 mM NaCl, 4.7 mM KCl, 8.7 mM CaCl₂.2H₂O, 1.17 mM MgSO₄.7H₂O, 1.2 mM KH₂PO₄, 25 mM NaHCO₃, 2% (weight/volume) bovine serum albumin, 2 mM sodium pyruvate). The cultures are gassed continuously with a 95% O₂/5% CO₂ air mix in a bath kept at 37° C. 2-deoxy-D-[1,2-[³H](N)]glucose, herein referred to as 2-[³H]DG is included to a final concentration of 1 mM (0.1 mCi/mmol). Insulin, betacellulin, or a combination of both of these proteins (or other combinations described throughout the specification), is added at the desired final concentrations. At the end of the desired incubation periods, muscles are removed from the bath, rinsed, blotted, and snap-frozen in liquid nitrogen. Muscles are then processed by heating for 10 min in 0.5 ml of 1 M NaOH at 90° C., transferred to an ice bath, centrifuged at 1000×g for 10 min, and the supernatant analyzed for ³H content in the digested muscle extract.

To assess the combined effect of insulin and betacellulin on glucose uptake during muscle contractions, tetanic contractions of abdominal muscle strips incubated in KHB media (with 2-[³H]DG) containing various concentrations of either insulin or betacellulin, or a combination of insulin and betacellulin, can be stimulated with platinum electrodes as described above for the EDL muscle, or following other described methods (Hansen, P. A. et al. J. Appl. Physiol., 76: 979-985 (1994)). Glucose uptake by contracting muscle explants can be assessed as described in the previous paragraph.

### Effect of Betacellulin on Cardiomyocytes

### Isolation of Murine Cardiomyocytes

Adult ventricular cardiomyocytes are isolated according to published methods (for example, Belke, D. D. et al. J Clin Invest., 109: 629-39 (2002)) from either mdx mice or C57BL/6J mice before or after treatment with insulin, myostatin, betacellulin or a combination of all of the above. Briefly, male mice (12 wk of age or at the end of the desired in vivo treatment) are injected intraperitoneally with 100 U of heparin 30 min before being anesthetized with an intraperitoneal administration of pentobarbital sodium (250 mg/kg) and euthanized by cervical dislocation. The heart is rapidly excised and arrested in ice-cold buffer A (120 mM NaCl, 5.4 mM KCl, 1.2 mM MgSO₄, 1.2 mM NaH₂PO₄, 5.6 mM glucose, 20 mM NaHCO₃, 0.6 mM CaCl₂, 10 mM 2,3-butanedione monoxime, and 5 mM taurine, pH 7.5). The aorta is then cannulated, and the heart is retrogradely perfused at 37° C. first with buffer A gassed with 95% O₂-5% CO₂ for 4 min, followed by 10-14 min with buffer A containing 25 uM CaCl₂ and 59 U/ml type II collagenase (Worthington Biochemical Corporation in Freehold, N.J., USA). The coronary flow rate is to be set at 2.5 ml/minute. The free wall of the right ventricle is then removed and digested at 37° C. for 5-10 min longer in presence of collagenase, 50 uM CaCl₂, and 1% (weight/volume) fatty acid-free bovine serum albumin. The heart was then minced with a sterile razor blade and the myocytes dissociated by sequential washing in buffer A with gradually increasing calcium concentration until a final concentration of 1 mM is achieved.

Dispersed myocytes are filtered through a nylon mesh with an 85-micrometer pore size (Tetko, Briarcliff Manor, N.Y.), pelleted by centrifugation at 40×g for 2 min, and resuspended in buffer A containing 100 uM CaCl₂ and 0.6% fatty acid-free bovine serum albumin. Freshly isolated cells are then used for the studies of glucose uptake, amino acid uptake, cell survival, and utrophin expression.

### Glucose Uptake by Freshly Isolated Adult Cardiomyocytes

Assays for the effect of various formulations and combinations described throughout the specification (with and without betacellulin, insulin, and the like) on glucose uptake by isolated mouse cardiomyocytes are done in triplicate in 12-well (22 mm diameter) laminin-coated (BD Biocoat) tissue culture plates (BD Biosciences, Bedford, Mass., USA). Laminin-plated isolated cardiomyocytes are washed twice with 1 ml of glucose-free DMEM. Then, 1 ml of glucose-free DMEM containing insulin (for example, at 1 nM), combined with various concentrations of betacellulin, as well as 1 mM pyruvate and 0.1% BSA are added. The cells are returned to the incubator and kept at 37° C. and 5% CO₂. After 40 min, 10 ul of a 2-deoxyglucose mix containing 130 ul of glucose-free DMEM, 15 ul of a 100-mM 2-deoxyglucose solution, and 5 ul of a 1 uCi/ul 2-deoxy-D-[1, 2-[³H](N)]glucose, herein referred to as 2-[³H]DG, are added. After 30 min, the medium is removed and the cells are washed twice with 1 ml of cold PBS. Cells are lysed in 500 ul of 1 M NaOH for 20 min at 37° C. A 40 ul aliquot of the lysed cells was used for measuring the total protein content of the solution using a Micro BCA Protein Assay Kit (Pierce Chemical Co., Rockford, Ill., USA). A 400 ul aliquot of the lysed cells is counted to determine the specific activity of 2-[³H]DG. Glucose uptake is then expressed as picomoles per minute per milligram of protein.

### Amino Acid Uptake by Freshly Isolated Adult Cardiomyocytes

Cardiomyocytes are cultured in serum-free Dulbecco's modified Eagle's medium. One microCi/ml [³H]phenylalanine is added to the culture medium 2 h before the cells were harvested. The cells are rapidly rinsed four times with ice-cold PBS and incubated for 20 min on ice with 1 ml of 20% trichloroacetic acid. The total radioactivity in each dish is determined by liquid scintillation counting. Amino acid uptake assays were also performed as described in detail in Example 31.

### Statistical Analysis

Unbiased analysis of the results were performed by two or more trained investigators. Results were expressed as means±SEM for each population, and were considered statistically significant for P<0.05. For comparison between unpaired groups, the Student's t-test or the Mann-Whitney test was used as appropriate.

### Example 41

### Properties of Betacellulin and other ErbB Ligands: Protein Sequence, Nucleotide Sequence, and Protein Domains

In Table 1, “Protein and Nucleotide Sequence Identification,” we provide some additional characteristics of a subset of the betacellulin polypeptides and other ErbB ligands of the invention. Each polypeptide is identified by the internal reference designation (FP ID), as shown in the first column. The nucleotide sequence identification number for the open reading frame of the nucleic acid sequence (N1) is shown in the second column. The amino acid sequence identification number for the polypeptide sequence (P1) is shown in the third column. The nucleotide sequence identification number for the entire nucleic acid sequence that contains UTR (N0) is shown in the fourth column. The fifth column shows an internal clone reference designation (Clone ID). The sixth column list annotations for some of the proteins.

TABLE 1
Protein and Nucleotide Sequence Identification

| | SEQ. ID | SEQ. ID | SEQ. ID | | |
|---|---|---|---|---|---|
| FP ID | NO. (N1) | NO. (P1) | NO. (N0) | CLONE ID | Notes |
| HG1015497 | 1 | 4 | 7 | 15079597/CLN00736345 | |
| HG1015498 | 2 | 5 | 8 | NP_001720 | |
| HG1019488 | 3 | 6 | 9 | 22218788 | |
| HG1015496 | 10 | 11 | | 00211466 | |
| HG1020193 | 12 | 13 | | 00902377 | BTC 32-111 |
| | | | | | (no Met) |
| HG1020377 | 14 | 18 | | 00902377_Met | BTC 32-111 |
| | | | | | (with Met) |
| HG1020378 | | 19 | | Seq1_from_US_6232288 | |
| HG1020379 | | 20 | | Seq2_from_US_6232288 | |
| HG1020380 | | 21 | | Seq3_from_US_5886141 | |
| HG1020381 | | 22 | | Seq14_from_US_5886141 | |
| HG1020382 | | 23 | | Seq17_from_US_5886141 | |
| HG1020383 | | 24 | | Seq18_from_US_5886141 | |
| HG1020384 | 15 | 25 | | NP_031594.1_1- | mouseBTC |
| | | | | 111_17939658_233- | fused to |
| | | | | 464_C237S | human Fc |
| HG1020385 | 16 | 26 | | NP_031594.1_1- | mouseBTC |
| | | | | 111_1799551_97-329 | fused to |
| | | | | | mouse Fc |
| HG1020386 | 17 | 27 | | 15079597_1- | human BTC |
| | | | | 111_17939658_233- | fused to |
| | | | | 464_C237S | human Fc |
| HG1021209 | 28 | 29 | | NP_001720_EGF | BTC EGF |
| | | | | | domain |
| HG1021210 | | 30 | | NP_003227 | TGF-alpha |
| HG1021211 | | 31 | | NP_039250 | NRG1-beta |
| HG1021212 | | 32 | | NP_039258 | NRG1-alpha |
| HG1021213 | | 33 | | NP_001936 | HB-EGF |
| HG1021214 | | 34 | | NP_001423 | Epiregulin |
| HG1021215 | | 35 | | NP_001954 | EGF |
| HG1021216 | | 36 | | NP_001648 | Amphiregulin |
| HG1021217 | | 37 | | 16716373 | Epigen |
| | | | | | (mouse) |
| HG1021218 | | 38 | | Q6UW88 | Epigen |
| | | | | | (human) |
| HG1021219 | | 39 | | NP_003227_EGF | TGF-alpha |
| HG1021220 | | 40 | | NP_039250_EGF | NRG1-beta |
| HG1021221 | | 41 | | NP_039258_EGF | NRG1-alpha |
| HG1021222 | | 42 | | NP_001936_EGF | HB-EGF |
| HG1021223 | | 43 | | NP_001423_EGF | Epiregulin |
| HG1021224 | | 44 | | NP_001954_EGF.1 | EGF |
| HG1021225 | | 45 | | NP_001954_EGF.2 | EGF |
| HG1021226 | | 46 | | NP_001954_EGF.3 | EGF |
| HG1021227 | | 47 | | NP_001954_EGF.4 | EGF |
| HG1021228 | | 48 | | NP_001954_EGF.5 | EGF |
| HG1021229 | | 49 | | NP_001954_EGF.6 | EGF |
| HG1021230 | | 50 | | NP_001954_EGF.7 | EGF |
| HG1021231 | | 51 | | NP_001954_EGF.8 | EGF |
| HG1021232 | | 52 | | NP_001648_EGF | Amphiregulin |
| HG1021233 | | 53 | | 16716373_EGF | Epigen |
| | | | | | (mouse) |
| HG1021234 | | 54 | | Q6UW88_EGF | Epigen |
| | | | | | (human) |
| HG1021235 | | 55 | | NP_003227_fragment | TGF-alpha |
| HG1021236 | | 56 | | NP_039250_fragment | NRG1-beta |
| HG1021237 | | 57 | | NP_039258_fragment | NRG1-alpha |
| HG1021238 | | 58 | | NP_001936_fragment | HB-EGF |
| HG1021239 | | 59 | | NP_001423_fragment | Epiregulin |
| HG1021240 | | 60 | | NP_001954_fragment | EGF |
| HG1021241 | | 61 | | NP_001648_fragment | Amphiregulin |
| HG1021242 | | 62 | | 16716373_fragment | Epigen |
| | | | | | (mouse) |
| HG1021243 | | 63 | | Q6UW88_fragment | Epigen |
| | | | | | (human) |
| HG1021244 | | 64 | | NP_003227_ECD.1 | TGF-alpha |
| HG1021245 | | 65 | | NP_003227_ECD.2 | TGF-alpha |
| HG1021246 | | 66 | | NP_039250_ECD | NRG1-beta |
| HG1021247 | | 67 | | NP_039258_ECD | NRG1-alpha |
| HG1021248 | | 68 | | NP_001936_ECD.1 | HB-EGF |
| HG1021249 | | 69 | | NP_001936_ECD.2 | HB-EGF |
| HG1021250 | | 60 | | NP_001936_ECD.3 | HB-EGF |
| HG1021251 | | 71 | | NP_001936_ECD.4 | HB-EGF |
| HG1021252 | | 72 | | NP_001423_ECD.1 | Epiregulin |
| HG1021253 | | 73 | | NP_001423_ECD.2 | Epiregulin |
| HG1021254 | | 74 | | NP_001423_ECD.3 | Epiregulin |
| HG1021255 | | 75 | | NP_001954_ECD.1 | EGF |
| HG1021256 | | 76 | | NP_001954_ECD.2 | EGF |
| HG1021257 | | 77 | | NP_001648_ECD.1 | Amphiregulin |
| HG1021258 | | 78 | | NP_001648_ECD.2 | Amphiregulin |
| HG1021259 | | 79 | | NP_001648_ECD.3 | Amphiregulin |
| HG1021260 | | 80 | | 16716373_ECD | Epigen |
| | | | | | (mouse) |
| HG1021261 | | 81 | | Q6UW88_ECD | Epigen |
| | | | | | (human) |
| | | 82 | | 15079597:15079596 | Betacellulin |
| | | | | | (human), res. |
| | | | | | 1-111 |
| | 83 | | | | Betacellulin |
| | | | | | (human), res. |
| | | | | | 1-111 |
| | | 84 | | | Betacellulin |
| | | | | | (mouse), res. |
| | | | | | 1-111 |
| | 85 | | | | Betacellulin |
| | | | | | (mouse), res. |
| | | | | | 1-111 |
| | | 86 | | | Betacellulin |
| | | | | | (mouse), res. |
| | | | | | 32-111 |
| | 87 | | | | Betacellulin |
| | | | | | (mouse), res. |
| | | | | | 32-111 |
| | | 88 | | | Betacellulin |
| | | | | | (mouse), Met |
| | | | | | followed by |
| | | | | | res. 32-111 |
| | 89 | | | | Betacellulin |
| | | | | | (mouse), Met |
| | | | | | followed by |
| | | | | | residues 32- |
| | | | | | 111 |

The Pfam system is an organization of protein sequence classification and analysis, based on conserved protein domains. We performed a Pfam analysis of betacellulin and other ErbB ligands to gather more information about their structure and possible activity. The Pfam system can be publicly accessed in a number of ways (for review and links to publicly available websites see Finn, R. D. et al. Nucleic Acids Res. 34:D247-D251, (2006)). Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families.

In Table 2, “Pfam Coordinates and Annotations of Betacellulin and other ErbB Ligand Sequences,” we provide the FP IDs of the proteins (FP ID) in the first column. The second column lists the Source ID. The third column lists the Pfam domains of each polypeptide (Pfam). The fourth column lists the coordinates of each Pfam domain, in terms of amino acid residues, beginning with “1” at the N-terminus of the full-length polypeptide. The fifth column lists an annotation from a public database.

TABLE 2
Pfam Protein Coordinates and Annotations of Betacellulin and other ErbB
Ligand Sequences

| FP ID | SOURCE ID | PFAM | COORDINATES | ANNOTATION |
|---|---|---|---|---|
| HG1015497 | 15079597 | EGF | (69-104) | Betacellulin |
| | | | | [Homo sapiens] |
| HG1015498 | NPP_001720 | EGF | (69-104) | Betacellulin |
| | | | | [Homo sapiens] |
| HG1019488 | 22218788 | EGF | (8-43) | Chain A, Nmr |
| | | | | Structure of Human |
| | | | | Betacellulin-2 |
| HG1021210 | NP_003227 | EGF | (47-82) | |
| HG1021211 | NP_039250 | I-set | (36-130) | |
| HG1021211 | NP_039250 | Neuregulin | (240-635) | |
| HG1021211 | NP_039250 | EGF | (182-221) | |
| HG1021211 | NP_039250 | ig | (50-114) | |
| HG1021212 | NP_039258 | I-set | (36-130) | |
| HG1021212 | NP_039258 | Neuregulin | (235-630) | |
| HG1021212 | NP_039258 | EGF | (182-221) | |
| HG1021212 | NP_039258 | ig | (50-114) | |
| HG1021213 | NP_001936 | EGF | (108-143) | |
| HG1021214 | NP_001423 | EGF | (68-103) | |
| HG1021215 | NP_001954 | EGF | (401-436) | |
| HG1021215 | NP_001954 | EGF | (976-1012) | |
| HG1021215 | NP_001954 | EGF | (835-868) | |
| HG1021215 | NP_001954 | EGF | (745-780) | |
| HG1021215 | NP_001954 | EGF | (318-354) | |
| HG1021215 | NP_001954 | EGF | (360-395) | |
| HG1021215 | NP_001954 | EGF | (887-910) | |
| HG1021215 | NP_001954 | EGF | (916-951) | |
| HG1021215 | NP_001954 | Ldl_recept_b | (654-694) | |
| HG1021215 | NP_001954 | Ldl_recept_b | (567-608) | |
| HG1021215 | NP_001954 | Ldl_recept_b | (524-565) | |
| HG1021215 | NP_001954 | Ldl_recept_b | (610-652) | |
| HG1021215 | NP_001954 | EGF_CA | (870-910) | |
| HG1021215 | NP_001954 | EGF_CA | (912-940) | |
| HG1021215 | NP_001954 | EGF_CA | (356-395) | |
| HG1021216 | NP_001648 | EGF | (146-181) | |
| HG1021217 | 16716373 | EGF | (55-95) | |
| HG1021218 | Q6UW88 | EGF | (47-87) | |

In Table 3, “Transmembrane Domain Coordinates for Betacellulin and other ErbB Ligands,” we provide some physical properties of a subset of proteins described throughout the specification. The first column lists the FP ID. The second column shows the cluster ID. The third column classifies betacellulin as a type 1 single transmembrane domain (STM) membrane protein. The fourth column shows the predicted length of each polypeptide, expressed as the number of amino acid residues. The fifth column specifies the result of an internally developed algorithm that predicts whether a sequence is secreted (Tree Vote), with “1” being a high probability that the polypeptide is secreted and “0” being a low probability that the polypeptide is secreted. The sixth column lists the number of transmembrane regions (TM). The seventh column list the amino acid coordinates of the transmembrane domains.

TABLE 3
Transmembrane Domain Coordinates for Betacellulin and other ErbB Ligands

| | CLUSTER | | | TREE | # OF TM | TM |
|---|---|---|---|---|---|---|
| FP ID | ID | CLASSIFICATION | LENGTH | VOTE | SEGMENTS | DOMAIN. |
| HG1015497 | 183727 | Type 1 STM | 178 | 0 | 2 | (9-31)(119-141) |
| HG1015498 | 183727 | Type 1 STM | 178 | 0 | 2 | (9-31)(119-141) |
| HG1019488 | 183727 | Type 1 STM | 50 | 0.01 | 0 | |
| HG1021210 | NP_003227 | Type 1 STM | 160 | 0 | 1 | (99-121) |
| HG1021211 | NP_039250 | Type 1 STM | 645 | 0 | 1 | (248-270) |
| HG1021212 | NP_039258 | Type 1 STM | 640 | 0 | 1 | (243-265) |
| HG1021213 | NP_001936 | Type 2 STM | 208 | 0 | 1 | (162-184) |
| HG1021214 | NP_001423 | Type 1 STM | 169 | 0 | 2 | (13-35) |
| | | | | | | (118-140) |
| HG1021215 | NP_001954 | Type 1 STM | 1207 | 0.04 | 1 | (1033-1055) |
| HG1021216 | NP_001648 | Type 1 STM | 252 | 0.1 | 1 | (199-221) |
| HG1021217 | 16716373 | Type 1 STM | 152 | 0.3 | 1 | (110-132) |
| HG1021218 | Q6UW88 | Type 1 STM | 133 | 0.44 | 1 | (102-121) |

In Table 4, “Signal-Peptide and Non-Transmembrane Domain Coordinates for Betacellulin and other ErbB ligands,” we provide some additional physical characteristics for these proteins. The first column lists the FP ID. The second column lists the lists the coordinates of the non-transmembrane regions (Non-TM Coordinates.). The third column lists a signal peptide (or secretory leader) position of each polypeptide (Signal Peptide coordinates) based on positions of the starting and end amino acid residues. The fourth column lists the corresponding mature protein coordinates, which are the amino acid residues of the mature polypeptide after cleavage of the signal peptide (or secretory leader) sequence of each polypeptide (Mature Protein coordinates). The fifth and six columns list possible alternative signal peptide and mature protein coordinates, respectively.

TABLE 4
Signal-Peptide and Non-Transmembrane Domain Coordinates for Betacellulin and
other ErbB ligands

| | | | | ALTERNATIVE | ALTERNATIVE |
|---|---|---|---|---|---|
| | NON-TM | SIGNAL | MATURE | SIGNAL | MATURE |
| FP ID | COORDINATES | PEPTIDE | PROTEIN | PEPTIDE | PROTEIN |
| HG1015497 | (1-8) (32-118) | (1-31) | (32-178) | | |
| | (142-178) | | | | |
| HG1015498 | (1-8) (32-118) | (1-31) | (32-178) | | |
| | (142-178) | | | | |
| HG1019488 | (1-8) (32-118) | (1-31) | (1-50) | | |
| | (142-178) | | | | |
| HG1021210 | (1-98)(122-160) | (1-22) | (23-160) | (6-18) | (19-160) |
| HG1021211 | (1-247)(271-645) | | (1-645) | | |
| HG1021212 | (1-242)(266-640) | | (1-640) | | |
| HG1021213 | (1-161)(185-208) | (1-25) | (26-208) | (6-18) (7-19) (11-23) | (19-208) |
| | | | | | (20-208) |
| | | | | | (24-208) |
| HG1021214 | (1-12)(36-117) | (12-29) | (30-169) | (20-32) | (33-169) |
| | (141-169) | | | | |
| HG1021215 | (1-1032)(1056-1207) | | (1-1207) | (1-13) | (14-1207) |
| HG1021216 | (1-198)(222-252) | (1-24) | (25-252) | (14-26) | (27-252) |
| | | | | (9-21) | (22-252) |
| HG1021217 | (1-109)(133-152) | (1-18) | (19-152) | | |
| HG1021218 | (1-101)(122-133) | (1-22) | | | |

### Example 42

### Betacellulin Fusion Proteins Have Extended Half-Lives

In this study, we demonstrated that pharmacokinetic properties of betacellulin could be improved by conjugating betacellulin with polyethylene glycol (PEG) or by fusing betacellulin to the Fc region of an immunoglobulin.

Part A: PEGylation of Betacellulin

Human betacellulin expressed in E. coli and purified as previously described (see Example 16) was pegylated as follows. A number of test reaction conditions were tested for two PEG reagents namely, mPEG-SMB-20K and mPEG-ButyrALD-20K (Nektar Therapeutics, Huntsville, Ala.) in order to identify conditions that provide the highest yield of active, mono-PEGylated betacellulin. For mPEG-SMB-20K, 18 reactions were performed as shown in the table below, varying betacellulin concentration (1 or 2.5 mg/mL), molar ratio of Betacellulin: PEG (1:1, 1:2 or 1:5), buffer (potassium phosphate pH 7.0, potassium phosphate pH 7.5, or borate pH 9.0). Aliquots were taken at 30 min, 1 hr, 4 hr, and 24 hr to monitor reaction progress.

Table with Reaction Conditions for PEGylation of BTC with mPEG-SMB-20K
50 uL reaction volumes. PEG-NHS BTC = 8964 g/mol,
PEG = 21,300 g/mol. BTC stock = 5 mg/mL, PEG stock = 100 mg/mL

| | BTC | | | | PEG | | | | | | |
|---|---|---|---|---|---|---|---|---|---|---|---|
| | (mg/mL), | BTC | BTC:PEG | PEG | (mg/mL), | | | uL 10x | | uL BTC | uL PEG |
| # | final | (nmoles) | ratio | (nmoles) | final | buffer | pH | buffer | uL water | stock | stock |
| 1 | 1 | 5.6 | 1:1 | 5.58 | 2.38 | KPI | 7 | 5 | 33.81 | 10 | 1.19 |
| 2 | 1 | 5.6 | 1:2 | 11.16 | 4.75 | KPI | 7 | 5 | 32.62 | 10 | 2.38 |
| 3 | 1 | 5.6 | 1:5 | 27.89 | 11.88 | KPI | 7 | 5 | 29.06 | 10 | 5.94 |
| 4 | 2.5 | 13.9 | 1:1 | 13.94 | 5.94 | KPI | 7 | 5 | 17.03 | 25 | 2.97 |
| 5 | 2.5 | 13.9 | 1:2 | 27.89 | 11.88 | KPI | 7 | 5 | 14.06 | 25 | 5.94 |
| 6 | 2.5 | 13.9 | 1:5 | 69.72 | 29.70 | KPI | 7 | 5 | 5.15 | 25 | 14.85 |
| 7 | 1 | 5.6 | 1:1 | 5.58 | 2.38 | KPI | 7.5 | 5 | 33.81 | 10 | 1.19 |
| 8 | 1 | 5.6 | 1:2 | 11.16 | 4.75 | KPI | 7.5 | 5 | 32.62 | 10 | 2.38 |
| 9 | 1 | 5.6 | 1:5 | 27.89 | 11.88 | KPI | 7.5 | 5 | 29.06 | 10 | 5.94 |
| 10 | 2.5 | 13.9 | 1:1 | 13.94 | 5.94 | KPI | 7.5 | 5 | 17.03 | 25 | 2.97 |
| 11 | 2.5 | 13.9 | 1:2 | 27.89 | 11.88 | KPI | 7.5 | 5 | 14.06 | 25 | 5.94 |
| 12 | 2.5 | 13.9 | 1:5 | 69.72 | 29.70 | KPI | 7.5 | 5 | 5.15 | 25 | 14.85 |
| 13 | 1 | 5.6 | 1:1 | 5.58 | 2.38 | borate | 9 | 5 | 33.81 | 10 | 1.19 |
| 14 | 1 | 5.6 | 1:2 | 11.16 | 4.75 | borate | 9 | 5 | 32.62 | 10 | 2.38 |
| 15 | 1 | 5.6 | 1:5 | 27.89 | 11.88 | borate | 9 | 5 | 29.06 | 10 | 5.94 |
| 16 | 2.5 | 13.9 | 1:1 | 13.94 | 5.94 | borate | 9 | 5 | 17.03 | 25 | 2.97 |
| 17 | 2.5 | 13.9 | 1:2 | 27.89 | 11.88 | borate | 9 | 5 | 14.06 | 25 | 5.94 |
| 18 | 2.5 | 13.9 | 1:5 | 69.72 | 29.70 | borate | 9 | 5 | 5.15 | 25 | 14.85 |
| 19 | 1 | 5.6 | — | 0.00 | 0.00 | KPI | 7 | 5 | 35.00 | 10 | 0.00 |

The progress of the PEGylation reaction was monitored by separating aliquots of each reaction at different time points by SDS-PAGE (4-12% Bis-Tris), and staining the proteins with Coomassie blue, following methods standard in the art. PEG addition was observed as decreased migration of the protein in the gel; unreacted BTC migrated to just above the dye front, monoPEGylated BTC migrated to near the about 51 kDa molecular weight marker, and multiply PEGylated species ran between the about 64 kDa and the about 191 kDa markers. The reactions proceeded quickly, with significant product observed even at 30 min. A variety of multiply PEGylated species were observed at 24 hr.

For PEGylation of betacellulin with the reagent mPEG-ButyrALD-20K, 18 reactions were performed, varying betacellulin concentration (1 or 2.5 mg/mL), molar ratio of Betacellulin: PEG (1:1, 1:2, or 1:5), and buffer (potassium phosphate pH 7.0, potassium phosphate pH 6.0, or acetate pH 5.0). In all cases, a five-fold molar excess (versus betacellulin) of sodium cyanoborohydride was used. Aliquots were taken at 30 min, 1 hr, 4 hr, and 24 hr to monitor reaction progress.

Table with Reaction Conditions for PEGylation of BTC with mPEG-ButyrALD-20K
50 uL reaction volumes. PEG-Nterm. 5-fold molar excess of CH3BN Na vs. BTC.
BTC = 8964 g/mol, PEG = 20,411 g/mol, CH3BN Na = 62.84 g/mol.
BTC stock = 5 mg/mL, PEG stock = 100 mg/mL, CH3BN Na stock = 1 mg/mL

| | BTC | | | | | | CH3BN | | | | | | | uL |
|---|---|---|---|---|---|---|---|---|---|---|---|---|---|---|
| | (mg/ | | | | PEG | CH3BN | Na | | | | | uL | uL | CH3BN |
| | mL), | BTC | BTC:PEG | PEG | (mg/mL), | Na | (mg/mL), | | | uL 10x | uL | BTC | PEG | Na |
| # | final | (nmoles) | ratio | (nmoles) | final | (nmoles) | final | buffer | pH | buffer | water | stock | stock | stock |
| 1 | 1 | 5.6 | 1:1 | 5.58 | 2.38 | 27.89 | 0.035 | acetate | 5 | 5 | 32.06 | 10 | 1.19 | 1.75 |
| 2 | 1 | 5.6 | 1:2 | 11.16 | 4.75 | 27.89 | 0.035 | acetate | 5 | 5 | 30.87 | 10 | 2.38 | 1.75 |
| 3 | 1 | 5.6 | 1:5 | 27.89 | 11.88 | 27.89 | 0.035 | acetate | 5 | 5 | 27.31 | 10 | 5.94 | 1.75 |
| 4 | 2.5 | 13.9 | 1:1 | 13.94 | 5.94 | 69.72 | 0.088 | acetate | 5 | 5 | 12.65 | 25 | 2.97 | 4.38 |
| 5 | 2.5 | 13.9 | 1:2 | 27.89 | 11.88 | 69.72 | 0.088 | acetate | 5 | 5 | 9.68 | 25 | 5.94 | 4.38 |
| 6 | 2.5 | 13.9 | 1:5 | 69.72 | 29.70 | 69.72 | 0.088 | acetate | 5 | 5 | 0.77 | 25 | 14.85 | 4.38 |
| 7 | 1 | 5.6 | 1:1 | 5.58 | 2.38 | 27.89 | 0.035 | KPI | 6 | 5 | 32.06 | 10 | 1.19 | 1.75 |
| 8 | 1 | 5.6 | 1:2 | 11.16 | 4.75 | 27.89 | 0.035 | KPI | 6 | 5 | 30.87 | 10 | 2.38 | 1.75 |
| 9 | 1 | 5.6 | 1:5 | 27.89 | 11.88 | 27.89 | 0.035 | KPI | 6 | 5 | 27.31 | 10 | 5.94 | 1.75 |
| 10 | 2.5 | 13.9 | 1:1 | 13.94 | 5.94 | 69.72 | 0.088 | KPI | 6 | 5 | 12.65 | 25 | 2.97 | 4.38 |
| 11 | 2.5 | 13.9 | 1:2 | 27.89 | 11.88 | 69.72 | 0.088 | KPI | 6 | 5 | 9.68 | 25 | 5.94 | 4.38 |
| 12 | 2.5 | 13.9 | 1:5 | 69.72 | 29.70 | 69.72 | 0.088 | KPI | 6 | 5 | 0.77 | 25 | 14.85 | 4.38 |
| 13 | 1 | 5.6 | 1:1 | 5.58 | 2.38 | 27.89 | 0.035 | KPI | 7 | 5 | 32.06 | 10 | 1.19 | 1.75 |
| 14 | 1 | 5.6 | 1:2 | 11.16 | 4.75 | 27.89 | 0.035 | KPI | 7 | 5 | 30.87 | 10 | 2.38 | 1.75 |
| 15 | 1 | 5.6 | 1:5 | 27.89 | 11.88 | 27.89 | 0.035 | KPI | 7 | 5 | 27.31 | 10 | 5.94 | 1.75 |
| 16 | 2.5 | 13.9 | 1:1 | 13.94 | 5.94 | 69.72 | 0.088 | KPI | 7 | 5 | 12.65 | 25 | 2.97 | 4.38 |
| 17 | 2.5 | 13.9 | 1:2 | 27.89 | 11.88 | 69.72 | 0.088 | KPI | 7 | 5 | 9.68 | 25 | 5.94 | 4.38 |
| 18 | 2.5 | 13.9 | 1:5 | 69.72 | 29.70 | 69.72 | 0.088 | KPI | 7 | 5 | 0.77 | 25 | 14.85 | 4.38 |

Reaction progress was also monitored by Coomassie blue stained SDS-PAGE (4-12% Bis-Tris). PEG addition to betacellulin was observed as decreased migration in the gel. These reactions proceeded more slowly and approached completion at about 24 hr. As expected, this reagent produced mostly mono-PEGylated betacellulin, which migrated near the 51 kDa molecular weight marker. At 24 hr, all the PEGylation the reactions were quenched by addition of excess glycine. The mPEG-SMB-20K and mPEG-ButyrALD-20K reactions were pooled and fractionated by size exclusion chromatography using S75 and S200 columns (Amersham Pharmacia Biotech, GE Healthcare Bio-Sciences Corp., Piscataway, N.J.). Peaks corresponding to PEGylated betacellulin were pooled, diluted to 40 microM based on absorbance at 280 nm), and tested for activity.

Betacellulin activity was determined using an in vitro HeLa 229 (ATCC number CCL2.1) cell based binding assays and a phospho-EGFR pY1068 ELISA based assays according to the manufacturer's instructions (Cat. Number: KHR9081, BioSource International, Inc. Camarillo, Calif.), and as described in Example 35. Under these reaction and assay conditions, the activity of the PEGylated betacellulin produced using the mPEG-SMB-20K reagent was approximately 3-fold lower than the activity of unreacted betacellulin, while the activity of the PEGylated betacellulin produced using the mPEG-ButyrALD-20K reagent was reduced by less than 50%.

| | | [BTC] nM submitted to | ELISA activity is equivalent to | |
|---|---|---|---|---|
| PEGylation chemistry | SEC column | assay | [BTC] nM | activity % |
| mPEG-SMB | S70 | 40 | 10 | 26% |
| mPEG-ButyrALD | S70 | 40 | 23 | 58% |
| Unreacted | S70 | 40 | 0 | 0% |
| mPEG-SMB | S200 | 40 | 14 | 34% |
| mPEG-ButyrALD | S200 | 40 | 22 | 55% |

Part B: Betacellulin-Fc Fusion Protein

Murine betacellulin (containing amino acid residues 1-111 of the full-length protein) was fused to the Fc portion of the human immunoglobin IgG1. The fusion construct was subcloned into pIRESpuro3 expression vector (Cat# 6986-1, Clonetech Laboratories, Inc., Mountain View, Calif.). The vector was stably transfected into CHO-S cells using standard transfection methods, and the protein was produced using a 10 L Wave fermenter (Cat# BASE2050EH, Biotech, LLC; Somerset, N.J.) and CD-CHO medium (Cat# 10743-029, Invitrogen Inc., Carlsbad, Calif.). After eight days of culturing under these conditions, the cell supernatants were harvested. The fusion protein mouse BTC-human Fc was purified by affinity chromatography using Protein A Sepharose 4 Fast Flow resin (Cat# 17-5280-02, GE Healthcare, Piscataway, N.J.) following the manufacturer's recommendations and dialyzed in PBS. The activity of the purified mouse BTC-human Fc fusion protein (betacellulin-Fc fusion) was also tested by the phospho-ErbB receptor assay described above and in Example 35.

Part C: Pharmacokinetic Assay of PEGylated and Fc-Fusion Betacellulin

To determine whether PEGylation or Fc fusion affects the pharmacokinetic properties of betacellulin, unreacted Betacellulin, betacellulin-Fc fusion, and PEGylated betacellulin were prepared, administered to mice, and monitored for disappearance from the bloodstream.

The PEGylation reaction conditions for the betacellulin protein used in this test were as follows: 2.5 mg/mL betacellulin, 5-fold molar excess of mPEG-ButryALD-20K and sodium cyanoborohydride, potassium phosphate pH 7.0 buffer, and 24 hr reaction time followed by quenching with excess glycine pH 7.0. The reaction products were prepared for injection by overnight dialysis against 2×PBS. The success of the reaction was confirmed by Coomassie-stained SDS-PAGE gels, as described in Parts B and C above. The concentration of the PEG-BTC, the BTC-Fc (prepared as described in Part C), and the BTC (prepared as described in Example 16) protein solutions used for this test was determined by Bradford assay. Samples were prepared for injection by diluting each of the betacellulin protein solutions to 0.125 mg/mL in PBS supplemented with 0.1% BSA (Sigma #A3059, St. Louis Mo.).

Eight-week old C57Bl/6 mice were injected intravenously with 200 microliter of BTC, PEG-BTC, or BTC-Fc at a dose of 1 mg/kg BTC, and blood samples were collected at 2, 30, 120, and 1440 min. For each betacellulin type tested, six mice were injected with the test material. Then, three of the six mice were bled from the retro-orbital sinus at 2 min and then again by cardiac puncture at the 120 min time point. For the other three mice, blood was collected from the retro-orbital sinus at 30 min and then by cardiac puncture at 1440 min. All blood samples were collected into plasma collection “Microtainer” tubes with EDTA from Becton Dickinson (Cat# 365973, Franklin Lakes, N.J.) and then spun immediately to obtain plasma.

Human betacellulin concentrations in the BTC and PEG-BTC plasma samples and murine betacellulin concentrations in the BTC-Fc plasma samples were determined using ELISA assays. Standard curves were generated using 0.34-250 pM of murine and human betacellulin. The plasma samples were diluted 10, 100, and 5000-fold in 10% FCS/PBS solution to ensure that the signal was in the linear region of the standard curve. ELISA concentrations, determined for each plasma sample at 2 min, 30 min, 120 min and 1080 min post-injection, were calculated to be as follows:

| | | | | | | | BTC-Fc (pM BTC) | | |
|---|---|---|---|---|---|---|---|---|---|
| | BTC (pM) | | | PEG-BTC (pM BTC) | | | mouse | mouse | mouse |
| | mouse 1 | mouse 2 | mouse 3 | mouse 4 | mouse 5 | mouse 6 | 10 | 11 | 12 |
| 2 min | 16584 | 21240 | 169442 | 121051 | 124586 | 96793 | 22030 | 21950 | 31183 |
| 30 min | 406 | 407 | 23147 | 40491 | 49102 | 2460 | 4159 | 6288 | 20663 |
| 120 min | 0 | 0 | 315 | 2157 | 3847 | 195 | 31 | 47 | 16592 |
| 1080 min | 0 | 0 | 0 | 0 | 0 | 0 | 16 | 0 | 2 |

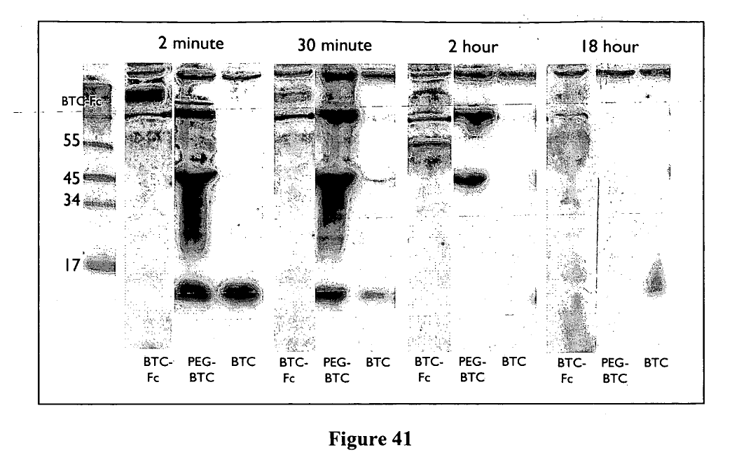

To prepare samples for the Western blot, 3.25 microliter plasma from each mouse from the same group at each timepoint was pooled. Plasma aliquots were separated in nonreducing Tris-Tricine gels (10-20%), and the proteins visualized by standard Western blot analysis. The results are shown in FIG. 41. Human betacellulin was detected using R&D Systems (Minneapolis, Minn.) antibody #261, and BTC-Fc was detected using an HRP-labeled anti-human Fc antibody (Cat#209-035-088; Jackson ImmunoResearch, West Grove, Pa.) combined with an ECL detection system GE Healthcare, Piscataway, N.J.). PEG-BTC migrated at approximately 45 kDa, unreacted BTC migrated at approximately 10 kDa, and the location of BTC-Fc is as shown on the left in FIG. 41.

From the results of both the ELISA and Western blot analyses, we determined that both PEG-BTC and BTC-Fc were cleared from mouse plasma significantly more slowly than unmodified betacellulin and thus have an extended pharmacokinetic half-life.

### Sequence Listing

Applicants include a Sequence Listing provided in both electronic format and in paper format and a Statement Accompanying Sequence Listing. The “Sequence Listing” provides the nucleic acid sequences and the amino acid sequences (SEQ. ID. NO. 1 through 91), of each betacellulin FP ID discussed in the specification and examples section (for more details, see Example 41; SEQ. ID. NO. 1 through 89), as well as that of other ErbB ligands described throughout the specification.

1. A pharmaceutical composition comprising a concentration of betacellulin or an active variant or fragment thereof, wherein the concentration is sufficient to acutely reduce the blood glucose level in a subject without inducing hypoglycemia, and a pharmaceutically acceptable carrier.

2. A pharmaceutical composition comprising a concentration of long-acting betacellulin fusion protein comprising a betacellulin polypeptide or an active variant or fragment thereof and a fusion partner, wherein the betacellulin fusion protein has an extended half-life in a subject when compared to the betacellulin polypeptide alone, wherein the concentration is sufficient to perform an action selected from stimulating glucose or amino acid uptake into muscle cells, promoting cell survival or inhibiting apoptosis of muscle cells, inducing utrophin expression, inhibiting muscle wasting or increasing muscle mass, reducing HbA1c, reducing hypoglycemia associated with insulin administration, reducing the basal blood glucose level, and/or acutely reducing the elevated blood glucose level in the subject; and a pharmaceutically acceptable carrier.

3. The long-acting betacellulin fusion protein of claim 2, wherein the extended half-life comprises at least 0.5 hr, at least 1 hr, at least 2 hr, at least 3 hr, at least 4 hr, or at least 5 hr longer than the half-life of the betacellulin polypeptide alone.

4. The long-acting betacellulin fusion protein of claim 2, wherein the fusion partner is a polymer, a polypeptide, a succinyl group, or an active variant or fragment of any of these.

5. The long-acting betacellulin fusion protein of claim 4, wherein the fusion partner polymer comprises a polyethylene glycol moiety either permanently or reversibly covalently attached to the betacellulin polypeptide.

6. The long-acting betacellulin fusion protein of claim 4, wherein the fusion partner polypeptide comprises an immunoglobulin fragment, albumin, or an oligomerization domain.

7. The long-acting betacellulin fusion protein of claim 6, wherein the immunoglobulin fragment comprises an Fc fragment.

8. A kit comprising:
(a) a pharmaceutical composition comprising a polypeptide of the ErbB ligand family or an active variant or fragment thereof, either alone or as part of a long-acting fusion protein, wherein the fusion protein has an extended half-life in a subject when compared to the ErbB ligand polypeptide alone;
and a pharmaceutically acceptable carrier; and
(b) instructions for administration into a subject in need of such a composition.

9. The kit of claim 8 wherein the polypeptide of the ErbB ligand family is selected from betacellulin, neuregulin1, HB-EGF, EGF, TGF-alpha, epiregulin, epigen, and amphiregulin.

10. The kit of claim 8 wherein the polypeptide is betacellulin.

11. The kit of claim 8, wherein the instructions describe a use for the composition selected from stimulating glucose or amino acid uptake into muscle cells, reducing HbA1c, reducing hypoglycemia associated with insulin administration, reducing the basal blood glucose level, acutely reducing the elevated blood glucose level, promoting cell survival or inhibiting apoptosis of muscle cells, inducing utrophin expression, inhibiting muscle wasting or increasing muscle mass, and treating the subject for obesity.

12. The kit of claim 8, further comprising a vial or cartridge.

13. The kit of claim 12, wherein the vial or cartridge comprises a concentration of the ErbB ligand polypeptide selected from about 50 micrograms/milliliter to about 100 micrograms/milliliter, from about 100 micrograms/milliliter to about 1 milligram/milliliter, from about 1 milligram/milliliter to about 5 milligrams/milliliter, and from about 5 milligrams/milliliter to about 500 milligrams/milliliter ErbB ligand polypeptide.

14. The kit of claim 13, wherein the vial or cartridge comprises from about 100 milligrams/milliliter to about 400 milligrams/milliliter ErbB ligand polypeptide.

15. The kit of claim 13, wherein the vial or cartridge comprises from about 200 milligrams/milliliter to about 300 milligrams/milliliter ErbB ligand polypeptide.

16. The kit of any of claims 12-15, wherein the vial or cartridge comprises a single dose of ErbB ligand polypeptide with a volume of about 0.5 milliliters, about 1.0 milliliter, or about 1.5 milliliters.

17. The kit of any of claims 12-15, wherein the vial or cartridge comprises a single dose, a double dose, or a triple dose of the ErbB ligand polypeptide.

18. The kit of claim 8, further comprising at least one second agent, wherein the second agent is an anti-diabetic agent.

19. A method of treating a disease in a subject comprising:
(a) providing a polypeptide of the ErbB ligand family; and
(b) administering the polypeptide to the subject,
wherein the subject has normal pancreatic function and/or a normal insulin level and wherein the subject would benefit from an action selected from stimulating glucose or amino acid uptake into muscle cells, reducing HbA1c, reducing hypoglycemia associated with insulin administration, reducing the basal blood glucose level, acutely reducing the elevated blood glucose level, promoting cell survival or inhibiting apoptosis of muscle cells, inducing utrophin expression, and inhibiting muscle wasting or increasing muscle mass.

20. The method of claim 19 wherein the polypeptide is selected from betacellulin, neuregulin1, HB-EGF, EGF, TGF-alpha, epiregulin, epigen, and amphiregulin.

21. The method of claim 19, wherein the polypeptide comprises betacellulin or an active variant or fragment thereof.

22. The method of claim 19, wherein the polypeptide comprises a long-acting fusion protein comprising a polypeptide of the ErbB ligand family or an active variant or fragment thereof and a fusion partner, wherein the ErbB ligand fusion protein has an extended half-life in a subject when compared to the ErbB ligand polypeptide alone.

23. The method of claim 19, wherein the disease comprises an elevated blood glucose level.

24. The method of claim 23, wherein the disease comprises Type I or Type II diabetes.

25. The method of claim 19, wherein the disease has symptoms selected from acute hyperglycemia, incipient diabetic ketoacidosis, diabetic ketoacidosis, and diabetic coma.

26. The method of claim 23, wherein the polypeptide is administered at a dose sufficient to produce a euglycemic level of blood glucose, to lower fasting blood glucose and/or lower the HbA1c level in the subject.

27. The method of claim 19, wherein the disease comprises obesity.

28. The method of claim 19, wherein the disease is selected from diabetic amyotrophy or other metabolic myopathy, cachexia, AIDS wasting, disuse atrophy, sarcopenia, rhabdomyolysis, myositis, diaphragmatic weakness due to muscular disorder, and muscular dystrophy.

29. The method of claim 19, wherein the muscle cells affected by the polypeptide are selected from skeletal, cardiac, and smooth muscle.

30. The method of claim 19, wherein the cells into which the uptake of glucose or amino acid is stimulated are cardiac cells and wherein the disease comprises a cardiac disease.

31. The method of claim 30, wherein the cardiac disease is selected from ischemia, congestive heart failure, myocardial infarction, and induced cardiotoxicity.

32. The method of claim 31, wherein the induced cardiotoxicity is induced by chemotherapy or is virally induced.

33. The method of claim 30, wherein the polypeptide is administered as a composition comprising a collagen or a gel.

34. The method of claim 19, wherein the subject is treated in an emergency setting.

35. The method of claim 34, wherein the emergency setting is selected from an emergency room, an intensive care setting, a setting wherein the subject is acutely ill, and a setting wherein the subject is suffering from a condition selected from respiratory failure, cardiac failure, kidney failure, diabetic ketoacidosis, and another life-threatening condition.

36. The method of claim 19, wherein the polypeptide is administered orally, subcutaneously, intravenously, transdermally, intraperitoneally, by inhalation, by implantation, intradermally, intramuscularly, intracardially, nasally, and/or by rectal suppository.

37. The method of claim 19, wherein the polypeptide is administered at a dose sufficient to produce a blood concentration of the polypeptide in a range from about 1 nanomolar to about 10 nanomolar or from about 10 nanograms/milliliter to about 100 nanograms/milliliter in the subject.

38. The method of claim 19, wherein the polypeptide is administered at least once a day, at least two times a day, or at least three times a day.

39. The method of claim 19, wherein one dose of the polypeptide is administered at or about meal time.

40. The method of claim 19, wherein the polypeptide is administered at a time selected from within about 120 minutes, about 90 minutes, about 60 minutes, about 30 minutes, about 15 minutes, or about 5 minutes before or after a meal; or during a meal.

41. The method of claim 19, wherein the benefit comprises acute reduction of elevated blood glucose level.

42. The method of claim 41, wherein the acute reduction occurs in a time period selected from within about 1 minute to about 120 minutes, within about 2 minutes to about 90 minutes, within about 3 minutes to about 60 minutes, within about 4 minutes to about 30 minutes, and within about 5 minutes to about 15 minutes.

43. The method of claim 19, wherein the polypeptide is administered in one or more doses, selected from a dose comprising from more than about 50 micrograms to less than about 2 milligrams, greater than about 2 milligrams to less than about 10 milligrams, and greater than about 10 milligrams to about 500 milligrams.

44. The method of claim 43, wherein the dose comprises from about 100 milligrams to about 400 milligrams.

45. The method of claim 44, wherein the dose comprises from about 200 milligrams to about 300 milligrams.

46. The method of claim 19, wherein the polypeptide is administered in one or more doses, selected from doses comprising from about 0.01 milligrams/kilogram to about 5 milligrams/kilogram, from about 0.1 milligrams/kilogram to about 2 milligrams/kilogram, from about 0.2 milligrams/kilogram to about 1 milligram/kilogram, from about 0.3 milligrams/kilogram to about 0.9 milligrams/kilogram, from about 0.4 milligrams/kilogram to about 0.8 milligrams/kilogram, and from about 0.5 milligrams/kilogram to about 0.7 milligrams/kilogram.

47. The method of claim 46, wherein the dose comprises no more than 1 milligram/kilogram.

48. The method of claim 19, wherein the polypeptide is administered in one or more doses, each comprising from about 1 microgram/kilogram to about 10 milligrams/kilogram.

49. The method of claim 48, wherein the polypeptide is administered in one or more doses, each comprising from about 10 micrograms/kilogram to about 1 milligram/kilogram.

50. The method of claim 19, further comprising:
(c) administering at least one second agent, wherein the second agent is another therapeutic agent.

51. The method of claim 50, wherein the second agent comprises an anti-diabetic agent.

52. The method of claim 50 or 51, wherein the second agent is administered orally, subcutaneously, intravenously, transdermally, intraperitoneally, by inhalation, by implantation, intradermally, intramuscularly, intracardially, nasally, and/or by rectal suppository.

53. The method of claim 50 or 51, wherein the second agent is administered before, after, or at the same time as the polypeptide.

54. The method of claim 50, wherein the second agent is selected from metformin, an insulin secretagogue, a glucosidase inhibitor, a PPAR gamma, and a dual PPAR gamma/alpha-agonist.

55. The method of claim 54, wherein the insulin secretagogue is selected from a sulfonylurea and a meglitinide.

56. The method of claim 50, wherein the second agent is selected from insulin, an insulin analogue, a co-secreted agent, pramlinitide, and a DPP4 antagonist.

57. The method of claim 50, wherein the second agent comprises a glucagon-like peptide.

58. The method of claim 57, wherein the glucagon-like peptide comprises exenatide.
